SEQUENCE LISTING (8 sequences)

SEQ ID NO: 1 - protein, 450 aa, Artificial Sequence
  Muromonab heavy chain
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, Artificial Sequence
  Muromonab light chain
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 134 aa, Artificial Sequence
  recombinant human IL-2
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 4 - protein, 132 aa, Artificial Sequence
  aldesleukin
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 5 - protein, 130 aa, Artificial Sequence
  recombinant human IL-4
MHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 6 - protein, 153 aa, Artificial Sequence
  recombinant human IL-7
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 7 - protein, 115 aa, Artificial Sequence
  recombinant human IL-15
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 8 - protein, 132 aa, Artificial Sequence
  IL-21
MQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

(19) United States
(12) Patent Application Publication
(10) Pub. No.: US 2020/0347350 A1
(43) Pub. Date: Nov. 5, 2020

(54) Expansion Of Peripheral Blood Lymphocytes (PBLS) From Peripheral Blood

(71) Applicant: Iovance Biotherapeutics, Inc., San Carlos, CA (US)

(72) Inventors: Lavakumar Karyampudi, Saint Petersburg, FL (US); Maria Fardis, San Carlos, CA (US)

(21) Appl. No.: 16/765,126

(22) PCT Filed: Nov. 9, 2018
(86) PCT No.: PCT/US2018/060183

§ 371 Date: May 18, 2020

Related U.S. Application Data
(63) Continuation-in-part of application No. PCT/US2018/032109, filed on May 10, 2018.
(60) Provisional application No. 62/647,367, filed on Mar. 23, 2018.
(60) Provisional application No. 62/621,798, filed on Jan. 25, 2018.
(60) Provisional application No. 62/621,462, filed on Jan. 24, 2018.
(60) Provisional application No. 62/590,034, filed on Nov. 22, 2017.
(60) Provisional application No. 62/550,398, filed on Aug. 25, 2017.
(60) Provisional application No. 62/530,681, filed on Jul. 10, 2017.
(60) Provisional application No. 62/504,337, filed on May 10, 2017.

(51) Int. Cl. C12N 5/0783; A61K 35/17

(52) U.S. Cl. CPC C12N 5/0636; A61K 35/17; C12N 2501/515; C12N 2501/2302; C12N 2501/51; C12N 2502/1107

(57) ABSTRACT

Methods of expanding tumor infiltrating lymphocytes (TILs), including peripheral blood lymphocytes (PBLs) and marrow infiltrating lymphocytes (MILs), from blood and/or bone marrow of patients with hematological malignancies, such as liquid tumors, including lymphomas and leukemias, and genetic modifications of expanded TILs, PBLs, and MILs to incorporate chimeric antigen receptors, genetically modified T-cell receptors, and other genetic modifications, and uses of such expanded and/or modified TILs, PBLs, and MILs in the treatment of diseases such as cancers and hematological malignancies are disclosed herein.

### CROSS REFERENCE TO RELATED APPLICATIONS

This application is a continuation-in-part of International Patent Application No. PCT/US18/032109 filed May 10, 2018, which claims priority to U.S. Provisional Application No. 62/504,337, filed May 10, 2017; U.S. Provisional Application No. 62/530,681, filed Jul. 10, 2017; U.S. Provisional Application No. 62/550,398, filed Aug. 25, 2017; U.S. Provisional Application No. 62/590,034, filed Nov. 22, 2017; U.S. Provisional Application No. 62/621,462, filed Jan. 24, 2018; U.S. Provisional Application No. 62/621,798, filed Jan. 25, 2018; and U.S. Provisional Application No. 62/647,367, filed Mar. 23, 2018, all of which are herein incorporated by reference in their entireties.

### FIELD OF THE INVENTION

Methods of expanding tumor infiltrating lymphocytes (TILs) derived from blood and/or bone marrow of a patient with a hematological malignancy, such as a liquid tumor, including lymphomas and leukemias, and compositions comprising populations of TILs obtained therefrom, are disclosed herein. In addition, therapeutic uses of TILs expanded from blood or bone marrow of a patient with a hematological malignancy, such as a liquid tumor, including in the treatment of such hematological malignancies, are disclosed herein.

### BACKGROUND OF THE INVENTION

Treatment of bulky, refractory cancers using adoptive autologous transfer of tumor infiltrating lymphocytes (TILs) represents a powerful approach to therapy for patients with poor prognoses. Gattinoni, et al., Nat. Rev. Immunol. 2006, 6, 383-393. TILs are dominated by T cells, and IL-2-based TIL expansion followed by a “rapid expansion process” (REP) has become a preferred method for TIL expansion because of its speed and efficiency. Dudley, et al., Science 2002, 298, 850-54; Dudley, et al., J. Clin. Oncol. 2005, 23, 2346-57; Dudley, et al., J. Clin. Oncol. 2008, 26, 5233-39; Riddell, et al., Science 1992, 257, 238-41; Dudley, et al., J. Immunother. 2003, 26, 332-42. A number of approaches to improve responses to TIL therapy in melanoma and to expand TIL therapy to other tumor types have been explored with limited success, and the field remains challenging. Goff, et al., J. Clin. Oncol. 2016, 34, 2389-97; Dudley, et al., J. Clin. Oncol. 2008, 26, 5233-39; Rosenberg, et al., Clin. Cancer Res. 2011, 17, 4550-57. Earlier approaches to expansions of TILs from B cell lymphomas yielded poor results, with only 2 of 12 attempts at TIL growth providing for potential activity against tumors. Schwartzentruber, et al., Blood 1993, 82, 1204-1211. There is an urgent need to provide for more efficacious therapies in many hematological malignancies, including acute myeloid leukemia (AML) and chronic lymphocytic leukemia (CLL).

The present invention provides the surprising finding that TIL expansion processes can result in efficacious TIL populations obtained from hematological malignancies, such as liquid tumors, including lymphomas or leukemias.

### SUMMARY OF THE INVENTION

In an embodiment, the invention provides a method of treating a cancer in a patient with a population of tumor infiltrating lymphocytes (TILs) comprising the steps of:
(a) optionally pre-treating the patient with a regimen comprising at least one kinase inhibitor;
(b) obtaining a tumor from the patient by resection, biopsy, needle aspiration, or apheresis, the tumor comprising a first population of TILs;
(c) optionally fragmenting or dissociating the tumor to obtain tumor fragments and contacting the tumor or tumor fragments with a first cell culture medium;
(d) performing an initial expansion of the first population of TILs in the first cell culture medium to obtain a second population of TILs, wherein the second population of TILs is at least 5-fold greater in number than the first population of TILs, wherein the first cell culture medium comprises IL-2, and wherein the initial expansion is performed over a period of 21 days or less;
(e) performing a second expansion of the second population of TILs in a second cell culture medium to obtain a third population of TILs, wherein the third population of TILs is at least 50-fold greater in number than the second population of TILs after 7 days from the start of the second expansion; wherein the second cell culture medium comprises IL-2, OKT-3 (anti-CD3 antibody), and irradiated allogeneic peripheral blood mononuclear cells (PBMCs), and wherein the second expansion is performed over a period of 14 days or less;
(f) harvesting the third population of TILs; and
(g) administering a therapeutically effective portion of the third population of TILs to the patient;
wherein the tumor is a liquid tumor, and wherein the cancer is a hematological malignancy.

In an embodiment, the invention provides a method of treating a cancer in a patient with a population of tumor infiltrating lymphocytes (TILs) comprising the steps of:
(a) optionally pre-treating the patient with a regimen comprising at least one kinase inhibitor;
(b) obtaining a tumor from the patient by resection, biopsy, needle aspiration, or apheresis, the tumor comprising a first population of TILs;
(c) optionally fragmenting or dissociating the tumor to obtain tumor fragments and contacting the tumor or tumor fragments with a first cell culture medium;
(d) performing an initial expansion of the first population of TILs in the first cell culture medium to obtain a second population of TILs, wherein the second population of TILs is at least 5-fold greater in number than the first population of TILs, wherein the first cell culture medium comprises IL-2, and wherein the initial expansion is performed over a period of 21 days or less;
(e) performing a second expansion of the second population of TILs in a second cell culture medium to obtain a third population of TILs, wherein the third population of TILs is at least 50-fold greater in number than the second population of TILs after 7 days from the start of the second expansion; wherein the second cell culture medium comprises IL-2, OKT-3 (anti-CD3 antibody), and irradiated allogeneic peripheral blood mononuclear cells (PBMCs), and wherein the second expansion is performed over a period of 14 days or less;
(f) harvesting the third population of TILs; and
(g) administering a therapeutically effective portion of the third population of TILs to the patient;
wherein the tumor is a liquid tumor, and wherein the cancer is a hematological malignancy selected from the group consisting of acute myeloid leukemia (AML), mantle cell lymphoma (MCL), follicular lymphoma (FL), diffuse large B cell lymphoma (DLBCL), activated B cell (ABC) DLBCL, germinal center B cell (GCB) DLBCL, chronic lymphocytic leukemia (CLL), small lymphocytic leukemia (SLL), non-Hodgkin's lymphoma (NHL), Hodgkin's lymphoma, relapsed and/or refractory Hodgkin's lymphoma, B cell acute lymphoblastic leukemia (B-ALL), mature B-ALL, Burkitt's lymphoma, Waldenström's macroglobulinemia (WM), multiple myeloma, myelodysplatic syndromes, myelofibrosis, chronic myelocytic leukemia, follicle center lymphoma, indolent NHL, human immunodeficiency virus (HIV) associated B cell lymphoma, and Epstein-Barr virus (EBV) associated B cell lymphoma.

In an embodiment of the invention, the method further comprises addition of an ITK inhibitor. In an embodiment, the ITK inhibitor is added to the cell culture medium during at least one of steps (d) and (e). In an embodiment of the invention, the ITK inhibitor is a covalent ITK inhibitor that covalently and irreversibly binds to ITK. In an embodiment of the invention, the ITK inhibitor is an allosteric ITK inhibitor that binds to ITK. In another embodiment, the ITK inhibitor is selected from the group consisting of aminothiazole-based ITK inhibitors, benzimidazole-based ITK inhibitors, aminopyrimidine-based ITK inhibitors, 3-aminopyride-2-ones-based ITK inhibitors, indolylndazole-based ITK inhibitors, pyrazolyl-indole-based inhibitors, thienopyrazole inhibitors, and ITK inhibitors targeting cysteine-442 in the ATP pocket. In another embodiment, ITK inhibitor is the ITK inhibitor is ibrutinib, dasatinib, bosutinib, nilotinib, erlotinib BMS509744, CTA056, GSK2250665A, PF06465469 ((R)-3-(1-(1-acryloylpiperidin-3-yl)-4-amino-1H-pyrazolo[3,4-d]pyrimidin-3-yl)-N-(3-methyl-4-(1-methylethyl))benzamide), and combinations thereof. In another embodiment, the ITK inhibitor is ibrutinib, also known as 1-[(3R)-3-[4-amino-3-(4-phenoxyphenyl)-1H-pyrazolo[3,4-d]pyrimidin-1-yl]-1-piperidinyl]-2-propen-1-one. In another embodiment, the ITK inhibitor is (R)-3-(1-(1-acryloylpiperidin-3-yl)-4-amino-1H-pyrazolo[3,4-d]pyrimidin-3-yl)-N-(3-methyl-4-(1-methylethyl))benzamide. The foregoing ITK inhibitors are available commercially from various sources, including Tocris Bioscience, Inc. (Minneapolis, Minn., USA), Selleckchem, Inc. (Houston, Tex., USA), and AK Scientific, Inc. (Union City, Calif., USA). In another embodiment, the ITK inhibitor is added at a concentration of from about 0.1 nM to about 5 μM. In another embodiment, the ITK inhibitor is added at a concentration of from about 0.1 nM to about 5 μM. In another embodiment, the ITK inhibitor is added at a concentration of from about 0.1 nM to about 100 nM. In another embodiment, the ITK inhibitor is added at a concentration of from about 0.5 nM to about 50 nM. In another embodiment, the ITK inhibitor is added at a concentration of from about 1 nM to about 10 nM. In an embodiment, the ITK inhibitor is added at a concentration of about 0.01 nM, 0.05 nM, 0.1 nM, 0.5 nM, 1 nM, 2 nM, 5 nM, 10 nM, 20 nM, 30 nM, 40 nM, 50 nM, 60 nM, 70 nM, 80 nM, 90 nM, 100 nM, 150 nM, 200 nM, 300 nM, 400 nM, 500 nM, 600 nM, 700 nM, 800 nM, 900 nM, 1 μM, 2 μM, 3 μM, 4 μM, 5 μM, 10 μM, 20 μM, 30 μM, 40 μM, and 50 μM.

In an embodiment of the invention, a method for expanding peripheral blood lymphocytes (PBLs) from peripheral blood comprises:
a. Obtaining a sample of peripheral blood mononuclear cells (PBMCs) from peripheral blood, wherein said sample is optionally cryopreserved;
b. Isolating PBLs from said sample by selecting and removing CD19+ B cells;
c. Optionally co-culturing said PBLs with said CD19+ B cells;
d. Stimulating said PBLs in a first cell culture medium with IL-2 and anti-CD3/anti-CD28 antibodies for a period of from about 2 days to about 6 days in a gas permeable container;
e. Culturing the PBLs from step (d) for a period of from about 2 days to about 6 days with IL-2 and anti-CD3/anti-CD28 antibodies;
f. Isolating the antibody-bound PBLs from the culture in step (e);
g. Removing the antibodies from the PBLs isolated in step (e); and
h. Harvesting the PBLs.

In an embodiment of the invention, the method further comprises addition of IL-2 after step (d), and exchanging the first culture medium to a second cell culture medium. In another embodiment, the method further comprises addition of IL-2 after step (e), and exchanging the second culture medium to a third culture medium. In an embodiment, the first cell culture medium, second cell culture medium, or third culture medium is selected from the group consisting of CM-2, CM-4, and AIM-V. In another embodiment, the first and second cell culture media are the same. In another embodiment, the first and second cell culture media are different. In an embodiment of the invention, one or more of the first, second and third cell culture media are the same. In another embodiment, the first, second, and third cell culture media are all different.

In an embodiment, the optional co-culturing of said PBLs with said CD19+ B cells is performed for a period of 1 hour to 3 days.

In an embodiment of the invention, the ratio of T-cells to B-cells in step (c) is from about 0.1:1 to about 10:1 (B-cells:T-cells). In another embodiment, the ratio of B-cells to T-cells in step (c) is selected from the group consisting of 0.1:1, 1:1, and 10:1 (B-cells:T-cells).

In an embodiment of the invention, the starting cell number of PBLs at the beginning of step (d) is at least from about 1×10⁵ to about 10×10⁵ PBLs. In another embodiment, the starting cell number of PBLs at the beginning of step (d) is at least from about 2.5×10⁵ to 10×10⁵ PBLs. In another embodiment, the starting cell number of PBLs at the beginning of step (d) is at least 5×10⁵PBLs.

In an embodiment of the invention, the IL-2 in each of steps (c) and (d) is used at a concentration of from about 1000 IU/mL to about 6000 IU/mL. In another embodiment, the IL-2 in each of steps (c) and (d) is used at a concentration of about 3000 IU/mL.

In an embodiment of the invention, the anti-CD3/anti-CD28 antibodies are coated onto beads. In an embodiment of the invention, the anti-CD3/anti-CD28 antibodies-coated beads are DynaBeads®. In an embodiment, the method includes co-culturing the anti-CD3/anti-CD28 antibody beads with the PBLs in about a 1:1 bead:PBL ratio in each of steps (c) and (d).

In an embodiment of the invention, the method comprises adding an ITK inhibitor. In an embodiment, the ITK inhibitor is added during at least one of steps (c), (d), and (e). In an embodiment of the invention, the ITK inhibitor is selected from the group consisting of aminothiazole-based ITK inhibitors, benzimidazole-based ITK inhibitors, aminopyrimidine-based ITK inhibitors, 3-aminopyride-2-ones-based ITK inhibitors, indolylndazole-based ITK inhibitors, pyrazolyl-indole-based inhibitors, thienopyrazole inhibitors, and ITK inhibitors targeting cysteine-442 in the ATP pocket. In another embodiment, the ITK inhibitor is ibrutinib, dasatinib, bosutinib, nilotinib, erlotinib BMS509744, CTA056, GSK2250665A, PF06465469, and combinations thereof. In another embodiment, the ITK inhibitor is ibrutinib.

In an embodiment of the invention, any of the foregoing methods for preparing PBLs is performed in a closed, sterile system.

In an embodiment of the present invention, a method for expanding peripheral blood lymphocytes (PBLs) from peripheral blood comprises:
a. Obtaining a sample of peripheral blood mononuclear cells (PBMCs) from peripheral blood, wherein said sample is optionally cryopreserved;
b. Isolating PBLs from said sample by selecting and removing CD19+ B cells;
c. Co-culturing said PBLs with said CD19+ B cells for a period of 4 days;
d. Adding from about 2.5×10⁵ to about 5×10⁵ PBLs to a gas-permeable container in CM-2 cell culture medium and stimulating said PBLs with 3000 IU/ml IL-2 and anti-CD3/anti-CD28 antibodies immobilized on beads for a period of about 4 days;
e. Exchanging the CM-2 cell culture medium with AIM-V cell culture medium and additional IL-2 at about 3000 IU/ml;
f. Culturing the PBLs in the AIM-V cell culture medium from step (e) for an additional period of about 3 days with IL-2 and anti-CD³/anti-CD28 antibodies immobilized on beads;
g. Isolating the antibody-bound PBLs from the culture in step (f);
h. Removing the antibodies from the PBLs isolated in step (g); and
i. Harvesting the PBLs.

In an embodiment of the invention, a method for treating a hematological malignancy comprises:
a. Obtaining a sample of peripheral blood mononuclear cells (PBMCs) from peripheral blood of a patient suffering from a hematological malignancy;
b. Isolating PBLs from said sample by selecting and removing CD19+ B cells;
c. Optionally co-culturing said PBLs with said CD19+ B cells;
d. Stimulating said PBLs in a first cell culture medium with IL-2 and anti-CD3/anti-CD28 antibodies for a period of at least about 4 days in a gas permeable container;
e. Culturing the PBLs from step (d) for a period of 3 days with IL-2 and anti-CD3/anti-CD28 antibodies;
f. Isolating the antibody-bound PBLs from the culture in step (e);
g. Removing the antibodies from the PBLs isolated in step (e); and
h. Harvesting the PBLs; and
i. Administering the PBLs to the patient in a therapeutically effective amount to treat said hematological malignancy.

In an embodiment of the invention, the method further comprises obtaining a PBMC sample from a patient that is pre-treated with an ITK inhibitor. In an embodiment of the invention, the ITK inhibitor is selected from the group consisting of aminothiazole-based ITK inhibitors, benzimidazole-based ITK inhibitors, aminopyrimidine-based ITK inhibitors, 3-aminopyride-2-ones-based ITK inhibitors, indolylndazole-based ITK inhibitors, pyrazolyl-indole-based inhibitors, thienopyrazole inhibitors, and ITK inhibitors targeting cysteine-442 in the ATP pocket. In an embodiment of the invention, the ITK inhibitor is ibrutinib, BMS509744, CTA056, GSK2250665A, PF06465469, and combinations thereof. In another embodiment, the ITK inhibitor is ibrutinib. In another embodiment, the patient is pre-treated with at least three rounds of an ibrutinib regimen.

In an embodiment of the invention, the hematological malignancy is selected from the group consisting of acute myeloid leukemia (AML), mantle cell lymphoma (MCL), follicular lymphoma (FL), diffuse large B cell lymphoma (DLBCL), activated B cell (ABC) DLBCL, germinal center B cell (GCB) DLBCL, chronic lymphocytic leukemia (CLL), CLL with Richter's transformation (Richter's syndrome), small lymphocytic leukemia (SLL), non-Hodgkin's lymphoma (NHL), Hodgkin's lymphoma, relapsed and/or refractory Hodgkin's lymphoma, B cell acute lymphoblastic leukemia (B-ALL), mature B-ALL, Burkitt's lymphoma, Waldenström's macroglobulinemia (WM), multiple myeloma, myelodysplatic syndromes, myelofibrosis, chronic myelocytic leukemia, follicle center lymphoma, indolent NHL, human immunodeficiency virus (HIV) associated B cell lymphoma, and Epstein-Barr virus (EBV) associated B cell lymphoma. In another embodiment, the hematological malignancy is chronic lymphocytic leukemia (CLL). In an embodiment of the invention, the PBLs are administered in an amount of from about 0.1×10⁹ to about 15×10⁹ PBLs.

In an embodiment of the invention, a method for expanding marrow-infiltrating lymphocytes (MILs) from bone marrow comprises:
a. Obtaining a sample of peripheral blood mononuclear cells (PBMCs) from bone marrow, wherein said sample is optionally cryopreserved;
b. Sorting a CD3+, CD33+, CD20+ and CD14+ cell fraction comprising MILs (MIL cell fraction) and a non-CD3+, non-CD33+, non-CD20+, non-CD14+ cell fraction (AML blast cell fraction) from the sample of PBMCs;
c. Optionally disrupting the AML blast cell fraction;
d. Adding the optionally disrupted AML blast cell fraction to the MIL cell fraction in a cell number ratio of from about 0.1:1 to about 10:1
e. Culturing the MIL cell fraction, with or without the AML blast cell fraction, in a gas permeable container in a first cell culture medium, comprising IL-2;
f. Stimulating the MILs with anti-CD3/anti-CD28 antibodies to obtain expansion of MILs;
g. Restimulating the MILs with IL-2 and anti-CD3/anti-CD28 antibodies for an additional period of about 2 to about 6 days;
h. Culturing the MILs with additional IL-2 for an additional period of from about 1 to about 3 days; and
i. Harvesting said MILs.

In an embodiment of the invention, the method further comprises addition IL-2 after step (e), and exchanging the first cell culture medium to a second cell culture medium. In an embodiment, the first cell culture medium and the second cell culture medium are each individually selected from the group consisting of CM-2, CM-4, and AIM-V. In another embodiment, the first and second cell culture media are the same. In another embodiment, the first and second cell culture media are different.

In an embodiment, there are at least from about 2×10⁴ to about 5×10⁵ MILs in the gas permeable container at the beginning of step (e). In another embodiment, there are at least from about 2.8×10⁴ to 3.4×10⁵ MILs in the gas permeable container at the beginning of step (e). In another embodiment, there are at least 5×10⁵ MILs in the gas permeable container at the beginning of step (e).

In an embodiment of the invention, the IL-2 is present in a concentration of between 1000 IU/ml and 6000 IL/ml in step (e). In another embodiment, the IL-2 is present in a concentration of about 6000 IU/ml. In another embodiment, the IL-2 is present in a concentration of about 3000 IU/ml in step (g). In another embodiment, the IL-2 is present in a concentration of about 3000 IU/ml in step (h).

In an embodiment of the invention, the culturing in step (e) is performed over a period of about 3 days. In an embodiment, the stimulation in step (f) is performed over a period of about 4 days. In an embodiment, the stimulation in step (g) is performed over a period of about 7 days.

In an embodiment of the invention, the optionally disrupted AML blast cell fraction is disrupted using a method selected from the group consisting of sonication, homogenization, vortexing, vibration, and lysis. In an embodiment of the invention, the non-CD3+, non-CD33+, non-CD20+, non-CD14+ cell fraction (AML blast cell fraction) is lysed using a suitable lysis method, including high temperature lysis, chemical lysis (such as organic alcohols), enzyme lysis, and other cell lysis methods known in the art.

In an embodiment of the invention, the anti-CD3/anti-CD28 antibodies are coated onto beads and the MILs:bead ratio is about 1:1 in each of steps (f) and (g).

In an embodiment of the invention, the method is performed in a closed, sterile system.

In an embodiment of the invention, a method for expanding marrow infiltrating lymphocytes (MILs) from bone marrow comprises:
a. Obtaining a sample of peripheral blood mononuclear cells (PBMCs) from bone marrow, wherein said sample is optionally cryopreserved;
b. Sorting a CD3+, CD33+, CD20+ and CD14+ cell fraction comprising MILs (MIL cell fraction) and a non-CD3+, non-CD33+, non-CD20+, non-CD14+ cell fraction (AML blast cell fraction) from the sample of PBMCs;
c. Disrupting the AML blast cell fraction and adding the disrupted AML blast cell fraction to a MIL cell fraction in a cell number ratio of about 1:1;
d. Culturing the MILs and AML blast cell fractions in a cell culture contained in a gas permeable container with a first cell culture medium comprising IL-2 at about 6000 IU/ml for a period of about 3 days;
e. Adding anti-CD3/anti-CD28 antibodies immobilized on beads to the cell culture at a ratio of about 1:1 (MILs:beads) and culturing the MILs and antibodies for a period of about 1 day;
f. Exchanging the first cell culture medium to a second cell culture medium comprising additional IL-2 at about 3000 IU/ml;
g. Culturing the antibodies and MILs for an additional period of about 3 days;
h. Restimulating the MILs with IL-2 and anti-CD3/anti-CD28 antibodies immobilized on beads for an additional period of at least about 4 days;
i. Exchanging the second cell culture medium to a third cell culture medium comprising additional IL-2 at about 3000 IU/ml and culturing for an additional period of at least about 3 days;
j. Harvesting said MILs.

In an embodiment of the invention, a method for treating a hematological malignancy in a patient comprises:
a. Obtaining a sample of peripheral blood mononuclear cells (PBMCs) from bone marrow of the patient, wherein said sample is optionally cryopreserved;
b. Sorting a CD3+, CD33+, CD20+ and CD14+ cell fraction comprising MILs (MIL cell fraction) and a non-CD3+, non-CD33+, non-CD20+, non-CD14+ cell fraction (AML blast cell fraction) from the sample of PBMCs;
c. optionally disrupting the AML blast cell fraction;
d. Adding the optionally disrupted AML blast cell fraction to the MIL cell fraction in a cell number ratio of from about 0.1:1 to about 10:1;
e. Culturing the MIL cell fraction, with or without the AML blast cell fraction, in a gas permeable container in a first cell culture medium comprising IL-2;
f. Stimulating the MILs with anti-CD3/anti-CD28 antibodies;
g. Restimulating the MILs with IL-2 and anti-CD3/anti-CD28 antibodies for an additional period of at least about 4 days;
h. Culturing the MILs with additional IL-2 for an additional period of at least about 3 days;
i. Harvesting said MILs; and
j. Administering said MILs to the patient in a therapeutically effective amount to treat the hematological malignancy.

In an embodiment of the invention, the hematological malignancy is selected from the group consisting of acute myeloid leukemia (AML), mantle cell lymphoma (MCL), follicular lymphoma (FL), diffuse large B cell lymphoma (DLBCL), activated B cell (ABC) DLBCL, germinal center B cell (GCB) DLBCL, chronic lymphocytic leukemia (CLL), CLL with Richter's transformation (Richter's syndrome), small lymphocytic leukemia (SLL), non-Hodgkin's lymphoma (NHL), Hodgkin's lymphoma, relapsed and/or refractory Hodgkin's lymphoma, B cell acute lymphoblastic leukemia (B-ALL), mature B-ALL, Burkitt's lymphoma, Waldenström's macroglobulinemia (WM), multiple myeloma, myelodysplatic syndromes, myelofibrosis, chronic myelocytic leukemia, follicle center lymphoma, indolent NHL, human immunodeficiency virus (HIV) associated B cell lymphoma, and Epstein-Barr virus (EBV) associated B cell lymphoma. In another embodiment, the hematological malignancy is acute myeloid leukemia (AML). In an embodiment of the invention, the MILs are administered in an amount of from about 4×10⁸ to about 2.5×10⁹ MILs.

### BRIEF DESCRIPTION OF THE DRAWINGS

The foregoing summary, as well as the following detailed description of the invention, will be better understood when read in conjunction with the appended drawings.

FIG. 1 illustrates pathology information for lymphoma tumors.

FIG. 2 illustrates a comparison of different subsets of lymphoma and melanoma TILs, showing that effector memory (EM) subsets in lymphoma TILs are significantly higher than EM subsets in melanoma TILs.

FIG. 3 illustrates a comparison of different subsets of lymphoma and melanoma TILs, showing that CD28⁺ CD4⁺ subsets in lymphoma TIL are significantly higher than these subsets in melanoma TILs.

FIG. 4 illustrates a comparison of CD4⁺ T cell subsets of non-Hodgkin's lymphoma TILs and melanoma TILs, showing differentiation markers. Red lines in the graphs represent median values. CM refers to central memory T cells, EM refers to effector memory T cells, and TEMRA refers to effector memory CD45RA⁺ T cells.

FIG. 5 illustrates a comparison of CD8⁺ T cell subsets of non-Hodgkin's lymphoma TILs and melanoma TILs, showing differentiation markers. Red lines in the graphs represent median values. CM refers to central memory T cells, EM refers to effector memory T cells, and TEMRA refers to effector memory CD45RA⁺ T cells.

FIG. 6 illustrates a comparison of CD4⁺ T cell subsets of non-Hodgkin's lymphoma TILs and melanoma TILs, showing exhaustion markers. Red lines in the graphs represent median values. LAG3 refers to lymphocyte-activation gene 3, PD1 refers to programmed death 1, and TIGIT refers to T cell immunoreceptor with Ig and ITIM domains.

FIG. 7 illustrates a comparison of CD8⁺ T cell subsets of non-Hodgkin's lymphoma TILs and melanoma TILs, showing exhaustion markers. Red lines in the graphs represent median values. LAG3 refers to lymphocyte-activation gene 3, PD1 refers to programmed death 1, and TIGIT refers to T cell immunoreceptor with Ig and ITIM domains.

FIG. 8 illustrates a comparison of cell types between non-Hodgkin's lymphoma TILs and melanoma TILs. NK refers to natural killer cells, and TCRab refers to cells expressing a T cell receptor with alpha and beta chains.

FIG. 9 illustrates bioluminescent redirected lysis assay (BRLA) results.

FIG. 10 illustrates interferon-γ (IFN-γ) enzyme-linked immunosorbent assay (ELISA) results for lymphoma TILs versus melanoma TILs.

FIG. 11 illustrates enzyme-linked immunospot (ELIspot) assay results for lymphoma TILs.

FIG. 12 illustrates ELIspot assay results for melanoma TILs.

FIG. 13 illustrates the results of NANOSTRING NCOUNTER analysis, showing that lymphoma TILs express higher levels of RORC IL17A (TH17 phenotype) and GATA3 (Th2 phenotype) compared to melanoma TILs. Respective genes are highlighted in red boxes in the heat map.

FIG. 14 illustrates a TIL expansion and treatment process. Step 1 refers to the addition of 4 tumor fragments into 10 G-Rex 10 flasks. At step 2, approximately 40×10⁶ TILs or greater are obtained. At step 3, a split occurs into 36 G-Rex 100 flasks for REP. TILs are harvested by centrifugation at step 4. Fresh TIL product is obtained at step 5 after a total process time of approximate 43 days, at which point TILs may be infused into a patient.

FIG. 15 illustrates a treatment protocol for use with TILs obtained from lymphomas of the present disclosure. Surgery (and tumor resection) occurs at the start, and lymphodepletion chemo refers to non-myeloablative lymphodepletion with chemotherapy as described elsewhere herein.

FIG. 16 demonstrates the results of the flow cytometry analysis using standard phenotype panel DF2 as described in Example 4, below. Lymphoma and melanoma TILs were stained using standard phenotype panel DF2 as described in Example 4. Data shown represents different subpopulations of total CD4 and CD8 T cells in TIL. FIG. 16A demonstrates the proportion of CD4 and CD8 cells for Naïve T-cell subsets; FIG. 16B for central memory T-cell subsets (CM); FIG. 16C for effector memory T-cell subsets (EM), and FIG. 16D for terminally differentiated effector memory (TEMRA) T-cell subsets. P-values were calculated using two-tailed Mann-Whitney Test (unpaired). The mean proportion of cell subsets is prepresented by horizontal bars.

FIG. 17 demonstrates the results of the flow cytometry analysis using the standard phenotype panel DF1 as described in Example 4, below. Lymphoma and melanoma TIL were stained using standard phenotype panel DF1, as described in Example 4. Data shown represents different CD27+ (FIG. 17A) and CD28+ (FIG. 17B) subpopulations of total CD4 and CD9 T-cells in TIL, which indicates a higher proportion of costimulatory molecule-CD28 expressing CD4 T-cells in lymphoma TIL. P values were calculated using the two-tailed Mann-Whitney Test (unpaired).

FIG. 18 demonstrates the results of an interferon-gamma (IFN-γ) test conducted in accordance with Example 4, below. FIG. 18A demonstrates the results using ELIspot. ELIspot data is expressed as IFN-γ producing cells per 10⁶ TIL. FIG. 18B demonstrates the results using ELISA. ELISA data is expressed as IFN-γ levels in the supernatants from TIL cultures at 5×10⁵ TIL/well) as measured by ELISA (logarithmic scale. P values were calculated using the two-tailed Mann-Whitney Test (unpaired).

FIG. 19 demonstrates the lytic potential of TIL. FIG. 19A shows the LU₅₀ of target cells normalized to 10⁶ TIL at 4 hours (FIG. 19A) and 24 hours (FIG. 19B) in co-culture (TIL effector cells with GFP+P815 target cells).

FIG. 20 demonstrates the cytolytic activity of different TIL against allogeneic and autologous tumor types. FIG. 20A shows the cytolytic activity of melanoma TIL against allogeneic 526 target cells. FIG. 20B shows the cytolytic activity of lymphoma TIL against autologous tumor cells determined by 7-AAD uptake. The data in FIGS. 20A and 20B FIG. are shown as percent dead cells in co-cultures with 50:1 effector cell:target cell (E:T) ratio. FIG. 20C represents percent killing of target cells induced by melanoma TIL. FIG. 20D represents percent killing of target cells induced by lymphoma TIL at different E:T ratios.

FIG. 21 is a heat map showing the gene expression profiles of lymphoma and melanoma TIL. The expression profiles were determined by 579 plex nCounter GX Human Immunology V2 CSO panel from NanoString. The heat map shows the fold change in expression of a particular set of genes in lymphoma TIL compared to melanoma TIL, and suggests a higher expression of IL-17A and RORC from lymphoma-derived TIL. The cancers shown in this figure include follicular lymphoma (FL), diffuse large B cell Lymphoma (DLBCL) and mantle cell lymphoma (MCL).

FIG. 22 is a schematic demonstrating the 2A process for preparing TIL, harvest, and ship schedule.

FIG. 23 is a flow chart demonstrating the 2A process for preparing TIL.

FIG. 24 is a flow chart demonstrating three different methods for expanding Peripheral Blood Lymphocytes (PBLs).

FIGS. 25A-25C represent three different methods for expanding marrow infiltrating lymphocytes (MILs) from bone marrow.

FIG. 26 represents a graph of the fold expansion for PBLs isolated from fresh peripheral blood mononuclear cells (PBMCs) and from cryopreserved PBMCs. The cryopreserved PBMCs are derived from patients with CLL who have not been (PreRx PBL) or who have been (PostRx PBL) treated with an ibrutinib regimen. For each of FIGS. 26-34, each dot is one patient. Shaded dots are patients whose PBLs were expanded using PBL Method 1; open dots are patients whose PBLs were expanded using PBL Method 2; black dots are patients whose PBLs were expanded using PBL Method 3.

FIG. 27 represents a graph of IFN-γ producing cells for PBLs isolated from fresh PBMCs and cryopreserved PBMCs. Within cryopreserved PBMCs, PreRx PBLs and PostRx PBLs are also represented.

FIG. 28 represents the proportion of CD4+ and CD8+ T cell subsets in PreRx PBL and PostRx PBL, using melanoma TIL as a comparator.

FIGS. 29A-29D and FIGS. 30A-30D represent a comparison between CD4 (FIG. 29) and CD8 (FIG. 30) memory subsets of PreRx PBLs and PostRx PBLs, using melanoma TIL as a comparator. FIGS. 29A and 30A show data for naïve (CCR7+/CD45RA+); FIGS. 29B and 30B show data for central memory t-cells (CM) (CCR7+/CD45RA−); FIGS. 29C and 30C show data for effector memory T-cells (EM) (CCR7−/CD45RA−); and FIGS. 29D and 30D show data for terminally differentiated effector memory cells (TEMRA) (CCR7−/CD45RA+).

FIGS. 31A and 31B represent a comparison of CD27 subsets of CD4 (FIG. 31A) and CD8 (FIG. 31B) subsets for PreRx PBLs and PostRx PBLs, using melanoma TIL as a comparator.

FIGS. 32A and 32B represent a comparison of CD28 subsets of CD4 (FIG. 32A) and CD8 (FIG. 32B) subsets for PreRx PBLs and PostRx PBLs, using melanoma TIL as a comparator.

FIGS. 33A and 33B represent a comparison of LAG3+ subsets within the CD4 (FIG. 33A) and CD8 (FIG. 33B) populations for both PreRx PBLs and PostRx PBLs.

FIGS. 34A and 34B represent a comparison of PD1+ subsets within the CD4 (FIG. 34A) and CD8 (FIG. 34B) populations for both PreRx PBLs and PostRx PBLs.

FIGS. 35A and 35B show results of cytolytic activity of PreRx PBLs (FIG. 35A) and PostRx PBLs (FIG. 35B), measured using an autologous tumor killing assay. The cytotoxicity is measured as the LU₅₀ (the number of PBLs required to kill 50% of the target cells).

FIGS. 36A and 36B represent graphs of the fold expansion for MILs (FIG. 36A) and PBLs (FIG. 36B) isolated from either bone marrow (MILs) or peripheral blood (PBLs) of AML patients. MIL 1.1 was expanded using MIL Method 1, MIL1.2 was expanded using MIL Method 2, and MIL1.3 was expanded using MIL Method 3. MIL2 and MIL3 were expanded using MIL Method 3. All PBLs were expanded using PBL Method 3. Starting cell number for MIL1.3 was 138,000 cells, for MIL2 was 62,000 and for MIL 3 was 28,000 cells. Starting cell number for PBL2 was 338,000 and for PBL3 was 336,000.

FIGS. 37A and 37B illustrate IFN-γ producing cells for each of MILs (FIG. 37A) and PBLs (FIG. 37B).

FIGS. 38A-38F represent graphs illustrating T cell subsets in MILs (FIGS. 38A-38C) and PBLs (FIGS. 38D-38F) isolated from AML patients. FIGS. 38A and 38D illustrate TCRαβ+ subsets, FIGS. 38B and 38E illustrate CD4+ subsets, and FIGS. 38C and 38F illustrate CD8 subsets. PBLs are shown at Day 0 and at Day 14.

FIGS. 39A-39D represent graphs illustrating CD4 memory subsets for MILs isolated from AML patients. FIG. 39A shows data for naïve (CCR7+/CD45RA+); FIG. 39B shows data for central memory t-cells (CM) (CCR7+/CD45RA−); FIG. 39C shows data for effector memory T-cells (EM) (CCR7−/CD45RA−); and FIG. 39D shows data for terminally differentiated effector memory cells (TEMRA) (CCR7−/CD45RA+).

FIGS. 40A-40D represent graphs illustrating CD4 memory subsets for PBLs isolated from AML patients. FIG. 40A shows data for naïve (CCR7+/CD45RA+); FIG. 40B shows data for central memory t-cells (CM) (CCR7+/CD45RA−); FIG. 40C shows data for effector memory T-cells (EM) (CCR7−/CD45RA−); and FIG. 40D shows data for terminally differentiated effector memory cells (TEMRA) (CCR7−/CD45RA+).

FIGS. 41A-41D represent graphs illustrating CD8 memory subsets for MILs isolated from AML patients. FIG. 41A shows data for naïve (CCR7+/CD45RA+); FIG. 41B shows data for central memory t-cells (CM) (CCR7+/CD45RA−); FIG. 41C shows data for effector memory T-cells (EM) (CCR7−/CD45RA−); and FIG. 41D shows data for terminally differentiated effector memory cells (TEMRA) (CCR7−/CD45RA+).

FIGS. 42A-42D represent graphs illustrating CD8 memory subsets for PBLs isolated from AML patients. FIG. 42A shows data for naïve (CCR7+/CD45RA+); FIG. 42B shows data for central memory t-cells (CM) (CCR7+/CD45RA−); FIG. 42C shows data for effector memory T-cells (EM) (CCR7−/CD45RA−); and FIG. 42D shows data for terminally differentiated effector memory cells (TEMRA) (CCR7−/CD45RA+).

FIGS. 43A and 43B represent graphs illustrating CD27 subsets of CD4 and CD8 cell populations for MILs (FIG. 43A) and PBLs (FIG. 43B).

FIGS. 44A and 44B represent graphs illustrating CD28 subsets of CD4 and CD8 cell populations for MILs (FIG. 44A) and PBLs (FIG. 44B).

FIGS. 45A and 45B represent graphs illustrating PD1+ subsets of CD4 and CD8 cell populations for MILs (FIG. 45A) and PBLs (FIG. 45B).

FIGS. 46A and 46B represent graphs illustrating LAG3+ subsets of CD4 and CD8 cell populations for MILs (FIG. 46A) and PBLs (FIG. 46B).

FIG. 47 is a timeline illustrating exemplary embodiments of PBL Method 1 and PBL Method 3. In this figure, the addition of IL-2 can take place at any point in time during the process, and in an exemplary embodiment, over the bracketed area.

FIG. 48 is a timeline illustrating an exemplary embodiment of the MIL Method 3. In this figure, the addition of IL-2 can take place at any point in time during the process, and in an exemplary embodiment, over the bracketed area.

FIG. 49 is an exemplary embodiment of the present invention, showing a method for expanding PBLs (including PBLs from a patient pre-treated with ibrutinib) useful for treatment of hematological malignancies, such as CLL, as described herein.

### BRIEF DESCRIPTION OF THE SEQUENCE LISTING

SEQ ID NO:1 is the amino acid sequence of the heavy chain of muromonab.

SEQ ID NO:2 is the amino acid sequence of the light chain of muromonab.

SEQ ID NO:3 is the amino acid sequence of a recombinant human IL-2 protein.

SEQ ID NO:4 is the amino acid sequence of aldesleukin.

SEQ ID NO:5 is the amino acid sequence of a recombinant human IL-4 protein.

SEQ ID NO:6 is the amino acid sequence of a recombinant human IL-7 protein.

SEQ ID NO:7 is the amino acid sequence of a recombinant human IL-15 protein.

SEQ ID NO:8 is the amino acid sequence of a recombinant human IL-21 protein.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entireties.

### Definitions

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The term “in vivo” refers to an event that takes place in a mammalian subject's body.

The term “ex vivo” refers to an event that takes place outside of a mammalian subject's body, in an artificial environment.

The term “in vitro” refers to an event that takes places in a test system. In vitro assays encompass cell-based assays in which alive or dead cells may be are employed and may also encompass a cell-free assay in which no intact cells are employed.

The term “rapid expansion” means an increase in the number of antigen-specific TILs of at least about 3-fold (or 4-, 5-, 6-, 7-, 8-, or 9-fold) over a period of a week, more preferably at least about 10-fold (or 20-, 30-, 40-, 50-, 60-, 70-, 80-, or 90-fold) over a period of a week, or most preferably at least about 100-fold over a period of a week. A number of rapid expansion protocols are described herein.

The terms “fragmenting,” “fragment,” and “fragmented,” as used herein to describe processes for disrupting a tumor, includes mechanical fragmentation methods such as crushing, slicing, dividing, and morcellating tumor tissue as well as any other method for disrupting the physical structure of tumor tissue.

The terms “peripheral blood mononuclear cells” and “PBMCs” refers to a peripheral blood cell having a round nucleus, including lymphocytes (T cells, B cells, NK cells) and monocytes. Optionally, the peripheral blood mononuclear cells are irradiated allogeneic peripheral blood mononuclear cells. PBMCs include antigen presenting cells. The term “PBLs” refers to peripheral blood lymphocytes and are T-cells expanded from peripheral blood. The terms PBL and TIL are used interchangeably herein.

The term “anti-CD3 antibody” refers to an antibody or variant thereof, e.g., a monoclonal antibody and including human, humanized, chimeric or murine antibodies which are directed against the CD3 receptor in the T cell antigen receptor of mature T cells. Anti-CD3 antibodies include OKT-3, also known as muromonab, and UCHT-1. Other anti-CD3 antibodies include, for example, otelixizumab, teplizumab, and visilizumab.

The term “OKT-3” (also referred to herein as “OKT3”) refers to a monoclonal antibody or biosimilar or variant thereof, including human, humanized, chimeric, or murine antibodies, directed against the CD3 receptor in the T cell antigen receptor of mature T cells, and includes commercially-available forms such as OKT-3 (30 ng/mL, MACS GMP CD3 pure, Miltenyi Biotech, Inc., San Diego, Calif., USA) and muromonab or variants, conservative amino acid substitutions, glycoforms, or biosimilars thereof. The amino acid sequences of the heavy and light chains of muromonab are given in Table 1 (SEQ ID NO:1 and SEQ ID NO:2). A hybridoma capable of producing OKT-3 is deposited with the American Type Culture Collection and assigned the ATCC accession number CRL 8001. A hybridoma capable of producing OKT-3 is also deposited with European Collection of Authenticated Cell Cultures (ECACC) and assigned Catalogue No. 86022706.

TABLE 1
Amino acid sequences of muromonab.

| Identifier | Sequence (One-Letter Amino Acid Symbols) | |
|---|---|---|
| SEQ ID NO: 1 | QVQLQQSGAE LARPGASVKM SCKASGYTFT RYTMHWVKQR PGQGLEWIGY INPSRGYTNY | 60 |
| Muromonab | NQKFKDKATL TTDKSSSTAY MQLSSLTSED SAVYYCARYY DDHYCLDYWG QGTTLTVSSA | 120 |
| heavy | KTTAPSVYPL APVCGGTTGS SVTLGCLVKG YFPEPVTLTW NSGSLSSGVH TFPAVLQSDL | 180 |
| chain | YTLSSSVTVT SSTWPSQSIT CNVAHPASST KVDKKIEPRP KSCDKTHTCP PCPAPELLGG | 240 |
| | PSVFLFPPKP KDTLMISRTP EVTCVVVDVS HEDPEVKFNW YVDGVEVHNA KTKPREEQYN | 300 |
| | STYRVVSVLT VLHQDWLNGK EYKCKVSNKA LPAPIEKTIS KAKGQPREPQ VYTLPPSRDE | 360 |
| | LTKNQVSLTC LVKGFYPSDI AVEWESNGQP ENNYKTTPPV LDSDGSFFLY SKLTVDKSRW | 420 |
| | QQGNVFSCSV MHEALHNHYT QKSLSLSPGK | 450 |
| SEQ ID NO: 2 | QIVLTQSPAI MSASPGEKVT MTCSASSSVS YMNWYQQKSG TSPKRWIYDT SKLASGVPAH | 60 |
| Muromonab | FRGSGSGTSY SLTISGMEAE DAATYYCQQW SSNPFTFGSG TKLEINRADT APTVSIFPPS | 120 |
| light | SEQLTSGGAS VVCFLNNFYP KDINVKWKID GSERQNGVLN SWTDQDSKDS TYSMSSTLTL | 180 |
| chain | TKDEYERHNS YTCEATHKTS TSPIVKSFNR NEC | 213 |

The term “IL-2” (also referred to herein as “IL2”) refers to the T cell growth factor known as interleukin-2, and includes all forms of IL-2 including human and mammalian forms, conservative amino acid substitutions, glycoforms, biosimilars, and variants thereof. IL-2 is described, e.g., in Nelson, J. Immunol. 2004, 172, 3983-88 and Malek, Annu. Rev. Immunol. 2008, 26, 453-79, the disclosures of which are incorporated by reference herein. The amino acid sequence of recombinant human IL-2 suitable for use in the invention is given in Table 2 (SEQ ID NO:3). For example, the term IL-2 encompasses human, recombinant forms of IL-2 such as aldesleukin (PROLEUKIN, available commercially from multiple suppliers in 22 million IU per single use vials), as well as the form of recombinant IL-2 commercially supplied by CellGenix, Inc., Portsmouth, N.H., USA (CELLGRO GMP) or ProSpec-Tany TechnoGene Ltd., East Brunswick, N.J., USA (Cat. No. CYT-209-b) and other commercial equivalents from other vendors. Aldesleukin (des-alanyl-1, serine-125 human IL-2) is a nonglycosylated human recombinant form of IL-2 with a molecular weight of approximately 15 kDa. The amino acid sequence of aldesleukin suitable for use in the invention is given in Table 2 (SEQ ID NO:4). The term IL-2 also encompasses pegylated forms of IL-2, as described herein, including the pegylated IL2 prodrug NKTR-214, available from Nektar Therapeutics, South San Francisco, Calif., USA. NKTR-214 and pegylated IL-2 suitable for use in the invention is described in U.S. Patent Application Publication No. US 2014/0328791 A1 and International Patent Application Publication No. WO 2012/065086 A1, the disclosures of which are incorporated by reference herein. Alternative forms of conjugated IL-2 suitable for use in the invention are described in U.S. Pat. Nos. 4,766,106, 5,206,344, 5,089,261 and 4,902,502, the disclosures of which are incorporated by reference herein. Formulations of IL-2 suitable for use in the invention are described in U.S. Pat. No. 6,706,289, the disclosure of which is incorporated by reference herein.

TABLE 2
Amino acid sequences of interleukins.

| Identifier | Sequence (One-Letter Amino Acid Symbols) | |
|---|---|---|
| SEQ ID NO: 3 | MAPTSSSTKK TQLQLEHLLL DLQMILNGIN NYKNPKLTRM LTFKFYMPKK ATELKHLQCL | 60 |
| recombinant | EEELKPLEEV LNLAQSKNFH LRPRDLISNI NVIVLELKGS ETTFMCEYAD ETATIVEFLN | 120 |
| human IL-2 | RWITFCQSII STLT | 134 |
| (rhIL-2) | | |
| SEQ ID NO: 4 | PTSSSTKKTQ LQLEHLLLDL QMILNGINNY KNPKLTRMLT FKFYMPKKAT ELKHLQCLEE | 60 |
| Aldesleukin | ELKPLEEVLN LAQSKNFHLR PRDLISNINV IVLELKGSET TFMCEYADET ATIVEFLNRW | 120 |
| | ITFSQSIIST LT | 132 |
| SEQ ID NO: 5 | MHKCDITLQE IIKTLNSLTE QKTLCTELTV TDIFAASKNT TEKETFCRAA TVLRQFYSHH | 60 |
| recombinant | EKDTRCLGAT AQQFHRHKQL IRFLKRLDRN LWGLAGLNSC PVKEANQSTL ENFLERLKTI | 120 |
| human IL-4 | MREKYSKCSS | 130 |
| (rhIL-4) | | |
| SEQ ID NO: 6 | MDCDIEGKDG KQYESVLMVS IDQLLDSMKE IGSNCLNNEF NFFKRHICDA NKEGMFLFRA | 60 |
| recombinant | ARKLRQFLKM NSTGDFDLHL LKVSEGTTIL LNCTGQVKGR KPAALGEAQP TKSLEENKSL | 120 |
| human IL-7 | KEQKKLNDLC FLKRLLQEIK TCWNKILMGT KEH | 153 |
| (rhIL-7) | | |
| SEQ ID NO: 7 | MNWVNVISDL KKIEDLIQSM HIDATLYTES DVHPSCKVTA MKCFLLELQV ISLESGDASI | 60 |
| recombinant | HDTVENLIIL ANNSLSSNGN VTESGCKECE ELEEKNIKEF LQSFVHIVQM FINTS | 115 |
| human IL-15 | | |
| (rhIL-15) | | |
| SEQ ID NO: 8 | MQDRHMIRMR QLIDIVDQLK NYVNDLVPEF LPAPEDVETN CEWSAFSCFQ KAQLKSANTG | 60 |
| recombinant | NNERIINVSI KKLKRKPPST NAGRRQKHRL TCPSCDSYEK KPPKEFLERF KSLLQKMIHQ | 120 |
| human IL-21 | HLSSRTHGSE DS | 132 |
| (rhIL-21) | | |

The term “IL-4” (also referred to herein as “IL4”) refers to the cytokine known as interleukin 4, which is produced by Th2 T cells and by eosinophils, basophils, and mast cells. IL-4 regulates the differentiation of naive helper T cells (Th0 cells) to Th2 T cells. Steinke and Borish, Respir. Res. 2001, 2, 66-70. Upon activation by IL-4, Th2 T cells subsequently produce additional IL-4 in a positive feedback loop. IL-4 also stimulates B cell proliferation and class II MHC expression, and induces class switching to IgE and IgG₁ expression from B cells. Recombinant human IL-4 suitable for use in the invention is commercially available from multiple suppliers, including ProSpec-Tany TechnoGene Ltd., East Brunswick, N.J., USA (Cat. No. CYT-211) and ThermoFisher Scientific, Inc., Waltham, Mass., USA (human IL-15 recombinant protein, Cat. No. Gibco CTP0043). The amino acid sequence of recombinant human IL-4 suitable for use in the invention is given in Table 2 (SEQ ID NO:5).

The term “IL-7” (also referred to herein as “IL7”) refers to a glycosylated tissue-derived cytokine known as interleukin 7, which may be obtained from stromal and epithelial cells, as well as from dendritic cells. Fry and Mackall, Blood 2002, 99, 3892-904. IL-7 can stimulate the development of T cells. IL-7 binds to the IL-7 receptor, a heterodimer consisting of IL-7 receptor alpha and common gamma chain receptor, which in a series of signals important for T cell development within the thymus and survival within the periphery. Recombinant human IL-7 suitable for use in the invention is commercially available from multiple suppliers, including ProSpec-Tany TechnoGene Ltd., East Brunswick, N.J., USA (Cat. No. CYT-254) and ThermoFisher Scientific, Inc., Waltham, Mass., USA (human IL-7 recombinant protein, Cat. No. Gibco PHC0071). The amino acid sequence of recombinant human IL-7 suitable for use in the invention is given in Table 2 (SEQ ID NO:6).

The term “IL-15” (also referred to herein as “IL15”) refers to the T cell growth factor known as interleukin-15, and includes all forms of IL-15 including human and mammalian forms, conservative amino acid substitutions, glycoforms, biosimilars, and variants thereof. IL-15 is described, e.g., in Fehniger and Caligiuri, Blood 2001, 97, 14-32, the disclosure of which is incorporated by reference herein. IL-15 shares β and γ signaling receptor subunits with IL-2. Recombinant human IL-15 is a single, non-glycosylated polypeptide chain containing 114 amino acids (and an N-terminal methionine) with a molecular mass of 12.8 kDa. Recombinant human IL-15 is commercially available from multiple suppliers, including ProSpec-Tany TechnoGene Ltd., East Brunswick, N.J., USA (Cat. No. CYT-230-b) and ThermoFisher Scientific, Inc., Waltham, Mass., USA (human IL-15 recombinant protein, Cat. No. 34-8159-82). The amino acid sequence of recombinant human IL-15 suitable for use in the invention is given in Table 2 (SEQ ID NO:7).

The term “IL-21” (also referred to herein as “IL21”) refers to the pleiotropic cytokine protein known as interleukin-21, and includes all forms of IL-21 including human and mammalian forms, conservative amino acid substitutions, glycoforms, biosimilars, and variants thereof. IL-21 is described, e.g., in Spolski and Leonard, Nat. Rev. Drug. Disc. 2014, 13, 379-95, the disclosure of which is incorporated by reference herein. IL-21 is primarily produced by natural killer T cells and activated human CD4⁺ T cells. Recombinant human IL-21 is a single, non-glycosylated polypeptide chain containing 132 amino acids with a molecular mass of 15.4 kDa. Recombinant human IL-21 is commercially available from multiple suppliers, including ProSpec-Tany TechnoGene Ltd., East Brunswick, N.J., USA (Cat. No. CYT-408-b) and ThermoFisher Scientific, Inc., Waltham, Mass., USA (human IL-21 recombinant protein, Cat. No. 14-8219-80). The amino acid sequence of recombinant human IL-21 suitable for use in the invention is given in Table 2 (SEQ ID NO:8).

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs, can also be incorporated into the described compositions and methods.

The terms “antibody” and its plural form “antibodies” refer to whole immunoglobulins and any antigen-binding fragment (“antigen-binding portion”) or single chains thereof. An “antibody” further refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions of an antibody may be further subdivided into regions of hypervariability, which are referred to as complementarity determining regions (CDR) or hypervariable regions (HVR), and which can be interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen epitope or epitopes. The constant regions of the antibodies may mediate the binding of the immunoglobulin to host tissues or factors, including various cells of the immune system (e.g., effector cells) and the first component (Clq) of the classical complement system.

The term “antigen” refers to a substance that induces an immune response. In some embodiments, an antigen is a molecule capable of being bound by an antibody or a TCR if presented by major histocompatibility complex (MHC) molecules. The term “antigen”, as used herein, also encompasses T cell epitopes. An antigen is additionally capable of being recognized by the immune system. In some embodiments, an antigen is capable of inducing a humoral immune response or a cellular immune response leading to the activation of B lymphocytes and/or T lymphocytes. In some cases, this may require that the antigen contains or is linked to a Th cell epitope. An antigen can also have one or more epitopes (e.g., B- and T-epitopes). In some embodiments, an antigen will preferably react, typically in a highly specific and selective manner, with its corresponding antibody or TCR and not with the multitude of other antibodies or TCRs which may be induced by other antigens.

The terms “monoclonal antibody,” “mAb,” “monoclonal antibody composition,” or their plural forms refer to a preparation of antibody molecules of single molecular composition. A monoclonal antibody composition displays a single binding specificity and affinity for a particular epitope. Monoclonal antibodies specific to certain receptors can be made using knowledge and skill in the art of injecting test subjects with suitable antigen and then isolating hybridomas expressing antibodies having the desired sequence or functional characteristics. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

The terms “antigen-binding portion” or “antigen-binding fragment” of an antibody (or simply “antibody portion” or “fragment”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and CH1 domains; (ii) a F(ab′)2 fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a domain antibody (dAb) fragment (Ward, et al., Nature, 1989, 341, 544-546), which may consist of a V_{H} or a V_{L} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv); see, e.g., Bird, et al., Science 1988, 242, 423-426; and Huston, et al., Proc. Natl. Acad. Sci. USA 1988, 85, 5879-5883). Such scFv antibodies are also intended to be encompassed within the terms “antigen-binding portion” or “antigen-binding fragment” of an antibody. These antibody fragments are obtained using conventional techniques known to those with skill in the art, and the fragments are screened for utility in the same manner as are intact antibodies.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). The term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In an embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (such as a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, including a conjugate of the antibody and another active pharmaceutical ingredient or antibody. The terms “conjugate,” “antibody-drug conjugate”, “ADC,” or “immunoconjugate” refers to an antibody, or a fragment thereof, conjugated to another therapeutic moiety, which can be conjugated to antibodies described herein using methods available in the art.

The terms “humanized antibody,” “humanized antibodies,” and “humanized” are intended to refer to antibodies in which CDR sequences derived from the germline of another mammalian species, such as a mouse, have been grafted onto human framework sequences. Additional framework region modifications may be made within the human framework sequences. Humanized forms of non-human (for example, murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a 15 hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones, et al., Nature 1986, 321, 522-525; Riechmann, et al., Nature 1988, 332, 323-329; and Presta, Curr. Op. Struct. Biol. 1992, 2, 593-596. The antibodies described herein may also be modified to employ any Fc variant which is known to impart an improvement (e.g., reduction) in effector function and/or FcR binding. The Fc variants may include, for example, any one of the amino acid substitutions disclosed in International Patent Application Publication Nos. WO 1988/07089 A1, WO 1996/14339 A1, WO 1998/05787 A1, WO 1998/23289 A1, WO 1999/51642 A1, WO 99/58572 A1, WO 2000/09560 A2, WO 2000/32767 A1, WO 2000/42072 A2, WO 2002/44215 A2, WO 2002/060919 A2, WO 2003/074569 A2, WO 2004/016750 A2, WO 2004/029207 A2, WO 2004/035752 A2, WO 2004/063351 A2, WO 2004/074455 A2, WO 2004/099249 A2, WO 2005/040217 A2, WO 2005/070963 A1, WO 2005/077981 A2, WO 2005/092925 A2, WO 2005/123780 A2, WO 2006/019447 A1, WO 2006/047350 A2, and WO 2006/085967 A2; and U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; and 7,083,784; the disclosures of which are incorporated by reference herein.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

A “diabody” is a small antibody fragment with two antigen-binding sites. The fragments comprises a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., European Patent No. EP 404,097, International Patent Publication No. WO 93/11161; and Bolliger, et al., Proc. Natl. Acad. Sci. USA 1993, 90, 6444-6448.

The term “glycosylation” refers to a modified derivative of an antibody. An aglycoslated antibody lacks glycosylation. Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Aglycosylation may increase the affinity of the antibody for antigen, as described in U.S. Pat. Nos. 5,714,350 and 6,350,861. Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8−/− cell lines were created by the targeted disruption of the FUT8 gene in CHO/DG44 cells using two replacement vectors (see e.g. U.S. Patent Publication No. 2004/0110704 or Yamane-Ohnuki, et al., Biotechnol. Bioeng., 2004, 87, 614-622). As another example, European Patent No. EP 1,176,195 describes a cell line with a functionally disrupted FUT8 gene, which encodes a fucosyl transferase, such that antibodies expressed in such a cell line exhibit hypofucosylation by reducing or eliminating the alpha 1,6 bond-related enzyme, and also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). International Patent Publication WO 03/035835 describes a variant CHO cell line, Lec 13 cells, with reduced ability to attach fucose to Asn(297)-linked carbohydrates, also resulting in hypofucosylation of antibodies expressed in that host cell (see also Shields, et al., J. Biol. Chem. 2002, 277, 26733-26740. International Patent Publication WO 99/54342 describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana, et al., Nat. Biotech. 1999, 17, 176-180). Alternatively, the fucose residues of the antibody may be cleaved off using a fucosidase enzyme. For example, the fucosidase alpha-L-fucosidase removes fucosyl residues from antibodies as described in Tarentino, et al., Biochem. 1975, 14, 5516-5523.

“Pegylation” refers to a modified antibody, or a fragment thereof, that typically is reacted with polyethylene glycol (PEG), such as a reactive ester or aldehyde derivative of PEG, under conditions in which one or more PEG groups become attached to the antibody or antibody fragment. Pegylation may, for example, increase the biological (e.g., serum) half life of the antibody. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀)alkoxy- or aryloxy-polyethylene glycol or polyethylene glycol-maleimide. The antibody to be pegylated may be an aglycosylated antibody. Methods for pegylation are known in the art and can be applied to the antibodies of the invention, as described for example in European Patent Nos. EP 0154316 and EP 0401384 and U.S. Pat. No. 5,824,778, the disclosures of each of which are incorporated by reference herein.

The terms “fusion protein” or “fusion polypeptide” refer to proteins that combine the properties of two or more individual proteins. Such proteins have at least two heterologous polypeptides covalently linked either directly or via an amino acid linker. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order and may include more than one of either or both of the constituent polypeptides. The term encompasses conservatively modified variants, polymorphic variants, alleles, mutants, subsequences, interspecies homologs, and immunogenic fragments of the antigens that make up the fusion protein. Fusion proteins of the disclosure can also comprise additional copies of a component antigen or immunogenic fragment thereof. The fusion protein may contain one or more binding domains linked together and further linked to an Fc domain, such as an IgG Fc domain. Fusion proteins may be further linked together to mimic a monoclonal antibody and provide six or more binding domains. Fusion proteins may be produced by recombinant methods as is known in the art. Preparation of fusion proteins are known in the art and are described, e.g., in International Patent Application Publication Nos. WO 1995/027735 A1, WO 2005/103077 A1, WO 2008/025516 A1, WO 2009/007120 A1, WO 2010/003766 A1, WO 2010/010051 A1, WO 2010/078966 A1, U.S. Patent Application Publication Nos. US 2015/0125419 A1 and US 2016/0272695 A1, and U.S. Pat. No. 8,921,519, the disclosures of each of which are incorporated by reference herein.

The term “heterologous” when used with reference to portions of a nucleic acid or protein indicates that the nucleic acid or protein comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source, or coding regions from different sources. Similarly, a heterologous protein indicates that the protein comprises two or more subsequences that are not found in the same relationship to each other in nature (e.g., a fusion protein).

The term “conservative amino acid substitutions” in means amino acid sequence modifications which do not abrogate the binding of an antibody or fusion protein to the antigen. Conservative amino acid substitutions include the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution. Thus, a predicted nonessential amino acid residue in an antibody is preferably replaced with another amino acid residue from the same class. Methods of identifying amino acid conservative substitutions which do not eliminate antigen binding are well-known in the art (see, e.g., Brummell, et al., Biochemistry 1993, 32, 1180-1187; Kobayashi, et al., Protein Eng. 1999, 12, 879-884 (1999); and Burks, et al., Proc. Natl. Acad. Sci. USA 1997, 94, 412-417.

The terms “sequence identity,” “percent identity,” and “sequence percent identity” (or synonyms thereof, e.g., “99% identical”) in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. Suitable programs to determine percent sequence identity include for example the BLAST suite of programs available from the U.S. Government's National Center for Biotechnology Information BLAST web site. Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, Calif.) or MegAlign, available from DNASTAR, are additional publicly available software programs that can be used to align sequences. One skilled in the art can determine appropriate parameters for maximal alignment by particular alignment software. In certain embodiments, the default parameters of the alignment software are used.

As used herein, the term “variant” encompasses but is not limited to antibodies or fusion proteins which comprise an amino acid sequence which differs from the amino acid sequence of a reference antibody by way of one or more substitutions, deletions and/or additions at certain positions within or adjacent to the amino acid sequence of the reference antibody. The variant may comprise one or more conservative substitutions in its amino acid sequence as compared to the amino acid sequence of a reference antibody. Conservative substitutions may involve, e.g., the substitution of similarly charged or uncharged amino acids. The variant retains the ability to specifically bind to the antigen of the reference antibody. The term variant also includes pegylated antibodies or proteins.

Nucleic acid sequences implicitly encompass conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues. Batzer, et al., Nucleic Acid Res. 1991, 19, 5081; Ohtsuka, et al., J. Biol. Chem. 1985, 260, 2605-2608; Rossolini, et al., Mol. Cell. Probes 1994, 8, 91-98. The term nucleic acid is used interchangeably with cDNA, mRNA, oligonucleotide, and polynucleotide.

The term “biosimilar” means a biological product, including a monoclonal antibody or protein, that is highly similar to a U.S. licensed reference biological product notwithstanding minor differences in clinically inactive components, and for which there are no clinically meaningful differences between the biological product and the reference product in terms of the safety, purity, and potency of the product. Furthermore, a similar biological or “biosimilar” medicine is a biological medicine that is similar to another biological medicine that has already been authorized for use by the European Medicines Agency. The term “biosimilar” is also used synonymously by other national and regional regulatory agencies. Biological products or biological medicines are medicines that are made by or derived from a biological source, such as a bacterium or yeast. They can consist of relatively small molecules such as human insulin or erythropoietin, or complex molecules such as monoclonal antibodies. For example, if the reference IL-2 protein is aldesleukin (PROLEUKIN), a protein approved by drug regulatory authorities with reference to aldesleukin is a “biosimilar to” aldesleukin or is a “biosimilar thereof” of aldesleukin. In Europe, a similar biological or “biosimilar” medicine is a biological medicine that is similar to another biological medicine that has already been authorized for use by the European Medicines Agency (EMA). The relevant legal basis for similar biological applications in Europe is Article 6 of Regulation (EC) No 726/2004 and Article 10(4) of Directive 2001/83/EC, as amended and therefore in Europe, the biosimilar may be authorized, approved for authorization or subject of an application for authorization under Article 6 of Regulation (EC) No 726/2004 and Article 10(4) of Directive 2001/83/EC. The already authorized original biological medicinal product may be referred to as a “reference medicinal product” in Europe. Some of the requirements for a product to be considered a biosimilar are outlined in the CHMP Guideline on Similar Biological Medicinal Products. In addition, product specific guidelines, including guidelines relating to monoclonal antibody biosimilars, are provided on a product-by-product basis by the EMA and published on its website. A biosimilar as described herein may be similar to the reference medicinal product by way of quality characteristics, biological activity, mechanism of action, safety profiles and/or efficacy. In addition, the biosimilar may be used or be intended for use to treat the same conditions as the reference medicinal product. Thus, a biosimilar as described herein may be deemed to have similar or highly similar quality characteristics to a reference medicinal product. Alternatively, or in addition, a biosimilar as described herein may be deemed to have similar or highly similar biological activity to a reference medicinal product. Alternatively, or in addition, a biosimilar as described herein may be deemed to have a similar or highly similar safety profile to a reference medicinal product. Alternatively, or in addition, a biosimilar as described herein may be deemed to have similar or highly similar efficacy to a reference medicinal product. As described herein, a biosimilar in Europe is compared to a reference medicinal product which has been authorized by the EMA. However, in some instances, the biosimilar may be compared to a biological medicinal product which has been authorized outside the European Economic Area (a non-EEA authorized “comparator”) in certain studies. Such studies include for example certain clinical and in vivo non-clinical studies. As used herein, the term “biosimilar” also relates to a biological medicinal product which has been or may be compared to a non-EEA authorized comparator. Certain biosimilars are proteins such as antibodies, antibody fragments (for example, antigen binding portions) and fusion proteins. A protein biosimilar may have an amino acid sequence that has minor modifications in the amino acid structure (including for example deletions, additions, and/or substitutions of amino acids) which do not significantly affect the function of the polypeptide. The biosimilar may comprise an amino acid sequence having a sequence identity of 97% or greater to the amino acid sequence of its reference medicinal product, e.g., 97%, 98%, 99% or 100%. The biosimilar may comprise one or more post-translational modifications, for example, although not limited to, glycosylation, oxidation, deamidation, and/or truncation which is/are different to the post-translational modifications of the reference medicinal product, provided that the differences do not result in a change in safety and/or efficacy of the medicinal product. The biosimilar may have an identical or different glycosylation pattern to the reference medicinal product. Particularly, although not exclusively, the biosimilar may have a different glycosylation pattern if the differences address or are intended to address safety concerns associated with the reference medicinal product. Additionally, the biosimilar may deviate from the reference medicinal product in for example its strength, pharmaceutical form, formulation, excipients and/or presentation, providing safety and efficacy of the medicinal product is not compromised. The biosimilar may comprise differences in for example pharmacokinetic (PK) and/or pharmacodynamic (PD) profiles as compared to the reference medicinal product but is still deemed sufficiently similar to the reference medicinal product as to be authorized or considered suitable for authorization. In certain circumstances, the biosimilar exhibits different binding characteristics as compared to the reference medicinal product, wherein the different binding characteristics are considered by a Regulatory Authority such as the EMA not to be a barrier for authorization as a similar biological product. The term “biosimilar” is also used synonymously by other national and regional regulatory agencies.

The term “hematological malignancy” refers to mammalian cancers and tumors of the hematopoietic and lymphoid tissues, including but not limited to tissues of the blood, bone marrow, lymph nodes, and lymphatic system. Hematological malignancies may result in the formation of a “liquid tumor.” Hematological malignancies include, but are not limited to, acute lymphoblastic leukemia (ALL), chronic lymphocytic lymphoma (CLL), small lymphocytic lymphoma (SLL), acute myeloid leukemia (AML), chronic myelogenous leukemia (CML), acute monocytic leukemia (AMoL), Hodgkin's lymphoma, and non-Hodgkin's lymphomas. The term “B cell hematological malignancy” refers to hematological malignancies that affect B cells.

The term “liquid tumor” refers to an abnormal mass of cells that is fluid in nature. Liquid tumor cancers include, but are not limited to, leukemias, myelomas, and lymphomas, as well as other hematological malignancies. TILs obtained from liquid tumors, including liquid tumors resident in bone marrow, may also be referred to herein as marrow infiltrating lymphocytes (MILs). TILs obtained from liquid tumors, including liquid tumors circulating in peripheral blood, may also be referred to herein as PBLs. The terms MIL, TIL, and PBL are used interchangeably herein and differ only based on the tissue type from which the cells are derived.

The term “biopsy” refers to any medical procedure used to obtain cancerous cells, including bone marrow biopsy.

The terms “acute myeloid leukemia” or “AML” refers to cancers of the myeloid blood cell lines, which are also known in the art as acute myelogenous leukemia and acute nonlymphocytic leukemia. Although AML is a liquid tumor, some manifestations of AML, including extramedullary manifestations such as chloroma, exhibit properties of a solid tumor, but are classified herein as a liquid tumor.

The term “microenvironment,” as used herein, may refer to the solid or hematological tumor microenvironment as a whole or to an individual subset of cells within the microenvironment. The tumor microenvironment, as used herein, refers to a complex mixture of “cells, soluble factors, signaling molecules, extracellular matrices, and mechanical cues that promote neoplastic transformation, support tumor growth and invasion, protect the tumor from host immunity, foster therapeutic resistance, and provide niches for dominant metastases to thrive,” as described in Swartz, et al., Cancer Res., 2012, 72, 2473. Although tumors express antigens that should be recognized by T cells, tumor clearance by the immune system is rare because of immune suppression by the microenvironment.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “treatment”, “treating”, “treat”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

By “tumor infiltrating lymphocytes” or “TILs” herein is meant a population of cells originally obtained as white blood cells that have left the bloodstream of a subject and migrated into a tumor. TILs include, but are not limited to, CD8+ cytotoxic T cells (lymphocytes), Th1 and Th17 CD4+ T cells, natural killer cells, dendritic cells and M1 macrophages. TILs include both primary and secondary TILs. “Primary TILs” are those that are obtained from patient tissue samples as outlined herein (sometimes referred to as “freshly harvested”), and “secondary TILs” are any TIL cell populations that have been expanded or proliferated as discussed herein, including, but not limited to bulk TILs, expanded TILs (“REP TILs”) as well as “reREP TILs” as discussed herein.

TILs can generally be defined either biochemically, using cell surface markers, or functionally, by their ability to infiltrate tumors and effect treatment. TILs can be generally categorized by expressing one or more of the following biomarkers: CD4, CD8, TCR αβ, CD27, CD28, CD56, CCR7, CD45Ra, CD95, PD-1, and CD25. Additionally and alternatively, TILs can be functionally defined by their ability to infiltrate solid tumors upon reintroduction into a patient. TILS may further be characterized by potency—for example, TILS may be considered potent if, for example, interferon (IFN) release is greater than about 50 pg/mL, greater than about 100 pg/mL, greater than about 150 pg/mL, or greater than about 200 pg/mL.

By “cryopreserved TILs” (or cryopreserved MILs or PBLs) herein is meant that TILs, either primary, bulk, or expanded (REP TILs), are treated and stored in the range of about −150° C. to −60° C. General methods for cryopreservation are also described elsewhere herein, including in the Examples. For clarity, “cryopreserved TILs” are distinguishable from frozen tissue samples which may be used as a source of primary TILs.

By “thawed cryopreserved TILs” (or thawed MILs or PBLs) herein is meant a population of TILs that was previously cryopreserved and then treated to return to room temperature or higher, including but not limited to cell culture temperatures or temperatures wherein TILs may be administered to a patient.

By “population of cells” (including TILs) herein is meant a number of cells that share common traits. In general, populations generally range from 1×10⁶ to 1×10¹⁰ in number, with different TIL populations comprising different numbers. For example, initial growth of primary TILs in the presence of IL-2 results in a population of bulk TILs of roughly 1×10⁸ cells. REP expansion is generally done to provide populations of 1.5×10⁹ to 1.5×10¹⁰ cells for infusion.

In general, TILs are initially obtained from a patient tumor sample (“primary TILs”) and then expanded into a larger population for further manipulation as described herein, optionally cyropreserved, restimulated as outlined herein and optionally evaluated for phenotype and metabolic parameters as an indication of TIL health.

In general, the harvested cell suspension is called a “primary cell population” or a “freshly harvested” cell population.

In general, as discussed herein, the TILs are initially prepared by obtaining a primary population of TILs from a tumor resected from a patient as discussed herein (the “primary cell population” or “first cell population”). This is followed with an initial bulk expansion utilizing a culturing of the cells with IL-2, forming a second population of cells (sometimes referred to herein as the “bulk TIL population” or “second population”).

The term “cytotoxic lymphocyte” includes cytotoxic T (CTL) cells (including CD8⁺ cytotoxic T lymphocytes and CD4⁺ T-helper lymphocytes), natural killer T (NKT) cells and natural killer (NK) cells. Cytotoxic lymphocytes can include, for example, peripheral blood-derived αβ TCR-positive or γδ TCR-positive T cells activated by tumor associated antigens and/or transduced with tumor specific chimeric antigen receptors or T-cell receptors, and tumor-infiltrating lymphocytes (TILs).

The term “central memory T cell” refers to a subset of T cells that in the human are CD45RO+ and constitutively express CCR7 (CCR7h i) and CD62L (CD62 hi). The surface phenotype of central memory T cells also includes TCR, CD3, CD127 (IL-7R), and IL-15R. Transcription factors for central memory T cells include BCL-6, BCL-6B, MBD2, and BMII. Central memory T cells primarily secret IL-2 and CD40L as effector molecules after TCR triggering. Central memory T cells are predominant in the CD4 compartment in blood, and in the human are proportionally enriched in lymph nodes and tonsils.

The term “effector memory T cell” refers to a subset of human or mammalian T cells that, like central memory T cells, are CD45R0+, but have lost the constitutive expression of CCR7 (CCR7lo) and are heterogeneous or low for CD62L expression (CD62Llo). The surface phenotype of central memory T cells also includes TCR, CD3, CD127 (IL-7R), and IL-15R. Transcription factors for central memory T cells include BLIMP1. Effector memory T cells rapidly secret high levels of inflammatory cytokines following antigenic stimulation, including interferon-γ, IL-4, and IL-5. Effector memory T cells are predominant in the CD8 compartment in blood, and in the human are proportionally enriched in the lung, liver, and gut. CD8+ effector memory T cells carry large amounts of perforin. The term “closed system” refers to a system that is closed to the outside environment. Any closed system appropriate for cell culture methods can be employed with the methods of the present invention. Closed systems include, for example, but are not limited to closed G-containers. Once a tumor segment is added to the closed system, the system is no opened to the outsside environment until the TILs are ready to be administered to the patient.

In some embodiments, methods of the present disclosure further include a “pre-REP” stage in which tumor tissue or cells from tumor tissue are grown in standard lab media (including without limitation RPMI) and treated the with reagents such as irradiated feeder cells and anti-CD3 antibodies to achieve a desired effect, such as increase in the number of TILS and/or an enrichment of the population for cells containing desired cell surface markers or other structural, biochemical or functional features. The pre-REP stage may utilize lab grade reagents (under the assumption that the lab grade reagents get diluted out during a later REP stage), making it easier to incorporate alternative strategies for improving TIL production. Therefore, in some embodiments, the disclosed TLR agonist and/or peptide or peptidomimetics can be included in the culture medium during the pre-REP stage. The pre-REP culture can in some embodiments, include IL-2.The present invention is directed in preferred aspects to novel methods of augmenting REPs with one or more additional restimulation protocols, also referred to herein as a “restimulation Rapid Expansion Protocol” or “reREP”, which leads surprisingly to expanded memory T cell subsets, including the memory effector T cell subset, and/or to markes enhancement in the glycolytic respiration as compared to freshly harvested TILs or thawed cryopreserved TILs for the restimulated TILs (sometimes referred to herein as “reTILs”). That is, by using a reREP procedure on cyropreserved TILs, patients can receive highly metabolically active, healthy TILs, leading to more favorable outcomes.

When “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the genetically modified cytotoxic lymphocytes described herein may be administered at a dosage of 10⁴ to 10¹¹ cells/kg body weight (e.g., 10⁵ to 10⁶, 10⁵ to 10¹⁰, 10⁵ to 10¹¹, 10⁶ to 10¹⁰, 10⁶ to 10¹¹, 10⁷ to 10¹¹, 10⁷ to 10¹⁰, 10⁸ to 10¹¹, 10⁸ to 10¹⁰, 10⁹ to 10¹¹, or 10⁹ to 10¹⁰ cells/kg body weight), including all integer values within those ranges. Genetically modified cytotoxic lymphocytes compositions may also be administered multiple times at these dosages. The genetically modified cytotoxic lymphocytes can be administered by using infusion techniques that are commonly known in immunotherapy (see, e.g., Rosenberg et al., New Eng. J. of Med. 319: 1676, 1988). The optimal dosage and treatment regime for a particular patient can readily be determined by one skilled in the art of medicine by monitoring the patient for signs of disease and adjusting the treatment accordingly.

For the avoidance of doubt, it is intended herein that particular features (for example integers, characteristics, values, uses, diseases, formulae, compounds or groups) described in conjunction with a particular aspect, embodiment or example of the invention are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any disclosed embodiments. The invention extends to any novel one, or novel combination, of the features disclosed in this specification (including any accompanying claims, abstract and drawings), or to any novel one, or any novel combination, of the steps of any method or process so disclosed.

The terms “about” and “approximately” mean within a statistically meaningful range of a value. Such a range can be within an order of magnitude, preferably within 50%, more preferably within 20%, more preferably still within 10%, and even more preferably within 5% of a given value or range. The allowable variation encompassed by the terms “about” or “approximately” depends on the particular system under study, and can be readily appreciated by one of ordinary skill in the art. Moreover, as used herein, the terms “about” and “approximately” mean that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All compositions, methods, and kits described herein that embody the present invention can, in alternate embodiments, be more specifically defined by any of the transitional terms “comprising,” “consisting essentially of,” and “consisting of.”

### Embodiments of Methods of Expanding Therapeutic T-Cells Including Peripheral Blood (PBLs) and/or Bone Marrow (MILs)

Methods of Expanding Peripheral Blood Lymphocytes (PBLs) from Peripheral Blood

PBL Method 1. In an embodiment of the invention, PBLs are expanded using the processes described herein. In an embodiment of the invention, the method comprises obtaining a PBMC sample from whole blood. In an embodiment, the method comprises enriching T-cells by isolating pure T-cells from PBMCs using negative selection of a non-CD19+ fraction. On Day 0, the pure T-cells are cultured with antiCD3/antiCD28 antibodies (DynaBeads®) in a 1:1 ratio (beads:cells) and IL-2 at 3000 IU/ml. On Day 4, additional IL-2 is added to the culture at 3000 IU/ml. On Day 7, the culture is again stimulated with antiCD3/antiCD28 antibodies (DynaBeads®) in a 1:1 ratio (beads:cells), and additional IL-2 at 3000 IU/ml is added to the culture. PBLs are harvested on Day 14, beads are removed, and PBLs are counted and phenotyped. In an embodiment, the method comprises enriching T-cells by isolating pure T-cells from PBMCs using magnetic bead-based negative selection of a non-CD19+ fraction.

In an embodiment of the invention, PBL Method 1 is performed as follows: On Day 0, a cryopreserved PBMC sample is thawed and PBMCs are counted. T-cells are isolated using a Human Pan T-Cell Isolation Kit and LS columns (Miltenyi Biotec). The isolated T cells are counted and seeded at 5×10⁵ cells per well of a GRex 24-well plate and are co-cultured with DynaBeads® (anti-CD3/anti-CD28) at a 1:1 ratio with IL-2 at 3000 IU/ml in a total of 8 ml of CM2 media per well. On Day 4, the media in each well is exchanged from CM2 to AIM-V with fresh IL-2 at 3000 IU/ml. On Day 7, the expanded cells are harvested, counted, then cultured at 15×10⁶ cells per flask in GRex I0M flasks with IL-2 at 3000 IU/ml and DynaBeads® at a 1:1 ratio (beads:cells) in a total of 100 ml AIM-V media. On Day 11, the media is exchanged to CM-4 media supplemented with fresh IL-2 at 3000 IU/ml. On Day 14, the DynaBeads are removed using a DynaMag Magnet (DynaMag™-15) and the cells are counted.

In an embodiment of the invention, PBL Method 1 is performed as follows: On Day 0, a cryopreserved PBMC sample is thawed and PBMCs are counted. T-cells are isolated using a Human Pan T-Cell Isolation Kit and LS columns (Miltenyi Biotec). The isolated T cells are counted and seeded at 5×10⁵ cells per well of a GRex 24-well plate and are co-cultured with DynaBeads® (anti-CD3/anti-CD28) at a 1:1 ratio with IL-2 at 3000 IU/ml in a total of 8 ml of CM2 media per well. On Day 4, the media in each well is exchanged from CM2 to AIM-V with fresh IL-2 at 3000 IU/ml. On Day 7, the PBLs are harvested, counted, then reseeded at 1×10⁶ cells per well of a new GRex-24 well plate with IL-2 at 3000 IU/ml and DynaBeads® at a 1:1 ratio (beads:cells) in a total of 8 ml AIM-V media. On Day 11, the media is exchanged to CM-4 media supplemented with fresh IL-2 at 3000 IU/ml. On Day 14, the DynaBeads® are removed using a DynaMag Magnet (DynaMag™-15) and the cells are counted.

PBL Method 2. In an embodiment of the invention, PBLs are expanded using PBL Method 2, which comprises obtaining a PBMC sample from whole blood. The T-cells from the PBMCs are enriched by incubating the PBMCs for at least three hours at 37° C. and then isolating the non-adherent cells. The non-adherent cells are the expanded similarly as PBL Method 1, that is, on Day 0, the non-adherent cells are cultured with antiCD3/antiCD28 antibodies (DynaBeads®) in a 1:1 ratio (beads:cells) and IL-2 at 3000 IU/ml. On Day 4, additional IL-2 is added to the culture at 3000 IU/ml. On Day 7, the culture is again stimulated with antiCD3/antiCD28 antibodies (DynaBeads®) in a 1:1 ratio (beads:cells), and additional IL-2 at 3000 IU/ml is added to the culture. PBLs are harvested on Day 14, beads are removed, and PBLs are counted and phenotyped.

In an embodiment of the invention, PBL Method 2 is performed as follows: On Day 0, the cryopreserved PMBC sample is thawed and the PBMC cells are seeded at 6 million cells per well in a 6 well plate in CM-2 media and incubated for 3 hours at 37 degrees Celsius. After 3 hours, the non-adherent cells, which are the PBLs, are removed and counted. The PBLs are cultured with anti-CD3/anti-CD28 DynaBeads® in a 1:1 ratio of beads:cells, at 1×10⁶ cells per well and IL-2 at 3000 IU/ml in a total of 7 ml of CM-2 media in each well of a GRex 24-well plate. On Day 4, the media in each well is exchanged with AIM-V media and fresh IL-2 at 3000 IU/ml. On Day 7, the expanded cells are harvested, counted, then cultured at 15×10⁶ cells per flask in GRex I0M flasks with IL-2 at 3000 IU/ml and DynaBeads at a 1:1 ratio (T-cells:beads) in a total of 100 ml AIM-V media. On Day 11, the media is changed to CM-4 media and supplemented with fresh IL-2 (3000 IU/ml). On Day 14, the DynaBeads are removed using a DynaMag™ Magnet (DynaMag™-15) and the cells are counted.

In an embodiment of the invention, PBL Method 2 is performed as follows: On Day 0, the cryopreserved PMBC sample is thawed and the PBMC cells are seeded at 6 million cells per well in a 6 well plate in CM-2 media and incubated for 3 hours at 37 degrees Celsius. After 3 hours, the non-adherent cells, which are the PBLs, are removed and counted. The PBLs are cultured with anti-CD3/anti-CD28 DynaBeads® in a 1:1 ratio of beads:cells, at 1×10⁶ cells per well and IL-2 at 3000 IU/ml in a total of 7 ml of CM-2 media in each well of a GRex 24-well plate. On Day 4, the media in each well is exchanged with AIM-V media and fresh IL-2 at 3000 IU/ml. On Day 7, the expanded cells are harvested, counted, then cultured at 1×10⁶ cells per well in a new GRex 24-well plate with IL-2 at 3000 IU/ml and DynaBeads® at a 1:1 ratio (T-cells:beads) in a total of 8 ml AIM-V media. On Day 11, the media is changed to CM-4 media and supplemented with fresh IL-2 (3000 IU/ml). On Day 14, the DynaBeads are removed using a DynaMag™ Magnet (DynaMag™-15) and the cells are counted.

PBL Method 3. In an embodiment of the invention, PBLs are expanded using PBL Method 3, which comprises obtaining a PBMC sample from peripheral blood. B-cells are isolated using a CD19+ selection and T-cells are selected using negative selection of the non-CD19+ fraction of the PBMC sample. On Day 0, the T-cells and B-cells are co-cultured with antiCD3/antiCD28 antibodies (DynaBeads®) in a 1:1 ratio (beads:cells) and IL-2 at 3000 IU/ml. On Day 4, additional IL-2 is added to the culture at 3000 IU/ml. On Day 7, the culture is again stimulated with antiCD3/antiCD28 antibodies (DynaBeads®) in a 1:1 ratio (beads:cells), and additional IL-2 at 3000 IU/ml is added to the culture. PBLs are harvested on Day 14, beads are removed, and PBLs are counted and phenotyped.

In an embodiment of the invention, PBL Method 3 is performed as follows: On Day 0, cryopreserved PBMCs derived from peripheral blood are thawed and counted. CD19+ B-cells are sorted using a CD19 Multisort Kit, Human (Miltenyi Biotec). Of the non-CD19+ cell fraction, T-cells are purified using the Human Pan T-cell Isolation Kit and LS Columns (Miltenyi Biotec). The T-cells (PBLs) and B-cells are co-cultured at different ratios in a Grex 24-well plate in about 8 ml of CM2 media in the presence of IL-2 at about 3000 IU/ml. B-cell:T-cell ratios are 0.1:1; 1:1, and 10:1. The T-cell/B-cell co-culture is stimulated with antiCD3/antiCD28 antibodies (DynaBeads®) in a 1:1 ratio (beads:cells). On Day 4, the media is exchanged from CM2 to AIM-V media and additional IL-2 is added to the culture at 3000 IU/ml. On Day 7, the cells are harvested and counted and re-seeded on a new Grex 24-well plate in AIM-V media at a cell range of from about 1.5×10⁵ to about 4×10⁵ cells per well and stimulated with antiCD3/antiCD28 antibodies (DynaBeads®) in a 1:1 ratio (beads:cells), with additional IL-2 at 3000 IU/ml. On Day 14, the DynaBeads are removed using a DynaMag™ Magnet (DynaMag™-15) and the cells are counted.

In an embodiment, PBMCs are isolated from a whole blood sample. In an embodiment, the PBMC sample is used as the starting material to expand the PBLs. In an embodiment, the sample is cryopreserved prior to the expansion process. In another embodiment, a fresh sample is used as the starting material to expand the PBLs. In an embodiment of the invention, T-cells are isolated from PBMCs using methods known in the art. In an embodiment, the T-cells are isolated using a Human Pan T-cell isolation kit and LS columns. In an embodiment of the invention, T-cells are isolated from PBMCs using antibody selection methods known in the art, for example, CD19 negative selection.

In an embodiment of the invention, the process is performed over about 7 days, about 8 days, about 9 days, about 10 days, about 11 days, about 12 days, about 13 days, or about 14 days. In another embodiment, the process is performed over about 7 days. In another embodiment, the process is performed over about 14 days.

In an embodiment of the invention, the PBMCs are cultured with antiCD3/antiCD28 antibodies. In an embodiment, any available antiCD3/antiCD28 product is useful in the present invention. In an embodiment of the invention, the commercially available product used are DynaBeads®. In an embodiment, the DynaBeads® are cultured with the PBMCs in a ratio of 1:1 (beads:cells). In another embodiment, the antibodies are DynaBeads® cultured with the PBMCs in a ratio of 1.5:1, 2:1, 2.5:1, 3:1, 3.5:1, 4:1, 4.5:1, or 5:1 (beads:cells). In an embodiment of the invention, the antibody culturing steps and/or the step of restimulating cells with antibody is performed over a period of from about 2 to about 6 days, from about 3 to about 5 days, or for about 4 days. In an embodiment of the invention, the antibody culturing step is performed over a period of about 2 days, 3 days, 4 days, 5 days, or 6 days.

In an embodiment, the PBMC sample is cultured with IL-2. In an embodiment of the invention, the cell culture medium used for expansion of the PBLs from PBMCs comprises IL-2 at a concentration selected from the group consisting of about 100 IU/mL, about 200 IU/mL, about 300 IU/mL, about 400 IU/mL, about 100 IU/mL, about 100 IU/mL, about 100 IU/mL, about 100 IU/mL, about 100 IU/mL, about 500 IU/mL, about 600 IU/mL, about 700 IU/mL, about 800 IU/mL, about 900 IU/mL, about 1,000 IU/mL, about 1,100 IU/mL, about 1,200 IU/mL, about 1,300 IU/mL, about 1,400 IU/mL, about 1,500 IU/mL, about 1,600 IU/mL, about 1,700 IU/mL, about 1,800 IU/mL, about 1,900 IU/mL, about 2,000 IU/mL, about 2,100 IU/mL, about 2,200 IU/mL, about 2,300 IU/mL, about 2,400 IU/mL, about 2,500 IU/mL, about 2,600 IU/mL, about 2,700 IU/mL, about 2,800 IU/mL, about 2,900 IU/mL, about 3,000 IU/mL, about 3,100 IU/mL, about 3,200 IU/mL, about 3,300 IU/mL, about 3,400 IU/mL, about 3,500 IU/mL, about 3,600 IU/mL, about 3,700 IU/mL, about 3,800 IU/mL, about 3,900 IU/mL, about 4,000 IU/mL, about 4,100 IU/mL, about 4,200 IU/mL, about 4,300 IU/mL, about 4,400 IU/mL, about 4,500 IU/mL, about 4,600 IU/mL, about 4,700 IU/mL, about 4,800 IU/mL, about 4,900 IU/mL, about 5,000 IU/mL, about 5,100 IU/mL, about 5,200 IU/mL, about 5,300 IU/mL, about 5,400 IU/mL, about 5,500 IU/mL, about 5,600 IU/mL, about 5,700 IU/mL, about 5,800 IU/mL, about 5,900 IU/mL, about 6,000 IU/mL, about 6,500 IU/mL, about 7,000 IU/mL, about 7,500 IU/mL, about 8,000 IU/mL, about 8,500 IU/mL, about 9,000 IU/mL, about 9,500 IU/mL, and about 10,000 IU/mL.

In an embodiment of the invention, the starting cell number of PBMCs for the expansion process is from about 25,000 to about 1,000,000, from about 30,000 to about 900,000, from about 35,000 to about 850,000, from about 40,000 to about 800,000, from about 45,000 to about 800,000, from about 50,000 to about 750,000, from about 55,000 to about 700,000, from about 60,000 to about 650,000, from about 65,000 to about 600,000, from about 70,000 to about 550,000, preferably from about 75,000 to about 500,000, from about 80,000 to about 450,000, from about 85,000 to about 400,000, from about 90,000 to about 350,000, from about 95,000 to about 300,000, from about 100,000 to about 250,000, from about 105,000 to about 200,000, or from about 110,000 to about 150,000. In an embodiment of the invention, the starting cell number of PBMCs is about 138,000, 140,000, 145,000, or more. In another embodiment, the starting cell number of PBMCs is about 28,000. In another embodiment, the starting cell number of PBMCs is about 62,000. In another embodiment, the starting cell number of PBMCs is about 338,000. In another embodiment, the starting cell number of PBMCs is about 336,000. In another embodiment, the starting cell number of PBMCs is 1 million, 2 million, 3 million, 4 million, 5 million, 6 million, 7 million, 8 million, 9 million, 10 million or more. In another embodiment, the starting cell number of PBMCs is 1 million to 10 million, 2 million to 9 million, 3 million to 8 million, 4 million to 7 million, or 5 million to 6 million. In another embodiment, the starting cell number of PBMCs is about 4 million. In yet another embodiment, the starting cell number of PBMCs is at least about 4 million, at least about 5 million, or at least about 6 million or more.

In an embodiment of the invention, the cells are grown in a GRex 24 well plate. In an embodiment of the invention, a comparable well plate is used. In an embodiment, the starting material for the expansion is about 5×10⁵ T-cells per well. In an embodiment of the invention, there are 1×10⁶ cells per well. In an embodiment of the invention, the number of cells per well is sufficient to seed the well and expand the T-cells.

In an embodiment of the invention, the fold expansion of PBLs is from about 20% to about 100%, 25% to about 95%, 30% to about 90%, 35% to about 85%, 40% to about 80%, 45% to about 75%, 50% to about 100%, or 25% to about 75%. In an embodiment of the invention, the fold expansion is about 25%. In another embodiment of the invention, the fold expansion is about 50%. In another embodiment, the fold expansion is about 75%.

In an embodiment of the invention, additional IL-2 may be added to the culture on one or more days throughout the process. In an embodiment of the invention, additional IL-2 is added on Day 4. In an embodiment of the invention, additional IL-2 is added on Day 7. In an embodiment of the invention, additional IL-2 is added on Day 11. In an other embodiment, additional IL-2 is added on Day 4, Day 7, and/or Day 11. In an embodiment of the invention, the cell culture medium may be changed on one or more days through the cell culture process. In an embodiment, the cell culture medium is changed on Day 4, Day 7, and/or Day 11 of the process. In an embodiment of the invention, the PBLs are cultured with additional IL-2 for a period of 1 day, 2 days, 3 days, 4 days, 5 days, 6 days, 7 days, 8 days, 9 days, 10 days, 11 days, 12 days, 13 days, or 14 days. In an embodiment of the invention, PBLs are cultured for a period of 3 days after each addition of IL-2.

In an embodiment, the cell culture medium is exchanged at least once time during the method. In an embodiment, the cell culture medium is exchanged at the same time that additional IL-2 is added. In another embodiment the cell culture medium is exchanged on at least one of Day 1, Day 2, Day 3, Day 4, Day 5, Day 6, Day 7, Day 8, Day 9, Day 10, Day 11, Day 12, Day 13, or Day 14. In an embodiment of the invention, the cell culture medium used throughout the method may be the same or different. In an embodiment of the invention, the cell culture medium is CM-2, CM-4, or AIM-V.

In an embodiment of the invention, T-cells may be restimulated with antiCD3/antiCD28 antibodies on one or more days throughout the 14-day expansion process. In an embodiment, the T-cells are restimulated on Day 7. In an embodiment, GRex 10M flasks are used for the restimulation step. In an embodiment of the invention, comparable flasks are used.

In an embodiment of the invention, the DynaBeads are removed using a DynaMag™ Magnet, the cells are counted, and the cells are analyzed using phenotypic and functional analysis as further described in the Examples below. In an embodiment of the invention, antibodies are separated from the PBLs or MILs using methods known in the art. In any of the foregoing embodiments, magnetic bead-based selection of TILs, PBLs, or MILs is used.

In an embodiment of the invention, the PBMC sample is incubated for a period of time at a desired temperature effective to identify the non-adherent cells. In an embodiment of the invention, the incubation time is about 3 hours. In an embodiment of the invention, the temperature is about 37° Celsius. The non-adherent cells are then expanded using the process described above.

In an embodiment of the invention, the PBMCs are obtained from a patient who has been treated with ibrutinib or another ITK or kinase inhibitor, such ITK and kinase inhibitors as described elsewhere herein. In an embodiment of the invention, the ITK inhibitor is a covalent ITK inhibitor that covalently and irreversibly binds to ITK. In an embodiment of the invention, the ITK inhibitor is an allosteric ITK inhibitor that binds to ITK. In an embodiment of the invention, the PBMCs are obtained from a patient who has been treated with ibrutinib or other ITK inhibitor, including ITK inhibitors as described elsewhere herein, prior to obtaining a PBMC sample for use with any of the foregoing methods, including PBL Method 1, PBL Method 2, or PBL Method 3. In an embodiment of the invention, the ITK inhibitor treatment has been administered at least 1 time, at least 2, times, or at least 3 times or more. In an embodiment of the invention, PBLs that are expanded from patients pretreated with ibrutinib or other ITK inhibitor comprise less LAG3+, PD-1+ cells than those expanded from patients not pretreated with ibrutinib or other ITK inhibitor. In an embodiment of the invention PBLs that are expanded from patients pretreated with ibrutinib or other ITK inhibitor comprise increased levels of IFNγ production than those expanded from patients not pretreated with ibrutinib or other ITK inhibitor. In an embodiment of the invention, PBLs that are expanded from patients pretreated with ibrutinib or other ITK inhibitor comprise increased lytic activity at lower Effector:Target cell ratios than those expanded from patients not pretreated with ibrutinib or other ITK inhibitor. In an embodiment of the invention, patients pretreated with ibrutinib or other ITK inhibitor have higher fold-expansion as compared with untreated patients.

In an embodiment of the invention, the method includes a step of adding an ITK inhibitor to the cell culture. In an embodiment, the ITK inhibitor is added on one or more of Day 0, Day 1, Day 2, Day 3, Day 4, Day 5, Day 6, Day 7, Day 8, Day 9, Day 10, Day 11, Day 12, Day 13, or Day 14 of the process. In an embodiment, the ITK inhibitor is added on the days during the method when cell culture medium is exchanged. In an embodiment, the ITK inhibitor is added on Day 0 and when cell culture medium is exchanged. In an embodiment, the ITK inhibitor is added during the method when IL-2 is added. In an embodiment, the ITK inhibitor is added on Day 0, Day 4, Day 7, and optionally Day 11 of the method. In an embodiment of the invention, the ITK inhibitor is added at Day 0 and at Day 7 of the method. In an embodiment of the invention, the ITK inhibitor is one known in the art. In an embodiment of the invention, the ITK inhibitor is one described elsewhere herein.

In an embodiment of the invention, the ITK inhibitor is used in the method at a concentration of from about 0.1 nM to about 5 uM. In an embodiment, the ITK inhibitor is used in the method at a concentration of about 0.1 nM, 0.5 nM, 1 nM, 5 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 uM, 2 uM, 3 uM, 4 uM, or 5 uM.

In an embodiment of the invention, the method includes a step of adding an ITK inhibitor when the PBMCs are derived from a patient who has no prior exposure to an ITK inhibitor treatment, such as ibrutinib.

In some embodiments, the PBMC sample is from a subject or patient who has been optionally pre-treated with a regimen comprising a kinase inhibitor or an ITK inhibitor. In some embodiments, the tumor sample is from a subject or patient who has been pre-treated with a regimen comprising a kinase inhibitor or an ITK inhibitor. In some embodiments, the PBMC sample is from a subject or patient who has been pre-treated with a regimen comprising a kinase inhibitor or an ITK inhibitor, has undergone treatment for at least 1 month, at least 2 months, at least 3 months, at least 4 months, at least 5 months, at least 6 months, or 1 year or more. In another embodiment, the PBMCs are derived from a patient who is currently on an ITK inhibitor regimen, such as ibrutinib.

In some embodiments, the PBMC sample is from a subject or patient who has been pre-treated with a regimen comprising a kinase inhibitor or an ITK inhibitor and is refractory to treatment with a kinase inhibitor or an ITK inhibitor, such as ibrutinib.

In some embodiments, the PBMC sample is from a subject or patient who has been pre-treated with a regimen comprising a kinase inhibitor or an ITK inhibitor but is no longer undergoing treatment with a kinase inhibitor or an ITK inhibitor. In some embodiments, the PBMC sample is from a subject or patient who has been pre-treated with a regimen comprising a kinase inhibitor or an ITK inhibitor but is no longer undergoing treatment with a kinase inhibitor or an ITK inhibitor and has not undergone treatment for at least 1 month, at least 2 months, at least 3 months, at least 4 months, at least 5 months, at least 6 months, or at least 1 year or more. In another embodiment, the PBMCs are derived from a patient who has prior exposure to an ITK inhibitor, but has not been treated in at least 3 months, at least 6 months, at least 9 months, or at least 1 year.

In an embodiment of the invention, at Day 0, cells are selected for CD19+ and sorted accordingly. In an embodiment of the invention, the selection is made using antibody binding beads. In an embodiment of the invention, pure T-cells are isolated on Day 0 from the PBMCs. In an embodiment of the invention, at Day 0, the CD19+ B cells and pure T cells are co-cultured with antiCD3/antiCD28 antibodies for a minimum of 4 days. In an embodiment of the invention, on Day 4, IL-2 is added to the culture. In an embodiment of the invention, on Day 7, the culture is restimulated with antiCD3/antiCD28 antibodies and additional IL-2. In an embodiment of the invention, on Day 14, the PBLs are harvested.

In an embodiment of the invention, for patients that are not pre-treated with ibrutinib or other ITK inhibitor, 10-15 ml of Buffy Coat will yield about 5×10⁹ PBMC, which, in turn, will yield about 5.5×10⁷ starting cell material, and about 11×10⁹ PBLs at the end of the expansion process. In an embodiment of the invention, about 54×10⁶ PBMCs will yield about 6×10⁵ starting material, and about 1.2×10⁸ MIL (about a 205-fold expansion).

In an embodiment of the invention, for patients that are pre-treated with ibrutinib or other ITK inhibitor, the expansion process will yield about 20×10⁹ PBLs. In an embodiment of the invention, 40.3×10⁶ PBMCs will yield about 4.7×10⁵ starting cell material, and about 1.6×10⁸ PBLs (about a 338-fold expansion).

In an embodiment of the invention, the clinical dose of PBLs useful in the present invention for patients with chronic lymphocytic leukemia (CLL) is from about 0.1×10⁹ to about 15×10⁹ PBLs, from about 0.1×10⁹ to about 15×10⁹ PBLs, from about 0.12×10⁹ to about 12×10⁹ PBLs, from about 0.15×10⁹ to about 11×10⁹ PBLs, from about 0.2×10⁹ to about 10×10⁹ PBLs, from about 0.3×10⁹ to about 9×10⁹ PBLs, from about 0.4×10⁹ to about 8×10⁹ PBLs, from about 0.5×10⁹ to about 7×10⁹ PBLs, from about 0.6×10⁹ to about 6×10⁹ PBLs, from about 0.7×10⁹ to about 5×10⁹ PBLs, from about 0.8×10⁹ to about 4×10⁹ PBLs, from about 0.9×10⁹ to about 3×10⁹ PBLs, or from about 1×10⁹ to about 2×10⁹ PBLs.

In any of the foregoing embodiments, PBMCs may be derived from a whole blood sample, by apheresis, from the buffy coat, or from any other method known in the art for obtaining PBMCs.

Methods of Expanding Marrow Infiltrating Lymphocytes (MILs) from PBMCs Derived from Bone Marrow

MIL Method 1. In an embodiment of the invention, a method for expanding MILs from PBMCs derived from bone marrow is described. In an embodiment of the invention, the method is performed over 14 days. In an embodiment, the method comprises obtaining bone marrow PBMCs and cryopreserving the PBMCs. On Day 0, the PBMCs are cultured with antiCD3/antiCD28 antibodies (DynaBeads®) in a 1:1 ratio (beads:cells) and IL-2 at 3000 IU/ml. On Day 4, additional IL-2 is added to the culture at 3000 IU/ml. On Day 7, the culture is again stimulated with antiCD3/antiCD28 antibodies (DynaBeads®) in a 1:1 ratio (beads:cells), and additional IL-2 at 3000 IU/ml is added to the culture. MILs are harvested on Day 14, beads are removed, and MILs are optionally counted and phenotyped.

In an embodiment of the invention, MIL Method 1 is performed as follows: On Day 0, a cryopreserved PBMC sample derived from bone marrow is thawed and the PBMCs are counted. The PBMCs are co-cultured in a GRex 24-well plate at 5×10⁵ cells per well with anti-CD3/anti-CD28 antibodies (DynaBeads®) at a 1:1 ratio in about 8 ml per well of CM-2 cell culture medium (comprised of RPMI-1640, human AB serum, 1-glutamine, 2-mercaptoethanol, gentamicin sulfate, AIM-V media) in the presence of IL-2 at 3000 IU/ml. On Day 4, the cell culture media is exchanged with AIM-V supplemented with additional IL-2 at 3000 IU/ml. On Day 7, the expanded MILs are counted. 1×10⁶ cells per well are transferred to a new GRex 24-well plate and cultured with anti-CD3/anti-CD28 antibodies (DynaBeads®) at a 1:1 ratio in about 8 ml per well of AIM-V media in the presence of IL-2 at 3000 IU/ml. On Day 11, the cell culture media is exchanged from AIM-V to CM-4 (comprised of AIM-V media, 2 mM Glutamax, and 3000 IU/ml IL2). On Day 14, the DynaBeads® are removed using a DynaMag Magnet (DynaMag™15) and the MILs are counted.

MIL Method 2. In an embodiment of the invention, the method is performed over 7 days. In an embodiment, the method comprises obtaining PMBCs derived from bone marrow and cryopreserving the PBMCs. On Day 0, the PBMCs are cultured with with antiCD3/antiCD28 antibodies (DynaBeads®) in a 3:1 ratio (beads:cells) and IL-2 at 3000 IU/ml. MILs are harvested on Day 7, beads are removed, and MILs are optionally counted and phenotyped.

In an embodiment of the invention, MIL Method 2 is performed as follows: On Day 0, a cryopreserved PBMC sample is thawed and the PBMCs are counted. The PBMCs are co-cultured in a GRex 24-well plate at 5×10⁵ cells per well with anti-CD3/anti-CD28 antibodies (DynaBeads®) at a 1:1 ratio in about 8 ml per well of CM-2 cell culture medium (comprised of RPMI-1640, human AB serum, 1-glutamine, 2-mercaptoethanol, gentamicin sulfate, AIM-V media) in the presence of IL-2 at 3000 IU/ml. On Day 7, the DynaBeads® are removed using a DynaMag Magnet (DynaMag™15) and the MILs are counted.

MIL Method 3. In an embodiment of the invention, the method comprises obtaining PBMCs from the bone marrow. On Day 0, the PBMCs are selected for CD3+/CD33+/CD20+/CD14+ and sorted, and the non-CD3+/CD33+/CD20+/CD14+ cell fraction is sonicated and a portion of the sonicated cell fraction is added back to the selected cell fraction. IL-2 is added to the cell culture at 3000 IU/ml. On Day 3, the PBMCs are cultured with antiCD3/antiCD28 antibodies (DynaBeads®) in a 1:1 ratio (beads:cells) and IL-2 at 3000 IU/ml. On Day 4, additional IL-2 is added to the culture at 3000 IU/ml. On Day 7, the culture is again stimulated with antiCD3/antiCD28 antibodies (DynaBeads®) in a 1:1 ratio (beads:cells), and additional IL-2 at 3000 IU/ml is added to the culture. On Day 11, IL-2 is added to the culture at 3000 IU/ml. MILs are harvested on Day 14, beads are removed, and MILs are optionally counted and phenotyped.

In an embodiment of the invention, MIL Method 3 is performed as follows: On Day 0, the PBMCs are selected for CD45hiCD3+ cells (immune cell fraction) by sorting method and CD45lowCD3-fraction (AML blast cell fraction) is sonicated and a portion of the sonicated cell fraction is added back to the selected cell fraction. IL-2 is added to the cell culture at 3000 IU/ml. On Day 3, the PBMCs are cultured with antiCD3/antiCD28 antibodies (DynaBeads®) in a 1:1 ratio (beads:cells) and IL-2 at 3000 IU/ml. On Day 4, additional IL-2 is added to the culture at 3000 IU/ml. On Day 7, the culture is again stimulated with antiCD3/antiCD28 antibodies (DynaBeads®) in a 1:1 ratio (beads:cells), and additional IL-2 at 3000 IU/ml is added to the culture. On Day 11, IL-2 is added to the culture at 3000 IU/ml. MILs are harvested on Day 14, beads are removed, and MILs are optionally counted and phenotyped.

In an embodiment of the invention, MIL Method 3 is performed as follows: On Day 0, a cryopreserved sample of PBMCs is thawed and PBMCs are counted. The cells are stained with CD3, CD33, CD20, and CD14 antibodies and sorted using a S3e cell sorted (Bio-Rad). The cells are sorted into two fractions—an immune cell fraction (or the MIL fraction) (CD3+CD33+CD20+CD14+) and an AML blast cell fraction (non-CD3+CD33+CD20+CD14+). A number of cells from the AML blast cell fraction that is about equal to the number of cells from the immune cell fraction (or MIL fraction) to be seeded on a Grex 24-well plate is suspended in 100 ul of media and sonicated. In this example, about 2.8×10⁴ to about 3.38×10⁵ cells from the AML blast cell fraction is taken and suspended in 100 ul of CM2 media and then sonicated for 30 seconds. The 100 ul of sonicated AML blast cell fraction is added to the immune cell fraction in a Grex 24-well plate. The immune cells are present in an amount of about 2.8×10⁴ to about 3.38×10⁵ cells per well in about 8 ml per well of CM-2 cell culture medium in the presence of IL-2 at 6000 IU/ml and are cultured with the portion of AML blast cell fraction for about 3 days. On Day 3, anti-CD3/anti-CD28 antibodies (DynaBeads®) at a 1:1 ratio are added to the each well and cultured for about 1 day. On Day 4, the cell culture media is exchanged with AIM-V supplemented with additional IL-2 at 3000 IU/ml. On Day 7, the expanded MILs are counted. About 1.5×10⁵ to 4×10⁵ cells per well are transferred to a new GRex 24-well plate and cultured with anti-CD3/anti-CD28 antibodies (DynaBeads®) at a 1:1 ratio in about 8 ml per well of AIM-V medium in the presence of IL-2 at 3000 IU/ml. On Day 11, the cell culture media is exchanged from AIM-V to CM-4 (supplemented with IL-2 at 3000 IU/ml). On Day 14, the DynaBeads® are removed using a DynaMag Magnet (DynaMag™15) and the MILs are optionally counted.

In an embodiment of the invention, PBMCs are obtained from bone marrow. In an embodiment, the PBMCs are obtained from the bone marrow through apheresis, aspiration, needle biopsy, or other similar means known in the art. In an embodiment, the PBMCs are fresh. In another embodiment, the PBMCs are cryopreserved.

In an embodiment of the invention, the method is performed over about 7 days, about 8 days, about 9 days, about 10 days, about 11 days, about 12 days, about 13 days, or about 14 days. In another embodiment, the method is performed over about 7 days. In another embodiment, the method is performed over about 14 days.

In an embodiment of the invention, the PBMCs are cultured with antiCD3/antiCD28 antibodies. In an embodiment, any available antiCD3/antiCD28 product is useful in the present invention. In an embodiment of the invention, the commercially available product used are DynaBeads®. In an embodiment, the DynaBeads® are cultured with the PBMCs in a ratio of 1:1 (beads:cells). In another embodiment, the antibodies are DynaBeads® cultured with the PBMCs in a ratio of 1.5:1, 2:1, 2.5:1, 3:1, 3.5:1, 4:1, 4.5:1, or 5:1 (beads:cells). In any of the foregoing embodiments, magnetic bead-based selection of an immune cell fraction (or MIL fraction) (CD3+CD33+CD20+CD14+) or an AML blast cell fraction (non-CD3+CD33+CD20+CD14+) is used. In an embodiment of the invention, the antibody culturing steps and/or the step of restimulating cells with antibody is performed over a period of from about 2 to about 6 days, from about 3 to about 5 days, or for about 4 days. In an embodiment of the invention, the antibody culturing step is performed over a period of about 2 days, 3 days, 4 days, 5 days, or 6 days.

In an embodiment of the invention, the ratio of the number of cells from the AML blast cell fraction to the number of cells from the immune cell fraction (or MIL fraction) is about 0.1:1 to about 10:1. In another embodiment, the ratio is about 0.1:1 to about 5:1, about 0.1:1 to about 2:1, or about 1:1. In an embodiment of the invention, the AML blast cell fraction is optionally disrupted to break up cell aggregation. In an embodiment, the AML blast cell fraction is disrupted using sonication, homogenization, cell lysis, vortexing, or vibration. In another embodiment, the AML blast cell fraction is disrupted using sonication. In an embodiment of the invention, the non-CD3+, non-CD33+, non-CD20+, non-CD14+ cell fraction (AML blast fraction) is lysed using a suitable lysis method, including high temperature lysis, chemical lysis (such as organic alcohols), enzyme lysis, and other cell lysis methods known in the art.

In an embodiment of the invention, the cells from AML blast cell fraction are suspended at a concentration of from about 0.2×10⁵ to about 2×10⁵ cells per 100 uL and added to the cell culture with the immune cell fraction. In another embodiment, the concentration is from about 0.5×10⁵ to about 2×10⁵ cells per 100 uL, from about 0.7×10⁵ to about 2×10⁵ cells per 100 uL, from about 1 ×10⁵ to about 2×10⁵ cells per 100 uL, or from about 1.5×10⁵ to about 2×10⁵ cells per 100 uL.

In an embodiment, the PBMC sample is cultured with IL-2. In an embodiment of the invention, the cell culture medium used for expansion of the MILs comprises IL-2 at a concentration selected from the group consisting of about 100 IU/mL, about 200 IU/mL, about 300 IU/mL, about 400 IU/mL, about 100 IU/mL, about 100 IU/mL, about 100 IU/mL, about 100 IU/mL, about 100 IU/mL, about 500 IU/mL, about 600 IU/mL, about 700 IU/mL, about 800 IU/mL, about 900 IU/mL, about 1,000 IU/mL, about 1,100 IU/mL, about 1,200 IU/mL, about 1,300 IU/mL, about 1,400 IU/mL, about 1,500 IU/mL, about 1,600 IU/mL, about 1,700 IU/mL, about 1,800 IU/mL, about 1,900 IU/mL, about 2,000 IU/mL, about 2,100 IU/mL, about 2,200 IU/mL, about 2,300 IU/mL, about 2,400 IU/mL, about 2,500 IU/mL, about 2,600 IU/mL, about 2,700 IU/mL, about 2,800 IU/mL, about 2,900 IU/mL, about 3,000 IU/mL, about 3,100 IU/mL, about 3,200 IU/mL, about 3,300 IU/mL, about 3,400 IU/mL, about 3,500 IU/mL, about 3,600 IU/mL, about 3,700 IU/mL, about 3,800 IU/mL, about 3,900 IU/mL, about 4,000 IU/mL, about 4,100 IU/mL, about 4,200 IU/mL, about 4,300 IU/mL, about 4,400 IU/mL, about 4,500 IU/mL, about 4,600 IU/mL, about 4,700 IU/mL, about 4,800 IU/mL, about 4,900 IU/mL, about 5,000 IU/mL, about 5,100 IU/mL, about 5,200 IU/mL, about 5,300 IU/mL, about 5,400 IU/mL, about 5,500 IU/mL, about 5,600 IU/mL, about 5,700 IU/mL, about 5,800 IU/mL, about 5,900 IU/mL, about 6,000 IU/mL, about 6,500 IU/mL, about 7,000 IU/mL, about 7,500 IU/mL, about 8,000 IU/mL, about 8,500 IU/mL, about 9,000 IU/mL, about 9,500 IU/mL, and about 10,000 IU/mL.

In an embodiment of the invention, additional IL-2 may be added to the culture on one or more days throughout the method. In an embodiment of the invention, additional IL-2 is added on Day 4. In an embodiment of the invention, additional IL-2 is added on Day 7. In an embodiment of the invention, additional IL-2 is added on Day 11. In another embodiment, additional IL-2 is added on Day 4, Day 7, and/or Day 11. In an embodiment of the invention, the MILs are cultured with additional IL-2 for a period of 1 day, 2 days, 3 days, 4 days, 5 days, 6 days, 7 days, 8 days, 9 days, 10 days, 11 days, 12 days, 13 days, or 14 days. In an embodiment of the invention, MILs are cultured for a period of 3 days after each addition of IL-2.

In an embodiment, the cell culture medium is exchanged at least once time during the method. In an embodiment, the cell culture medium is exchanged at the same time that additional IL-2 is added. In another embodiment the cell culture medium is exchanged on at least one of Day 1, Day 2, Day 3, Day 4, Day 5, Day 6, Day 7, Day 8, Day 9, Day 10, Day 11, Day 12, Day 13, or Day 14. In an embodiment of the invention, the cell culture medium used throughout the method may be the same or different. In an embodiment of the invention, the cell culture medium is CM-2, CM-4, or AIM-V. In an embodiment of the invention, the cell culture medium exchange step on Day 11 is optional. In an embodiment of the invention, the starting cell number of PBMCs for the expansion process is from about 25,000 to about 1,000,000, from about 30,000 to about 900,000, from about 35,000 to about 850,000, from about 40,000 to about 800,000, from about 45,000 to about 800,000, from about 50,000 to about 750,000, from about 55,000 to about 700,000, from about 60,000 to about 650,000, from about 65,000 to about 600,000, from about 70,000 to about 550,000, preferably from about 75,000 to about 500,000, from about 80,000 to about 450,000, from about 85,000 to about 400,000, from about 90,000 to about 350,000, from about 95,000 to about 300,000, from about 100,000 to about 250,000, from about 105,000 to about 200,000, or from about 110,000 to about 150,000. In an embodiment of the invention, the starting cell number of PBMCs is about 138,000, 140,000, 145,000, or more. In another embodiment, the starting cell number of PBMCs is about 28,000. In another embodiment, the starting cell number of PBMCs is about 62,000. In another embodiment, the starting cell number of PBMCs is about 338,000. In another embodiment, the starting cell number of PBMCs is about 336,000.

In an embodiment of the invention, the fold expansion of MILs is from about 20% to about 100%, 25% to about 95%, 30% to about 90%, 35% to about 85%, 40% to about 80%, 45% to about 75%, 50% to about 100%, or 25% to about 75%. In an embodiment of the invention, the fold expansion is about 25%. In another embodiment of the invention, the fold expansion is about 50%. In another embodiment, the fold expansion is about 75%.

In an embodiment of the invention, MILs are expanded from 10-50 ml of bone marrow aspirate. In an embodiment of the invention, 10 ml of bone marrow aspirate is obtained from the patient. In another embodiment, 20 ml of bone marrow aspirate is obtained from the patient. In another embodiment, 30 ml of bone marrow aspirate is obtained from the patient. In another embodiment, 40 ml of bone marrow aspirate is obtained from the patient. In another embodiment, 50 ml of bone marrow aspirate is obtained from the patient.

In an embodiment of the invention, the number of PBMCs yielded from about 10-50 ml of bone marrow aspirate is about 5×10⁷ to about 10×10⁷ PBMCs. In another embodiment, the number of PMBCs yielded is about 7×10⁷ PBMCs.

In an embodiment of the invention, about 5×10⁷ to about 10×10⁷ PBMCs, yields about 0.5×10⁶ to about 1.5×10⁶ expansion starting cell material. In an embodiment of the invention, about 1×10⁶ expansion starting cell material is yielded.

In an embodiment of the invention, the total number of MILs harvested at the end of the expansion period is from about 0.01×10⁹ to about 1×10⁹, from about 0.05×10⁹ to about 0.9×10⁹, from about 0.1×10⁹ to about 0.85×10⁹, from about 0.15×10⁹ to about 0.7×10⁹, from about 0.2×10⁹ to about 0.65×10⁹, from about 0.25×10⁹ to about 0.6×10⁹, from about 0.3×10⁹ to about 0.55×10⁹, from about 0.35×10⁹ to about 0.5×10⁹, or from about 0.4×10⁹ to about 0.45×10⁹.

In an embodiment of the invention, 12×10⁶ PBMC derived from bone marrow aspirate yields approximately 1.4×10⁵ starting cell material, which yields about 1.1×10⁷ MILs at the end of the expansion process.

In an embodiment of the invention, the MILs expanded from bone marrow PBMCs using MIL Method 3 described above comprise a high proportion of CD8+ cells and lower number of LAG3+ and PD1+ cells as compared with MILs expanded using MIL Method 1 or MIL Method 2. In an embodiment of the invention, PBLs expanded from blood PBMC using MIL Method 3 described above comprise a high proportion of CD8+ cells and increased levels of IFNγ production as compared with PBLs expanded using MIL Method 1 or MIL Method 2.

In an embodiment of the invention, the clinical dose of MILs useful for patients with acute myeloid leukemia (AML) is in the range of from about 4×10⁸ to about 2.5×10⁹ MILs. In another embodiment, the number of MILs provided in the pharmaceutical compositions of the invention is 9.5×10⁸ MILs. In another embodiment, the number of MILs provided in the pharmaceutical compositions of the invention is 4.1×10⁸. In another embodiment, the number of MILs provided in the pharmaceutical compositions of the invention is 2.2×10⁹.

In any of the foregoing embodiments, PBMCs may be derived from a whole blood sample, from bone marrow, by apheresis, from the buffy coat, or from any other method known in the art for obtaining PBMCs.

Methods for Expansion of TILs Using the “2A Process”

In an embodiment of the present invention, the invention provides devices and methods to expand T cells derived from bone marrow and/or peripheral blood. In an embodiment of the invention, the T cells have a heightened tumor specificity from the bone marrow microenvironment in a polyclonal but highly tumor-specific manner. In an embodiment, the bone marrow microenvironment is used to sustain and expand the T-cells. In an embodiment of the invention, there is roughly a 25 to 100-fold expansion of TILs in a 7-day or 14-day expansion process. In an embodiment, the fold expansion of TILs is from about 30-90-fold. In an embodiment, the fold expansion is from about 35-85-fold. In an embodiment, the fold expansion is from about 40-80-fold. In an embodiment, the fold expansion is from about 45-75-fold. In another embodiment, the fold expansion is from about 40-70-fold. In another embodiment, the fold expansion is from about 45-65-fold. In another embodiment, the fold expansion is about 25-fold, about 30-fold, about 35-fold, about 40-fold, about 45-fold, and 50-fold, about 55-fold, about 60-fold, about 65-fold, about 70 fold, about 75-fold, about 80-fold, about 85-fold, about 90-fold, about 95-fold, or about 100-fold expansion.

In an embodiment of the invention, the T-cell manufacturing process does not require any intervention to select for tumor specificity. In an embodiment of the invention, the T-cell manufacturing process does not require the presence of tumor in the marrow and/or peripheral blood at the time of T-cell expansion. In an embodiment, the T-cells are expanded in the presence of almost complete bone marrow.

In an embodiment, the invention provides a method for extracting T-cells from bone marrow and/or peripheral blood as described in the Examples, and in particular, Example 21, set forth in WO2010/062742, which is incorporated herein by reference. In an embodiment, the invention provides a method for extracting T-cells from bone marrow and/or peripheral blood as described in, for example, Noonan, et al., 2005, Cancer Res. 65:2026-2034, which is incorporated herein by reference.

In an embodiment, methods for obtaining bone marrow and/or peripheral blood that are known to someone skilled in the art are useful in the present invention. In an embodiment of the invention, bone marrow and/or peripheral blood is obtained using needle aspiration. In an embodiment of the invention, bone marrow from a patient is aspirated into heparin-containing syringes and stored overnight at room temperature. In an embodiment of the invention, after storage, the contents of the syringes are pooled together into a sterile container and quality tested. The bone marrow is enriched for mononuclear cells (MNCs) using lymphocyte separation media (LSM) and centrifugation with a COBE Spectra. Cells in the gradient are collected down to the red blood cells and washed using HBSS. The MNCs are cryopreserved using a hetastarch-based cryoprotectant supplemented with 2% HSA and 5% DMSO, reserving some of the MNCs for quality control. The QC vial is thawed to determine the CD3⁺ and CD38⁺/138⁺ cell content of the MNC product. It is important to note that the collection of bone marrow is not a limitation to the present invention.

In an embodiment of the invention, bone marrow is aspirated and fractionated on a Lymphocyte Separation Medium density gradient and cells are collected almost to the level of the red cell pellet. In an embodiment, this fractionation method substantially removes red blood cells and neutrophils, providing nearly complete bone marrow. In an embodiment, the resulting fractionated material is T-cells and tumor cells. In an embodiment of the invention, the methods may be practiced without a T-cell specific separation step, and without a tumor cell separation step, such as, for example, without labeling T-cells with antibodies or other cell-type specific detectable labels, and without sorting using fluorescence activated cell sorting (FACS).

In an embodiment of the invention, the obtained bone marrow is Ficolled or the peripheral blood is suspended in serum-free conditions at 1×10⁶ cells/mL in AIM-V medium at 200 uL/well.

In an embodiment of the invention, the bone marrow is collected from a subject who is not in complete remission. In an embodiment of the invention, the bone marrow is collected from a subject who is in complete remission.

In an embodiment of the invention, the bone marrow may be obtained and frozen. In an embodiment, the bone marrow may be obtained and immediately used to extract T-cells.

In further embodiments and in accordance with any of the above, the invention provides a method of expanding TILs, the method comprising contacting a population of TILs comprising at least one TIL obtained from a liquid tumor. All discussion of expanding TILs herein are applicable to expansion of TILs obtained from bone marrow, peripheral blood, and/or a hematological malignancy, including a liquid tumor.

In an embodiment, the invention provides a process for the preparation of a population of tumor infiltrating lymphocytes (TILs) from a tumor obtained from a cancer, the process comprising the steps of:
(a) contacting a fragmented tumor comprising a first population of TILs with a first cell culture medium;
(b) performing an initial expansion (pre-REP) of the first population of TILs in the first cell culture medium to obtain a second population of TILs, wherein the second population of TILs is at least 5-fold greater in number than the first population of TILs, wherein the first cell culture medium comprises IL-2;
(c) performing a second expansion of the second population of TILs in a second cell culture medium to obtain a third population of TILs, wherein the third population of TILs is at least 50-fold greater in number than the second population of TILs after 7 days from the start of the second expansion; wherein the second cell culture medium comprises IL-2, OKT-3 (anti-CD3 antibody), irradiated allogeneic peripheral blood mononuclear cells (PBMCs); and wherein the second expansion is performed over a period of 14 days or less;
(d) harvesting the third population of TILs; and
   wherein the tumor is a liquid tumor, and wherein the cancer is a hematological malignancy.

In an embodiment, the invention provides a process for expanding a population of TILs including a first pre-rapid expansion (pre-REP) process and then a second expansion process (which can be a rapid expansion process—REP), wherein the cell culture medium used for expansion comprises IL-2 at a concentration selected from the group consisting of between 100 IU/mL and 10,000 IU/mL, between 200 IU/mL and 5,000 IU/mL, between 300 IU/mL and 4,800 IU/mL, between 400 IU/mL and 4,600 IU/mL, between 500 IU/mL and 4,400 IU/mL, between 600 IU/mL and 4,200 IU/mL, between 700 IU/mL and 4,000 IU/mL, between 800 IU/mL and 3,800 IU/mL, between 900 IU/mL and 3,600 IU/mL, between 1,000 IU/mL and 3,400 IU/mL, between 1,100 IU/mL and 3,200 IU/mL, between 1,200 IU/mL and 3,000 IU/mL, between 1,300 IU/mL and 2,800 IU/mL, between 1,400 IU/mL and 2,600 IU/mL, between 1,500 IU/mL and 2,400 IU/mL, between 1,600 IU/mL and 2,200 IU/mL, between 1,700 IU/mL and 2,000 IU/mL, between 5,500 IU/mL and 9,500 IU/mL, between 6,000 IU/mL and 9,000 IU/mL, between 6500 IU/mL and 8,500 IU/mL, between 7,000 IU/mL and 8,000 IU/mL, and between 7,500 IU/mL and 8,000 IU/mL.

In an embodiment, the invention provides a process for expanding a population of TILs including a pre-rapid expansion (pre-REP) process and a rapid expansion process (REP), wherein the cell culture medium used for expansion comprises IL-2 at a concentration selected from the group consisting of about 100 IU/mL, about 200 IU/mL, about 300 IU/mL, about 400 IU/mL, about 100 IU/mL, about 100 IU/mL, about 100 IU/mL, about 100 IU/mL, about 100 IU/mL, about 500 IU/mL, about 600 IU/mL, about 700 IU/mL, about 800 IU/mL, about 900 IU/mL, about 1,000 IU/mL, about 1,100 IU/mL, about 1,200 IU/mL, about 1,300 IU/mL, about 1,400 IU/mL, about 1,500 IU/mL, about 1,600 IU/mL, about 1,700 IU/mL, about 1,800 IU/mL, about 1,900 IU/mL, about 2,000 IU/mL, about 2,100 IU/mL, about 2,200 IU/mL, about 2,300 IU/mL, about 2,400 IU/mL, about 2,500 IU/mL, about 2,600 IU/mL, about 2,700 IU/mL, about 2,800 IU/mL, about 2,900 IU/mL, about 3,000 IU/mL, about 3,100 IU/mL, about 3,200 IU/mL, about 3,300 IU/mL, about 3,400 IU/mL, about 3,500 IU/mL, about 3,600 IU/mL, about 3,700 IU/mL, about 3,800 IU/mL, about 3,900 IU/mL, about 4,000 IU/mL, about 4,100 IU/mL, about 4,200 IU/mL, about 4,300 IU/mL, about 4,400 IU/mL, about 4,500 IU/mL, about 4,600 IU/mL, about 4,700 IU/mL, about 4,800 IU/mL, about 4,900 IU/mL, about 5,000 IU/mL, about 5,100 IU/mL, about 5,200 IU/mL, about 5,300 IU/mL, about 5,400 IU/mL, about 5,500 IU/mL, about 5,600 IU/mL, about 5,700 IU/mL, about 5,800 IU/mL, about 5,900 IU/mL, about 6,000 IU/mL, about 6,500 IU/mL, about 7,000 IU/mL, about 7,500 IU/mL, about 8,000 IU/mL, about 8,500 IU/mL, about 9,000 IU/mL, about 9,500 IU/mL, and about 10,000 IU/mL.

In an embodiment, the invention provides a process for expanding a population of TILs including a pre-rapid expansion (pre-REP) process. In an embodiment, the invention provides a pre-REP process of expanding a population of TILs, the pre-REP process comprising the steps of contacting the population of TILs obtained from a liquid tumor with a cell culture medium, wherein the cell culture medium further comprises IL-2 at an initial concentration of between 1000 IU/mL and 6000 IU/mL.

In an embodiment, the invention provides a pre-REP process for expanding a population of TILs, the process comprising the steps of contacting the population of TILs obtained from a liquid tumor with a cell culture medium, wherein the cell culture medium further comprises IL-2 at an initial concentration of about 6000 IU/mL.

In an embodiment, REP can be performed in a gas permeable container using the TILs obtained from a liquid tumor according to the present disclosure by any suitable method. For example, TILs can be rapidly expanded using non-specific T cell receptor stimulation in the presence of interleukin-2 (IL-2) or interleukin-15 (IL-15). The non-specific T cell receptor stimulus can include, for example, about 30 ng/mL of OKT-3, a monoclonal anti-CD3 antibody (commercially available from Ortho-McNeil, Raritan, N.J. or Miltenyi Biotech, Auburn, Calif.). TILs can be rapidly expanded by further stimulation of the TILs in vitro with one or more antigens, including antigenic portions thereof, such as epitope(s), of the cancer, which can be optionally expressed from a vector, such as a human leukocyte antigen A2 (HLA-A2) binding peptide, e.g., 0.3 μM MART-1: 26-35 (27 L) or gpl 00:209-217 (210M), optionally in the presence of a T-cell growth factor, such as 300 IU/mL IL-2 or IL-15. Other suitable antigens may include, e.g., NY-ESO-1, TRP-1, TRP-2, tyrosinase cancer antigen, MAGE-A3, SSX-2, and VEGFR2, or antigenic portions thereof. TIL may also be rapidly expanded by re-stimulation with the same antigen(s) of the cancer pulsed onto HLA-A2-expressing antigen-presenting cells. Alternatively, the TILs can be further re-stimulated with, e.g., example, irradiated, autologous lymphocytes or with irradiated HLA-A2+ allogeneic lymphocytes and IL-2.

In an embodiment, a method for expanding TILs may include using about 5000 mL to about 25000 mL of cell culture medium, about 5000 mL to about 10000 mL of cell culture medium, or about 5800 mL to about 8700 mL of cell culture medium. In an embodiment, a method for expanding TILs may include using about 1000 mL to about 2000 mL of cell medium, about 2000 mL to about 3000 mL of cell culture medium, about 3000 mL to about 4000 mL of cell culture medium, about 4000 mL to about 5000 mL of cell culture medium, about 5000 mL to about 6000 mL of cell culture medium, about 6000 mL to about 7000 mL of cell culture medium, about 7000 mL to about 8000 mL of cell culture medium, about 8000 mL to about 9000 mL of cell culture medium, about 9000 mL to about 10000 mL of cell culture medium, about 10000 mL to about 15000 mL of cell culture medium, about 15000 mL to about 20000 mL of cell culture medium, or about 20000 mL to about 25000 mL of cell culture medium. In an embodiment, expanding the number of TILs uses no more than one type of cell culture medium. Any suitable cell culture medium may be used, e.g., AIM-V cell medium (L-glutamine, 50 μM streptomycin sulfate, and 10 μM gentamicin sulfate) cell culture medium (Invitrogen, Carlsbad Calif.). In this regard, the inventive methods advantageously reduce the amount of medium and the number of types of medium required to expand the number of TIL. In an embodiment, expanding the number of TIL may comprise feeding the cells no more frequently than every third or fourth day. Expanding the number of cells in a gas permeable container simplifies the procedures necessary to expand the number of cells by reducing the feeding frequency necessary to expand the cells.

In an embodiment, a second expansion is performed using a gas permeable container. Such embodiments allow for cell populations to expand from about 5×10⁵ cells/cm² to between 10×10⁶ and 30×10⁶ cells/cm². In an embodiment, this expansion occurs without feeding. In an embodiment, this expansion occurs without feeding so long as medium resides at a height of about 10 cm in a gas-permeable flask. In an embodiment this is without feeding but with the addition of one or more cytokines. In an embodiment, the cytokine can be added as a bolus without any need to mix the cytokine with the medium. Such containers, devices, and methods are known in the art and have been used to expand TILs, and include those described in U.S. Patent Application Publication No. US 2014/0377739 A1, International Patent Application Publication No. WO 2014/210036 A1, U.S. Patent Application Publication No. US 2013/0115617 A1, International Publication No. WO 2013/188427 A1, U.S. Patent Application Publication No. US 2011/0136228 A1, U.S. Pat. No. 8,809,050, International Patent Application Publication No. WO 2011/072088 A2, U.S. Patent Application Publication No. US 2016/0208216 A1, U.S. Patent Application Publication No. US 2012/0244133 A1, International Patent Application Publication No. WO 2012/129201 A1, U.S. Patent Application Publication No. US 2013/0102075 A1, U.S. Pat. No. 8,956,860, International Patent Application Publication No. WO 2013/173835 A1, and U.S. Patent Application Publication No. US 2015/0175966 A1, the disclosures of which are incorporated herein by reference. Such processes are also described in Jin, et al., J. Immunotherapy 2012, 35, 283-292, the disclosure of which is incorporated by reference herein.

In an embodiment, the gas permeable container is a G-Rex 10 flask (Wilson Wolf Manufacturing Corporation, New Brighton, Minn., USA). In an embodiment, the gas permeable container includes a 10 cm² gas permeable culture surface. In an embodiment, the gas permeable container includes a 40 mL cell culture medium capacity. In an embodiment, the gas permeable container provides 100 to 300 million TILs after 2 medium exchanges.

In an embodiment, the gas permeable container is a G-Rex 100 flask (Wilson Wolf Manufacturing Corporation, New Brighton, Minn., USA). In an embodiment, the gas permeable container includes a 100 cm² gas permeable culture surface. In an embodiment, the gas permeable container includes a 450 mL cell culture medium capacity. In an embodiment, the gas permeable container provides 1 to 3 billion TILs after 2 medium exchanges.

In an embodiment, the gas permeable container is a G-Rex 100M flask (Wilson Wolf Manufacturing Corporation, New Brighton, Minn., USA). In an embodiment, the gas permeable container includes a 100 cm² gas permeable culture surface. In an embodiment, the gas permeable container includes a 1000 mL cell culture medium capacity. In an embodiment, the gas permeable container provides 1 to 3 billion TILs without medium exchange.

In an embodiment, the gas permeable container is a G-Rex 100L flask (Wilson Wolf Manufacturing Corporation, New Brighton, Minn., USA). In an embodiment, the gas permeable container includes a 100 cm² gas permeable culture surface. In an embodiment, the gas permeable container includes a 2000 mL cell culture medium capacity. In an embodiment, the gas permeable container provides 1 to 3 billion TILs without medium exchange.

In an embodiment, the gas permeable container is a G-Rex 24 well plate (Wilson Wolf Manufacturing Corporation, New Brighton, Minn., USA). In an embodiment, the gas permeable container includes a plate with wells, wherein each well includes a 2 cm² gas permeable culture surface. In an embodiment, the gas permeable container includes a plate with wells, wherein each well includes an 8 mL cell culture medium capacity. In an embodiment, the gas permeable container provides 20 to 60 million cells per well after 2 medium exchanges.

In an embodiment, the gas permeable container is a G-Rex 6 well plate (Wilson Wolf Manufacturing Corporation, New Brighton, Minn., USA). In an embodiment, the gas permeable container includes a plate with wells, wherein each well includes a 10 cm² gas permeable culture surface. In an embodiment, the gas permeable container includes a plate with wells, wherein each well includes a 40 mL cell culture medium capacity. In an embodiment, the gas permeable container provides 100 to 300 million cells per well after 2 medium exchanges.

In an embodiment, the cell medium in the first and/or second gas permeable container is unfiltered. The use of unfiltered cell medium may simplify the procedures necessary to expand the number of cells. In an embodiment, the cell medium in the first and/or second gas permeable container lacks beta-mercaptoethanol (BME).

In an embodiment, the duration of the method comprising obtaining a tumor tissue sample from the mammal; culturing the tumor tissue sample in a first gas permeable container containing cell medium therein; obtaining TILs from the tumor tissue sample; expanding the number of TILs in a second gas permeable container containing cell medium therein for a duration of about 14 to about 42 days, e.g., about 28 days.

In an embodiment, the cell culture medium comprises IL-2. In a preferred embodiment, the cell culture medium comprises about 3000 IU/mL of IL-2. In an embodiment, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL of IL-2. In an embodiment, the cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or between 8000 IU/mL of IL-2.

In an embodiment, the cell culture medium comprises OKT-3 antibody. In a preferred embodiment, the cell culture medium comprises about 30 ng/mL of OKT-3 antibody. In an embodiment, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, and about 1 μg/mL of OKT-3 antibody. In an embodiment, the cell culture medium comprises between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, and between 50 ng/mL and 100 ng/mL of OKT-3 antibody.

In an embodiment, TILs are expanded in gas-permeable containers. Gas-permeable containers have been used to expand TILs using PBMCs using methods, compositions, and devices known in the art, including those described in U.S. Patent Application Publication No. U.S. Patent Application Publication No. 2005/0106717 A1, the disclosures of which are incorporated herein by reference. In an embodiment, TILs are expanded in gas-permeable bags. In an embodiment, TILs are expanded using a cell expansion system that expands TILs in gas permeable bags, such as the Xuri Cell Expansion System W25 (GE Healthcare). In an embodiment, TILs are expanded using a cell expansion system that expands TILs in gas permeable bags, such as the WAVE Bioreactor System, also known as the Xuri Cell Expansion System W5 (GE Healthcare). In an embodiment, the cell expansion system includes a gas permeable cell bag with a volume selected from the group consisting of about 100 mL, about 200 mL, about 300 mL, about 400 mL, about 500 mL, about 600 mL, about 700 mL, about 800 mL, about 900 mL, about 1 L, about 2 L, about 3 L, about 4 L, about 5 L, about 6 L, about 7 L, about 8 L, about 9 L, about 10 L, about 11 L, about 12 L, about 13 L, about 14 L, about 15 L, about 16 L, about 17 L, about 18 L, about 19 L, about 20 L, about 25 L, and about 30 L. In an embodiment, the cell expansion system includes a gas permeable cell bag with a volume range selected from the group consisting of between 50 and 150 mL, between 150 and 250 mL, between 250 and 350 mL, between 350 and 450 mL, between 450 and 550 mL, between 550 and 650 mL, between 650 and 750 mL, between 750 and 850 mL, between 850 and 950 mL, and between 950 and 1050 mL. In an embodiment, the cell expansion system includes a gas permeable cell bag with a volume range selected from the group consisting of between 1 L and 2 L, between 2 L and 3 L, between 3 L and 4 L, between 4 L and 5 L, between 5 L and 6 L, between 6 L and 7 L, between 7 L and 8 L, between 8 L and 9 L, between 9 L and 10 L, between 10 L and 11 L, between 11 L and 12 L, between 12 L and 13 L, between 13 L and 14 L, between 14 L and 15 L, between 15 L and 16 L, between 16 L and 17 L, between 17 L and 18 L, between 18 L and 19 L, and between 19 L and 20 L. In an embodiment, the cell expansion system includes a gas permeable cell bag with a volume range selected from the group consisting of between 0.5 L and 5 L, between 5 L and 10 L, between 10 L and 15 L, between 15 L and 20 L, between 20 L and 25 L, and between 25 L and 30 L. In an embodiment, the cell expansion system utilizes a rocking time of about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, and about 28 days. In an embodiment, the cell expansion system utilizes a rocking time of between 30 minutes and 1 hour, between 1 hour and 12 hours, between 12 hours and 1 day, between 1 day and 7 days, between 7 days and 14 days, between 14 days and 21 days, and between 21 days and 28 days. In an embodiment, the cell expansion system utilizes a rocking rate of about 2 rocks/minute, about 5 rocks/minute, about 10 rocks/minute, about 20 rocks/minute, about 30 rocks/minute, and about 40 rocks/minute. In an embodiment, the cell expansion system utilizes a rocking rate of between 2 rocks/minute and 5 rocks/minute, 5 rocks/minute and 10 rocks/minute, 10 rocks/minute and 20 rocks/minute, 20 rocks/minute and 30 rocks/minute, and 30 rocks/minute and 40 rocks/minute. In an embodiment, the cell expansion system utilizes a rocking angle of about 2°, about 3°, about 4°, about 5°, about 6°, about 7°, about 8°, about 9°, about 10°, about 11°, and about 12°. In an embodiment, the cell expansion system utilizes a rocking angle of between 2° and 3°, between 3° and 4°, between 4° and 5°, between 5° and 6°, between 6° and 7°, between 7° and 8°, between 8° and 9°, between 9° and 10°, between 10° and 11°, and between 11° and 12°.

In an embodiment, a method of expanding TILs obtained from a liquid tumor further comprises a step wherein TILs are selected for superior tumor reactivity. Any selection method known in the art may be used. For example, the methods described in U.S. Patent Application Publication No. 2016/0010058 A1, the disclosures of which are incorporated herein by reference, may be used for selection of TILs for superior tumor reactivity.

In an embodiment, the invention provides a method of expanding a population of TILs from a liquid tumor, the method comprising the steps as described in Jin, et al., J. Immunotherapy 2012, 35, 283-292, the disclosure of which is incorporated by reference herein. For example, the tumor or portion thereof may be placed in enzyme media and mechanically dissociated for approximately 1 minute. The mixture may then be incubated for 30 minutes at 37° C. in 5% CO₂ and then mechanically disrupted again for approximately 1 minute. After incubation for 30 minutes at 37° C. in 5% CO₂, the tumor or portion thereof may be mechanically disrupted a third time for approximately 1 minute. If after the third mechanical disruption, large pieces of tissue are present, 1 or 2 additional mechanical dissociations may be applied to the sample, with or without 30 additional minutes of incubation at 37° C. in 5% CO₂. At the end of the final incubation, if the cell suspension contains a large number of red blood cells or dead cells, a density gradient separation using Ficoll may be performed to remove these cells. TIL cultures were initiated in 24-well plates (Costar 24-well cell culture cluster, flat bottom; Corning Incorporated, Corning, N.Y.), each well may be seeded with 1×10⁶tumor digest cells or one tumor fragment approximately 1 to 8 mm³ in size in 2 mL of complete medium (CM) with IL-2 (6000 IU/mL; Chiron Corp., Emeryville, Calif.). CM comprises Roswell Park Memorial Institute (RPMI) 1640 buffer with GlutaMAX, supplemented with 10% human AB serum, 25 mM Hepes, and 10 mg/mL gentamicin. Cultures may be initiated in gas-permeable flasks with a 40 mL capacity and a 10 cm² gas-permeable silicon bottom (G-Rex 10; Wilson Wolf Manufacturing, New Brighton, each flask may be loaded with 10-40×10⁶ viable tumor digest cells or 5-30 tumor fragments in 10-40 mL of CM with IL-2. G-Rex 10 and 24-well plates may be incubated in a humidified incubator at 37° C. in 5% CO₂ and 5 days after culture initiation, half the media may be removed and replaced with fresh CM and IL-2 and after day 5, half the media may be changed every 2-3 days. A second expansion protocol (REP) of TILs may be performed using T-175 flasks and gas-permeable bags or gas-permeable G-Rex flasks, as described elsewhere herein, using TILs obtains from the liquid tumors of the present disclosure. For REP in T-175 flasks, 1×10⁶ TILs may be suspended in 150 mL of media in each flask. The TIL may be cultured in a 1 to 1 mixture of CM and AIM-V medium (50/50 medium), supplemented with 3000 IU/mL of IL-2 and 30 ng/mL of anti-CD3 antibody (OKT-3). The T-175 flasks may be incubated at 37° C. in 5% CO2. Half the media may be changed on day 5 using 50/50 medium with 3000 IU/mL of IL-2. On day 7, cells from 2 T-175 flasks may be combined in a 3 L bag and 300 mL of AIM-V with 5% human AB serum and 3000 IU/mL of IL-2 may be added to the 300 mL of TIL suspension. The number of cells in each bag may be counted every day or two days, and fresh media may be added to keep the cell count between 0.5 and 2.0×10⁶ cells/mL. For REP in 500 mL capacity flasks with 100 cm² gas-permeable silicon bottoms (e.g., G-Rex 100, Wilson Wolf Manufacturing, as described elsewhere herein), 5×10⁶ or 10×10⁶ TILs may be cultured in 400 mL of 50/50 medium, supplemented with 3000 IU/mL of IL-2 and 30 ng/mL of anti-CD3 antibody (OKT-3). The G-Rex100 flasks may be incubated at 37° C. in 5% CO₂. On day five, 250 mL of supernatant may be removed and placed into centrifuge bottles and centrifuged at 1500 rpm (491 g) for 10 minutes. The obtained TIL pellets may be resuspended with 150 mL of fresh 50/50 medium with 3000 IU/mL of IL-2 and added back to the G-Rex 100 flasks. When TIL are expanded serially in G-Rex 100 flasks, on day seven the TIL in each G-Rex100 are suspended in the 300 mL of media present in each flask and the cell suspension may be divided into three 100 mL aliquots that may be used to seed 3 G-Rex100 flasks. About 150 mL of AIM-V with 5% human AB serum and 3000 IU/mL of IL-2 may then be added to each flask. G-Rex 100 flasks may then be incubated at 37° C. in 5% CO₂, and after four days, 150 mL of AIM-V with 3000 IU/mL of IL-2 may be added to each G-Rex 100 flask. After this, the REP may be completed by harvesting cells on day 14 of culture.

In an embodiment, a method of expanding or treating a cancer includes a step wherein TILs are obtained from a patient tumor sample. A patient tumor sample may be obtained using methods known in the art. For example, TILs may be cultured from enzymatic tumor digests and tumor fragments (about 1 to about 8 mm³ in size) from sharp dissection. Such tumor digests may be produced by incubation in enzymatic media (e.g., Roswell Park Memorial Institute (RPMI) 1640 buffer, 2 mM glutamate, 10 mcg/mL gentamicine, 30 units/mL of DNase and 1.0 mg/mL of collagenase) followed by mechanical dissociation (e.g., using a tissue dissociator). Tumor digests may be produced by placing the tumor in enzymatic media and mechanically dissociating the tumor for approximately 1 minute, followed by incubation for 30 minutes at 37° C. in 5% CO₂, followed by repeated cycles of mechanical dissociation and incubation under the foregoing conditions until only small tissue pieces are present. At the end of this process, if the cell suspension contains a large number of red blood cells or dead cells, a density gradient separation using FICOLL branched hydrophilic polysaccharide may be performed to remove these cells. Alternative methods known in the art may be used, such as those described in U.S. Patent Application Publication No. 2012/0244133 A1, the disclosure of which is incorporated by reference herein. Any of the foregoing methods may be used in any of the embodiments described herein for methods of expanding TILs or methods treating a cancer.

In an embodiment, the second/REP expansion process for TILs may be performed using T-175 flasks and gas permeable bags as previously described (Tran, et al., J. Immunother. 2008, 31, 742-51; Dudley, et al., J. Immunother. 2003, 26, 332-42) or gas permeable cultureware (G-Rex flasks, commercially available from Wilson Wolf Manufacturing Corporation, New Brighton, Minn., USA). For TIL expansion in T-175 flasks, 1×10⁶ TILs suspended in 150 mL of media may be added to each T-175 flask. The TILs may be cultured in a 1 to 1 mixture of CM and AIM-V medium, supplemented with 3000 IU (international units) per mL of IL-2 and 30 ng per ml of anti-CD3 antibody (e.g., OKT-3). The T-175 flasks may be incubated at 37° C. in 5% CO₂. Half the media may be exchanged on day 5 using 50/50 medium with 3000 IU per mL of IL-2. On day 7 cells from two T-175 flasks may be combined in a 3 L bag and 300 mL of AIM V with 5% human AB serum and 3000 IU per mL of IL-2 was added to the 300 ml of TIL suspension. The number of cells in each bag was counted every day or two and fresh media was added to keep the cell count between 0.5 and 2.0×10⁶ cells/mL.

In an embodiment, for second/REP TIL expansions in 500 mL capacity gas permeable flasks with 100 cm² gas-permeable silicon bottoms (G-Rex 100, commercially available from Wilson Wolf Manufacturing Corporation, New Brighton, Minn., USA), 5×10⁶ or 10×10⁶ TIL may be cultured in 400 mL of 50/50 medium, supplemented with 5% human AB serum, 3000 IU per mL of IL-2 and 30 ng per mL of anti-CD3 (OKT-3). The G-Rex 100 flasks may be incubated at 37° C. in 5% CO₂. On day 5, 250 mL of supernatant may be removed and placed into centrifuge bottles and centrifuged at 1500 rpm (revolutions per minute; 491× g) for 10 minutes. The TIL pellets may be re-suspended with 150 mL of fresh medium with 5% human AB serum, 3000 IU per mL of IL-2, and added back to the original G-Rex 100 flasks. When TILs are expanded serially in G-Rex 100 flasks, on day 7 the TILs in each G-Rex 100 flask may be suspended in the 300 mL of media present in each flask and the cell suspension may be divided into 3 100 mL aliquots that may be used to seed 3 G-Rex 100 flasks. Then 150 mL of AIM-V with 5% human AB serum and 3000 IU per mL of IL-2 may be added to each flask. The G-Rex 100 flasks may be incubated at 37° C. in 5% CO₂ and after 4 days 150 mL of AIM-V with 3000 IU per mL of IL-2 may be added to each G-Rex 100 flask. The cells may be harvested on day 14 of culture.

In an embodiment, TILs may be prepared as follows. 2 mm³ tumor fragments are cultured in complete media (CM) comprised of AIM-V medium (Invitrogen Life Technologies, Carlsbad, Calif.) supplemented with 2 mM glutamine (Mediatech, Inc. Manassas, Va.), 100 U/mL penicillin (Invitrogen Life Technologies), 100 μg/mL streptomycin (Invitrogen Life Technologies), 5% heat-inactivated human AB serum (Valley Biomedical, Inc. Winchester, Va.) and 600 IU/mL rhIL-2 (Chiron, Emeryville, Calif.). For enzymatic digestion of liquid tumors, tumor specimens are diced into RPMI-1640, washed and centrifuged at 800 rpm for 5 minutes at 15-22° C., and resuspended in enzymatic digestion buffer (0.2 mg/mL Collagenase and 30 units/ml of DNase in RPMI-1640) followed by overnight rotation at room temperature. TILs established from fragments may be grown for 3-4 weeks in CM and expanded fresh or cryopreserved in heat-inactivated HAB serum with 10% dimethylsulfoxide (DMSO) and stored at −180° C. until the time of study. Tumor associated lymphocytes (TAL) obtained from ascites collections were seeded at 3×10⁶ cells/well of a 24 well plate in CM. TIL growth was inspected about every other day using a low-power inverted microscope.

An Exemplary Embodiment of the TIL Manufacturing Process (the “2A Process”)

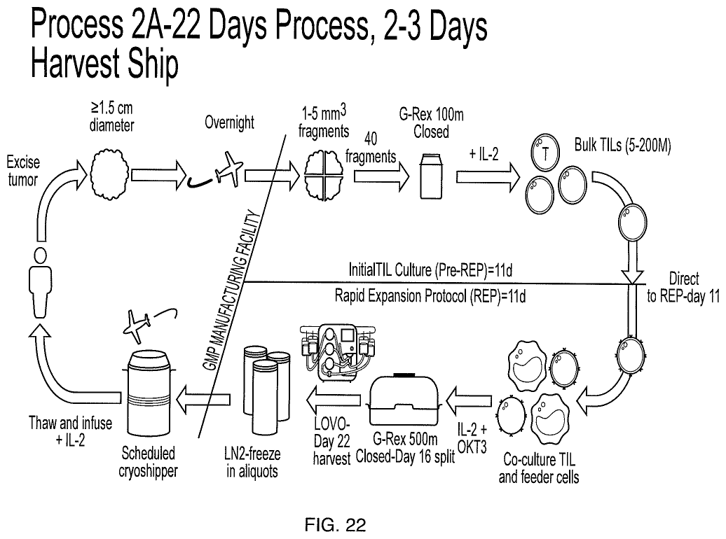

An exemplary TIL manufacturing/expansion process known as process 2A is schematically illustrated in FIG. 22. In certain aspects, the present methods produce TILs which are capable of increased replication cycles upon administration to a subject/patient and as such may provide additional therapeutic benefits over older TILs (i.e., TILs which have further undergone more rounds of replication prior to administration to a subject/patient). Features of younger TILs have been described in the literature, for example Donia, at al., Scandinavian Journal of Immunology, 75:157-167 (2012); Dudley et al., Clin Cancer Res, 16:6122-6131 (2010); Huang et al., J Immunother, 28(3):258-267 (2005); Besser et al., Clin Cancer Res, 19(17):OF1-OF9 (2013); Besser et al., J Immunother 32:415-423 (2009); Robbins, et al., J Immunol 2004; 173:7125-7130; Shen et al., J Immunother, 30:123-129 (2007); Zhou, et al., J Immunother, 28:53-62 (2005); and Tran, et al., J Immunother, 31:742-751 (2008), all of which are incorporated herein by reference in their entireties.

As discussed herein, the present invention can include a step relating to the restimulation of cyropreserved TILs to increase their metabolic activity and thus relative health prior to transplant into a patient, and methods of testing said metabolic health. As generally outlined herein, TILs are generally taken from a patient sample and manipulated to expand their number prior to transplant into a patient. In some embodiments, the TILs may be optionally genetically manipulated as discussed below.

In some embodiments, the TILs may be cryopreserved. Once thawed, they may also be restimulated to increase their metabolism prior to infusion into a patient.

In some embodiments, the first expansion (including processes referred to as the preREP) is shortened in comparison to conventional expansion methods to 7-14 days and the second expansion (including processes referred to as the REP) is shortened to 7-14 days, as discussed in detail below as well as in the examples and figures.

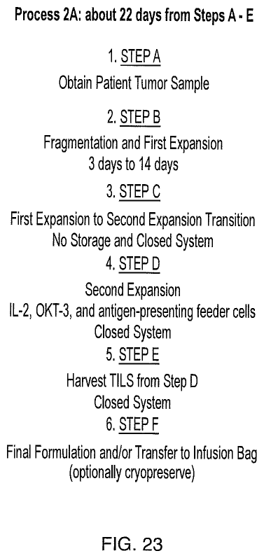

FIG. 23 illustrates an exemplary 2A process. As illustrated in FIG. 23 and further explained in detail below, in some embodiments, the first expansion (Step B) is shortened to 11 days and the second expansion (Step D) is shortened to 11 days. In some embodiments, the combination of the first and second expansions (Step B and Step D) is shortened to 22 days, as discussed in detail herein. As will be appreciated, the process illustrated in FIG. 23 and described below is exemplary and the methods described herein encompass alterations and additions to the described steps as well as any combinations. An exemplary embodiment of this process is described in U.S. Patent Application No. US2018/0282694 A1, which is herein incorporated by reference in its entirety.

Step A: Obtain Patient Tumor Sample

In general, TILs are initially obtained from a patient tumor sample (“primary TILs”) and then expanded into a larger population for further manipulation as described herein, optionally cyropreserved, restimulated as outlined herein and optionally evaluated for phenotype and metabolic parameters as an indication of TIL health.

A patient tumor sample may be obtained using methods known in the art, generally via surgical resection, needle biopsy, apheresis or other means for obtaining a sample that contains a mixture of tumor and TIL cells. In general, the tumor sample may be from any solid tumor, including primary tumors, invasive tumors or metastatic tumors. The tumor sample may also be a liquid tumor, such as a tumor obtained from a hematological malignancy. The solid tumor may be of any cancer type, including, but not limited to, breast, pancreatic, prostate, colorectal, lung, brain, renal, stomach, and skin (including but not limited to squamous cell carcinoma, basal cell carcinoma, and melanoma). In some embodiments, useful TILs are obtained from malignant melanoma tumors, as these have been reported to have particularly high levels of TILs. In some embodiments, the tumor is greater than about 1.5 cm but less than about 4 cm. In some embodiments, the tumor is less than 4 cm.

Once obtained, the tumor sample is generally fragmented using sharp dissection into small pieces of between 1 to about 8 mm³, with from about 2-3 mm³ being particularly useful. The TILs are cultured from these fragments using enzymatic tumor digests. Such tumor digests may be produced by incubation in enzymatic media (e.g., Roswell Park Memorial Institute (RPMI) 1640 buffer, 2 mM glutamate, 10 mcg/mL gentamicine, 30 units/mL of DNase and 1.0 mg/mL of collagenase) followed by mechanical dissociation (e.g., using a tissue dissociator). Tumor digests may be produced by placing the tumor in enzymatic media and mechanically dissociating the tumor for approximately 1 minute, followed by incubation for 30 minutes at 37° C. in 5% CO₂, followed by repeated cycles of mechanical dissociation and incubation under the foregoing conditions until only small tissue pieces are present. At the end of this process, if the cell suspension contains a large number of red blood cells or dead cells, a density gradient separation using FICOLL branched hydrophilic polysaccharide may be performed to remove these cells. Alternative methods known in the art may be used, such as those described in U.S. Patent Application Publication No. 2012/0244133 A1, the disclosure of which is incorporated by reference herein. Any of the foregoing methods may be used in any of the embodiments described herein for methods of expanding TILs or methods treating a cancer.

In general, the harvested cell suspension is called a “primary cell population” or a “freshly harvested” cell population.

In an embodiment, TILs can be initially cultured from enzymatic tumor digests and tumor fragments obtained from patients.

In some embodiments, the TILs, are obtained from tumor fragments. In some embodiments, the tumor fragment is obtained sharp dissection. In some embodiments, the tumor fragment is between about 1 mm³ and 10 mm³. In some embodiments, the tumor fragment is between about 1 mm³ and 8 mm³. In some embodiments, the tumor fragment is about 1 mm³. In some embodiments, the tumor fragment is about 2 mm³. In some embodiments, the tumor fragment is about 3 mm³. In some embodiments, the tumor fragment is about 4 mm³. In some embodiments, the tumor fragment is about 5 mm³. In some embodiments, the tumor fragment is about 6 mm³. In some embodiments, the tumor fragment is about 7 mm³. In some embodiments, the tumor fragment is about 8 mm³. In some embodiments, the tumor fragment is about 9 mm³. In some embodiments, the tumor fragment is about 10 mm³. In some embodiments, about the tumor fragment is about 8-27 mm³. In some embodiments, about the tumor fragment is about 10-25 mm³. In some embodiments, about the tumor fragment is about 15-25 mm³. In some embodiments, the tumor fragment is about 8-20 mm³. In some embodiments, the tumor fragment is about 15-20 mm³. In some embodiments, the tumor fragment is about 8-15 mm³. In some embodiments, the tumor fragment is about 8-10 mm³.

In some embodiments, the number of tumor fragments is about 40 to about 50 tumor fragments. In some embodiments, the number of tumor fragments is about 40 tumor fragments. In some embodiments, the number of tumor fragments is about 50 tumor fragments. In some embodiments, the tumor fragment size is about 8-27 mm³ and there are less than about 50 tumor fragments.

In some embodiments, the TILs, are obtained from tumor digests. In some embodiments, tumor digests were generated by incubation in enzyme media, for example but not limited to RPMI 1640, 2 mM GlutaMAX, 10 mg/mL gentamicin, 30 U/mL DNase, and 1.0 mg/mL collagenase, followed by mechanical dissociation (GentleMACS, Miltenyi Biotec, Auburn, Calif.). After placing the tumor in enzyme media, the tumor can be mechanically dissociated for approximately 1 minute. The solution can then be incubated for 30 minutes at 37° C. in 5% CO₂ and it then mechanically disrupted again for approximately 1 minute. After being incubated again for 30 minutes at 37° C. in 5% CO₂, the tumor can be mechanically disrupted a third time for approximately 1 minute. In some embodiments, after the third mechanical disruption if large pieces of tissue were present, 1 or 2 additional mechanical dissociations were applied to the sample, with or without 30 additional minutes of incubation at 37° C. in 5% CO₂. In some embodiments, at the end of the final incubation if the cell suspension contained a large number of red blood cells or dead cells, a density gradient separation using Ficoll can be performed to remove these cells.

Step B: First Expansion

After dissection or digestion of tumor fragments in Step A, the resulting cells are cultured in serum containing IL-2 under conditions that favor the growth of TILs over tumor and other cells. In some embodiments, the tumor digests are incubated in 2 mL wells in media comprising inactivated human AB serum with 6000 IU/mL of IL-2. This primary cell population is cultured for a period of days, generally from 3 to 14 days, resulting in a bulk TIL population, generally about 1×10⁸ bulk TIL cells. In some embodiments, this primary cell population is cultured for a period of 7 to 14 days, resulting in a bulk TIL population, generally about 1×10⁸ bulk TIL cells. In some embodiments, this primary cell population is cultured for a period of 10 to 14 days, resulting in a bulk TIL population, generally about 1×10⁸ bulk TIL cells. In some embodiments, this primary cell population is cultured for a period of about 11 days, resulting in a bulk TIL population, generally about 1×10⁸ bulk TIL cells. In some embodiments, this primary cell population is cultured for a period of about 11 days, resulting in a bulk TIL population, generally less than or equal to about 200×10⁶ bulk TIL cells.

In a preferred embodiment, expansion of TILs may be performed using an initial bulk TIL expansion step (Step B as pictured in FIG. 23, which can include processes referred to as pre-REP) as described below and herein, followed by a second expansion (Step D, including processes referred to as rapid expansion protocol (REP) steps) as described below under Step D and herein, followed by optional cryopreservation, and followed by a second Step D (including processes referred to as restimulation REP steps) as described below and herein. The TILs obtained from this process may be optionally characterized for phenotypic characteristics and metabolic parameters as described herein.

In embodiments where TIL cultures are initiated in 24-well plates, for example, using Costar 24-well cell culture cluster, flat bottom (Corning Incorporated, Corning, N.Y., each well can be seeded with 1×10⁶ tumor digest cells or one tumor fragment in 2mL of complete medium (CM) with IL-2 (6000 IU/mL; Chiron Corp., Emeryville, Calif.). In some embodiments, the tumor fragment is between about 1 mm³ and 10 mm³.

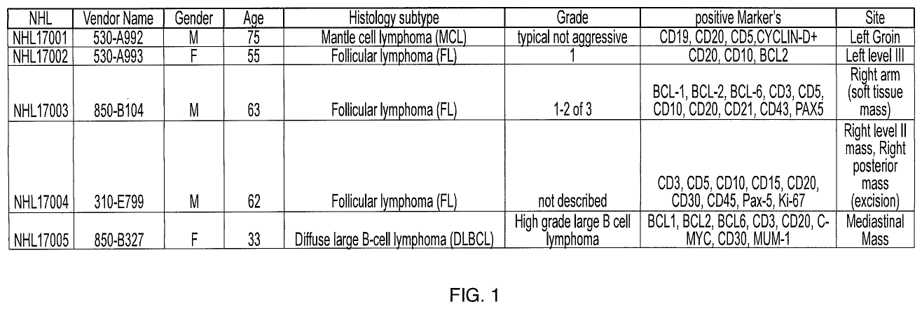

In some embodiments, CM for Step B consists of RPMI 1640 with GlutaMAX, supplemented with 10% human AB serum, 25 mM HEPES, and 10 mg/mL gentamicin. In embodiments where cultures are initiated in gas-permeable flasks with a 40 mL capacity and a 10 cm² gas-permeable silicon bottom (for example, G-Rex10; Wilson Wolf Manufacturing, New Brighton, Minn.) (FIG. 1), each flask was loaded with 10-40×10⁶ viable tumor digest cells or 5-30 tumor fragments in 10-40 mL of CM with IL-2. Both the G-Rex10 and 24-well plates were incubated in a humidified incubator at 37° C. in 5% CO₂ and 5 days after culture initiation, half the media was removed and replaced with fresh CM and IL-2 and after day 5, half the media was changed every 2-3 days.

In an embodiment, the cell culture medium further comprises IL-2. In a preferred embodiment, the cell culture medium comprises about 3000 IU/mL of IL-2. In an embodiment, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL of IL-2. In an embodiment, the cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or between 8000 IU/mL of IL-2.

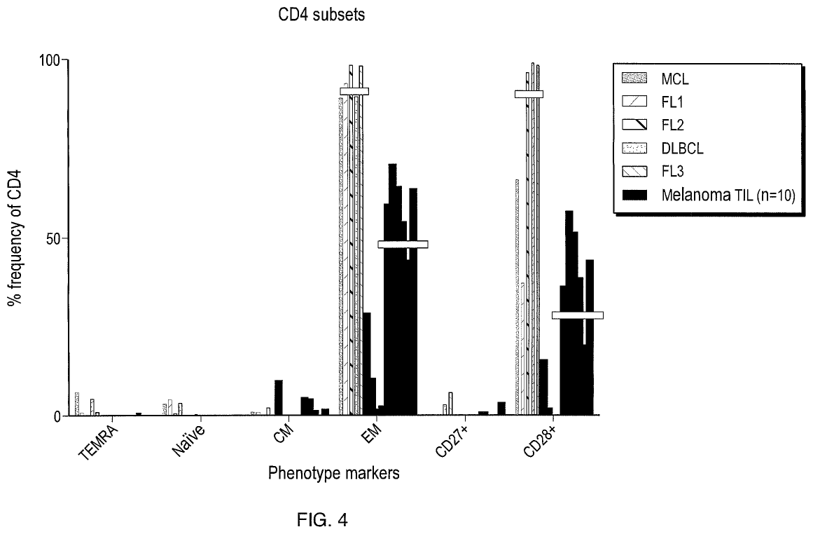

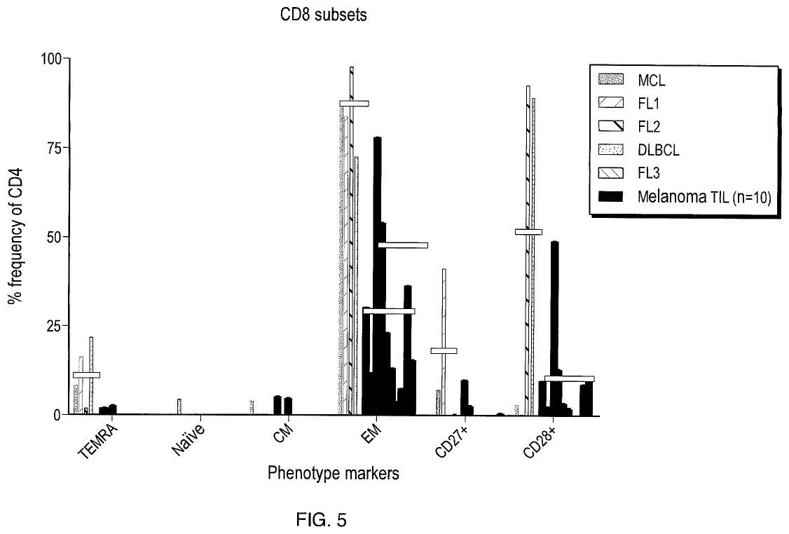

In some embodiments, the first expansion (including processes referred to as the pre-REP; Step B) process is shortened to 3-14 days, as discussed in the examples and figures. In some embodiments, the first expansion of Step B is shortened to 7-14 days, as discussed in the Examples and shown in FIGS. 4 and 5. In some embodiments, the first expansion of Step B is shortened to 10-14 days, as discussed in the Examples. In some embodiments, the first expansion of Step B is shortened to 11 days, as discussed in the Examples.

In some embodiments, IL-2, IL-7, IL-15, and IL-21 as well as combinations thereof can be included during Step B processes as described herein.

In some embodiments, Step B is performed in a closed system bioreactor. In some embodiments, a closed system is employed for the TIL expansion, as described herein. In some embodiments, a single bioreactor is employed. In some embodiments, the single bioreactor employed is for example a GREX-10 or a GREX-100.

Step C: First Expansion to Second Expansion Transition

In some embodiments, the bulk TIL population from Step B can be cryopreserved immediately, using methods known in the art and described herein. Alternatively, the bulk TIL population can be subjected to a second expansion (REP) and then cryopreserved as discussed below.

In some embodiments, the Step B TILs are not stored and the Step B TILs proceed directly to Step D. In some embodiments, the transition occurs in a closed system, as further described herein.

Step D: Second Expansion

In some embodiments, the TIL cell population is expanded in number after harvest and initial bulk processing (i.e., after Step A and Step B). This is referred to herein as the second expansion, which can include expansion processes generally referred to in the art as a rapid expansion process (REP). The second expansion is generally accomplished using culture media comprising a number of components, including feeder cells, a cytokine source, and an anti-CD3 antibody, in a gas-permeable container. In some embodiments, the second expansion can include scaling-up in order to increase the number of TILs obtained in the second expansion.

In an embodiment, REP and/or the second expansion can be performed in a gas permeable container using the methods of the present disclosure. For example, TILs can be rapidly expanded using non-specific T-cell receptor stimulation in the presence of interleukin-2 (IL-2) or interleukin-15 (IL-15). The non-specific T-cell receptor stimulus can include, for example, about 30 ng/ml of OKT3, a mouse monoclonal anti-CD3 antibody (commercially available from Ortho-McNeil, Raritan, N.J. or Miltenyi Biotech, Auburn, Calif.). TILs can be rapidly expanded further stimulation of the TILs in vitro with one or more antigens, including antigenic portions thereof, such as epitope(s), of the cancer, which can be optionally expressed from a vector, such as a human leukocyte antigen A2 (HLA-A2) binding peptide, e.g., 0.3μM MART-1 :26-35 (27 L) or gpl 00:209-217 (210M), optionally in the presence of a T-cell growth factor, such as 300 IU/mL IL-2 or IL-15. Other suitable antigens may include, e.g., NY-ESO-1, TRP-1, TRP-2, tyrosinase cancer antigen, MAGE-A3, SSX-2, and VEGFR2, or antigenic portions thereof. TIL may also be rapidly expanded by re-stimulation with the same antigen(s) of the cancer pulsed onto HLA-A2-expressing antigen-presenting cells. Alternatively, the TILs can be further re-stimulated with, e.g., example, irradiated, autologous lymphocytes or with irradiated HLA-A2+ allogeneic lymphocytes and IL-2.

In an embodiment, the cell culture medium further comprises IL-2. In a preferred embodiment, the cell culture medium comprises about 3000 IU/mL of IL-2. In an embodiment, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL of IL-2. In an embodiment, the cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or between 8000 IU/mL of IL-2.

In an embodiment, the cell culture medium comprises OKT3 antibody. In a preferred embodiment, the cell culture medium comprises about 30 ng/mL of OKT3 antibody. In an embodiment, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, and about 1 μg/mL of OKT3 antibody. In an embodiment, the cell culture medium comprises between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, and between 50 ng/mL and 100 ng/mL of OKT3 antibody.

In some embodiments, IL-2, IL-7, IL-15, and IL-21 as well as combinations thereof can be included during the second expansion in Step D processes as described herein.

In some embodiments, the second expansion can be conducted in a supplemented cell culture medium comprising IL-2, OKT-3, and antigen-presenting feeder cells.

In some embodiments the antigen-presenting feeder cells (APCs) are PBMCs. In an embodiment, the ratio of TILs to PBMCs and/or antigen-presenting cells in the rapid expansion and/or the second expansion is about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500. In an embodiment, the ratio of TILs to PBMCs in the rapid expansion and/or the second expansion is between 1 to 50 and 1 to 300. In an embodiment, the ratio of TILs to PBMCs in the rapid expansion and/or the second expansion is between 1 to 100 and 1 to 200.

In an embodiment, REP and/or the second expansion is performed in flasks with the bulk TILs being mixed with a 100- or 200-fold excess of inactivated feeder cells, 30 mg/mL OKT3 anti-CD3 antibody and 3000 IU/mL IL-2 in 150 ml media. Media replacement is done (generally ⅔ media replacement via respiration with fresh media) until the cells are transferred to an alternative growth chamber. Alternative growth chambers include GRex flasks and gas permeable containers as more fully discussed below.

In some embodiments, the second expansion (also referred to as the REP process) is shortened to 7-14 days, as discussed in the examples and figures. In some embodiments, the second expansion is shortened to 11 days.

In an embodiment, REP and/or the second expansion may be performed using T-175 flasks and gas permeable bags as previously described (Tran, et al., J. Immunother. 2008, 31, 742-51; Dudley, et al., J. Immunother. 2003, 26, 332-42) or gas permeable cultureware (G-Rex flasks). For TIL rapid expansion and/or second expansion in T-175 flasks, 1×10⁶ TILs suspended in 150 mL of media may be added to each T-175 flask. The TILs may be cultured in a 1 to 1 mixture of CM and AIM-V medium, supplemented with 3000 IU per mL of IL-2 and 30 ng per ml of anti-CD3. The T-175 flasks may be incubated at 37° C. in 5% CO₂. Half the media may be exchanged on day 5 using 50/50 medium with 3000 IU per mL of IL-2. On day 7 cells from two T-175 flasks may be combined in a 3 L bag and 300 mL of AIM V with 5% human AB serum and 3000 IU per mL of IL-2 was added to the 300 ml of TIL suspension. The number of cells in each bag was counted every day or two and fresh media was added to keep the cell count between 0.5 and 2.0×10⁶ cells/mL.

In an embodiment, REP and/or the second expansion may be performed in 500 mL capacity gas permeable flasks with 100 cm gas-permeable silicon bottoms (G-Rex 100, commercially available from Wilson Wolf Manufacturing Corporation, New Brighton, Minn., USA), 5×10⁶ or 10×10⁶ TIL may be cultured with PBMCs in 400 mL of 50/50 medium, supplemented with 5% human AB serum, 3000 IU per mL of IL-2 and 30 ng per ml of anti-CD3 (OKT3). The G-Rex 100 flasks may be incubated at 37° C. in 5% CO₂. On day 5, 250 mL of supernatant may be removed and placed into centrifuge bottles and centrifuged at 1500 rpm (491× g) for 10 minutes. The TIL pellets may be re-suspended with 150 mL of fresh medium with 5% human AB serum, 3000 IU per mL of IL-2, and added back to the original G-Rex 100 flasks. When TIL are expanded serially in G-Rex 100 flasks, on day 7 the TIL in each G-Rex 100 may be suspended in the 300 mL of media present in each flask and the cell suspension may be divided into 3 100 mL aliquots that may be used to seed 3 G-Rex 100 flasks. Then 150 mL of AIM-V with 5% human AB serum and 3000 IU per mL of IL-2 may be added to each flask. The G-Rex 100 flasks may be incubated at 37° C. in 5% CO₂ and after 4 days 150 mL of AIM-V with 3000 IU per mL of IL-2 may be added to each G-Rexl OO flask. The cells may be harvested on day 14 of culture.

In an embodiment, REP and/or the second expansion is performed in flasks with the bulk TILs being mixed with a 100- or 200-fold excess of inactivated feeder cells, 30 mg/mL OKT3 anti-CD3 antibody and 3000 IU/mL IL-2 in 150 ml media. Media replacement is done (generally ⅔ media replacement via respiration with fresh media) until the cells are transferred to an alternative growth chamber. Alternative growth chambers include GRex flasks and gas permeable containers as more fully discussed below.

In an embodiment, REP and/or the second expansion is performed and further comprises a step wherein TILs are selected for superior tumor reactivity. Any selection method known in the art may be used. For example, the methods described in U.S. Patent Application Publication No. 2016/0010058 A1, the disclosures of which are incorporated herein by reference, may be used for selection of TILs for superior tumor reactivity.

REP and/or the second expansion of TIL can be performed using T-175 flasks and gas-permeable bags as previously described (Tran K Q, Zhou J, Durflinger K H, et al., 2008, J Immunother., 31:742-751, and Dudley M E, Wunderlich J R, Shelton T E, et al. 2003, J Immunother., 26:332-342) or gas-permeable G-Rex flasks. In some embodiments, REP and/or the second expansion is performed using flasks. In some embodiments, REP is performed using gas-permeable G-Rex flasks. For TIL REP and/or the second expansion in T-175 flasks, about 1×10⁶ TIL are suspended in about 150 mL of media and this is added to each T-175 flask. The TIL are cultured with irradiated (50 Gy) allogeneic PBMC as “feeder” cells at a ratio of 1 to 100 and the cells were cultured in a 1 to 1 mixture of CM and AIM-V medium (50/50 medium), supplemented with 3000 IU/mL of IL-2 and 30 ng/mL of anti-CD3. The T-175 flasks are incubated at 37° C. in 5% CO₂. In some embodiments, half the media is changed on day 5 using 50/50 medium with 3000 IU/mL of IL-2. In some embodiments, on day 7, cells from 2 T-175 flasks are combined in a 3 L bag and 300 mL of AIM-V with 5% human AB serum and 3000 IU/mL of IL-2 is added to the 300 mL of TIL suspension. The number of cells in each bag can be counted every day or two and fresh media can be added to keep the cell count between about 0.5 and about 2.0×10⁶ cells/mL.

For TIL REP and/or the second expansion in 500 mL capacity flasks with 100 cm² gas-permeable silicon bottoms (G-Rex100,Wilson Wolf), about 5×10⁶ or 10×10⁶ TIL are cultured with irradiated allogeneic PBMC at a ratio of 1 to 100 in 400 mL of 50/50 medium, supplemented with 3000 IU/mL of IL-2 and 30 ng/mL of anti-CD3. The G-Rex100 flasks are incubated at 37° C. in 5% CO₂. In some embodiments, on day 5, 250 mL of supernatant is removed and placed into centrifuge bottles and centrifuged at 1500 rpm (491 g) for 10 minutes. The TIL pellets can then be resuspended with 150 mL of fresh 50/50 medium with 3000 IU/mL of IL-2 and added back to the original G-Rex100 flasks. In embodiments where TILs are expanded serially in G-Rex100 flasks, on day 7 the TIL in each G-Rex100 are suspended in the 300 mL of media present in each flask and the cell suspension was divided into three 100 mL aliquots that are used to seed 3 G-Rex100 flasks. Then 150 mL of AIM-V with 5% human AB serum and 3000 IU/mL of IL-2 is added to each flask. The G-Rex100 flasks are incubated at 37° C. in 5% CO₂ and after 4 days 150 mL of AIM-V with 3000 IU/mL of IL-2 is added to each G-Rex100 flask. The cells are harvested on day 14 of culture.

Feeder Cells and Antigen Presenting Cells

In an embodiment, the second expansion procedures described herein (Step D, including REP) require an excess of feeder cells during REP TIL expansion and/or during the second expansion. In many embodiments, the feeder cells are peripheral blood mononuclear cells (PBMCs) obtained from standard whole blood units from healthy blood donors. The PBMCs are obtained using standard methods such as Ficoll-Paque gradient separation.

In general, the allogenic PBMCs are inactivated, either via irradiation or heat treatment, and used in the REP procedures, as described in the examples, in particular example 14, which provides an exemplary protocol for evaluating the replication incompetence of irradiate allogeneic PBMCs.

In some embodiments, PBMCs are considered replication incompetent and accepted for use in the TIL expansion procedures described herein if the total number of viable cells on day 14 is less than the initial viable cell number put into culture on day 0 of the REP and/or day 0 of the second expansion (i.e., the start day of the second expansion).

In some embodiments, PBMCs are considered replication incompetent and accepted for use in the TIL expansion procedures described herein if the total number of viable cells, cultured in the presence of OKT3 and IL-2, on day 7 and day 14 has not increased from the initial viable cell number put into culture on day 0 of the REP and/or day 0 of the second expansion (i.e., the start day of the second expansion). In some embodiments, the PBMCs are cultured in the presence of 30 ng/ml OKT3 antibody and 3000 IU/ml IL-2.

In some embodiments, PBMCs are considered replication incompetent and accepted for use in the TIL expansion procedures described herein if the total number of viable cells, cultured in the presence of OKT3 and IL-2, on day 7 and day 14 has not increased from the initial viable cell number put into culture on day 0 of the REP and/or day 0 of the second expansion (i.e., the start day of the second expansion). In some embodiments, the PBMCs are cultured in the presence of 5-60 ng/ml OKT3 antibody and 1000-6000 IU/ml IL-2. In some embodiments, the PBMCs are cultured in the presence of 10-50 ng/ml OKT3 antibody and 2000-5000 IU/ml IL-2. In some embodiments, the PBMCs are cultured in the presence of 20-40 ng/ml OKT3 antibody and 2000-4000 IU/ml IL-2. In some embodiments, the PBMCs are cultured in the presence of 25-35 ng/ml OKT3 antibody and 2500-3500 IU/ml IL-2.

In an embodiment, artificial antigen presenting cells are used in the REP stage as a replacement for, or in combination with, PBMCs.

Cytokines

The expansion methods described herein generally use culture media with high doses of a cytokine, in particular IL-2, as is known in the art.

Alternatively, using combinations of cytokines for the rapid expansion and or second expansion of TILs is additionally possible, with combinations of two or more of IL-2, IL-15 and IL-21 as is generally outlined in U.S. Patent Application Publication No. US 2017/0107490 A1, International Publication No. WO 2015/189356, U.S. Patent Application Publication No. US 2017/0107490 A1, and International Publication No. WO 2015/189357, each of which is hereby expressly incorporated by reference in their entirety. Thus, possible combinations include IL-2 and IL-15, IL-2 and IL-21, IL-15 and IL-21 and IL-2, IL-15 and IL-21, with the latter finding particular use in many embodiments. The use of combinations of cytokines specifically favors the generation of lymphocytes, and in particular T-cells as described therein.

Anti-CD3 Antibodies

In some embodiments, the culture media used in expansion methods described herein (including REP) also includes an anti-CD3 antibody. An anti-CD3 antibody in combination with IL-2 induces T cell activation and cell division in the TIL population. This effect can be seen with full length antibodies as well as Fab and F(ab′)2 fragments, with the former being generally preferred; see, e.g., Tsoukas et al., J. Immunol. 1985, 135, 1719, hereby incorporated by reference in its entirety.

As will be appreciated by those in the art, there are a number of suitable anti-human CD3 antibodies that find use in the invention, including anti-human CD3 polyclonal and monoclonal antibodies from various mammals, including, but not limited to, murine, human, primate, rat, and canine antibodies. In particular embodiments, the OKT3 anti-CD3 antibody is used (commercially available from Ortho-McNeil, Raritan, N.J. or Miltenyi Biotech, Auburn, Calif.).

Step E: Harvest TILs

After the second expansion step, cells can be harvested. In some embodiments the TILs are harvested after one, two, three, four or more second expansion steps.

TILs can be harvested in any appropriate and sterile manner, including for example by centrifugation. Methods for TIL harvesting are well known in the art and any such know methods can be employed with the present process. In some embodiments, TILs are harvest using an automated system. In some embodiments, TILs are harvest using a semi-automated system. In some embodiments, TILs are harvested using a semi-automated system. In some embodiments, the TILs from the second expansion are harvested using a semi-automated machine. In some embodiments, the LOVO system is employed (commercially available from Benchmark Electronics, for example). In some embodiments, the harvesting step includes wash the TILs, formulating the TILs, and/or aliquoting the TILs. In some embodiments, the cells are optionally frozen after harvesting or as part of harvesting.

Step F: Final Formulation/Transfer to Infusion Bag

After Steps A through E are complete, cells are transferred to a container for use in administration to a patient.

In an embodiment, TILs expanded using APCs of the present disclosure are administered to a patient as a pharmaceutical composition. In an embodiment, the pharmaceutical composition is a suspension of TILs in a sterile buffer. TILs expanded using PBMCs of the present disclosure may be administered by any suitable route as known in the art. In some embodiments, the T-cells are administered as a single intra-arterial or intravenous infusion, which preferably lasts approximately 30 to 60 minutes. Other suitable routes of administration include intraperitoneal, intrathecal, and intralymphatic.

Additional Expansion Steps

As will be appreciated, any of the steps A through F described above can be repeated any number of times and may in addition be conducted in different orders than described above.

In some embodiments, one or more of the expansion steps may be repeated prior to the Final Formulation Step F. Such additional expansion steps may include the elements of the first and/or second expansion steps described above (e.g., include the described components in the cell culture medium). The additional expansion steps may further include additional elements, including additional components in the cell culture medium that are supplemented into the cell culture medium before and/or during the additional expansion steps.

In further embodiments, any of the expansion steps described in FIG. 23 and in the above paragraphs may be preceded or followed by a cryopreservation step in which the cells produced during an expansion step are preserved using methods known in the art for storage until needed for the remaining steps of the manufacturing/expansion process.

### Pharmaceutical Compositions, Dosages, and Dosing Regimens for TILs, MILs, and PBLs

In an embodiment, the invention provides a therapeutic population of TILs prepared by any method of expanding TILs described herein, optionally modified to express a chimeric antigen receptor (CAR) and/or express a modified T-cell receptor and/or suppress or reduce expression of one or more immune checkpoint genes in a transient or stable manner as described herein.

In another embodiment, the invention provides a therapeutic population of MILs prepared by any method of expanding MILs described herein, optionally modified to express a chimeric antigen receptor (CAR) and/or express a modified T-cell receptor and/or suppress or reduce expression of one or more immune checkpoint genes as described herein.

In another embodiment, the invention provides a therapeutic population of PBLs prepared by any method of expanding PBLs described herein, optionally modified to express a chimeric antigen receptor (CAR) and/or express a modified T-cell receptor and/or suppress or reduce expression of one or more immune checkpoint genes as described herein.

In another embodiment, the invention provides a pharmaceutical composition comprising a therapeutic population of TILs prepared by any method of expanding TILs described herein, optionally modified to express a chimeric antigen receptor (CAR) and/or express a modified T-cell receptor and/or suppress or reduce expression of one or more immune checkpoint genes as described herein, and a pharmaceutically acceptable carrier.

In another embodiment, the invention provides a pharmaceutical composition comprising a therapeutic population of MILs prepared by any method of expanding MILs described herein, optionally modified to express a chimeric antigen receptor (CAR) and/or express a modified T-cell receptor and/or suppress or reduce expression of one or more immune checkpoint genes as described herein, and a pharmaceutically acceptable carrier.

In another embodiment, the invention provides a pharmaceutical composition comprising a therapeutic population of PBLs prepared by any method of expanding PBLs described herein, optionally modified to express a chimeric antigen receptor (CAR) and/or express a modified T-cell receptor and/or suppress or reduce expression of one or more immune checkpoint genes as described herein, and a pharmaceutically acceptable carrier.

In an embodiment, TILs expanded using methods of the present disclosure are administered to a patient as a pharmaceutical composition. In an embodiment, the pharmaceutical composition is a suspension of TILs in a sterile buffer. TILs expanded using methods of the present disclosure may be administered by any suitable route as known in the art. Preferably, the TILs are administered as a single intra-arterial or intravenous infusion, which preferably lasts approximately 30 to 60 minutes. Other suitable routes of administration include intraperitoneal, intrathecal, and intralymphatic administration.

Any suitable dose of TILs can be administered. Preferably, from about 2.3×10¹⁰ to about 13.7×10¹⁰ TILs are administered, with an average of around 7.8×10¹⁰ TILs, particularly if the cancer is a hematological malignancy. In an embodiment, about 1.2×10¹⁰ to about 4.3×10¹⁰ of TILs are administered.

In some embodiments, the number of the TILs provided in the pharmaceutical compositions of the invention is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In an embodiment, the number of the TILs provided in the pharmaceutical compositions of the invention is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³. In an embodiment of the invention, the number of TILs provided in the pharmaceutical compositions of the invention is in the range of from about 4×10⁸ to about 2.5×10⁹. In another embodiment, the number of TILs provided in the pharmaceutical compositions of the invention is 9.5×10⁸. In another embodiment, the number of TILs provided in the pharmaceutical compositions of the invention is 4.1×10⁸. In another embodiment, the number of TILs provided in the pharmaceutical compositions of the invention is 2.2×10⁹.

In an embodiment of the invention, the number of TILs provided in the pharmaceutical compositions of the invention is in the range of from about 0.1×10⁹ to about 15×10⁹ TILs, from about 0.1×10⁹ to about 15×10⁹ TILs, from about 0.12×10⁹ to about 12×10⁹ TILs, from about 0.15×10⁹ to about 11×10⁹ TILs, from about 0.2×10⁹ to about 10×10⁹ TILs, from about 0.3×10⁹ to about 9×10⁹ TILs, from about 0.4×10⁹ to about 8×10⁹ TILs, from about 0.5×10⁹ to about 7×10⁹ TILs, from about 0.6×10⁹ to about 6×10⁹ TILs, from about 0.7×10⁹ to about 5×10⁹ TILs, from about 0.8×10⁹ to about 4×10⁹ TILs, from about 0.9×10⁹ to about 3×10⁹ TILs, or from about 1×10⁹ to about 2×10⁹ TILs.

In some embodiments, the concentration of the TILs provided in the pharmaceutical compositions of the invention is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of the TILs provided in the pharmaceutical compositions of the invention is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of the TILs provided in the pharmaceutical compositions of the invention is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of the TILs provided in the pharmaceutical compositions of the invention is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of the TILs provided in the pharmaceutical compositions of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the TILs provided in the pharmaceutical compositions of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

The TILs provided in the pharmaceutical compositions of the invention are effective over a wide dosage range. The exact dosage will depend upon the route of administration, the form in which the compound is administered, the gender and age of the subject to be treated, the body weight of the subject to be treated, and the preference and experience of the attending physician. The clinically-established dosages of the TILs may also be used if appropriate. The amounts of the pharmaceutical compositions administered using the methods herein, such as the dosages of TILs, will be dependent on the human or mammal being treated, the severity of the disorder or condition, the rate of administration, the disposition of the active pharmaceutical ingredients and the discretion of the prescribing physician.

In some embodiments, TILs may be administered in a single dose. Such administration may be by injection, e.g., intravenous injection. In some embodiments, TILs may be administered in multiple doses. Dosing may be once, twice, three times, four times, five times, six times, or more than six times per year. Dosing may be once a month, once every two weeks, once a week, or once every other day. Administration of TILs may continue as long as necessary.

In some embodiments, an effective dosage of TILs is about 1×10⁶, 2×10⁶, 3 ×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In some embodiments, an effective dosage of TILs is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³.

In an embodiment of the invention, the clinical dose of MILs useful for patients with acute myeloid leukemia (AML) is in the range of from about 4×10⁸ to about 2.5×10⁹ MILs. In another embodiment, the number of MILs provided in the pharmaceutical compositions of the invention is 9.5×10⁸ MILs. In another embodiment, the number of MILs provided in the pharmaceutical compositions of the invention is 4.1×10⁸. In another embodiment, the number of MILs provided in the pharmaceutical compositions of the invention is 2.2×10⁹.

In some embodiments, an effective dosage of TILs is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some embodiments, an effective dosage of TILs is in the range of about 1 mg to about 500 mg, about 10 mg to about 300 mg, about 20 mg to about 250 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg.

An effective amount of the TILs may be administered in either single or multiple doses by any of the accepted modes of administration of agents having similar utilities, including intranasal and transdermal routes, by intra-arterial injection, intravenously, intraperitoneally, parenterally, intramuscularly, subcutaneously, topically, by transplantation or direct injection into tumor, or by inhalation.

Optional Genetic Engineering of TILs, PBLs, and/or MILs

In some embodiments, the expanded TILs, PBLs, and/or MILs of the present invention are further manipulated before, during, or after an expansion step, including during closed, sterile manufacturing processes, each as provided herein, in order to alter protein expression in a transient manner. In some embodiments, the transiently altered protein expression is due to transient gene editing. In some embodiments, the expanded TILs, PBLs, and/or MILs of the present invention are treated with transcription factors (TFs) and/or other molecules capable of transiently altering protein expression in the TILs, PBLs, and/or MILs. In some embodiments, the TFs and/or other molecules that are capable of transiently altering protein expression provide for altered expression of tumor antigens and/or an alteration in the number of tumor antigen-specific T cells in a population of TILs, PBLs, and/or MILs.

In certain embodiments, the method comprises genetically editing a population of TILs, PBLs, and/or MILs. In certain embodiments, the method comprises genetically editing the first population of TILs, PBLs, and/or MILs, the second population of TILs, PBLs, and/or MILs and/or the third population of TILs, PBLs, and/or MILs.

In some embodiments, the present invention includes genetic editing through nucleotide insertion, such as through ribonucleic acid (RNA) insertion, including insertion of messenger RNA (mRNA) or small (or short) interfering RNA (siRNA), into a population of TILs, PBLs, and/or MILs for promotion of the expression of one or more proteins or inhibition of the expression of one or more proteins, as well as simultaneous combinations of both promotion of one set of proteins with inhibition of another set of proteins.

In some embodiments, the expanded TILs, PBLs, and/or MILs of the present invention undergo transient alteration of protein expression. In some embodiments, the transient alteration of protein expression occurs at any time before, during, or after the expansion process. In some embodiments, the transient alteration of protein expression occurs at any step within the expansion process. In some embodiments, the transient alteration of protein expression occurs in the bulk TIL, PBL, and/or MIL population prior to a first expansion. In some embodiments, the transient alteration of protein expression occurs during the first expansion. In some embodiments, the transient alteration of protein expression occurs after the first expansion, including, for example in the TIL, PBL, and/or MIL population in transition between the first and second expansion (e.g. the second population of TILs, PBLs, and/or MILs as described herein. In some embodiments, the transient alteration of protein expression occurs in the bulk TIL, PBL, and/or MIL population prior to second expansion. In some embodiments, the transient alteration of protein expression occurs during the second expansion, including, for example in the TIL, PBL, and/or MIL population being expanded (e.g. the third population of TILs, PBLs, and/or MILs). In some embodiments, the transient alteration of protein expression occurs after the second expansion.

In an embodiment, a method of transiently altering protein expression in a population of TILs, PBLs, and/or MILs includes the step of electroporation. Electroporation methods are known in the art and are described, e.g., in Tsong, Biophys. 1 1991, 60, 297-306, and U.S. Patent Application Publication No. 2014/0227237 A1, the disclosures of each of which are incorporated by reference herein. In an embodiment, a method of transiently altering protein expression in population of TILs, PBLs, and/or MILs includes the step of calcium phosphate transfection. Calcium phosphate transfection methods (calcium phosphate DNA precipitation, cell surface coating, and endocytosis) are known in the art and are described in Graham and van der Eb, Virology 1973, 52, 456-467; Wigler, et al., Proc. Natl. Acad. Sci. 1979, 76, 1373-1376; and Chen and Okayarea, Mol. Cell. Biol. 1987, 7, 2745-2752; and in U.S. Pat. No. 5,593,875, the disclosures of each of which are incorporated by reference herein. In an embodiment, a method of transiently altering protein expression in a population of TILs, PBLs, and/or MILs includes the step of liposomal transfection. Liposomal transfection methods, such as methods that employ a 1:1 (w/w) liposome formulation of the cationic lipid N-[1-(2,3-dioleyloxy)propyl]-n,n,n-trimethylammonium chloride (DOTMA) and dioleoyl phophotidylethanolamine (DOPE) in filtered water, are known in the art and are described in Rose, et al., Biotechniques 1991, 10, 520-525 and Felgner, et al., Proc. Natl. Acad. Sci. USA, 1987, 84, 7413-7417 and in U.S. Pat. Nos. 5,279,833; 5,908,635; 6,056,938; 6,110,490; 6,534,484; and 7,687,070, the disclosures of each of which are incorporated by reference herein. In an embodiment, a method of transiently altering protein expression in a population of TILs, PBLs, and/or MILs includes the step of transfection using methods described in U.S. Pat. Nos. 5,766,902; 6,025,337; 6,410,517; 6,475,994; and 7,189,705; the disclosures of each of which are incorporated by reference herein.

In some embodiments, transient alteration of protein expression results in an increase in Stem Memory T cells (TSCMs). TSCMs are early progenitors of antigen-experienced central memory T cells. TSCMs generally display the long-term survival, self-renewal, and multipotency abilities that define stem cells, and are generally desirable for the generation of effective TIL products. TSCM have shown enhanced anti-tumor activity compared with other T cell subsets in mouse models of adoptive cell transfer (Gattinoni et al. Nat Med 2009, 2011; Gattinoni, Nature Rev. Cancer, 2012; Cieri et al. Blood 2013). In some embodiments, transient alteration of protein expression results in a TIL population with a composition comprising a high proportion of TSCM. In some embodiments, transient alteration of protein expression results in an at least 5%, at least 10%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% increase in TSCM percentage. In some embodiments, transient alteration of protein expression results in an at least a 1-fold, 2-fold, 3-fold, 4-fold, 5-fold, or 10-fold increase in TSCMs in the TIL population. In some embodiments, transient alteration of protein expression results in a TIL population with at least at least 5%, at least 10%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% TSCMs. In some embodiments, transient alteration of protein expression results in a therapeutic TIL population with at least at least 5%, at least 10%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% TSCMs.

In some embodiments, transient alteration of protein expression results in rejuvenation of antigen-experienced T-cells. In some embodiments, rejuvenation includes, for example, increased proliferation, increased T-cell activation, and/or increased antigen recognition.

In some embodiments, transient alteration of protein expression alters the expression in a large fraction of the T-cells in order to preserve the tumor-derived TCR repertoire. In some embodiments, transient alteration of protein expression does not alter the tumor-derived TCR repertoire. In some embodiments, transient alteration of protein expression maintains the tumor-derived TCR repertoire.

In some embodiments, transient alteration of protein results in altered expression of a particular gene. In some embodiments, the transient alteration of protein expression targets a gene including but not limited to PD-1 (also referred to as PDCD1 or CC279), TGFBR2, CCR4/5, CBLB (CBL-B), CISH, CCRs (chimeric co-stimulatory receptors), IL-2, IL-12, IL-15, IL-21, NOTCH 1/2 ICD, TIM3, LAG3, TIGIT, TGFβ, CCR2, CCR4, CCR5, CXCR1, CXCR2, CSCR3, CCL2 (MCP-1), CCL3 (MIP-1α), CCL4 (MIP1-β), CCL5 (RANTES), CXCL1/CXCL8, CCL22, CCL17, CXCL1/CXCL8, VHL, CD44, PIK3CD, SOCS1, and/or cAMP protein kinase A (PKA). In some embodiments, the transient alteration of protein expression targets a gene selected from the group consisting of PD-1, TGFBR2, CCR4/5, CBLB (CBL-B), CISH, CCRs (chimeric co-stimulatory receptors), IL-2, IL-12, IL-15, IL-21, NOTCH 1/2 ICD, TIM3, LAG3, TIGIT, TGFβ, CCR2, CCR4, CCR5, CXCR1, CXCR2, CSCR3, CCL2 (MCP-1), CCL3 (MIP-1α), CCL4 (MIP1-β), CCL5 (RANTES), CXCL1/CXCL8, CCL22, CCL17, CXCL1/CXCL8, VHL, CD44, PIK3CD, SOCS1, and/or cAMP protein kinase A (PKA). In some embodiments, the transient alteration of protein expression targets PD-1. In some embodiments, the transient alteration of protein expression targets TGFBR2. In some embodiments, the transient alteration of protein expression targets CCR4/5. In some embodiments, the transient alteration of protein expression targets CBLB. In some embodiments, the transient alteration of protein expression targets CISH. In some embodiments, the transient alteration of protein expression targets CCRs (chimeric co-stimulatory receptors). In some embodiments, the transient alteration of protein expression targets IL-2. In some embodiments, the transient alteration of protein expression targets IL-12. In some embodiments, the transient alteration of protein expression targets IL-15. In some embodiments, the transient alteration of protein expression targets IL-21. In some embodiments, the transient alteration of protein expression targets NOTCH 1/2 ICD. In some embodiments, the transient alteration of protein expression targets TIM3. In some embodiments, the transient alteration of protein expression targets LAG3. In some embodiments, the transient alteration of protein expression targets TIGIT. In some embodiments, the transient alteration of protein expression targets TGFβ. In some embodiments, the transient alteration of protein expression targets CCR1. In some embodiments, the transient alteration of protein expression targets CCR2. In some embodiments, the transient alteration of protein expression targets CCR4. In some embodiments, the transient alteration of protein expression targets CCR5. In some embodiments, the transient alteration of protein expression targets CXCR1. In some embodiments, the transient alteration of protein expression targets CXCR2. In some embodiments, the transient alteration of protein expression targets CSCR3. In some embodiments, the transient alteration of protein expression targets CCL2 (MCP-1). In some embodiments, the transient alteration of protein expression targets CCL3 (MIP-1α). In some embodiments, the transient alteration of protein expression targets CCL4 (MIP1-β). In some embodiments, the transient alteration of protein expression targets CCL5 (RANTES). In some embodiments, the transient alteration of protein expression targets CXCL1. In some embodiments, the transient alteration of protein expression targets CXCL8. In some embodiments, the transient alteration of protein expression targets CCL22. In some embodiments, the transient alteration of protein expression targets CCL17. In some embodiments, the transient alteration of protein expression targets VHL. In some embodiments, the transient alteration of protein expression targets CD44. In some embodiments, the transient alteration of protein expression targets PIK3CD. In some embodiments, the transient alteration of protein expression targets SOCS1. In some embodiments, the transient alteration of protein expression targets cAMP protein kinase A (PKA).

In some embodiments, the transient alteration of protein expression results in increased and/or overexpression of a chemokine receptor. In some embodiments, the chemokine receptor that is overexpressed by transient protein expression includes a receptor with a ligand that includes but is not limited to CCL2 (MCP-1), CCL3 (MIP-1α), CCL4 (MIP1-β), CCL5 (RANTES), CXCL1, CXCL8, CCL22, and/or CCL17.

In some embodiments, the transient alteration of protein expression results in a decrease and/or reduced expression of PD-1, CTLA-4, TIM-3, LAG-3, TIGIT, TGFβR2, and/or TGFβ (including resulting in, for example, TGFβ pathway blockade). In some embodiments, the transient alteration of protein expression results in a decrease and/or reduced expression of CBLB (CBL-B). In some embodiments, the transient alteration of protein expression results in a decrease and/or reduced expression of CISH.

In some embodiments, the transient alteration of protein expression results in increased and/or overexpression of chemokine receptors in order to, for example, improve TIL trafficking or movement to the tumor site. In some embodiments, the transient alteration of protein expression results in increased and/or overexpression of a CCR (chimeric co-stimulatory receptor). In some embodiments, the transient alteration of protein expression results in increased and/or overexpression of a chemokine receptor selected from the group consisting of CCR1, CCR2, CCR4, CCR5, CXCR1, CXCR2, and/or CSCR3.

In some embodiments, the transient alteration of protein expression results in increased and/or overexpression of an interleukin. In some embodiments, the transient alteration of protein expression results in increased and/or overexpression of an interleukin selected from the group consisting of IL-2, IL-12, IL-15, and/or IL-21.

In some embodiments, the transient alteration of protein expression results in increased and/or overexpression of NOTCH 1/2 ICD. In some embodiments, the transient alteration of protein expression results in increased and/or overexpression of VHL. In some embodiments, the transient alteration of protein expression results in increased and/or overexpression of CD44. In some embodiments, the transient alteration of protein expression results in increased and/or overexpression of PIK3CD. In some embodiments, the transient alteration of protein expression results in increased and/or overexpression of SOCS1,

In some embodiments, the transient alteration of protein expression results in decreased and/or reduced expression of cAMP protein kinase A (PKA).

In some embodiments, the transient alteration of protein expression results in decreased and/or reduced expression of a molecule selected from the group consisting of PD-1, LAG3, TIM3, CTLA-4, TIGIT, CISH, TGFβR2, PKA, CBLB, BAFF (BR3), and combinations thereof. In some embodiments, the transient alteration of protein expression results in decreased and/or reduced expression of two molecules selected from the group consisting of PD-1, LAG3, TIM3, CTLA-4, TIGIT, CISH, TGFβR2, PKA, CBLB, BAFF (BR3), and combinations thereof. In some embodiments, the transient alteration of protein expression results in decreased and/or reduced expression of PD-1 and one molecule selected from the group consisting of LAG3, TIM3, CTLA-4, TIGIT, CISH, TGFβR2, PKA, CBLB, BAFF (BR3), and combinations thereof. In some embodiments, the transient alteration of protein expression results in decreased and/or reduced expression of PD-1, LAG-3, CISH, CBLB, TIM3, and combinations thereof. In some embodiments, the transient alteration of protein expression results in decreased and/or reduced expression of PD-1 and one of LAG3, CISH, CBLB, TIM3, and combinations thereof. In some embodiments, the transient alteration of protein expression results in decreased and/or reduced expression of PD-1 and LAG3. In some embodiments, the transient alteration of protein expression results in decreased and/or reduced expression of PD-1 and CISH. In some embodiments, the transient alteration of protein expression results in decreased and/or reduced expression of PD-1 and CBLB. In some embodiments, the transient alteration of protein expression results in decreased and/or reduced expression of LAG3 and CISH. In some embodiments, the transient alteration of protein expression results in decreased and/or reduced expression of LAG3 and CBLB. In some embodiments, the transient alteration of protein expression results in decreased and/or reduced expression of CISH and CBLB. In some embodiments, the transient alteration of protein expression results in decreased and/or reduced expression of TIM3 and PD-1. In some embodiments, the transient alteration of protein expression results in decreased and/or reduced expression of TIM3 and LAG3. In some embodiments, the transient alteration of protein expression results in decreased and/or reduced expression of TIM3 and CISH. In some embodiments, the transient alteration of protein expression results in decreased and/or reduced expression of TIM3 and CBLB.

In some embodiments, an adhesion molecule selected from the group consisting of CCR2, CCR4, CCR5, CXCR2, CXCR3, CX3CR1, and combinations thereof, is inserted by a gammaretroviral or lentiviral method into the first population of TILs, PBLs, and/or MILs, second population of TILs, PBLs, and/or MILs, or harvested population of TILs, PBLs, and/or MILs (e.g., the expression of the adhesion molecule is increased).

In some embodiments, the transient alteration of protein expression results in decreased and/or reduced expression of a molecule selected from the group consisting of PD-1, LAG3, TIM3, CTLA-4, TIGIT, CISH, TGFβR2, PKA, CBLB, BAFF (BR3), and combinations thereof, and increased and/or enhanced expression of CCR2, CCR4, CCR5, CXCR2, CXCR3, CX3CR1, and combinations thereof. In some embodiments, the transient alteration of protein expression results in decreased and/or reduced expression of a molecule selected from the group consisting of PD-1, LAG3, TIM3, CISH, CBLB, and combinations thereof, and increased and/or enhanced expression of CCR2, CCR4, CCR5, CXCR2, CXCR3, CX3CR1, and combinations thereof.

In some embodiments, there is a reduction in expression of about 5%, about 10%, about 10%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, there is a reduction in expression of at least about 65%, about 70%, about 75%, about 80%, about 85%, about 90%, or about 95%. In some embodiments, there is a reduction in expression of at least about 75%, about 80%, about 85%, about 90%, or about 95%. In some embodiments, there is a reduction in expression of at least about 80%, about 85%, about 90%, or about 95%. In some embodiments, there is a reduction in expression of at least about 85%, about 90%, or about 95%. In some embodiments, there is a reduction in expression of at least about 80%. In some embodiments, there is a reduction in expression of at least about 85%, In some embodiments, there is a reduction in expression of at least about 90%. In some embodiments, there is a reduction in expression of at least about 95%. In some embodiments, there is a reduction in expression of at least about 99%.

In some embodiments, there is an increase in expression of about 5%, about 10%, about 10%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, there is an increase in expression of at least about 65%, about 70%, about 75%, about 80%, about 85%, about 90%, or about 95%. In some embodiments, there is an increase in expression of at least about 75%, about 80%, about 85%, about 90%, or about 95%. In some embodiments, there is an increase in expression of at least about 80%, about 85%, about 90%, or about 95%. In some embodiments, there is an increase in expression of at least about 85%, about 90%, or about 95%. In some embodiments, there is an increase in expression of at least about 80%. In some embodiments, there is an increase in expression of at least about 85%, In some embodiments, there is an increase in expression of at least about 90%. In some embodiments, there is an increase in expression of at least about 95%. In some embodiments, there is an increase in expression of at least about 99%.

In some embodiments, transient alteration of protein expression is induced by treatment of the TILs, PBLs, and/or MILs with transcription factors (TFs) and/or other molecules capable of transiently altering protein expression in the TILs, PBLs, and/or MILs. In some embodiments, the SQZ vector-free microfluidic platform is employed for intracellular delivery of the transcription factors (TFs) and/or other molecules capable of transiently altering protein expression. Such methods demonstrating the ability to deliver proteins, including transcription factors, to a variety of primary human cells, including T cells (Sharei et al. PNAS 2013, as well as Sharei et al. PLOS ONE 2015 and Greisbeck et al. J. Immunology vol. 195, 2015) have been described; see, for example, International Patent Publications WO 2013/059343A1, WO 2017/008063A1, and WO 2017/123663A1, all of which are incorporated by reference herein in their entireties. Such methods as described in International Patent Publications WO 2013/059343A1, WO 2017/008063A1, and WO 2017/123663A1 can be employed with the present invention in order to expose a population of TILs, PBLs, and/or MILs to transcription factors (TFs) and/or other molecules capable of inducing transient protein expression, wherein said TFs and/or other molecules capable of inducing transient protein expression provide for increased expression of tumor antigens and/or an increase in the number of tumor antigen-specific T cells in the population of TILs, PBLs, and/or MILs, thus resulting in reprogramming of the TIL population and an increase in therapeutic efficacy of the reprogrammed TIL population as compared to a non-reprogrammed TIL population. In some embodiments, the reprogramming results in an increased subpopulation of effector T cells and/or central memory T cells relative to the starting or prior population (i.e., prior to reprogramming) population of TILs, PBLs, and/or MILs, as described herein.

In some embodiments, the transcription factor (TF) includes but is not limited to TCF-1, NOTCH 1/2 ICD, and/or MYB. In some embodiments, the transcription factor (TF) is TCF-1. In some embodiments, the transcription factor (TF) is NOTCH 1/2 ICD. In some embodiments, the transcription factor (TF) is MYB. In some embodiments, the transcription factor (TF) is administered with induced pluripotent stem cell culture (iPSC), such as the commercially available KNOCKOUT Serum Replacement (Gibco/ThermoFisher), to induce additional TIL reprogramming. In some embodiments, the transcription factor (TF) is administered with an iPSC cocktail to induce additional TIL reprogramming. In some embodiments, the transcription factor (TF) is administered without an iPSC cocktail. In some embodiments, reprogramming results in an increase in the percentage of TSCMs. In some embodiments, reprogramming results in an increase in the percentage of TSCMs by about 5%, about 10%, about 10%, about 20%, about 25%, about 30%, about 35%, about 40%, about 45%, about 50%, about 55%, about 60%, about 65%, about 70%, about 75%, about 80%, about 85%, about 90%, or about 95% TSCMs.

In some embodiments, a method of transient altering protein expression, as described above, may be combined with a method of genetically modifying a population of TILs, PBLs, and/or MILs including the step of stable incorporation of genes for production of one or more proteins. In certain embodiments, the method comprises a step of genetically modifying a population of TILs, PBLs, and/or MILs. In certain embodiments, the method comprises genetically modifying the first population of TILs, PBLs, and/or MILs, the second population of TILs, PBLs, and/or MILs and/or the third population of TILs, PBLs, and/or MILs. In an embodiment, a method of genetically modifying a population of TILs, PBLs, and/or MILs includes the step of retroviral transduction. In an embodiment, a method of genetically modifying a population of TILs, PBLs, and/or MILs includes the step of lentiviral transduction. Lentiviral transduction systems are known in the art and are described, e.g., in Levine, et al., Proc. Nat'l Acad. Sci. 2006, 103, 17372-77; Zufferey, et al., Nat. Biotechnol. 1997, 15, 871-75; Dull, et al., J. Virology 1998, 72, 8463-71, and U.S. Pat. No. 6,627,442, the disclosures of each of which are incorporated by reference herein. In an embodiment, a method of genetically modifying a population of TILs, PBLs, and/or MILs includes the step of gamma-retroviral transduction. Gamma-retroviral transduction systems are known in the art and are described, e.g., Cepko and Pear, Cur. Prot. Mol. Biol. 1996, 9.9.1-9.9.16, the disclosure of which is incorporated by reference herein. In an embodiment, a method of genetically modifying a population of TILs, PBLs, and/or MILs includes the step of transposon-mediated gene transfer. Transposon-mediated gene transfer systems are known in the art and include systems wherein the transposase is provided as DNA expression vector or as an expressible RNA or a protein such that long-term expression of the transposase does not occur in the transgenic cells, for example, a transposase provided as an mRNA (e.g., an mRNA comprising a cap and poly-A tail). Suitable transposon-mediated gene transfer systems, including the salmonid-type Tel-like transposase (SB or Sleeping Beauty transposase), such as SB10, SB11, and SB100x, and engineered enzymes with increased enzymatic activity, are described in, e.g., Hackett, et al., Mol. Therapy 2010, 18, 674-83 and U.S. Pat. No. 6,489,458, the disclosures of each of which are incorporated by reference herein.

In some embodiments, transient alteration of protein expression is a reduction in expression induced by self-delivering RNA interference (sdRNA), which is a chemically-synthesized asymmetric siRNA duplex with a high percentage of 2′-OH substitutions (typically fluorine or —OCH₃) which comprises a 20-nucleotide antisense (guide) strand and a 13 to 15 base sense (passenger) strand conjugated to cholesterol at its 3′ end using a tetraethylenglycol (TEG) linker. In some embodiments, the method comprises transient alteration of protein expression in a population of TILs, PBLs, and/or MILs, comprising the use of self-delivering RNA interference (sdRNA), which is a chemically-synthesized asymmetric siRNA duplex with a high percentage of 2′-OH substitutions (typically fluorine or —OCH₃) which comprises a 20-nucleotide antisense (guide) strand and a 13 to 15 base sense (passenger) strand conjugated to cholesterol at its 3′ end using a tetraethylenglycol (TEG) linker. Methods of using sdRNA have been described in Khvorova and Watts, Nat. Biotechnol. 2017, 35, 238-248; Byrne, et al., J. Ocul. Pharmacol. Ther. 2013, 29, 855-864; and Ligtenberg, et al., Mol. Therapy, 2018, 26, 1482-1493, the disclosures of which are incorporated by reference herein. In an embodiment, delivery of sdRNA to a TIL population is accomplished without use of electroporation, SQZ, or other methods, instead using a 1 to 3 day period in which a TIL population is exposed to sdRNA at a concentration of 1 μM/10,000 TILs, PBLs, and/or MILs in medium. In certain embodiments, the method comprises delivery sdRNA to a TILs, PBLs, and/or MILs population comprising exposing the TILs, PBLs, and/or MILs population to sdRNA at a concentration of 1 μM/10,000 TILs, PBLs, and/or MILs in medium for a period of between 1 to 3 days. In an embodiment, delivery of sdRNA to a TIL population is accomplished using a 1 to 3 day period in which a TIL population is exposed to sdRNA at a concentration of 10 μM/10,000 TILs, PBLs, and/or MILs in medium. In an embodiment, delivery of sdRNA to a TIL population is accomplished using a 1 to 3 day period in which a TIL population is exposed to sdRNA at a concentration of 50 μM/10,000 TILs, PBLs, and/or MILs in medium. In an embodiment, delivery of sdRNA to a TIL population is accomplished using a 1 to 3 day period in which a TIL population is exposed to sdRNA at a concentration of between 0.1 μM/10,000 TILs, PBLs, and/or MILs and 50 μM/10,000 TILs, PBLs, and/or MILs in medium. In an embodiment, delivery of sdRNA to a TIL population is accomplished using a 1 to 3 day period in which a TIL population is exposed to sdRNA at a concentration of between 0.1 μM/10,000 TILs, PBLs, and/or MILs and 50 μM/10,000 TILs, PBLs, and/or MILs in medium, wherein the exposure to sdRNA is performed two, three, four, or five times by addition of fresh sdRNA to the media. Other suitable processes are described, for example, in U.S. Patent Application Publication No. US 2011/0039914 A1, US 2013/0131141 A1, and US 2013/0131142 A1, and U.S. Pat. No. 9,080,171, the disclosures of which are incorporated by reference herein.

In some embodiments, sdRNA is inserted into a population of TILs, PBLs, and/or MILs during manufacturing. In some embodiments, the sdRNA encodes RNA that interferes with NOTCH 1/2 ICD, PD-1, CTLA-4 TIM-3, LAG-3, TIGIT, TGFβ, TGFBR2, cAMP protein kinase A (PKA), BAFF BR3, CISH, and/or CBLB. In some embodiments, the reduction in expression is determined based on a percentage of gene silencing, for example, as assessed by flow cytometry and/or qPCR. In some embodiments, there is a reduction in expression of about 5%, about 10%, about 10%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, there is a reduction in expression of at least about 65%, about 70%, about 75%, about 80%, about 85%, about 90%, or about 95%. In some embodiments, there is a reduction in expression of at least about 75%, about 80%, about 85%, about 90%, or about 95%. In some embodiments, there is a reduction in expression of at least about 80%, about 85%, about 90%, or about 95%. In some embodiments, there is a reduction in expression of at least about 85%, about 90%, or about 95%. In some embodiments, there is a reduction in expression of at least about 80%. In some embodiments, there is a reduction in expression of at least about 85%. In some embodiments, there is a reduction in expression of at least about 90%. In some embodiments, there is a reduction in expression of at least about 95%. In some embodiments, there is a reduction in expression of at least about 99%.

The self-deliverable RNAi technology based on the chemical modification of siRNAs can be employed with the methods of the present invention to successfully deliver the sdRNAs to the TILs, PBLs, and/or MILs as described herein. The combination of backbone modifications with asymmetric siRNA structure and a hydrophobic ligand (see, for example, Ligtenberg, et al., Mol. Therapy, 2018 and US20160304873) allow sdRNAs to penetrate cultured mammalian cells without additional formulations and methods by simple addition to the culture media, capitalizing on the nuclease stability of sdRNAs. This stability allows the support of constant levels of RNAi-mediated reduction of target gene activity simply by maintaining the active concentration of sdRNA in the media. While not being bound by theory, the backbone stabilization of sdRNA provides for extended reduction in gene expression effects which can last for months in non-dividing cells.

In some embodiments, over 95% transfection efficiency of TILs, PBLs, and/or MILs and a reduction in expression of the target by various specific sdRNA occurs. In some embodiments, sdRNAs containing several unmodified ribose residues were replaced with fully modified sequences to increase potency and/or the longevity of RNAi effect. In some embodiments, a reduction in expression effect is maintained for 12 hours, 24 hours, 36 hours, 48 hours, 5 days, 6 days, 7 days, or 8 days or more. In some embodiments, the reduction in expression effect decreases at 10 days or more post sdRNA treatment of the TILs, PBLs, and/or MILs. In some embodiments, more than 70% reduction in expression of the target expression is maintained. In some embodiments, more than 70% reduction in expression of the target expression is maintained in TILs, PBLs, and/or MILs. In some embodiments, a reduction in expression in the PD-1/PD-L1 pathway allows for the TILs, PBLs, and/or MILs to exhibit a more potent in vivo effect, which is in some embodiments, due to the avoidance of the suppressive effects of the PD-1/PD-L1 pathway. In some embodiments, a reduction in expression of PD-1 by sdRNA results in an increase TIL proliferation.

Small interfering RNA (siRNA), sometimes known as short interfering RNA or silencing RNA, is a double stranded RNA molecule, generally 19-25 base pairs in length. siRNA is used in RNA interference (RNAi), where it interferes with expression of specific genes with complementary nucleotide sequences.

Double stranded DNA (dsRNA) can be generally used to define any molecule comprising a pair of complementary strands of RNA, generally a sense (passenger) and antisense (guide) strands, and may include single-stranded overhang regions. The term dsRNA, contrasted with siRNA, generally refers to a precursor molecule that includes the sequence of an siRNA molecule which is released from the larger dsRNA molecule by the action of cleavage enzyme systems, including Dicer.

sdRNA (self-deliverable RNA) are a new class of covalently modified RNAi compounds that do not require a delivery vehicle to enter cells and have improved pharmacology compared to traditional siRNAs. “Self-deliverable RNA” or “sdRNA” is a hydrophobically modified RNA interfering-antisense hybrid, demonstrated to be highly efficacious in vitro in primary cells and in vivo upon local administration. Robust uptake and/or silencing without toxicity has been demonstrated. sdRNAs are generally asymmetric chemically modified nucleic acid molecules with minimal double stranded regions. sdRNA molecules typically contain single stranded regions and double stranded regions, and can contain a variety of chemical modifications within both the single stranded and double stranded regions of the molecule. Additionally, the sdRNA molecules can be attached to a hydrophobic conjugate such as a conventional and advanced sterol-type molecule, as described herein. sdRNAs and associated methods for making such sdRNAs have also been described extensively in, for example, US20160304873, WO2010033246, WO2017070151, WO2009102427, WO2011119887, WO2010033247A2, WO2009045457, WO2011119852, all of which are incorporated by reference herein in their entireties for all purposes. To optimize sdRNA structure, chemistry, targeting position, sequence preferences, and the like, a proprietary algorithm has been developed and utilized for sdRNA potency prediction (see, for example, US 20160304873). Based on these analyses, functional sdRNA sequences have been generally defined as having over 70% reduction in expression at 1μM concentration, with a probability over 40%.

In some embodiments, the sdRNA sequences used in the invention exhibit a 70% reduction in expression of the target gene. In some embodiments, the sdRNA sequences used in the invention exhibit a 75% reduction in expression of the target gene. In some embodiments, the sdRNA sequences used in the invention exhibit an 80% reduction in expression of the target gene. In some embodiments, the sdRNA sequences used in the invention exhibit an 85% reduction in expression of the target gene. In some embodiments, the sdRNA sequences used in the invention exhibit a 90% reduction in expression of the target gene. In some embodiments, the sdRNA sequences used in the invention exhibit a 95% reduction in expression of the target gene. In some embodiments, the sdRNA sequences used in the invention exhibit a 99% reduction in expression of the target gene. In some embodiments, the sdRNA sequences used in the invention exhibit a reduction in expression of the target gene when delivered at a concentration of about 0.25 μM to about 4 μM. In some embodiments, the sdRNA sequences used in the invention exhibit a reduction in expression of the target gene when delivered at a concentration of about 0.25 μM. In some embodiments, the sdRNA sequences used in the invention exhibit a reduction in expression of the target gene when delivered at a concentration of about 0.5 μM. In some embodiments, the sdRNA sequences used in the invention exhibit a reduction in expression of the target gene when delivered at a concentration of about 0.75 μM. In some embodiments, the sdRNA sequences used in the invention exhibit a reduction in expression of the target gene when delivered at a concentration of about 1.0 μM. In some embodiments, the sdRNA sequences used in the invention exhibit a reduction in expression of the target gene when delivered at a concentration of about 1.25 μM. In some embodiments, the sdRNA sequences used in the invention exhibit a reduction in expression of the target gene when delivered at a concentration of about 1.5 μM. In some embodiments, the sdRNA sequences used in the invention exhibit a reduction in expression of the target gene when delivered at a concentration of about 1.75 μM. In some embodiments, the sdRNA sequences used in the invention exhibit a reduction in expression of the target gene when delivered at a concentration of about 2.0 μM. In some embodiments, the sdRNA sequences used in the invention exhibit a reduction in expression of the target gene when delivered at a concentration of about 2.25 μM. In some embodiments, the sdRNA sequences used in the invention exhibit a reduction in expression of the target gene when delivered at a concentration of about 2.5 μM. In some embodiments, the sdRNA sequences used in the invention exhibit a reduction in expression of the target gene when delivered at a concentration of about 2.75 μM. In some embodiments, the sdRNA sequences used in the invention exhibit a reduction in expression of the target gene when delivered at a concentration of about 3.0 μM. In some embodiments, the sdRNA sequences used in the invention exhibit a reduction in expression of the target gene when delivered at a concentration of about 3.25 μM. In some embodiments, the sdRNA sequences used in the invention exhibit a reduction in expression of the target gene when delivered at a concentration of about 3.5 μM. In some embodiments, the sdRNA sequences used in the invention exhibit a reduction in expression of the target gene when delivered at a concentration of about 3.75 μM. In some embodiments, the sdRNA sequences used in the invention exhibit a reduction in expression of the target gene when delivered at a concentration of about 4.0 μM.

In some embodiments, the oligonucleotide agents comprise one or more modification to increase stability and/or effectiveness of the therapeutic agent, and to effect efficient delivery of the oligonucleotide to the cells or tissue to be treated. Such modifications can include a 2′-O-methyl modification, a 2′-O-Fluro modification, a diphosphorothioate modification, 2′ F modified nucleotide, a2′-O-methyl modified and/or a 2′deoxy nucleotide. In some embodiments, the oligonucleotide is modified to include one or more hydrophobic modifications including, for example, sterol, cholesterol, vitamin D, naphtyl, isobutyl, benzyl, indol, tryptophane, and/or phenyl. In an additional particular embodiment, chemically modified nucleotides are combination of phosphorothioates, 2′-O-methyl, 2′deoxy, hydrophobic modifications and phosphorothioates. In some embodiments, the sugars can be modified and modified sugars can include but are not limited to D-ribose, 2′-O-alkyl (including 2′-O-methyl and 2′-O-ethyl), i.e., 2′-alkoxy, 2′-amino, 2′-S-alkyl, 2′-halo (including 2′-fluoro), T-methoxyethoxy, 2′-allyloxy (—OCH₂CH═CH₂), 2′-propargyl, 2′-propyl, ethynyl, ethenyl, propenyl, and cyano and the like. In one embodiment, the sugar moiety can be a hexose and incorporated into an oligonucleotide as described (Augustyns, K., et al., Nucl. Acids. Res. 18:4711 (1992)).

In some embodiments, the double-stranded oligonucleotide of the invention is double-stranded over its entire length, i.e., with no overhanging single-stranded sequence at either end of the molecule, i.e., is blunt-ended. In some embodiments, the individual nucleic acid molecules can be of different lengths. In other words, a double-stranded oligonucleotide of the invention is not double-stranded over its entire length. For instance, when two separate nucleic acid molecules are used, one of the molecules, e.g., the first molecule comprising an antisense sequence, can be longer than the second molecule hybridizing thereto (leaving a portion of the molecule single-stranded). In some embodiments, when a single nucleic acid molecule is used a portion of the molecule at either end can remain single-stranded.

In some embodiments, a double-stranded oligonucleotide of the invention contains mismatches and/or loops or bulges, but is double-stranded over at least about 70% of the length of the oligonucleotide. In some embodiments, a double-stranded oligonucleotide of the invention is double-stranded over at least about 80% of the length of the oligonucleotide. In another embodiment, a double-stranded oligonucleotide of the invention is double-stranded over at least about 90%-95% of the length of the oligonucleotide. In some embodiments, a double-stranded oligonucleotide of the invention is double-stranded over at least about 96%-98% of the length of the oligonucleotide. In some embodiments, the double-stranded oligonucleotide of the invention contains at least or up to 1, 2, 3, 4, 5, 6, 7, 8, 9, 10, 11, 12, 13, 14, or 15 mismatches.

In some embodiments, the oligonucleotide can be substantially protected from nucleases e.g., by modifying the 3′ or 5′ linkages (e.g., U.S. Pat. No. 5,849,902 and WO 98/13526). For example, oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH₂—CH₂—CH₃), glycol (—O—CH₂—CH₂—O—) phosphate (PO₃^{2″}), hydrogen phosphonate, or phosphoramidite). “Blocking groups” can also include “end blocking groups” or “exonuclease blocking groups” which protect the 5′ and 3′ termini of the oligonucleotide, including modified nucleotides and non-nucleotide exonuclease resistant structures.

In some embodiments, at least a portion of the contiguous polynucleotides within the sdRNA are linked by a substitute linkage, e.g., a phosphorothioate linkage.

In some embodiments, chemical modification can lead to at least a 1.5, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 350, 375, 400, 425, 450, 475, 500 enhancements in cellular uptake. In some embodiments, at least one of the C or U residues includes a hydrophobic modification. In some embodiments, a plurality of Cs and Us contain a hydrophobic modification. In some embodiments, at least 10%, 15%, 20%, 30%, 40%, 50%, 55%, 60% 65%, 70%, 75%, 80%, 85%, 90% or at least 95% of the Cs and Us can contain a hydrophobic modification. In some embodiments, all of the Cs and Us contain a hydrophobic modification.

In some embodiments, the sdRNA or sd-rxRNAs exhibit enhanced endosomal release of sd-rxRNA molecules through the incorporation of protonatable amines. In some embodiments, protonatable amines are incorporated in the sense strand (in the part of the molecule which is discarded after RISC loading). In some embodiments, the sdRNA compounds of the invention comprise an asymmetric compound comprising a duplex region (required for efficient RISC entry of 10-15 bases long) and single stranded region of 4-12 nucleotides long; with a 13 nucleotide duplex. In some embodiments, a 6 nucleotide single stranded region is employed. In some embodiments, the single stranded region of the sdRNA comprises 2-12 phosphorothioate intemucleotide linkages (referred to as phosphorothioate modifications). In some embodiments, 6-8 phosphorothioate intemucleotide linkages are employed. In some embodiments, the sdRNA compounds of the invention also include a unique chemical modification pattern, which provides stability and is compatible with RISC entry.

The guide strand, for example, may also be modified by any chemical modification which confirms stability without interfering with RISC entry. In some embodiments, the chemical modification pattern in the guide strand includes the majority of C and U nucleotides being 2′ F modified and the 5′ end being phosphorylated.

In some embodiments, at least 30% of the nucleotides in the sdRNA or sd-rxRNA are modified. In some embodiments, at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the nucleotides in the sdRNA or sd-rxRNA are modified. In some embodiments, 100% of the nucleotides in the sdRNA or sd-rxRNA are modified.

In some embodiments, the sdRNA molecules have minimal double stranded regions. In some embodiments the region of the molecule that is double stranded ranges from 8-15 nucleotides long. In some embodiments, the region of the molecule that is double stranded is 8, 9, 10, 11, 12, 13, 14 or 15 nucleotides long. In some embodiments the double stranded region is 13 nucleotides long. There can be 100% complementarity between the guide and passenger strands, or there may be one or more mismatches between the guide and passenger strands. In some embodiments, on one end of the double stranded molecule, the molecule is either blunt-ended or has a one-nucleotide overhang. The single stranded region of the molecule is in some embodiments between 4-12 nucleotides long. In some embodiments, the single stranded region can be 4, 5, 6, 7, 8, 9, 10, 11 or 12 nucleotides long. In some embodiments, the single stranded region can also be less than 4 or greater than 12 nucleotides long. In certain embodiments, the single stranded region is 6 or 7 nucleotides long.

In some embodiments, the sdRNA molecules have increased stability. In some instances, a chemically modified sdRNA or sd-rxRNA molecule has a half-life in media that is longer than 1, 2, 3, 4, 5, 6, 7, 8, 9, 10, 11, 12, 13, 14, 15, 16, 17, 18, 19, 20, 21, 22, 23, 24 or more than 24 hours, including any intermediate values. In some embodiments, the sd-rxRNA has a half-life in media that is longer than 12 hours.

In some embodiments, the sdRNA is optimized for increased potency and/or reduced toxicity. In some embodiments, nucleotide length of the guide and/or passenger strand, and/or the number of phosphorothioate modifications in the guide and/or passenger strand, can in some aspects influence potency of the RNA molecule, while replacing 2′-fluoro (2′F) modifications with 2′-O-methyl (2′OMe) modifications can in some aspects influence toxicity of the molecule. In some embodiments, reduction in 2′F content of a molecule is predicted to reduce toxicity of the molecule. In some embodiments, the number of phosphorothioate modifications in an RNA molecule can influence the uptake of the molecule into a cell, for example the efficiency of passive uptake of the molecule into a cell. In some embodiments, the sdRNA has no 2′F modification and yet are characterized by equal efficacy in cellular uptake and tissue penetration.

In some embodiments, a guide strand is approximately 18-19 nucleotides in length and has approximately 2-14 phosphate modifications. For example, a guide strand can contain 2, 3, 4, 5, 6, 7, 8, 9, 10, 11, 12, 13, 14 or more than 14 nucleotides that are phosphate-modified. The guide strand may contain one or more modifications that confer increased stability without interfering with RISC entry. The phosphate modified nucleotides, such as phosphorothioate modified nucleotides, can be at the 3′ end, 5′ end or spread throughout the guide strand. In some embodiments, the 3′ terminal 10 nucleotides of the guide strand contain 1, 2, 3, 4, 5, 6, 7, 8, 9 or 10 phosphorothioate modified nucleotides. The guide strand can also contain 2′F and/or 2′OMe modifications, which can be located throughout the molecule. In some embodiments, the nucleotide in position one of the guide strand (the nucleotide in the most 5′ position of the guide strand) is 2′OMe modified and/or phosphorylated. C and U nucleotides within the guide strand can be 2′F modified. For example, C and U nucleotides in positions 2-10 of a 19 nt guide strand (or corresponding positions in a guide strand of a different length) can be 2′F modified. C and U nucleotides within the guide strand can also be 2′OMe modified. For example, C and U nucleotides in positions 11-18 of a 19 nt guide strand (or corresponding positions in a guide strand of a different length) can be 2′OMe modified. In some embodiments, the nucleotide at the most 3′ end of the guide strand is unmodified. In certain embodiments, the majority of Cs and Us within the guide strand are 2′F modified and the 5′ end of the guide strand is phosphorylated. In other embodiments, position 1 and the Cs or Us in positions 11-18 are 2′OMe modified and the 5′ end of the guide strand is phosphorylated. In other embodiments, position 1 and the Cs or Us in positions 11-18 are 2′OMe modified, the 5′ end of the guide strand is phosphorylated, and the Cs or Us in position 2-10 are 2′F modified.

The self-deliverable RNAi technology provides a method of directly transfecting cells with the RNAi agent, without the need for additional formulations or techniques. The ability to transfect hard-to-transfect cell lines, high in vivo activity, and simplicity of use, are characteristics of the compositions and methods that present significant functional advantages over traditional siRNA-based techniques, and as such, the sdRNA methods are employed in several embodiments related to the methods of reduction in expression of the target gene in the TILs, PBLs, and/or MILs of the present invention. The sdRNAi methods allow direct delivery of chemically synthesized compounds to a wide range of primary cells and tissues, both ex-vivo and in vivo. The sdRNAs described in some embodiments of the invention herein are commercially available from Advirna LLC, Worcester, Mass., USA.

The sdRNA are formed as hydrophobically-modified siRNA-antisense oligonucleotide hybrid structures, and are disclosed, for example in Byrne et al., December 2013, J. Ocular Pharmacology and Therapeutics, 29(10): 855-864, incorporated by reference herein in its entirety.

In some embodiments, the sdRNA oligonucleotides can be delivered to the TILs, PBLs, and/or MILs described herein using sterile electroporation. In certain embodiments, the method comprises sterile electroporation of a population of TILs, PBLs, and/or MILs to deliver sdRNA oligonucleotides.

In some embodiments, the oligonucleotides can be delivered to the cells in combination with a transmembrane delivery system. In some embodiments, this transmembrane delivery system comprises lipids, viral vectors, and the like. In some embodiments, the oligonucleotide agent is a self-delivery RNAi agent, that does not require any delivery agents. In certain embodiments, the method comprises use of a transmembrane delivery system to deliver sdRNA oligonucleotides to a population of TILs, PBLs, and/or MILs.

Oligonucleotides and oligonucleotide compositions are contacted with (e.g., brought into contact with, also referred to herein as administered or delivered to) and taken up by TILs, PBLs, and/or MILs described herein, including through passive uptake by TILs, PBLs, and/or MILs. The sdRNA can be added to the TILs, PBLs, and/or MILs as described herein during the first expansion, for example Step B, after the first expansion, for example, during Step C, before or during the second expansion, for example before or during Step D, after Step D and before harvest in Step E, during or after harvest in Step F, before or during final formulation and/or transfer to infusion Bag in Step F, as well as before any optional cryopreservation step in Step F. Moreover, sdRNA can be added after thawing from any cryopreservation step in Step F. In an embodiment, one or more sdRNAs targeting genes as described herein, including PD-1, LAG-3, TIM-3, CISH, and CBLB, may be added to cell culture media comprising TILs, PBLs, and/or MILs and other agents at concentrations selected from the group consisting of 100 nM to 20 mM, 200 nM to 10 mM, 500 nm to 1 mM, 1 μM to 100 μM, and 1 μM to 100 μM. In an embodiment, one or more sdRNAs targeting genes as described herein, including PD-1, LAG-3, TIM-3, CISH, and CBLB, may be added to cell culture media comprising TILs, PBLs, and/or MILs and other agents at amounts selected from the group consisting of 0.1 μM sdRNA/10,000 TILs, PBLs, and/or MILs/100 μL media, 0.5 μM sdRNA/10,000 TILs, PBLs, and/or MILs/100 μL media, 0.75 μM sdRNA/10,000 TILs, PBLs, and/or MILs/100 μL media, 1 μM sdRNA/10,000 TILs, PBLs, and/or MILs/100 μL media, 1.25 μM sdRNA/10,000 TILs, PBLs, and/or MILs/100 μL media, 1.5 μM sdRNA/10,000 TILs, PBLs, and/or MILs/100 μL media, 2 μM sdRNA/10,000 TILs, PBLs, and/or MILs/100 μL media, 5 μM sdRNA/10,000 TILs, PBLs, and/or MILs/100 μL media, or 10 μM sdRNA/10,000 TILs, PBLs, and/or MILs/100 μL media. In an embodiment, one or more sdRNAs targeting genes as described herein, including PD-1, LAG-3, TIM-3, CISH, and CBLB, may be added to TIL cultures during the pre-REP or REP stages twice a day, once a day, every two days, every three days, every four days, every five days, every six days, or every seven days.

Oligonucleotide compositions of the invention, including sdRNA, can be contacted with TILs, PBLs, and/or MILs as described herein during the expansion process, for example by dissolving sdRNA at high concentrations in cell culture media and allowing sufficient time for passive uptake to occur. In certain embodiments, the method of the present invention comprises contacting a population of TILs, PBLs, and/or MILs with an oligonucleotide composition as described herein. In certain embodiments, the method comprises dissolving an oligonucleotide e.g. sdRNA in a cell culture media and contacting the cell culture media with a population of TILs, PBLs, and/or MILs. The TILs, PBLs, and/or MILs may be a first population, a second population and/or a third population as described herein.

In some embodiments, delivery of oligonucleotides into cells can be enhanced by suitable art recognized methods including calcium phosphate, DMSO, glycerol or dextran, electroporation, or by transfection, e.g., using cationic, anionic, or neutral lipid compositions or liposomes using methods known in the art (see, e.g., WO 90/14074; WO 91/16024; WO 91/17424; U.S. Pat. No. 4,897,355; Bergan et a 1993. Nucleic Acids Research. 21 :3567).

In some embodiments, more than one sdRNA is used to reduce expression of a target gene. In some embodiments, one or more of PD-1, TIM-3, CBLB, LAG3 and/or CISH targeting sdRNAs are used together. In some embodiments, a PD-1 sdRNA is used with one or more of TIM-3, CBLB, LAG3 and/or CISH in order to reduce expression of more than one gene target. In some embodiments, a LAG3 sdRNA is used in combination with a CISH targeting sdRNA to reduce gene expression of both targets. In some embodiments, the sdRNAs targeting one or more of PD-1, TIM-3, CBLB, LAG3 and/or CISH herein are commercially available from Advirna LLC, Worcester, Mass., USA.

In some embodiments, the sdRNA targets a gene selected from the group consisting of PD-1, LAG3, TIM3, CTLA-4, TIGIT, CISH, TGFβR2, PKA, CBLB, BAFF (BR3), and combinations thereof. In some embodiments, the sdRNA targets a gene selected from the group consisting of PD-1, LAG3, TIM3, CTLA-4, TIGIT, CISH, TGFβR2, PKA, CBLB, BAFF (BR3), and combinations thereof. In some embodiments, one sdRNA targets PD-1 and another sdRNA targets a gene selected from the group consisting of LAG3, TIM3, CTLA-4, TIGIT, CISH, TGFβR2, PKA, CBLB, BAFF (BR3), and combinations thereof. In some embodiments, the sdRNA targets a gene selected from PD-1, LAG-3, CISH, CBLB, TIM3, and combinations thereof. In some embodiments, the sdRNA targets a gene selected from PD-1 and one of LAG3, CISH, CBLB, TIM3, and combinations thereof. In some embodiments, one sdRNA targets PD-1 and one sdRNA targets LAG3. In some embodiments, one sdRNA targets PD-1 and one sdRNA targets CISH. In some embodiments, one sdRNA targets PD-1 and one sdRNA targets CBLB. In some embodiments, one sdRNA targets LAG3 and one sdRNA targets CISH. In some embodiments, one sdRNA targets LAG3 and one sdRNA targets CBLB. In some embodiments, one sdRNA targets CISH and one sdRNA targets CBLB. In some embodiments, one sdRNA targets TIM3 and one sdRNA targets PD-1. In some embodiments, one sdRNA targets TIM3 and one sdRNA targets LAG3. In some embodiments, one sdRNA targets TIM3 and one sdRNA targets CISH. In some embodiments, one sdRNA targets TIM3 and one sdRNA targets CBLB.

As discussed above, embodiments of the present invention provide TILs, PBLs, and/or MILs that have been genetically modified via gene-editing to enhance their therapeutic effect. Embodiments of the present invention embrace genetic editing through nucleotide insertion (RNA or DNA) into a population of TILs, PBLs, and/or MILs for both promotion of the expression of one or more proteins and inhibition of the expression of one or more proteins, as well as combinations thereof. Embodiments of the present invention also provide methods for expanding TILs, PBLs, and/or MILs into a therapeutic population, wherein the methods comprise gene-editing the TILs, PBLs, and/or MILs. There are several gene-editing technologies that may be used to genetically modify a population of TILs, PBLs, and/or MILs, which are suitable for use in accordance with the present invention.

In some embodiments, the method comprises a method of genetically modifying a population of TILs, PBLs, and/or MILs which include the step of stable incorporation of genes for production of one or more proteins. In an embodiment, a method of genetically modifying a population of TILs, PBLs, and/or MILs includes the step of retroviral transduction. In an embodiment, a method of genetically modifying a population of TILs, PBLs, and/or MILs includes the step of lentiviral transduction. Lentiviral transduction systems are known in the art and are described, e.g., in Levine, et al., Proc. Nat'l Acad. Sci. 2006, 103, 17372-77; Zufferey, et al., Nat. Biotechnol. 1997, 15, 871-75; Dull, et al., J. Virology 1998, 72, 8463-71, and U.S. Pat. No. 6,627,442, the disclosures of each of which are incorporated by reference herein. In an embodiment, a method of genetically modifying a population of TILs, PBLs, and/or MILs includes the step of gamma-retroviral transduction. Gamma-retroviral transduction systems are known in the art and are described, e.g., Cepko and Pear, Cur. Prot. Mol. Biol. 1996, 9.9.1-9.9.16, the disclosure of which is incorporated by reference herein. In an embodiment, a method of genetically modifying a population of TILs, PBLs, and/or MILs includes the step of transposon-mediated gene transfer. Transposon-mediated gene transfer systems are known in the art and include systems wherein the transposase is provided as DNA expression vector or as an expressible RNA or a protein such that long-term expression of the transposase does not occur in the transgenic cells, for example, a transposase provided as an mRNA (e.g., an mRNA comprising a cap and poly-A tail). Suitable transposon-mediated gene transfer systems, including the salmonid-type Tel-like transposase (SB or Sleeping Beauty transposase), such as SB10, SB11, and SB100x, and engineered enzymes with increased enzymatic activity, are described in, e.g., Hackett, et al., Mol. Therapy 2010, 18, 674-83 and U.S. Pat. No. 6,489,458, the disclosures of each of which are incorporated by reference herein.

In an embodiment, the method comprises a method of genetically modifying a population of TILs, PBLs, and/or MILs e.g. a first population, a second population and/or a third population as described herein. In an embodiment, a method of genetically modifying a population of TILs, PBLs, and/or MILs includes the step of stable incorporation of genes for production or inhibition (e.g., silencing) of one ore more proteins. In an embodiment, a method of genetically modifying a population of TILs, PBLs, and/or MILs includes the step of electroporation. Electroporation methods are known in the art and are described, e.g., in Tsong, Biophys. J. 1991, 60, 297-306, and U.S. Patent Application Publication No. 2014/0227237 A1, the disclosures of each of which are incorporated by reference herein. Other electroporation methods known in the art, such as those described in U.S. Pat. Nos. 5,019,034; 5,128,257; 5,137,817; 5,173,158; 5,232,856; 5,273,525; 5,304,120; 5,318,514; 6,010,613 and 6,078,490, the disclosures of which are incorporated by reference herein, may be used. In an embodiment, the electroporation method is a sterile electroporation method. In an embodiment, the electroporation method is a pulsed electroporation method. In an embodiment, the electroporation method is a pulsed electroporation method comprising the steps of treating TILs, PBLs, and/or MILs with pulsed electrical fields to alter, manipulate, or cause defined and controlled, permanent or temporary changes in the TILs, PBLs, and/or MILs, comprising the step of applying a sequence of at least three single, operator-controlled, independently programmed, DC electrical pulses, having field strengths equal to or greater than 100 V/cm, to the TILs, PBLs, and/or MILs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses. In an embodiment, the electroporation method is a pulsed electroporation method comprising the steps of treating TILs, PBLs, and/or MILs with pulsed electrical fields to alter, manipulate, or cause defined and controlled, permanent or temporary changes in the TILs, PBLs, and/or MILs, comprising the step of applying a sequence of at least three single, operator-controlled, independently programmed, DC electrical pulses, having field strengths equal to or greater than 100 V/cm, to the TILs, PBLs, and/or MILs, wherein at least two of the at least three pulses differ from each other in pulse amplitude. In an embodiment, the electroporation method is a pulsed electroporation method comprising the steps of treating TILs, PBLs, and/or MILs with pulsed electrical fields to alter, manipulate, or cause defined and controlled, permanent or temporary changes in the TILs, PBLs, and/or MILs, comprising the step of applying a sequence of at least three single, operator-controlled, independently programmed, DC electrical pulses, having field strengths equal to or greater than 100 V/cm, to the TILs, PBLs, and/or MILs, wherein at least two of the at least three pulses differ from each other in pulse width. In an embodiment, the electroporation method is a pulsed electroporation method comprising the steps of treating TILs, PBLs, and/or MILs with pulsed electrical fields to alter, manipulate, or cause defined and controlled, permanent or temporary changes in the TILs, PBLs, and/or MILs, comprising the step of applying a sequence of at least three single, operator-controlled, independently programmed, DC electrical pulses, having field strengths equal to or greater than 100 V/cm, to the TILs, PBLs, and/or MILs, wherein a first pulse interval for a first set of two of the at least three pulses is different from a second pulse interval for a second set of two of the at least three pulses. In an embodiment, the electroporation method is a pulsed electroporation method comprising the steps of treating TILs, PBLs, and/or MILs with pulsed electrical fields to induce pore formation in the TILs, PBLs, and/or MILs, comprising the step of applying a sequence of at least three DC electrical pulses, having field strengths equal to or greater than 100 V/cm, to TILs, PBLs, and/or MILs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses, such that induced pores are sustained for a relatively long period of time, and such that viability of the TILs, PBLs, and/or MILs is maintained. In an embodiment, a method of genetically modifying a population of TILs, PBLs, and/or MILs includes the step of calcium phosphate transfection. Calcium phosphate transfection methods (calcium phosphate DNA precipitation, cell surface coating, and endocytosis) are known in the art and are described in Graham and van der Eb, Virology 1973, 52, 456-467; Wigler, et al., Proc. Natl. Acad. Sci. 1979, 76, 1373-1376; and Chen and Okayarea, Mol. Cell. Biol. 1987, 7, 2745-2752; and in U.S. Pat. No. 5,593,875, the disclosures of each of which are incorporated by reference herein. In an embodiment, a method of genetically modifying a population of TILs, PBLs, and/or MILs includes the step of liposomal transfection. Liposomal transfection methods, such as methods that employ a 1:1 (w/w) liposome formulation of the cationic lipid N-[1-(2,3-dioleyloxy)propyl]-n,n,n-trimethylammonium chloride (DOTMA) and dioleoyl phophotidylethanolamine (DOPE) in filtered water, are known in the art and are described in Rose, et al., Biotechniques 1991, 10, 520-525 and Felgner, et al., Proc. Natl. Acad. Sci. USA, 1987, 84, 7413-7417 and in U.S. Pat. Nos. 5,279,833; 5,908,635; 6,056,938; 6,110,490; 6,534,484; and 7,687,070, the disclosures of each of which are incorporated by reference herein. In an embodiment, a method of genetically modifying a population of TILs, PBLs, and/or MILs includes the step of transfection using methods described in U.S. Pat. Nos. 5,766,902; 6,025,337; 6,410,517; 6,475,994; and 7,189,705; the disclosures of each of which are incorporated by reference herein. The TILs, PBLs, and/or MILs may be a first population, a second population and/or a third population of TILs, PBLs, and/or MILs as described herein.

According to an embodiment, the gene-editing process may comprise the use of a programmable nuclease that mediates the generation of a double-strand or single-strand break at one or more immune checkpoint genes. Such programmable nucleases enable precise genome editing by introducing breaks at specific genomic loci, i.e., they rely on the recognition of a specific DNA sequence within the genome to target a nuclease domain to this location and mediate the generation of a double-strand break at the target sequence. A double-strand break in the DNA subsequently recruits endogenous repair machinery to the break site to mediate genome editing by either non-homologous end-joining (NHEJ) or homology-directed repair (HDR). Thus, the repair of the break can result in the introduction of insertion/deletion mutations that disrupt (e.g., silence, repress, or enhance) the target gene product.

Major classes of nucleases that have been developed to enable site-specific genomic editing include zinc finger nucleases (ZFNs), transcription activator-like nucleases (TALENs), and CRISPR-associated nucleases (e.g., CRISPR/Cas9). These nuclease systems can be broadly classified into two categories based on their mode of DNA recognition: ZFNs and TALENs achieve specific DNA binding via protein-DNA interactions, whereas CRISPR systems, such as Cas9, are targeted to specific DNA sequences by a short RNA guide molecule that base-pairs directly with the target DNA and by protein-DNA interactions. See, e.g., Cox et al., Nature Medicine, 2015, Vol. 21, No. 2.

Non-limiting examples of gene-editing methods that may be used in accordance with TIL expansion methods of the present invention include CRISPR methods, TALE methods, and ZFN methods, which are described in more detail below. According to an embodiment, a method for expanding TILs, PBLs, and/or MILs into a therapeutic population may be carried out in accordance with any embodiment of the methods described herein (e.g., GEN 3 process) or as described in PCT/US2017/058610, PCT/US2018/012605, or PCT/US2018/012633, wherein the method further comprises gene-editing at least a portion of the TILs, PBLs, and/or MILs by one or more of a CRISPR method, a TALE method or a ZFN method, in order to generate TILs, PBLs, and/or MILs that can provide an enhanced therapeutic effect. According to an embodiment, gene-edited TILs, PBLs, and/or MILs can be evaluated for an improved therapeutic effect by comparing them to non-modified TILs, PBLs, and/or MILs in vitro, e.g., by evaluating in vitro effector function, cytokine profiles, etc. compared to unmodified TILs, PBLs, and/or MILs. In certain embodiments, the method comprises gene editing a population of TILs, PBLs, and/or MILs using CRISPR, TALE and/or ZFN methods.

In some embodiments of the present invention, electroporation is used for delivery of a gene editing system, such as CRISPR, TALEN, and ZFN systems. In some embodiments of the present invention, the electroporation system is a flow electroporation system. An example of a suitable flow electroporation system suitable for use with some embodiments of the present invention is the commercially-available MaxCyte STX system. There are several alternative commercially-available electroporation instruments which may be suitable for use with the present invention, such as the AgilePulse system or ECM 830 available from BTX-Harvard Apparatus, Cellaxess Elektra (Cellectricon), Nucleofector (Lonza/Amaxa), GenePulser MXcell (BIORAD), iPorator-96 (Primax) or siPORTer96 (Ambion). In some embodiments of the present invention, the electroporation system forms a closed, sterile system with the remainder of the TIL expansion method. In some embodiments of the present invention, the electroporation system is a pulsed electroporation system as described herein, and forms a closed, sterile system with the remainder of the TIL expansion method.

A method for expanding TILs, PBLs, and/or MILs into a therapeutic population may be carried out in accordance with any embodiment of the methods described herein (e.g., process GEN 3) or as described in PCT/US2017/058610, PCT/US2018/012605, or PCT/US2018/012633, wherein the method further comprises gene-editing at least a portion of the TILs, PBLs, and/or MILs by a CRISPR method (e.g., CRISPR/Cas9 or CRISPR/Cpf1). According to particular embodiments, the use of a CRISPR method during the TIL expansion process causes expression of one or more immune checkpoint genes to be silenced or reduced in at least a portion of the therapeutic population of TILs, PBLs, and/or MILs. Alternatively, the use of a CRISPR method during the TIL expansion process causes expression of one or more immune checkpoint genes to be enhanced in at least a portion of the therapeutic population of TILs, PBLs, and/or MILs.

CRISPR stands for “Clustered Regularly Interspaced Short Palindromic Repeats.” A method of using a CRISPR system for gene editing is also referred to herein as a CRISPR method. There are three types of CRISPR systems which incorporate RNAs and Cas proteins, and which may be used in accordance with the present invention: Types I, II, and III. The Type II CRISPR (exemplified by Cas9) is one of the most well-characterized systems.

CRISPR technology was adapted from the natural defense mechanisms of bacteria and archaea (the domain of single-celled microorganisms). These organisms use CRISPR-derived RNA and various Cas proteins, including Cas9, to foil attacks by viruses and other foreign bodies by chopping up and destroying the DNA of a foreign invader. A CRISPR is a specialized region of DNA with two distinct characteristics: the presence of nucleotide repeats and spacers. Repeated sequences of nucleotides are distributed throughout a CRISPR region with short segments of foreign DNA (spacers) interspersed among the repeated sequences. In the type II CRISPR/Cas system, spacers are integrated within the CRISPR genomic loci and transcribed and processed into short CRISPR RNA (crRNA). These crRNAs anneal to trans-activating crRNAs (tracrRNAs) and direct sequence-specific cleavage and silencing of pathogenic DNA by Cas proteins. Target recognition by the Cas9 protein requires a “seed” sequence within the crRNA and a conserved dinucleotide-containing protospacer adjacent motif (PAM) sequence upstream of the crRNA-binding region. The CRISPR/Cas system can thereby be retargeted to cleave virtually any DNA sequence by redesigning the crRNA. The crRNA and tracrRNA in the native system can be simplified into a single guide RNA (sgRNA) of approximately 100 nucleotides for use in genetic engineering. The CRISPR/Cas system is directly portable to human cells by co-delivery of plasmids expressing the Cas9 endo-nuclease and the necessary crRNA components. Different variants of Cas proteins may be used to reduce targeting limitations (e.g., orthologs of Cas9, such as Cpf1).

Non-limiting examples of genes that may be silenced or inhibited by permanently gene-editing TILs, PBLs, and/or MILs via a CRISPR method include PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, and GUCY1B3.

Non-limiting examples of genes that may be enhanced by permanently gene-editing TILs, PBLs, and/or MILs via a CRISPR method include CCR2, CCR4, CCR5, CXCR2, CXCR3, CX3CR1, IL-2, IL12, IL-15, and IL-21.

Examples of systems, methods, and compositions for altering the expression of a target gene sequence by a CRISPR method, and which may be used in accordance with embodiments of the present invention, are described in U.S. Pat. Nos. 8,697,359; 8,993,233; 8,795,965; 8,771,945; 8,889,356; 8,865,406; 8,999,641; 8,945,839; 8,932,814; 8,871,445; 8,906,616; and 8,895,308, which are incorporated by reference herein. Resources for carrying out CRISPR methods, such as plasmids for expressing CRISPR/Cas9 and CRISPR/Cpf1, are commercially available from companies such as GenScript.

In an embodiment, genetic modifications of populations of TILs, PBLs, and/or MILs, as described herein, may be performed using the CRISPR/Cpf1 system as described in U.S. Pat. No. 9,790,490, the disclosure of which is incorporated by reference herein.

A method for expanding TILs, PBLs, and/or MILs into a therapeutic population may be carried out in accordance with any embodiment of the methods described herein (e.g., process 2A) or as described in PCT/US2017/058610, PCT/US2018/012605, or PCT/US2018/012633, wherein the method further comprises gene-editing at least a portion of the TILs, PBLs, and/or MILs by a TALE method. According to particular embodiments, the use of a TALE method during the TIL expansion process causes expression of one or more immune checkpoint genes to be silenced or reduced in at least a portion of the therapeutic population of TILs, PBLs, and/or MILs. Alternatively, the use of a TALE method during the TIL expansion process causes expression of one or more immune checkpoint genes to be enhanced in at least a portion of the therapeutic population of TILs, PBLs, and/or MILs.

TALE stands for “Transcription Activator-Like Effector” proteins, which include TALENs (“Transcription Activator-Like Effector Nucleases”). A method of using a TALE system for gene editing may also be referred to herein as a TALE method. TALEs are naturally occurring proteins from the plant pathogenic bacteria genus Xanthomonas, and contain DNA-binding domains composed of a series of 33-35-amino-acid repeat domains that each recognizes a single base pair. TALE specificity is determined by two hypervariable amino acids that are known as the repeat-variable di-residues (RVDs). Modular TALE repeats are linked together to recognize contiguous DNA sequences. A specific RVD in the DNA-binding domain recognizes a base in the target locus, providing a structural feature to assemble predictable DNA-binding domains. The DNA binding domains of a TALE are fused to the catalytic domain of a type IIS FokI endonuclease to make a targetable TALE nuclease. To induce site-specific mutation, two individual TALEN arms, separated by a 14-20 base pair spacer region, bring FokI monomers in close proximity to dimerize and produce a targeted double-strand break.

Several large, systematic studies utilizing various assembly methods have indicated that TALE repeats can be combined to recognize virtually any user-defined sequence. Custom-designed TALE arrays are also commercially available through Cellectis Bioresearch (Paris, France), Transposagen Biopharmaceuticals (Lexington, Ky., USA), and Life Technologies (Grand Island, N.Y., USA). TALE and TALEN methods suitable for use in the present invention are described in U.S. Patent Application Publication Nos. US 2011/0201118 A1; US 2013/0117869 A1; US 2013/0315884 A1; US 2015/0203871 A1 and US 2016/0120906 A1, the disclosures of which are incorporated by reference herein.

Non-limiting examples of genes that may be silenced or inhibited by permanently gene-editing TILs, PBLs, and/or MILs via a TALE method include PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, and GUCY1B3.

Non-limiting examples of genes that may be enhanced by permanently gene-editing TILs, PBLs, and/or MILs via a TALE method include CCR2, CCR4, CCR5, CXCR2, CXCR3, CX3CR1, IL-2, IL12, IL-15, and IL-21.

Examples of systems, methods, and compositions for altering the expression of a target gene sequence by a TALE method, and which may be used in accordance with embodiments of the present invention, are described in U.S. Pat. No. 8,586,526, which is incorporated by reference herein.

A method for expanding TILs, PBLs, and/or MILs into a therapeutic population may be carried out in accordance with any embodiment of the methods described herein (e.g., process GEN 3) or as described in PCT/US2017/058610, PCT/US2018/012605, or PCT/US2018/012633, wherein the method further comprises gene-editing at least a portion of the TILs, PBLs, and/or MILs by a zinc finger or zinc finger nuclease method. According to particular embodiments, the use of a zinc finger method during the TIL expansion process causes expression of one or more immune checkpoint genes to be silenced or reduced in at least a portion of the therapeutic population of TILs, PBLs, and/or MILs. Alternatively, the use of a zinc finger method during the TIL expansion process causes expression of one or more immune checkpoint genes to be enhanced in at least a portion of the therapeutic population of TILs, PBLs, and/or MILs.

An individual zinc finger contains approximately 30 amino acids in a conserved ββα configuration. Several amino acids on the surface of the α-helix typically contact 3 bp in the major groove of DNA, with varying levels of selectivity. Zinc fingers have two protein domains. The first domain is the DNA binding domain, which includes eukaryotic transcription factors and contain the zinc finger. The second domain is the nuclease domain, which includes the FokI restriction enzyme and is responsible for the catalytic cleavage of DNA.

The DNA-binding domains of individual ZFNs typically contain between three and six individual zinc finger repeats and can each recognize between 9 and 18 base pairs. If the zinc finger domains are specific for their intended target site then even a pair of 3-finger ZFNs that recognize a total of 18 base pairs can, in theory, target a single locus in a mammalian genome. One method to generate new zinc-finger arrays is to combine smaller zinc-finger “modules” of known specificity. The most common modular assembly process involves combining three separate zinc fingers that can each recognize a 3 base pair DNA sequence to generate a 3-finger array that can recognize a 9 base pair target site. Alternatively, selection-based approaches, such as oligomerized pool engineering (OPEN) can be used to select for new zinc-finger arrays from randomized libraries that take into consideration context-dependent interactions between neighboring fingers. Engineered zinc fingers are available commercially; Sangamo Biosciences (Richmond, Calif., USA) has developed a propriety platform (CompoZr®) for zinc-finger construction in partnership with Sigma-Aldrich (St. Louis, Mo., USA).

Non-limiting examples of genes that may be silenced or inhibited by permanently gene-editing TILs, PBLs, and/or MILs via a zinc finger method include PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, and GUCY1B3.

Non-limiting examples of genes that may be enhanced by permanently gene-editing TILs, PBLs, and/or MILs via a zinc finger method include CCR2, CCR4, CCR5, CXCR2, CXCR3, CX3CR1, IL-2, IL12, IL-15, and IL-21.

Examples of systems, methods, and compositions for altering the expression of a target gene sequence by a zinc finger method, which may be used in accordance with embodiments of the present invention, are described in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, which are incorporated by reference herein.

Other examples of systems, methods, and compositions for altering the expression of a target gene sequence by a zinc finger method, which may be used in accordance with embodiments of the present invention, are described in Beane, et al., Mol. Therapy, 2015, 23 1380-1390, the disclosure of which is incorporated by reference herein.

### Chimeric Antigen Receptors and Genetically-Modified T-Cell Receptors

In some embodiments, the TILs, PBLs, and/or MILs are optionally genetically engineered to include additional functionalities, including, but not limited to, a high-affinity T cell receptor (TCR), e.g., a TCR targeted at a tumor-associated antigen such as MAGE-1, HER2, or NY-ESO-1, or a chimeric antigen receptor (CAR) which binds to a tumor-associated cell surface molecule (e.g., mesothelin) or lineage-restricted cell surface molecule (e.g., CD19). In certain embodiments, the method comprises genetically engineering a population of TILs, PBLs, and/or MILs to include a high-affinity T cell receptor (TCR), e.g., a TCR targeted at a tumor-associated antigen such as MAGE-1, HER2, or NY-ESO-1, or a CAR which binds to a tumor-associated cell surface molecule (e.g., mesothelin) or lineage-restricted cell surface molecule (e.g., CD19). In certain embodiments, the method comprises genetically engineering a population of TILs, PBLs, and/or MILs to include a CAR specific for CD19, CD20, CD19 and CD20 (bispecific), CD30, CD33, CD123, PSMA, mesothelin, CE7, HER2/neu BCMA, EGFRvIII, HER2/CMV, IL13Rα2, human C4 folate receptor-alpha (αFR), or GD2.

In some embodiments, the TILs, PBLs, and/or MILs expanded according to the methods of the present invention are genetically modified to target antigens through expression of CARs. In some embodiments, the TILs, PBLs, and/or MILs of the present invention are transduced with an expression vector comprising a nucleic acid encoding a CAR comprising a single chain variable fragment antibody fused with at least one endodomain of a T-cell signaling molecule. In some embodiments, the transducing step takes place at any time during the expansion process. In some embodiments, the transducing step takes place after the expanded cells are harvested. In some embodiments, the TILs, PBLs, and/or MILs of the present invention include a polynucleotide capable of expression of a CAR.

In one embodiment, the CARs or nucleotides encoding CARs are prepared and transduced according to the disclosure in U.S. Pat. Nos. 9,328,156; 8,399,645; 7,446,179; 6,410,319; 7,446,190, and U.S. Patent Application Publication Nos. US 2015/0038684; US 2015/0031624; US 2014/0301993 A1; US 2014/0271582 A1; US 2015/0051266 A1; US 2014/0322275 A1; and US 2014/0004132 A1, the disclosures of each of which is incorporated by reference herein. In U.S. Pat. No. 9,328,156, CAR-T cells are prepared to treat patients with B-cell lymphomas, and particularly CLL, and the embodiments discussed therein are useful in the present invention. For example, a CAR-T cell expressing a CD19 antigen binding domain, a transmembrane domain, a 4-1BB costimulatory signaling region, and a CD3 zeta signaling domain is useful in the present invention. In an embodiment of the invention, the CAR comprises a target-specific binding element, or antibody binding domain, a transmembrane domain, and a cytoplasmic domain. Hematopoietic tumor antigens (for the antimbody binding domain) are well known in the art and include, for example, CD19, CD20, CD22, ROR1, Mesothelin, CD33/IL3Ra, c-Met, PSMA, Glycolipid F77, EGFRvIII, GD-2, NY-ESO-1 TCR, MAGE A3 TCR, and the like. In an embodiment, the transmembrane domain comprises the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, and may be synthetic. In an embodiment, the cytoplasmic or signaling domain comprise a portion or all of the TCR zeta, FcR gamma, FcR beta, CD3 gamma, CD3 delta, CD3 epsilon, CD3 zeta, CD5, CD22, CD79a, CD79b, or CD66d domains. In an embodiment of the invention, the cytoplasmic or signaling domain may also include a co-stimulatory molecule, for example, CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, MC, or a ligand that specifically binds with CD83, and the like.

In an embodiment of the invention, the CAR modified TILs, PBLs, and/or MILs comprise an antigen binding domain, a costimulatory signaling region, and a CD3 zeta signaling domain. In an embodiment of the invention, the CAR-modified TILs, PBLs, and/or MILs comprise a CD19-directed antigen binding domain, a 4-1BB or CD28 costimulatory signaling region, and a CD3 zeta signaling domain. In an embodiment of the invention, the CAR-modified TILs include a suicide switch (such as a Caspase-9/rimiducid) or an activation switch (such as an inducible MyD88/CD40 activation switch). In an embodiment of the invention, the CAR-modified TILs are modified using a lentiviral vector expressing a CAR.

In some embodiments of the invention, the TILs, PBLs, and/or MILs expanded according to the methods of the present invention are used in a method to modify signaling in the cells using modified T-cell receptors (TCRs), including genetically altered TCRs. In some embodiments, the TILs, PBLs, and/or MILs of the present invention are modified to include additional functionalities, including, but not limited to, a high-affinity T cell receptor (TCR), e.g., a TCR targeted at a tumor-associated antigen such as MAGE-1, MAGE-3, MAGE-A3, MAGE-A4, MAGE-A10, MART-1, CEA, gp100, alpha-fetoprotein (AFP), HER2, PRAME, CT83, SSX2, or NY-ESO-1. Methods for modifying TCRs and methods for creating artificial TCRs are known in the art, and are disclosed, for example, in U.S. Pat. Nos. 6,811,785; 7,569,664; 7,666,604; 8,143,376; 8,283,446; 9,181,527; 7,329,731; 7,070,995; 7,265,209; 8,361,794; and 8,697,854; and U.S. Patent Application Publication Nos. US 2017/0051036 A1; US 2010/0034834 A1; US 2011/0014169 A1; US 2016/0200824 A1; and US 2002/0058253 A1, the disclosures of each of which are incorporated by reference herein. In some embodiments of the invention, the TILs, PBLs, and/or MILs expanded according to the methods of the present invention are used in a method to modify signaling in the cells using modified TCRs against a tumor-associated antigen. In some embodiments of the invention, the TILs, PBLs, and/or MILs expanded according to the methods of the present invention are used in a method to modify signaling in the cells using modified TCRs, including genetically altered TCRs wherein the TILs, PBLs, and/or MILs are modified to reduce the presence of endogenous TCRs.

In some embodiments of the invention, the TILs, PBLs, and/or MILs expanded according to the methods of the present invention comprise transiently or stably modified TCRs, such as TCRs modified to be specific for a cancer testis antigen, such as a MAGE-A antigen. In some embodiments, the TILs, PBLs, and/or MILs may include at least one TCR comprising a modified complementarity determining region (CDR). In some embodiments, the TILs, PBLs, and/or MILs may include at least one TCR comprising a modified CDR2, with retention of the wild type sequences in the beta chain to increase the TCR affinity. In some embodiments, the TILs, PBLs, and/or MILs may include TCRs which are mutated relative to the native TCR α chain variable domain and/or β chain variable domain (see FIG. 1b and SEQ ID NO: 2) in at least one CDR (such as CDR2), variable domain framework region, or other hypervariable regions in the variable domains of the TCRs (such as the hypervariable 4 (HV4) regions), such that the mutants produce a high affinity TCR. The TILs, PBLs, and/or MILs may include at least one TCR anchored to the membrane by a transmembrane sequence, said TCR comprising an interchain disulfide bond between extracellular constant domain residues which is not present in native TCRs, as described in U.S. Pat. No. 8,361,794, the disclosure of which is incorporated by reference herein. In some embodiments, the TILs, PBLs, and/or MILs may include at least one TCR having the property of binding to a specific human leukocyte antigen (HLA)-A1 complex and comprising a specified wild type TCR which has specific mutations in the TCR alpha variable domain and/or the TCR beta variable domain to increase affinity. In some embodiments of the invention, the TILs, PBLs, and/or MILs expanded according to the methods of the present invention comprise a stably modified TCR with increased affinity to NY-ESO-1, MART-1, CEA, gp100, alpha-fetoprotein (AFP), HER2, PRAME (preferentially-expressed antigen in melanoma), CT83, SSX2, MAGE-1, MAGE-3, MAGE-A3, MAGE-A4, or MAGE-A10.

Immune Checkpoints

In an embodiment of the present invention, one or more immune checkpoint genes may be modified. Immune checkpoints are molecules expressed by lymphocytes that regulate an immune response via inhibitory or stimulatory pathways. In the case of cancer, immune checkpoint pathways are often activated to inhibit the anti-tumor response, i.e., the expression of certain immune checkpoints by malignant cells inhibits the anti-tumor immunity and favors the growth of cancer cells. See, e.g., Marin-Acevedo et al., Journal of Hematology & Oncology (2018) 11:39. Thus, certain inhibitory checkpoint molecules serve as targets for immunotherapies of the present invention. According to particular embodiments, cells are modified through CAR or TCR to block or stimulate certain immune checkpoint pathways and thereby enhance the body's immunological activity against tumors.

The most broadly studied checkpoints include programmed cell death receptor-1 (PD-1) and cytotoxic T lymphocyte-associated molecule-4 (CTLA-4), which are inhibitory receptors on immune cells that inhibit key effector functions (e.g., activation, proliferation, cytokine release, cytoxicity, etc.) when they interact with an inhibitory ligand. Numerous checkpoint molecules, in addition to PD-1 and CTLA-4, have emerged as potential targets for immunotherapy, as discussed in more detail below.

Non-limiting examples of immune checkpoint genes that may be silenced or inhibited include PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, BAFF (BR3), CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, and GUCY1B3. For example, immune checkpoint genes that may be silenced or inhibited may be selected from the group comprising PD-1, CTLA-4, LAG-3, TIM-3, Cish, TGFβ, and PKA. BAFF (BR3) is described in Bloom, et al., J. Immunother., 2018, in press. According to another example, immune checkpoint genes that may be silenced or inhibited in TILs of the present invention may be selected from the group comprising PD-1, LAG-3, TIM-3, CTLA-4, TIGIT, CISH, TGFβR2, PRA, CBLB, BAFF (BR3), and combinations thereof.

PD-1

One of the most studied targets for the induction of checkpoint blockade is the programmed death receptor (PD1 or PD-1, also known as PDCD1), a member of the CD28 super family of T-cell regulators. Its ligands, PD-L1 and PD-L2, are expressed on a variety of tumor cells, including melanoma. The interaction of PD-1 with PD-L1 inhibits T-cell effector function, results in T-cell exhaustion in the setting of chronic stimulation, and induces T-cell apoptosis in the tumor microenvironment. PD1 may also play a role in tumor-specific escape from immune surveillance.

According to particular embodiments, expression of PD1 in TILs is silenced or reduced in accordance with compositions and methods of the present invention.

CTLA-4

CTLA-4 expression is induced upon T-cell activation on activated T-cells, and competes for binding with the antigen presenting cell activating antigens CD80 and CD86. Interaction of CTLA-4 with CD80 or CD86 causes T-cell inhibition and serves to maintain balance of the immune response. However, inhibition of the CTLA-4 interaction with CD80 or CD86 may prolong T-cell activation and thus increase the level of immune response to a cancer antigen.

According to particular embodiments, expression of CTLA-4 in TILs is silenced or reduced in accordance with compositions and methods of the present invention.

LAG-3

Lymphocyte activation gene-3 (LAG-3, CD223) is expressed by T cells and natural killer (NK) cells after major histocompatibility complex (MHC) class II ligation. Although its mechanism remains unclear, its modulation causes a negative regulatory effect over T cell function, preventing tissue damage and autoimmunity. LAG-3 and PD-1 are frequently co-expressed and upregulated on TILs, leading to immune exhaustion and tumor growth. Thus, LAG-3 blockade improves anti-tumor responses. See, e.g., Marin-Acevedo et al., Journal of Hematology & Oncology (2018) 11:39.

According to particular embodiments, expression of LAG-3 in TILs is silenced or reduced in accordance with compositions and methods of the present invention.

TIM-3

T cell immunoglobulin-3 (TIM-3) is a direct negative regulator of T cells and is expressed on NK cells and macrophages. TIM-3 indirectly promotes immunosuppression by inducing expansion of myeloid-derived suppressor cells (MDSCs). Its levels have been found to be particularly elevated on dysfunctional and exhausted T-cells, suggesting an important role in malignancy.

According to particular embodiments, expression of TIM-3 in TILs is silenced or reduced in accordance with compositions and methods of the present invention.

Cish

Cish, a member of the suppressor of cytokine signaling (SOCS) family, is induced by TCR stimulation in CD8+ T cells and inhibits their functional avidity against tumors. Genetic deletion of Cish in CD8+ T cells may enhance their expansion, functional avidity, and cytokine polyfunctionality, resulting in pronounced and durable regression of established tumors. See, e.g., Palmer et al., Journal of Experimental Medicine, 212 (12): 2095 (2015).

According to particular embodiments, expression of Cish in TILs is silenced or reduced in accordance with compositions and methods of the present invention.

TGFβ

The TGFβ signaling pathway has multiple functions in regulating cell growth, differentiation, apoptosis, motility and invasion, extracellular matrix production, angiogenesis, and immune response. TGFβ signaling deregulation is frequent in tumors and has crucial roles in tumor initiation, development and metastasis. At the microenvironment level, the TGFβ pathway contributes to generate a favorable microenvironment for tumor growth and metastasis throughout carcinogenesis. See, e.g., Neuzillet et al., Pharmacology & Therapeutics, Vol. 147, pp. 22-31 (2015).

According to particular embodiments, expression of TGFβ in TILs, PBLs, and/or MILs is silenced or reduced in accordance with compositions and methods of the present invention.

PKA

Protein Kinase A (PKA) is a well-known member of the serine-threonin protein kinase superfamily. PKA, also known as cAMP-dependent protein kinase, is a multi-unit protein kinase that mediates signal transduction of G-protein coupled receptors through its activation upon cAMP binding. It is involved in the control of a wide variety of cellular processes from metabolism to ion channel activation, cell growth and differentiation, gene expression and apoptosis. Importantly, PKA has been implicated in the initiation and progression of many tumors. See, e.g., Sapio et al., EXCLI Journal; 2014; 13: 843-855.

According to particular embodiments, expression of PKA in TILs is silenced or reduced in accordance with compositions and methods of the present invention.

CBLB

CBLB (or CBL-B) is a E3 ubiquitin-protein ligase and is a negative regulator of T cell activation. Bachmaier, et al., Nature, 2000, 403, 211-216; Wallner, et al., Clin. Dev. Immunol. 2012, 692639.

According to particular embodiments, expression of CBLB in TILs is silenced or reduced in accordance with compositions and methods of the present invention.

Overexpression of Co-Stimulatory Receptors or Adhesion Molecules

According to additional embodiments, one or more immune checkpoint genes are enhanced. Non-limiting examples of immune checkpoint genes that may exhibit enhanced expression include certain chemokine receptors and interleukins, such as CCR2, CCR4, CCR5, CXCR2, CXCR3, CX3CR1, IL-2, IL-4, IL-7, IL-15, and IL-21.

CCRs

For adoptive T cell immunotherapy to be effective, T cells need to be trafficked properly into tumors by chemokines. A match between chemokines secreted by tumor cells, chemokines present in the periphery, and chemokine receptors expressed by T cells is important for successful trafficking of T cells into a tumor bed.

According to particular embodiments, an increase in the expression of certain chemokine receptors in the TILs, such as one or more of CCR2, CCR4, CCR5, CXCR2, CXCR3 and CX3CR1 is contemplated. Over-expression of CCRs may help promote effector function and proliferation of TILs following adoptive transfer.

According to particular embodiments, expression of one or more of CCR2, CCR4, CCR5, CXCR2, CXCR3 and CX3CR1 is enhanced.

In an embodiment, CCR4 and/or CCR5 adhesion molecules are inserted into a TIL population using a gamma-retroviral or lentiviral method as described herein. In an embodiment, CXCR2 adhesion molecule are inserted into a TIL population using a gamma-retroviral or lentiviral method as described in Forget, et al., Frontiers Immunology 2017, 8, 908 or Peng, et al., Clin. Cancer Res. 2010, 16, 5458, the disclosures of which are incorporated by reference herein.

Interleukins

According to additional embodiments, gene-editing methods of the present invention may be used to increase the expression of certain interleukins, such as one or more of IL-2, IL-4, IL-7, IL-15, and IL-21. Certain interleukins have been demonstrated to augment effector functions of T cells and mediate tumor control.

According to particular embodiments, expression of one or more of IL-2, IL-4, IL-7, IL-15, and IL-21 is enhanced in accordance with compositions and methods of the present invention.

Aptly, the population of TILs, PBLs, and/or MILs may be a first population, a second population and/or a third population as described herein.

Methods of Treating Cancers

The compositions and combinations of TILs, PBLs, and/or MILs (and populations thereof) described above can be used in a method for treating hyperproliferative disorders. In a preferred embodiment, they are for use in treating cancers. In a preferred embodiment, the invention provides a method of treating a cancer, wherein the cancer is a hematological malignancy, such as a liquid tumor. In a preferred embodiment, the invention provides a method of treating a cancer, wherein the cancer is a hematological malignancy selected from the group consisting of acute myeloid leukemia (AML), mantle cell lymphoma (MCL), follicular lymphoma (FL), diffuse large B cell lymphoma (DLBCL), activated B cell (ABC) DLBCL, germinal center B cell (GCB) DLBCL, chronic lymphocytic leukemia (CLL), CLL with Richter's transformation (or Richter's syndrome), small lymphocytic leukemia (SLL), non-Hodgkin's lymphoma (NHL), Hodgkin's lymphoma, relapsed and/or refractory Hodgkin's lymphoma, B cell acute lymphoblastic leukemia (B-ALL), mature B-ALL, Burkitt's lymphoma, Waldenström's macroglobulinemia (WM), multiple myeloma, myelodysplatic syndromes, myelofibrosis, chronic myelocytic leukemia, follicle center lymphoma, indolent NHL, human immunodeficiency virus (HIV) associated B cell lymphoma, and Epstein-Barr virus (EBV) associated B cell lymphoma, including subpopulations of patients with the foregoing diseases that are refractory to, intolerant to, or relapsed from treatment with a BTK inhibitor, including ibrutinib.

In an embodiment of the present invention, CLL patients who have been pretreated with ibrutinib represent a subpopulation of patients that can be successfully treated with the PBLs of the present invention. In particular, CLL patients who have been pretreated with ibrutinib, and who are no longer responsive to ibrutinib treatment, represent a subpopulation of patients that can be successfully treated with the PBLs of the present invention. In another embodiment, CLL patients who have been pretreated with ibrutinib and who have developed Richter's transformation (or Richter's syndrome), represent a subpopulation of patients that can be successfully treated with the PBLs of the present invention. In another embodiment, CLL patients who have been pretreated with ibrutinib, who have developed Richter's transformation (or Richter's syndrome) and who are no longer responsive to ibrutinib treatment, represent a subpopulation of patients that can be successfully treated with the PBLs of the present invention.

In an embodiment, the invention provides a method of treating a cancer, wherein the cancer is a hematological malignancy that responds to therapy with PD-1 and/or PD-L1 inhibitors including pembrolizumab, nivolumab, durvalumab, avelumab, or atezolizumab.

In an embodiment, the invention provides a method of treating a cancer in a patient with a population of tumor infiltrating lymphocytes (TILs) comprising the steps of:
(a) obtaining a tumor from the patient by resection, biopsy, needle aspiration, or apheresis, the tumor comprising a first population of TILs;
(b) optionally fragmenting or dissociating the tumor to obtain tumor fragments and contacting the tumor or tumor fragments with a first cell culture medium;
(c) performing an initial expansion of the first population of TILs in the first cell culture medium to obtain a second population of TILs, wherein the second population of TILs is at least 5-fold greater in number than the first population of TILs, wherein the first cell culture medium comprises IL-2;
(d) performing a second expansion of the second population of TILs in a second cell culture medium to obtain a third population of TILs, wherein the third population of TILs is at least 50-fold greater in number than the second population of TILs after 7 days from the start of the second expansion; wherein the second cell culture medium comprises IL-2, OKT-3 (anti-CD3 antibody), and irradiated allogeneic peripheral blood mononuclear cells (PBMCs); and wherein the second expansion is performed over a period of 14 days or less;
(e) harvesting the third population of TILs; and
(f) administering a therapeutically effective portion of the third population of TILs to the patient;
wherein the tumor is a liquid tumor, and wherein the cancer is a hematological malignancy.

In an embodiment, the invention provides a method of treating a cancer in a patient with a population of tumor infiltrating lymphocytes (TILs) comprising the steps of:
(a) obtaining a tumor from the patient by resection, biopsy, needle aspiration, or apheresis, the tumor comprising a first population of TILs;
(b) optionally fragmenting or dissociating the tumor to obtain tumor fragments and contacting the tumor or tumor fragments with a first cell culture medium;
(c) performing an initial expansion of the first population of TILs in the first cell culture medium to obtain a second population of TILs, wherein the second population of TILs is at least 5-fold greater in number than the first population of TILs, wherein the first cell culture medium comprises IL-2;
(d) performing a second expansion of the second population of TILs in a second cell culture medium to obtain a third population of TILs, wherein the third population of TILs is at least 50-fold greater in number than the second population of TILs after 7 days from the start of the second expansion; wherein the second cell culture medium comprises IL-2, OKT-3 (anti-CD3 antibody), and irradiated allogeneic peripheral blood mononuclear cells (PBMCs); and wherein the second expansion is performed over a period of 14 days or less;
(e) harvesting the third population of TILs; and
(f) administering a therapeutically effective portion of the third population of TILs to the patient;
wherein the tumor is a liquid tumor, and wherein the cancer is a hematological malignancy selected from the group consisting of acute myeloid leukemia (AML), mantle cell lymphoma (MCL), follicular lymphoma (FL), diffuse large B cell lymphoma (DLBCL), activated B cell (ABC) DLBCL, germinal center B cell (GCB) DLBCL, chronic lymphocytic leukemia (CLL), CLL with Richter's transformation (or Richter's syndrome), small lymphocytic leukemia (SLL), non-Hodgkin's lymphoma (NHL), Hodgkin's lymphoma, relapsed and/or refractory Hodgkin's lymphoma, B cell acute lymphoblastic leukemia (B-ALL), mature B-ALL, Burkitt's lymphoma, Waldenström's macroglobulinemia (WM), multiple myeloma, myelodysplatic syndromes, myelofibrosis, chronic myelocytic leukemia, follicle center lymphoma, indolent NHL, human immunodeficiency virus (HIV) associated B cell lymphoma, and Epstein-Barr virus (EBV) associated B cell lymphoma.

In an embodiment, the invention provides a method of treating a cancer in a patient with a population of tumor infiltrating lymphocytes (TILs) comprising the steps of:
(a) pre-treating the patient with a regimen comprising a kinase inhibitor or an ITK inhibitor;
(b) obtaining a tumor from the patient by resection, biopsy, needle aspiration, or apheresis, the tumor comprising a first population of TILs;
(c) optionally fragmenting or dissociating the tumor to obtain tumor fragments and contacting the tumor or tumor fragments with a first cell culture medium;
(d) performing an initial expansion of the first population of TILs in the first cell culture medium to obtain a second population of TILs, wherein the second population of TILs is at least 5-fold greater in number than the first population of TILs, wherein the first cell culture medium comprises IL-2;
(e) performing a second expansion of the second population of TILs in a second cell culture medium to obtain a third population of TILs, wherein the third population of TILs is at least 50-fold greater in number than the second population of TILs after 7 days from the start of the second expansion; wherein the second cell culture medium comprises IL-2, OKT-3 (anti-CD3 antibody), and irradiated allogeneic peripheral blood mononuclear cells (PBMCs); and wherein the second expansion is performed over a period of 14 days or less;
(f) harvesting the third population of TILs; and
(g) administering a therapeutically effective portion of the third population of TILs to the patient;
wherein the tumor is a liquid tumor, and wherein the cancer is a hematological malignancy.

In an embodiment, the invention provides a method of treating a cancer in a patient with a population of tumor infiltrating lymphocytes (TILs) comprising the steps of:
(a) pre-treating the patient with a regimen comprising a kinase inhibitor or an ITK inhibitor;
(b) obtaining a tumor from the patient by resection, biopsy, needle aspiration, or apheresis, the tumor comprising a first population of TILs;
(c) optionally fragmenting or dissociating the tumor to obtain tumor fragments and contacting the tumor or tumor fragments with a first cell culture medium;
(d) performing an initial expansion of the first population of TILs in the first cell culture medium to obtain a second population of TILs, wherein the second population of TILs is at least 5-fold greater in number than the first population of TILs, wherein the first cell culture medium comprises IL-2;
(e) performing a second expansion of the second population of TILs in a second cell culture medium to obtain a third population of TILs, wherein the third population of TILs is at least 50-fold greater in number than the second population of TILs after 7 days from the start of the second expansion; wherein the second cell culture medium comprises IL-2, OKT-3 (anti-CD3 antibody), and irradiated allogeneic peripheral blood mononuclear cells (PBMCs); and wherein the second expansion is performed over a period of 14 days or less;
(f) harvesting the third population of TILs; and
(g) administering a therapeutically effective portion of the third population of TILs to the patient;
wherein the tumor is a liquid tumor, and wherein the cancer is a hematological malignancy selected from the group consisting of acute myeloid leukemia (AML), mantle cell lymphoma (MCL), follicular lymphoma (FL), diffuse large B cell lymphoma (DLBCL), activated B cell (ABC) DLBCL, germinal center B cell (GCB) DLBCL, chronic lymphocytic leukemia (CLL), CLL with Richter's transformation (or Richter's syndrome), small lymphocytic leukemia (SLL), non-Hodgkin's lymphoma (NHL), Hodgkin's lymphoma, relapsed and/or refractory Hodgkin's lymphoma, B cell acute lymphoblastic leukemia (B-ALL), mature B-ALL, Burkitt's lymphoma, Waldenström's macroglobulinemia (WM), multiple myeloma, myelodysplatic syndromes, myelofibrosis, chronic myelocytic leukemia, follicle center lymphoma, indolent NHL, human immunodeficiency virus (HIV) associated B cell lymphoma, and Epstein-Barr virus (EBV) associated B cell lymphoma.

In an embodiment of the invention, TILs are expanded using MIL Method 1 and administered to a patient in accordance with the present invention.

In an embodiment of the invention, TILs are expanded using MIL Method 2 and administered to a patient in accordance with the present invention to treat cancer.

In an embodiment of the invention, TILs are expanded using MIL Method 3 and administered to a patient in accordance with the present invention to treat cancer.

In an embodiment of the invention, TILs expanded using MIL Method 1, MIL Method 2, or MIL Method 3 are administered to a patient in accordance with the present invention to treat AML.

In an embodiment of the invention, TILs are expanded using PBL Method 2 and administered to a patient in accordance with the present invention to treat cancer.

In an embodiment of the invention, TILs are expanded using PBL Method 2 and administered to a patient in accordance with the present invention to treat cancer.

In an embodiment of the invention, TILs are expanded using PBL Method 2 and administered to a patient in accordance with the present invention to treat cancer.

In an embodiment of the invention, TILs expanded using PBL Method 1, PBL Method 2, or PBL Method 3 are administered to a patient in accordance with the present invention to treat CLL.

In any of the foregoing embodiments of the invention, pre-treatment with a kinase inhibitor is described. In an embodiment, the kinase inhibitor is selected from the group consisting of imatinib, dasatinib, ibrutinib, bosutinib, nilotinib, erlotinib, or other kinase inhibitors, tyrosine kinase inhibitors, or serine/threonine kinase inhibitors known in the art. In an embodiment, pre-treatment regimens with a kinase inhbitor are as known in the art and/or as prescribed by a physician.

In any of the foregoing embodiments of the invention, pre-treatment with an IL-2-inducible T-cell kinase (ITK) inhibitor is described. Interleukin-2-inducible T cell kinase (ITK) is a non-receptor tyrosine kinase expressed in T-cells and regulates various pathways. Any ITK inhibitor known in the art may be used in embodiments of the present invention (see, for example, Lo, et al., Expert Opinion on Therapeutic Patents, 20:459-469 (2010); Vargas, et al., Scandinavian Journal of Immunology, 78(2):130-139 (2013); WO2015112847; WO2016118951; WO2007136790, US20120058984A1, and U.S. Pat. Nos. 9,531,689 and 9,695,200; all of which are incorporated by reference herein in their entireties). In an embodiment of the invention, the ITK inhibitor is a covalent ITK inhibitor that covalently and irreversibly binds to ITK. In an embodiment of the invention, the ITK inhibitor is an allosteric ITK inhibitor that binds to ITK. In an embodiment of the invention, the ITK inhibitor is selected from the group consisting of aminothiazole-based ITK inhibitors, 5-aminomethylbenzimdazoles-based ITK inhibitors, 3-Aminopyrid-2-ones-based ITK inhibitors, (4 or 5-aryl)pyrazolyl-indole-based ITK inhibitors, benzimidazole-based ITK inhibitors, aminobenzimidazole-based ITK inhibitors, aminopyrimidine-based ITK inhibitors, aminopyridine-based ITK inhibitors, diazolodiazine-based ITK inhibitors, triazole-based ITK inhibitors, 3-aminopyride-2-ones-based ITK inhibitors, indolylindazole-based ITK inhibitors, indole-based ITK inhibitors, aza-indole-based ITK inhibitors, pyrazolyl-indole-based inhibitors, thienopyrazole-based ITK inhibitors, heterocyclic ITK inhibitors, and ITK inhibitors targeting cysteine-442 in the ATP pocket (such as ibrutinib), aza-benzimidazole-based ITK inhibitors, benzothiazole-based ITK inhibitors, indole-based ITK inhibitors, pyridone-based ITK inhibitors, sulfoximine-substituted pyrimidine ITK inhibitors, arylpyridinone-based ITK inhibitors, and any other ITK inhibitors known in the art. In an embodiment of the invention, pre-treatment regimens with an ITK inhibitor are as known in the art and/or as prescribed by a physician. In an embodiment of the invention, the ITK inhibitor is selected from the group consisting of:

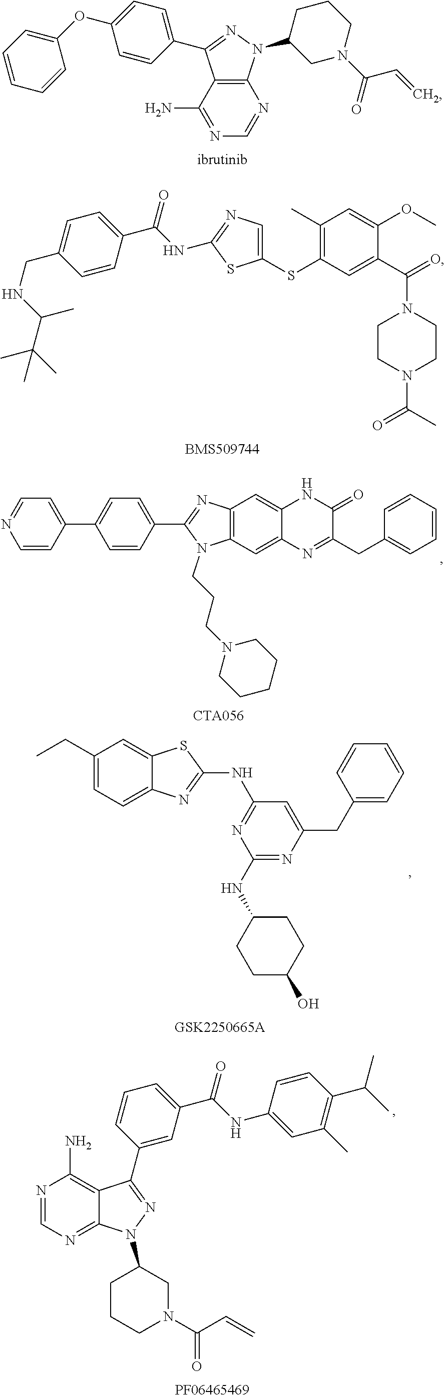

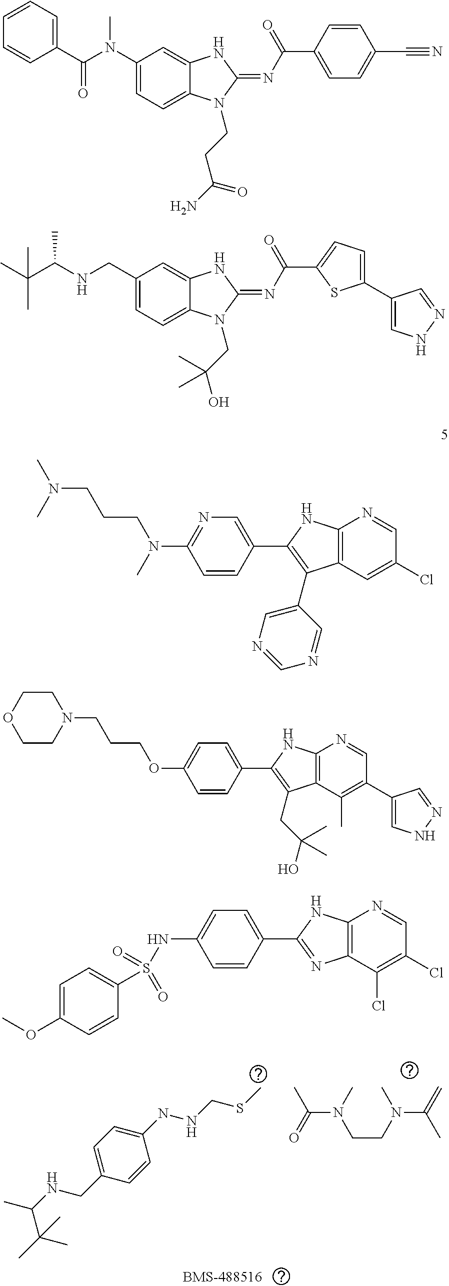

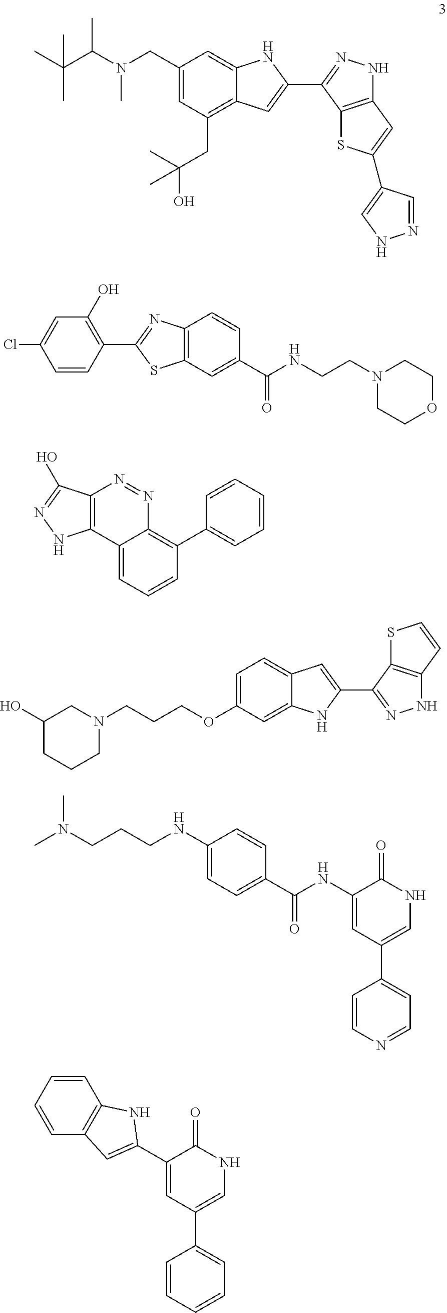

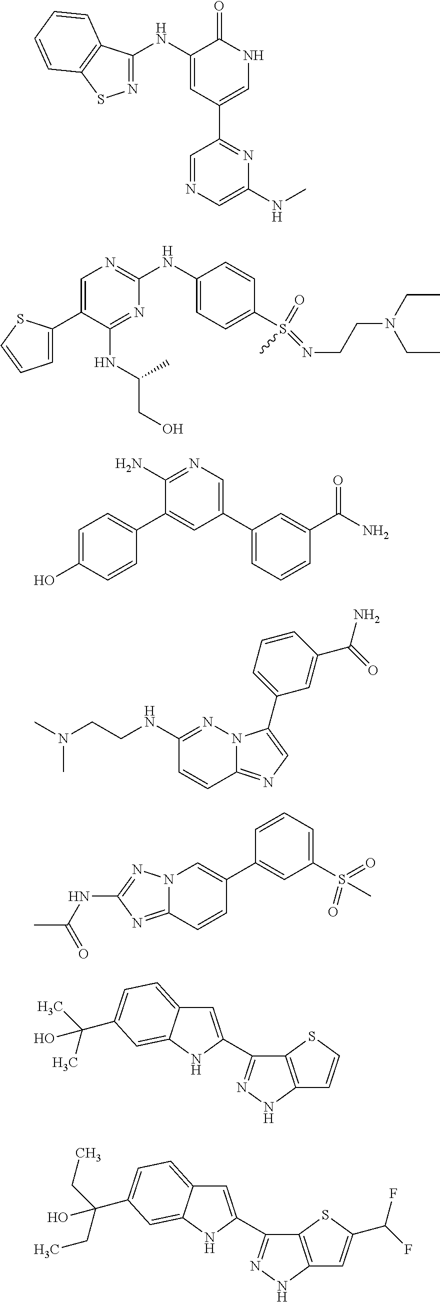

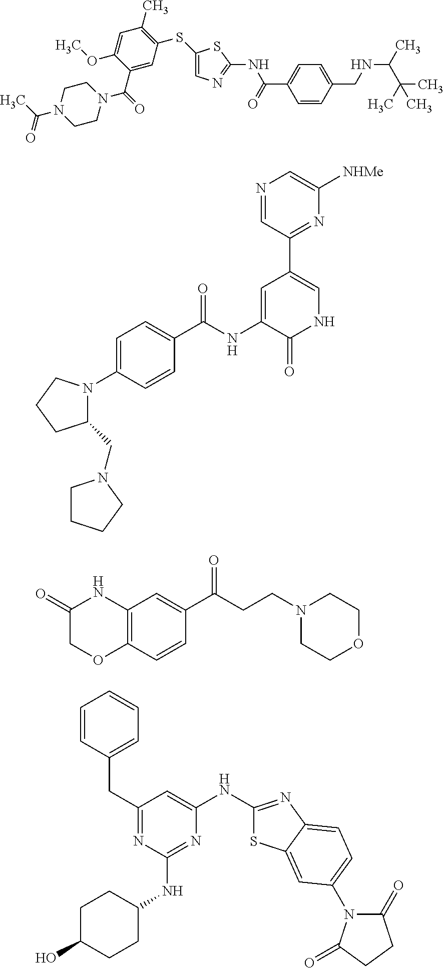

and combinations thereof. In an embodiment of the invention, the ITK inhibitor is selected from the group consisting of imatinib, dasatinib (BMS-354825), Sprycel [N-(2-chloro-6-methylphenyl)-2-(6-(4-(2-hydroxyethyl)-piperazin-1-yl)-2-meth-ylpyrimidin-4-ylamino)thiazole-5-carboxamide), ibrutinib ((1-{(3R)-3-[4-amino-3-(4-phenoxyphenyl)-1H-pyrazolo[3,4-d]pyrimidin-1-yl]piperidin-1-yl}prop-2-en-1-one), bosutinib, nilotinib, erlotinib, 1H-pyrazolo[4,3-c]cinnolin-3-ol, CTA056 (7-benzyl-1-(3-(piperidin-1-yl)propyl)-2-(4-(pyridin-4-yl)phenyl)-1H-imidazo[4,5-g]quinoxalin-6(5H)-one), Compound 10 (Boehringer Ingelheim from Moriarty, et al., Bioorg Med Chem Lett, 18:5537-40 (2008)), Compound 19 (Boehringer Ingelheim from Moriarty, et al., Bioorg Med Chem Lett., 18:5537-40 (2008)), Compound 27 (Boehringer Ingelheim from Moriarty, et al., Bioorg Med Chem Lett., 18:5537-40 (2008)), Compound 26 (Boehringer Ingelheim from Winters, et al., Bioorg Med Chem Lett., 18:5541-4 (2008)), Compound 37 (Boehringer Ingelheim from Cook, et al., Bioorg Med Chem Lett., 19:773-7 (2009)), Compound 41 (Boehringer Ingelheim from Cook, et al., Bioorg Med Chem Lett., 19:773-7 (2009)), Compound 48 (Boehringer Ingelheim from Cook, et al., Bioorg Med Chem Lett., 19:773-7 (2009)), Compound 51 (Boehringer Ingelheim from Cook, et al., Bioorg Med Chem Lett., 19:773-7 (2009)), Compound 10n (Boehringer Ingelheim from Riethe, et al., Bioorg Med Chem Lett., 19:1588-91 (2009)), Compound 10o (Boehringer Ingelheim from Riethe, et al., Bioorg Med Chem Lett., 19:1588-91 (2009)), Compound 7v (Vertex from Charrier, et al., J Med Chem., 54:2341-50 (2011)), Compound 7w (Vertex from Charrier, et al., J Med Chem., 54:2341-50 (2011)), Compound 7x (Vertex from Charrier, et al., J Med Chem., 54:2341-50 (2011)), Compound 7y (Vertex from Charrier, et al., J Med Chem., 54:2341-50 (2011)), Compound 44 (Bayer Schering Pharma from vonBonin, et al., Exp Dermatol., 20:41-7 (2011)), Compound 13 (Nycomed from Velankar, et al., Bioorg Med Chem., 18:4547-59 (2010)), Compound 24 (Nycomed from Velankar, et al., Bioorg Med Chem., 18:4547-59 (2010)), Compound 34 (Nycomed from Velankar, et al., Bioorg Med Chem., 18:4547-59 (2010)), Compound 10o (Nycomed from Herdemann, et al., Bioorg Med Chem Lett., 21:1852-6 (2011)), Compound 3 (Sanofi US from McLean, et al., Bioorg Med Chem Lett., 22:3296-300 (2012)), Compound 7 (Sanofi US from McLean, et al., Bioorg Med Chem Lett., 22:3296-300 (2012), and/or or other kinase inhibitors, tyrosine kinase inhibitors, or serine/threonine kinase inhibitors known in the art, as well as any combinations thereof.

In any of the foregoing embodiments, pre-treatment regimens comprising ibrutinib (commercially available as IMBRUVICA, and which has the chemical name 1-[(3R)-3-[4-amino-3-(4-phenoxyphenyl)-1H-pyrazolo[3,4-d]pyrimidin-1-yl]-1-piperidinyl]-2-propen-1-one) may include orally administering one 140 mg capsule q.d., orally administering two 140 mg capsules q.d., orally administering three 140 mg capsules q.d., or orally administering four 140 mg capsules q.d., for a duration of about one day, two days, three days, four days, five days, six days, seven days, eight days, nine days, ten days, eleven days, twelve days, two weeks, three weeks, one month, two months, three months, four months, five months, or six months. In the foregoing embodiments, pre-treatment regimens comprising ibrutinib may also comprise orally administering an ibrutinib dose selected from the group consisting of 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, and 500 mg, wherein the administering occurs once daily, twice daily, three times daily, or four times daily, and wherein the duration of administration is selected from the group consisting of about one day, two days, three days, four days, five days, six days, seven days, eight days, nine days, ten days, eleven days, twelve days, two weeks, three weeks, one month, two months, three months, four months, five months, and six months.

In any of the foregoing embodiments, the cancer to be treated is a hematological malignancy selected from the group consisting of acute myeloid leukemia (AML), mantle cell lymphoma (MCL), follicular lymphoma (FL), diffuse large B cell lymphoma (DLBCL), activated B cell (ABC) DLBCL, germinal center B cell (GCB) DLBCL, chronic lymphocytic leukemia (CLL), CLL with Richter's transformation (or Richter's syndrome), small lymphocytic leukemia (SLL), non-Hodgkin's lymphoma (NHL), Hodgkin's lymphoma, relapsed and/or refractory Hodgkin's lymphoma, B cell acute lymphoblastic leukemia (B-ALL), mature B-ALL, Burkitt's lymphoma, Waldenström's macroglobulinemia (WM), multiple myeloma, myelodysplatic syndromes, myelofibrosis, chronic myelocytic leukemia, follicle center lymphoma, indolent NHL, human immunodeficiency virus (HIV) associated B cell lymphoma, and Epstein-Barr virus (EBV) associated B cell lymphoma.

Efficacy of the methods and compositions described herein in treating, preventing and/or managing the indicated diseases or disorders can be tested using various animal models known in the art.

Non-Myeloablative Lymphodepletion with Chemotherapy

In an embodiment, the invention provides a method of treating a cancer with a population of TILs, wherein a patient is pre-treated with non-myeloablative chemotherapy prior to an infusion of TILs according to the present disclosure. In an embodiment, the non-myeloablative chemotherapy is one or more chemotherapeutic agents. In an embodiment, the non-myeloablative chemotherapy is cyclophosphamide 60 mg/kg/d for 2 days (days 27 and 26 prior to TIL infusion) and fludarabine 25 mg/m²/d for 5 days (days 27 to 23 prior to TIL infusion). In an embodiment, after non-myeloablative chemotherapy and TIL infusion (at day 0) according to the present disclosure, the patient receives an intravenous infusion of IL-2 intravenously at 720,000 IU/kg every 8 hours to physiologic tolerance.

Experimental findings indicate that lymphodepletion prior to adoptive transfer of tumor-specific T lymphocytes plays a key role in enhancing treatment efficacy by eliminating regulatory T cells and competing elements of the immune system (“cytokine sinks”). Accordingly, some embodiments of the invention utilize a lymphodepletion step (sometimes also referred to as “immunosuppressive conditioning”) on the patient prior to the introduction of the TILs of the invention.

In general, lymphodepletion is achieved using administration of fludarabine or cyclophosphamide (the active form being referred to as mafosfamide) and combinations thereof. Such methods are described in Gassner, et al., Cancer Immunol. Immunother. 2011, 60, 75-85, Muranski, et al., Nat. Clin. Pract. Oncol., 2006, 3, 668-681, Dudley, et al., J. Clin. Oncol. 2008, 26, 5233-5239, and Dudley, et al., J. Clin. Oncol. 2005, 23, 2346-2357, all of which are incorporated by reference herein in their entireties.

In some embodiments, the fludarabine is administered at a concentration of 0.5 μg/mL-10 μg/mL fludarabine. In some embodiments, the fludarabine is administered at a concentration of 1 μg/mL fludarabine. In some embodiments, the fludarabine treatment is administered for 1 day, 2 days, 3 days, 4 days, 5 days, 6 days, or 7 days or more. In some embodiments, the fludarabine is administered at a dosage of 10 mg/kg/day, 15 mg/kg/day, 20 mg/kg/day, 25 mg/kg/day, 30 mg/kg/day, 35 mg/kg/day, 40 mg/kg/day, or 45 mg/kg/day. In some embodiments, the fludarabine treatment is administered for 2-7 days at 35 mg/kg/day. In some embodiments, the fludarabine treatment is administered for 4-5 days at 35 mg/kg/day. In some embodiments, the fludarabine treatment is administered for 4-5 days at 25 mg/kg/day.

In some embodiments, the mafosfamide, the active form of cyclophosphamide, is obtained at a concentration of 0.5 μg/mL-10 μg/mL by administration of cyclophosphamide. In some embodiments, mafosfamide, the active form of cyclophosphamide, is obtained at a concentration of 1 μg/mL by administration of cyclophosphamide. In some embodiments, the cyclophosphamide treatment is administered for 1 day, 2 days, 3 days, 4 days, 5 days, 6 days, or 7 days or more. In some embodiments, the cyclophosphamide is administered at a dosage of 100 mg/m²/day, 150 mg/m²/day, 175 mg/m²/day 200 mg/m²/day, 225 mg/m²/day, 250 mg/m²/day, 275 mg/m²/day, or 300 mg/m²/day. In some embodiments, the cyclophosphamide is administered intravenously (i.e., i.v.) In some embodiments, the cyclophosphamide treatment is administered for 2-7 days at 35 mg/kg/day. In some embodiments, the cyclophosphamide treatment is administered for 4-5 days at 250 mg/m²/day i.v. In some embodiments, the cyclophosphamide treatment is administered for 4 days at 250 mg/m²/day i.v.

In some embodiments, lymphodepletion is performed by administering the fludarabine and the cyclophosphamide are together to a patient. In some embodiments, fludarabine is administered at 25 mg/m²/day i.v. and cyclophosphamide is administered at 250 mg/m²/day i.v. over 4 days.

In an embodiment, the lymphodepletion is performed by administration of cyclophosphamide at a dose of 60 mg/m²/day for two days followed by administration of fludarabine at a dose of 25 mg/m²/day for five days. Several methods of expanding TILs obtained from bone marrow or peripheral blood are described herein. In an embodiment of the invention, the lymphodepletion is performed by administration of cyclophosphamide at a dose of 60 mg/m²/day for two days followed by administration of fludarabine at a dose of 25 mg/m²/day for five days. Several methods of expanding TILs obtained from bone marrow or peripheral blood are described herein.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1

### Expansion of TILs from Non-Hodgkin's Lymphomas

TILs were expanded from five non-Hodgkin's lymphoma tumors (one mantle cell lymphoma tumor, three follicular lymphoma tumors, and one ABC-type diffuse large B cell lymphoma tumor) with the pathologies given in FIG. 1, using IL-2 for 11 to 14 days in a pre-REP stage, followed by subsequent REP for 14 days using IL-2, mitogenic anti-CD3 antibody, and irradiated allogeneic peripheral blood mononuclear cell (PBMC) feeders. TILs were successfully generated from all 5 lymphoma tumors with maximum expansion index of 680 fold, significantly higher than previously observed using other methods. Schwartzentruber, et al., Blood 1993, 82, 1204-1211. Further, mean CD3⁺ T cell population was 95% (versus 75% using the method of Schwartzentruber, et al., Blood 1993, 82, 1204-1211).

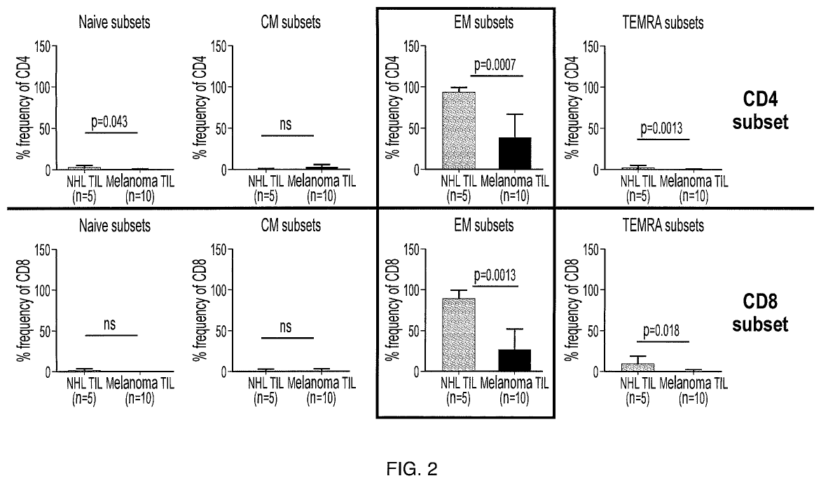

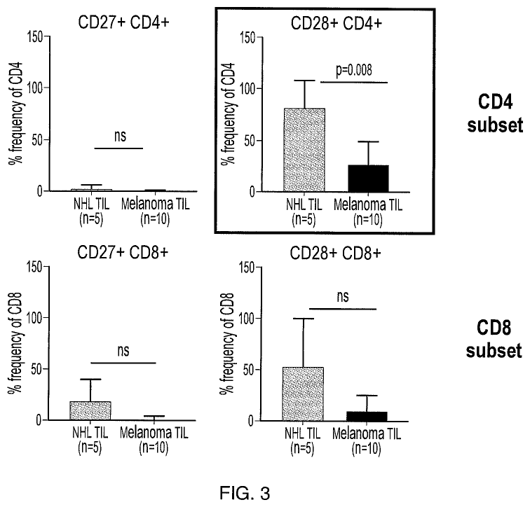

Cell sorting and flow cytometry was performed using a Becton, Dickinson & Co. (BD) FACS CANTO II system. A marked relative increase in effector memory cells that was comparable to that in melanoma TILs was observed by flow cytometry analysis (FIG. 2). A significant increase in effector memory CD45RA⁺ (TEMRA) cells (p=0.0013; CD4, CD8) and CD28+CD4+ (p=0.008) subsets was observed in lymphoma compared with melanoma TIL cultures (FIG. 3).

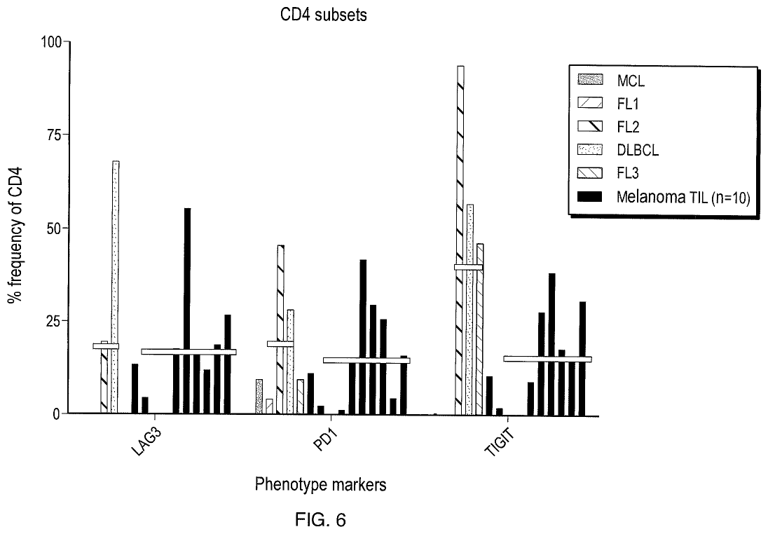

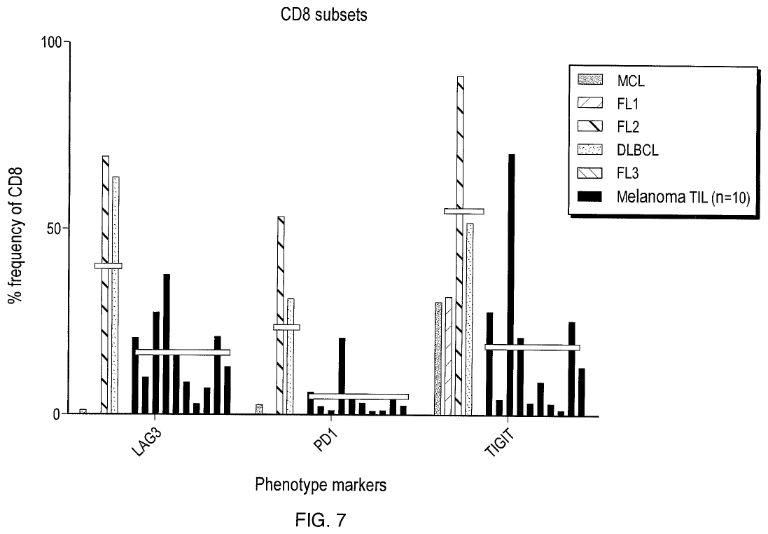

Comparisons of phenotypic markers of T cell differentiation in CD4⁺ and CD8⁺ subsets are shown in FIG. 4 and FIG. 5, respectively. Comparisons of phenotypic markers of T cell exhaustion in CD4⁺ and CD8⁺ subsets are shown in FIG. 6 and FIG. 7, respectively.

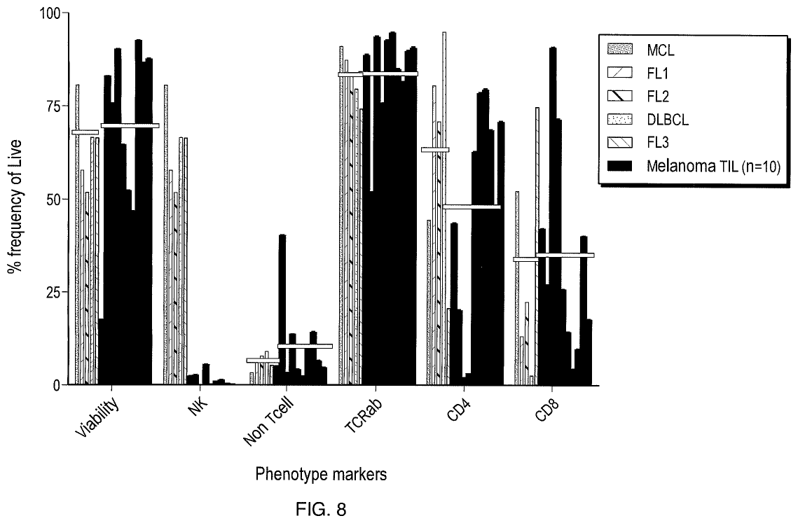

FIG. 8 illustrates a comparison of cell types between non-Hodgkin's lymphoma TILs and melanoma TILs. An increasing trend in the number of CD4⁺ T cells in lymphoma TILs compared to melanoma TILs is shown.

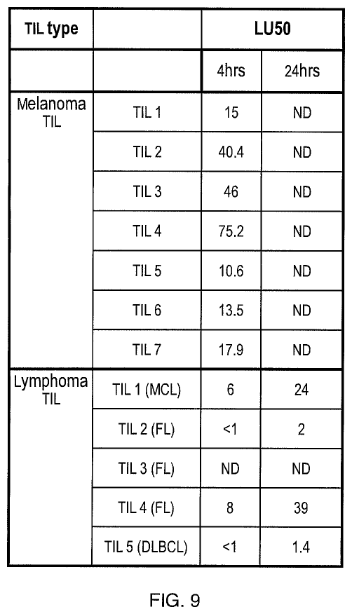

FIG. 9 illustrates bioluminescent redirected lysis assay (BRLA) results. Minimal cytolytic activity of TIL measured by BRLA as LU₅₀/10⁶ at 4 hrs ranged from <1-6 LU₅₀ and at 24hrs, 1-39 LU₅₀ in lymphoma TIL compared to melanoma TIL (11-75 LU₅₀, 4 hrs).

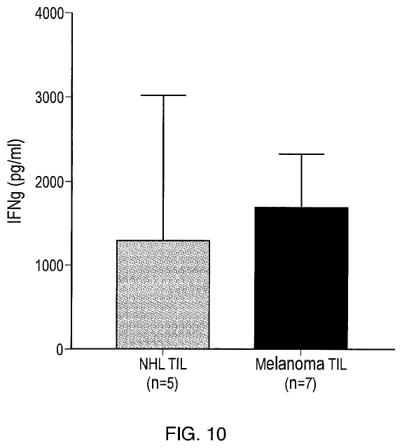

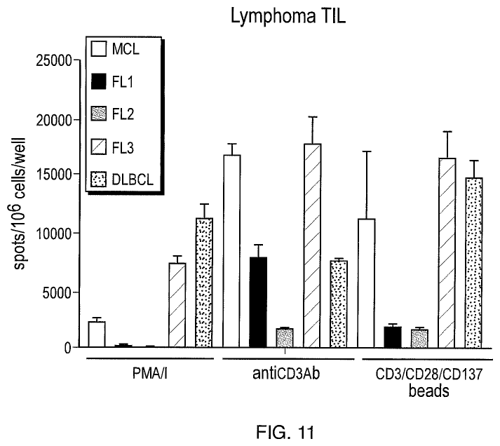

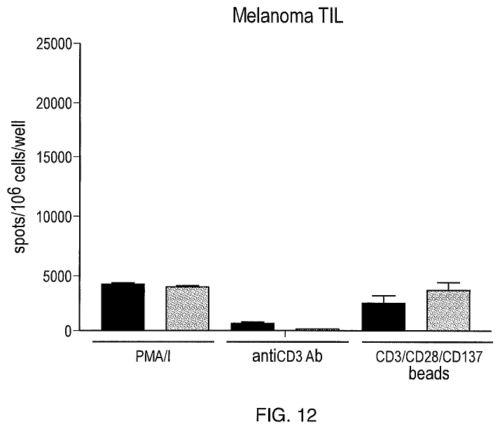

FIG. 10 illustrates interferon-γ (IFN-γ) enzyme-linked immunosorbent assay (ELISA) results for lymphoma TILs versus melanoma TILs. Showing comparable results. ELIspot assay results for the lymphoma TILs are shown in FIG. 11 and are compared to results of the same assay for melanoma TILs in FIG. 12. In the ELIspot assay, a wide range of IFN-γ production by lymphoma TILs was observed upon stimulation with phorbol 12-myristate 13-acetate/ionomycin, anti-CD3 antibody, or CD3/CD28/4-1BB beads, and IFN-γ produced by some lymphoma TILs under these conditions was comparable to the IFN-γ produced by melanoma TILs, and in several cases, IFN-γ production in lymphoma TILs was much higher.

FIG. 13 illustrates the results of a NANOSTRING NCOUNTER analysis (Nanostring Technologies, Inc., Seattle, Wash.), showing that lymphoma TILs express higher levels of RORC IL17A (TH17 phenotype) and GATA3 (Th2 phenotype) compared to melanoma TILs. This finding is consistent with the observation that lymphoma-reactive T cells are primarily TH2 and TH17.

Overall, the results provide evidence that TIL cell therapy may be used to treat patients with lymphoma.

### Example 2

### Phenotypic and Functional Characterization of Marrow Infiltrating Lymphocytes (MIL) Grown from Bone Marrow of AML Patients and Peripheral Blood Lymphocytes (PBL) Grown from Peripheral Blood of AML Patients

Samples of bone marrow and as available a related blood sample were obtained from patients with acute myeloid leukemia (AML), including patients pre-treated with at least three rounds of a regimen comprising ibrutinib (1-[(3R)-3-[4-amino-3-(4-phenoxyphenyl)-1H-pyrazolo[3,4-d]pyrimidin-1-yl]-1-piperidinyl]-2-propen-1-one), accompanied by information about the patient's age, gender, stage, tumor type, site of cancer, treatment history, a de-identified pathology report, and any molecular tests performed (e.g., MSI expression and Raf/Ras expression). MILs and PBLs were expanded using one of MIL Method 1, MIL Method 2, or MIL Method 3, or PBL Method 1, PBL Method 2, or PBL Method 3, and the MILs and PBLs were phenotypically and functionally characterized.

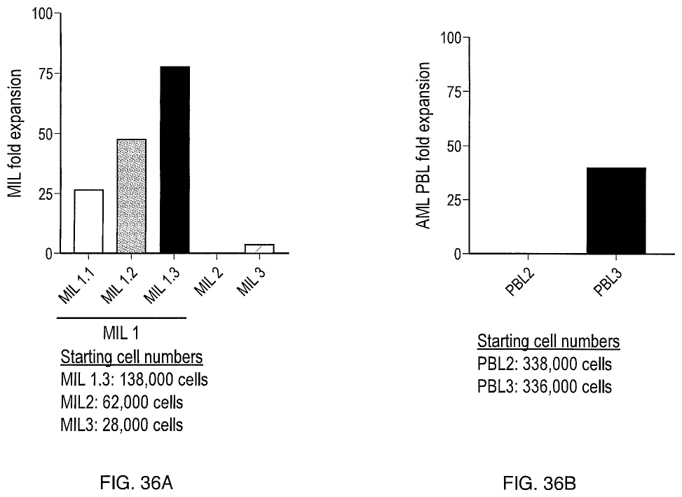

FIGS. 36A and 36B illustrate the fold expansion for MILs and PBLs. FIG. 36A shows the fold expansion for 3 patients (MIL1, MIL2, MIL3) and FIG. 36B shows the fold expansion for the matched PBLs for patients 2 and 3 (PBL2, PBL3). MIL1.1 was expanded using MIL Method 1, MIL1.2 was expanded using MIL Method 2, and MIL1.3 was expanded using MIL Method 3, and PBLs were expanded using PBL Method 3. MIL1 fold expansion shows 25 (MIL1.1), 50 (MIL1.2), and 75 (MIL1.3) fold increases for each sample within MIL1. This preliminarily demonstrates that MIL Method 3 may be a preferred expansion method. MIL2 and MIL3 fold expansion data appears poor, possibly due to low starting cell number. For comparison, the starting cell number for sample 3 of patient MIL1 (MIL1.3) was 138,000 cells, while the starting cells numbers for MIL2 and MIL 3 were 62,000 and 28,000 respectively. PBL fold expansion shown in FIG. 36B for MIL2 and MIL3 was about 10-fold and 40-fold, respectively, with similar starting cell numbers (338,000 for PBL2 and 336,000 for PBL3).

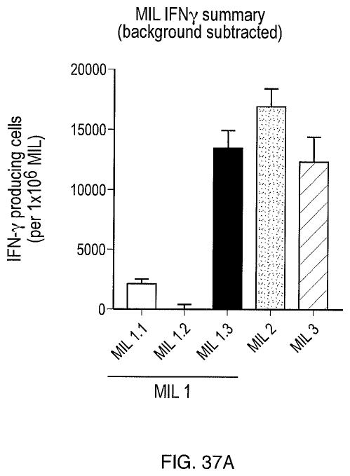

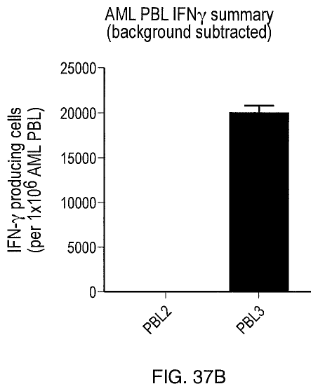

FIGS. 37A and 37B illustrate the number of IFN-γ producing cells for MILs (FIG. 37A) and matched PBLs (FIG. 37B). MIL1.3, MIL2, and MIL3 show significant increases in IFN-γ secretion, indicating that MIL Method 3 is a preferred expansion method. The data for PBLs is inconclusive.

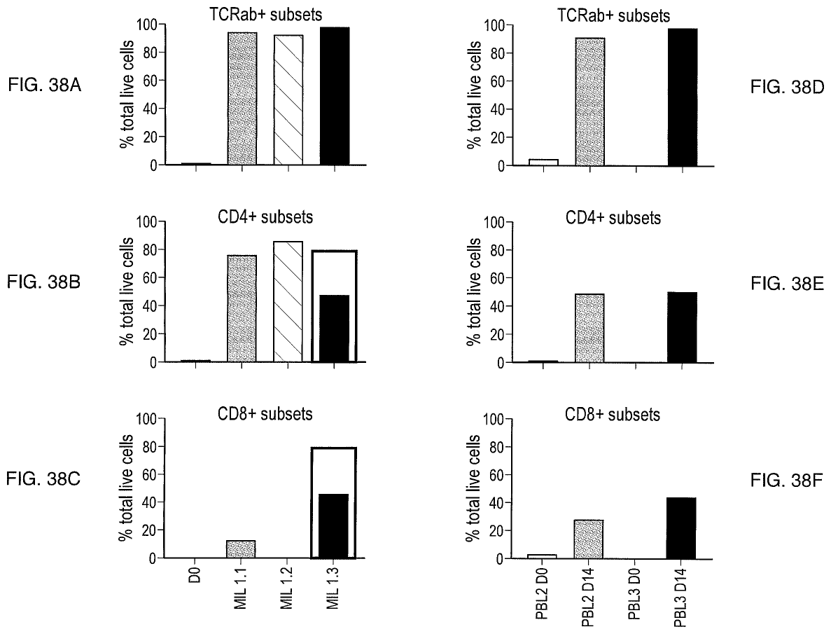

FIGS. 38A-38F show TCRαβ+, CD4+, and CD8+ subsets for MILs and PBLs. FIGS. 38A and 38D show TCRab+ subsets for MILs (FIG. 38A) expanded using all 3 methods (MIL1.1, MIL1.2, MIL1.3) and for PBLs (FIG. 38D) expanded using PBL Method 3. The data show that TCRαβ+ subsets are at almost 100% for all MILs and PBLs, which indicates that the expansion process was successful in expanding almost all T-cells. FIGS. 38B and 38E show CD4 subsets are decreased for MIL expanded by MIL Method 3 (which correlates to the increase in CD8 subsets in FIG. 38C). PBL data in FIGS. 38E and 38F appear consistent with the MIL1.3 data.

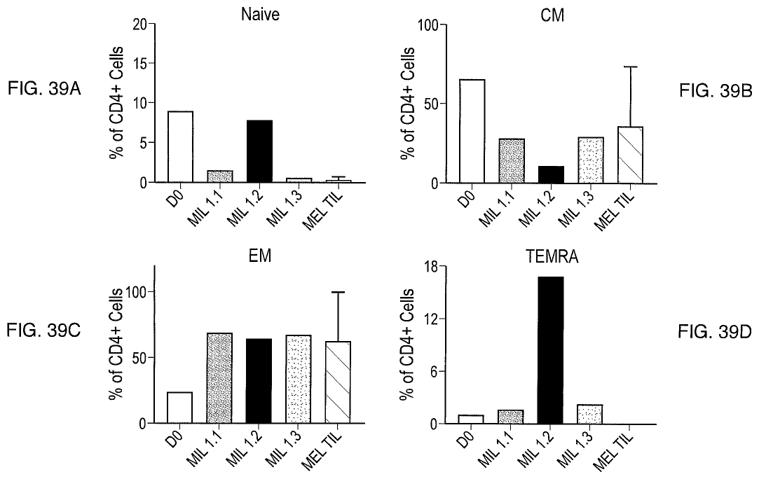

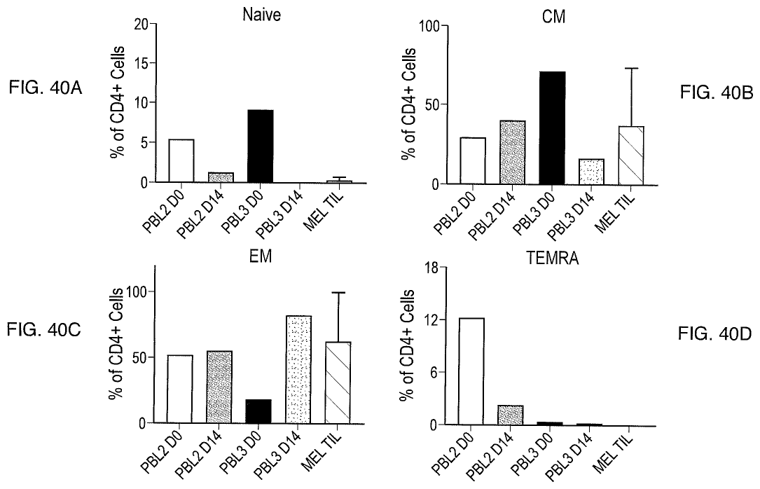

FIGS. 39A-D and 40A-D show data for CD4 subsets in MIL (FIG. 39) and PBL (FIG. 40). FIGS. 39A and 40A show data for naïve (CCR7+/CD45RA+); FIGS. 39B and 40B show data for central memory t-cells (CM) (CCR7+/CD45RA−); FIGS. 39C and 40C show data for effector memory T-cells (EM) (CCR7−/CD45RA−); and FIGS. 39D and 40D show data for terminally differentiated effector memory cells (TEMRA) (CCR7−/CD45RA+). All samples expanded using MIL Method 3 (MIL1.3) and PBL Method 3 (PBL2 and PBL3) are consistent with CD4 subsets in the comparator, melanoma TIL.

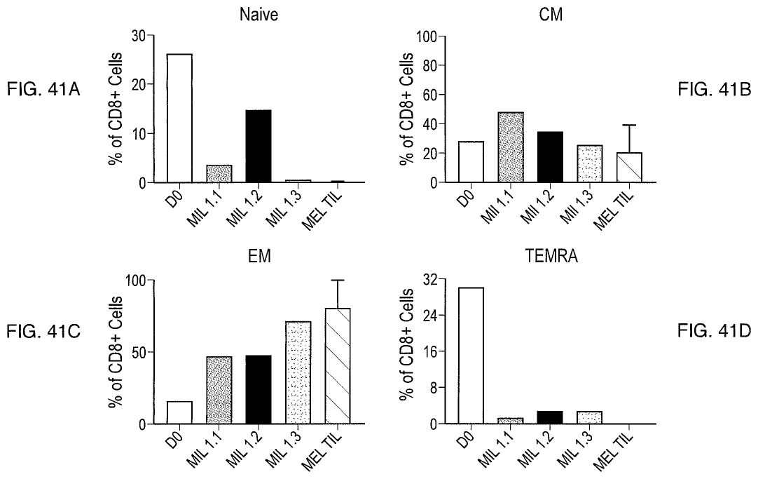

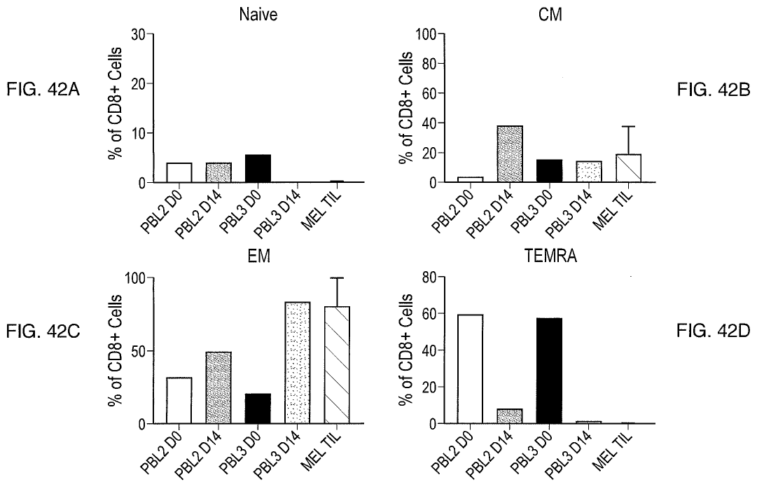

FIGS. 41A-D and 42A-D show data for CD8 subsets in MIL (FIG. 41) and PBL (FIG. 42). FIGS. 41A and 42A show data for naïve (CCR7+/CD45RA+); FIGS. 41B and 42B show data for central memory t-cells (CM) (CCR7+/CD45RA−); FIGS. 41C and 42C show data for effector memory T-cells (EM) (CCR7−/CD45RA−); and FIGS. 41D and 42D show data for terminally differentiated effector memory cells (TEMRA) (CCR7−/CD45RA+). The samples expanded using MIL Method 3 (MIL1.3) are consistent with CD4 subsets in the comparator, melanoma TIL. The data for PBL2 and PBL3 was used as a control.

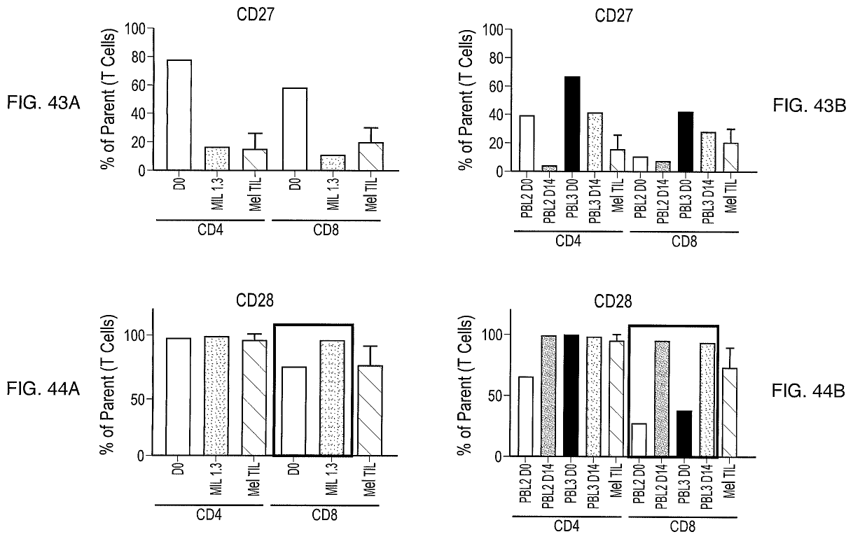

FIGS. 43A and 43B show data for CD4CD27 and CD8CD27 subsets for MILs (FIG. 43A) and PBLs (FIG. 43B). FIGS. 44A and 44B show data for CD4CD28 and CD8CD28 subsets for MILs (FIG. 44A) and PBLs (FIG. 44B). The data for PBLs is shown for Day 0 and Day 14 of the expansion process for each sample, as compared with melanoma TIL. The data for MILs is shown at Day 0 and Day 14 for MIL1.3 only, as compared with melanoma TIL. CD28 subsets in MIL and PBL are similar to melanoma TIL.

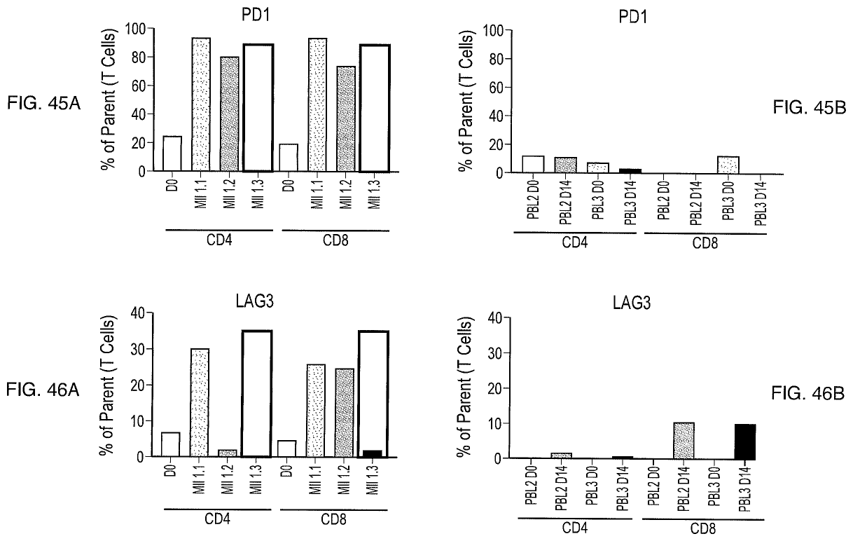

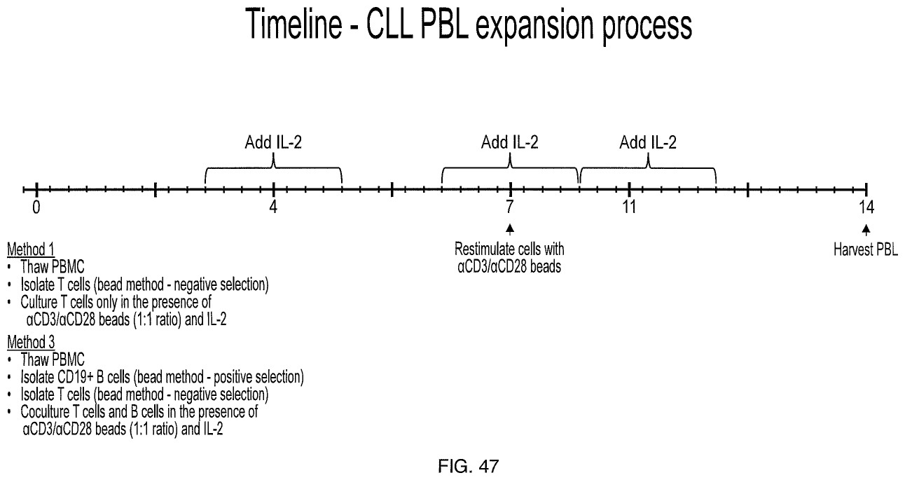

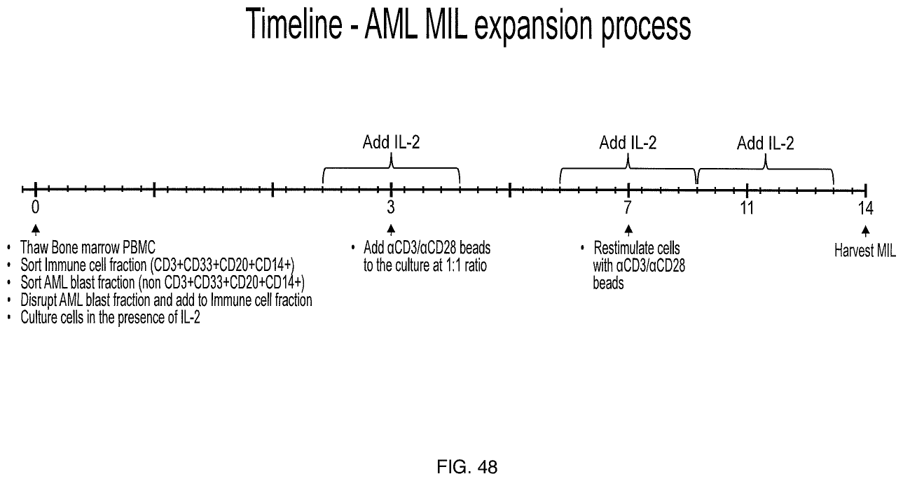

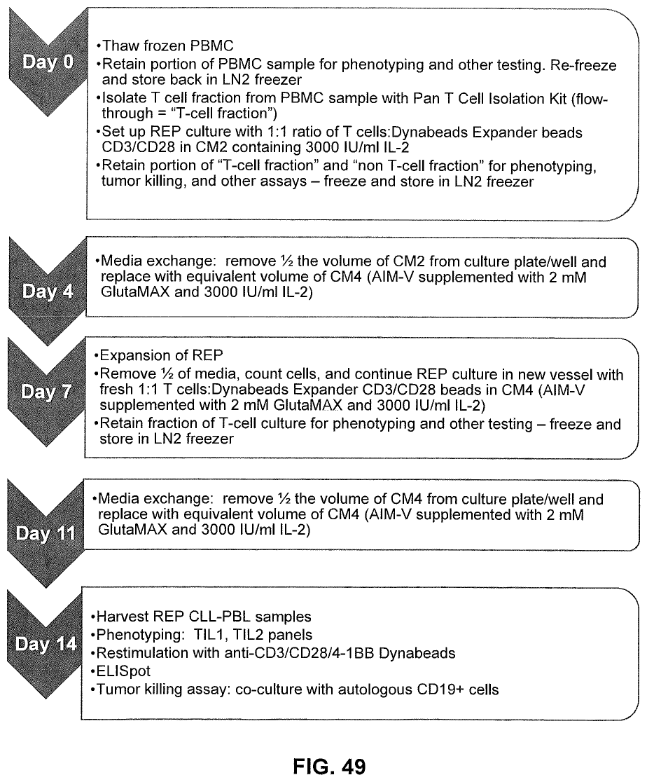

FIGS. 45A and 45B represent a comparison of PD1+ cells within each of CD4 and CD8 subsets for MILs (FIG. 45A) and PBLs (FIG. 45B). FIGS. 46A and 46B represent a comparison of LAG3+ cells within each of CD4 and CD8 subsets for MILs (FIG. 46A) and PBLs (FIG. 46B). The data for both PD1+ and LAG3+ show a substantial decrease in for the MIL1.3 sample over the Day 0 measurement, while MIL1.1 and MIL1.2 appeared to trend toward an increase for both PD1 and LAG3 over Day 0. The PBL data was used as a control.

The experiments from this Example demonstrate that MILs expanded with MIL Method 3 had a higher fold expansion, were highly functional, had a higher proportion of CD8 subsets, and had less LAG3+ and PD1+ T cell subsets. The data also showed that the memory subsets were similar to melanoma TIL. The data also showed that cryopreserved samples appeared to have higher fold expansion as compared with fresh samples. Much of the data for the PBL samples appears to be inconclusive, likely based on the small sample size.

### Example 3

### Methods of Expanding TILs and Treating Cancers with Expanded TILs

Bone marrow is obtained using needle aspiration. The bone marrow sample is aspirated into heparin-containing syringes and stored overnight at room temperature. After storage, the contents of the syringes are pooled together into a sterile container and quality tested. The bone marrow is enriched for mononuclear cells (MNCs) using lymphocyte separation media (LSM) and centrifugation with a COBE Spectra. Cells in the gradient are collected down to the red blood cells and washed using HBSS. The MNCs are cryopreserved using a hetastarch-based cryoprotectant supplemented with 2% HSA and 5% DMSO, reserving some of the MNCs for quality control. The QC vial is thawed to determine the CD3⁺ and CD38⁺/138⁺ cell content of the MNC product.

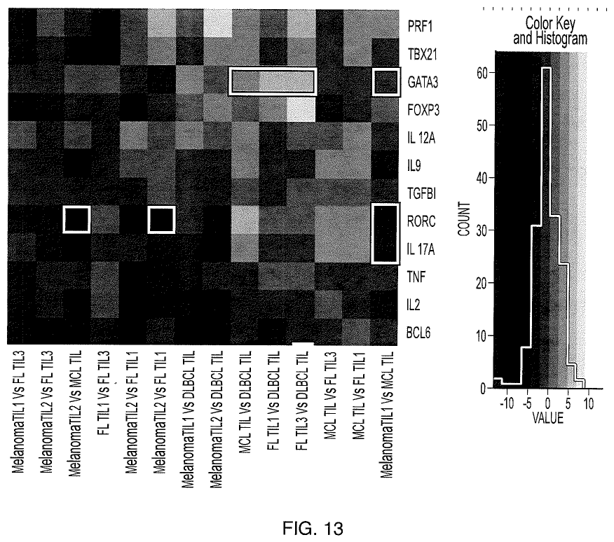

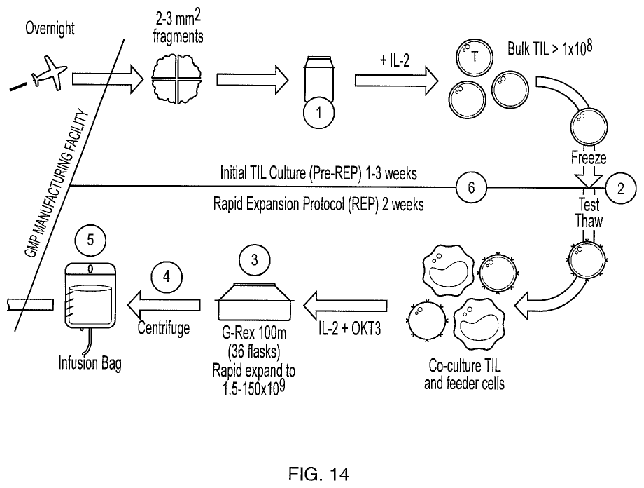

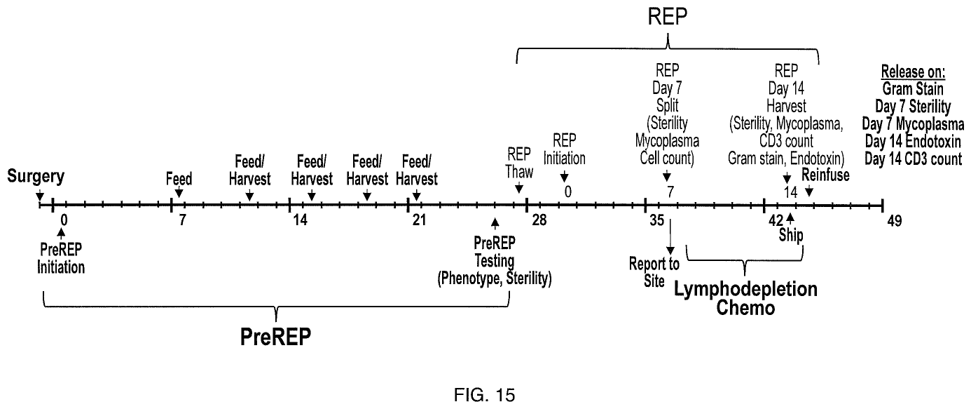

The bone marrow is aspirated and fractionated on a Lymphocyte Separation Medium density gradient and cells are collected almost to the level of the red cell pellet. This fractionation method substantially removes red blood cells and neutrophils, providing nearly complete bone marrow. The resulting fractionated material is T-cells and tumor cells. The bone marrow is Ficolled, and TILs are expanded using methods known in the art and any method described herein. For example, an exemplary method for expanding TILs is depicted in FIG. 14. An exemplary method for expanding TILs and treating a cancer patient with expanded TILs is shown in FIG. 15.

### Example 4

### Phenotypic and Functional Characterization of Tumor Infiltrating Lymphocytes (TIL) Grown from Non-Hodgkin Lymphoma Tumors

The goals of the experiments described in this Example include determining whether TIL with therapeutic potential can be isolated and cultured from NHL tumors and to compare characteristics of NHL-derived TIL with melanoma-derived TIL.

Materials and methods for extraction and expansion of TILs from a patient are as described herein. Patients' TIL were extracted from a suppressive tumor microenvironment by surgical resection of a lesion, in this case, lymph tissue. TILs were expanded using the expansion processes disclosed herein to yield 10⁹ to 10¹¹ TILs.

NHL-derived TILs (1 mantle cell lymphoma (MCL), 3 follicular lymphomas (FL), 3 diffuse large B cell lymphomas (DLBCL)) were analyzed for markers of differentiation against melanoma-derived TILs using flow cytometry. TILs were analyzed for anti-CD56, anti-TCRab, anti-CD3, anti-CD4, anti-CD8, anti-CD27 and anti-CD28 antibodies. These antibodies were used as Differentiation Panel 1 (DF1). Anti-CD3, anti-CD4, anti-CD9, anti-CD38, and anti-HLA-DR, anti-CCR7, and anti-CD45RA antibodies were used as differentiation panel 2 (DF2). DF2 was used to identify the following T-cell subsets: Naïve (CCR7+/CD45RA+); central memory t-cells (CM) (CCR7+/CD45RA−); effector memory T-cells (EM) (CCR7−/CD45RA−); and terminally differentiated effector memory cells (TEMRA) (CCR7−/CD45RA+).

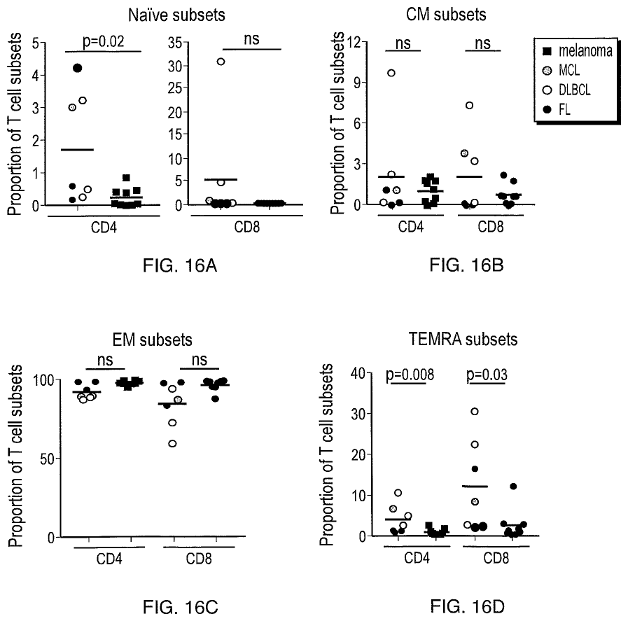

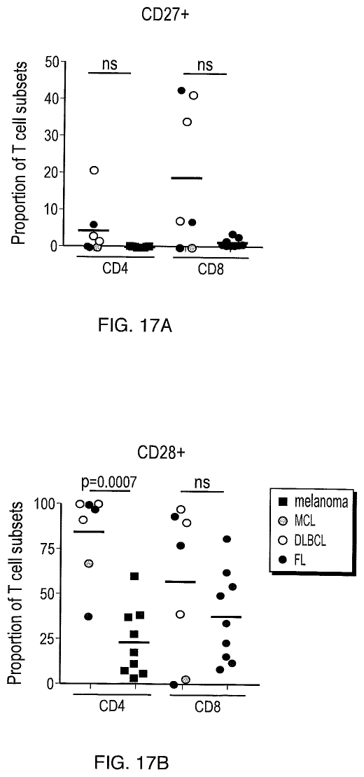

FIG. 16 shows CD4 and CD8 T-cells in different cell subpopulations in different cancer types. Melanoma (square), mantle cell (shaded circle), diffuse large B cell lymphoma (open circle) and follicular lymphoma (black circle) cancer types were tested. FIGS. 16A-16D generally demonstrate a trend for lymphoma TIL to be more highly proliferative and therefore have higher anti-tumor activity as compared with melanoma TIL. Likewise, FIG. 17B shows that CD4/CD28 expressing lymphoma T-cells have higher proliferative capacity than CD4/CD28 expressing melanoma T-cells.

Interferon gamma (IFNγ) production by TILs was measured by stimulating TILs with mAB-coated Dynabeads™ (CD3, CD28, and CD137), then using ELIspot™ (Immunospot CTL) and enumerated using Immunospot™ S6 entry analyzer, and also by ELISA using DuoSet™ ELISA kit (R&D systems following the manufacturer's instructions.

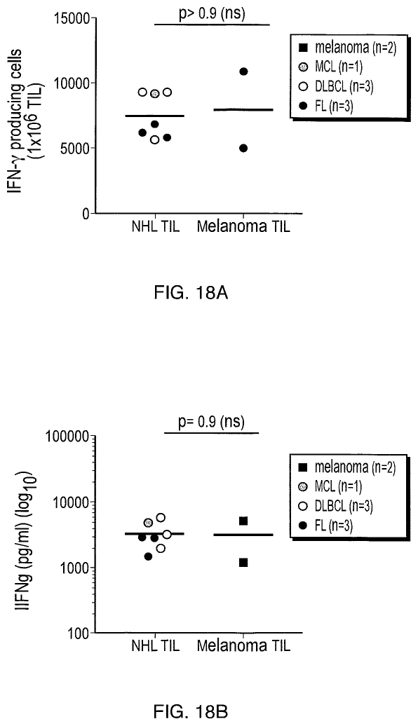

FIGS. 18A and 18B demonstrate that IFNγ production by NHL TIL and Melanoma TIL are similar, indicating a similar cytotoxicity functionality between the two TIL types.

Lytic potential of TILs was determined using bioluminescent Redirected Lysis Assay (BRLA). P815 cells transduced with lentiviral vector encoding eGFP and firefly luciferase were used as target cells. TILs and target cells were cocultured for 4 hours/24 hours in the presence of OKT3. Luciferin was then added and cells were incubated for 5 minutes. Bioluminescence was measured using a luminometer. Percent survival and percent cytotoxicity were calculated as follows:

% Survival=(experimental survival−minimum)/(maximum signal−minimum signal)×100%

% Cytotoxicity=100−(% Survival)

Lytic potential of TILs was expressed as a lytic unit, LU50, which represents 50 percent cytotoxicity of target cells induced by effector cells.

TILs were assayed to determine their tumor-killing ability on both autologous and allogeneic tumors. TILs were mixed with autologous lymphoma cells or allogeneic melanoma cell lines (526 melanoma cell line) at different effector cell to target cell ratios (E:T ratio)—either 10:1, 20:1, 50:1, or 100:1. Tumor cells were labeled with CellTrace Violet dye (ThermoFisher) prior to coculture. After 24 hours, cells were stained with 7-AAD to determine cell death. The proportion of tumor cells killed by TILs were represented as 7-AAD positive tumor cells that were gated on CellTrace Violet dye versus CD19 for the lymphoma cells and CellTrace Violet Vs MCSP for melanoma cells.

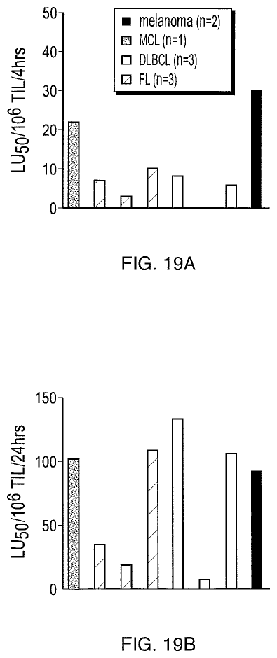

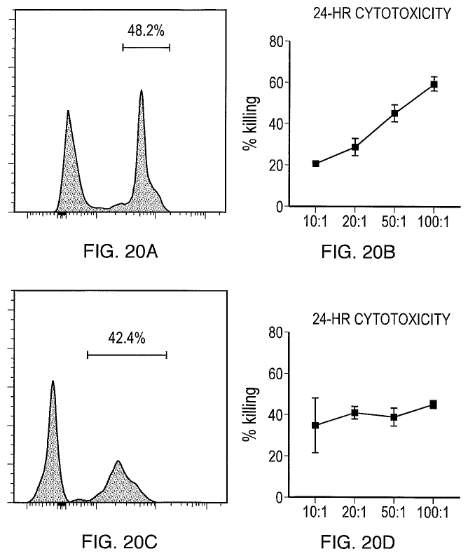

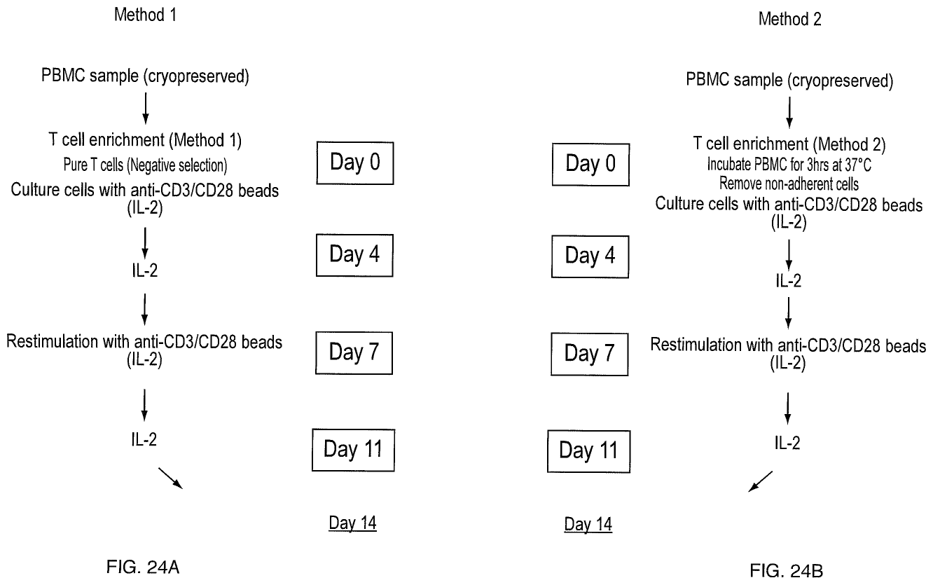

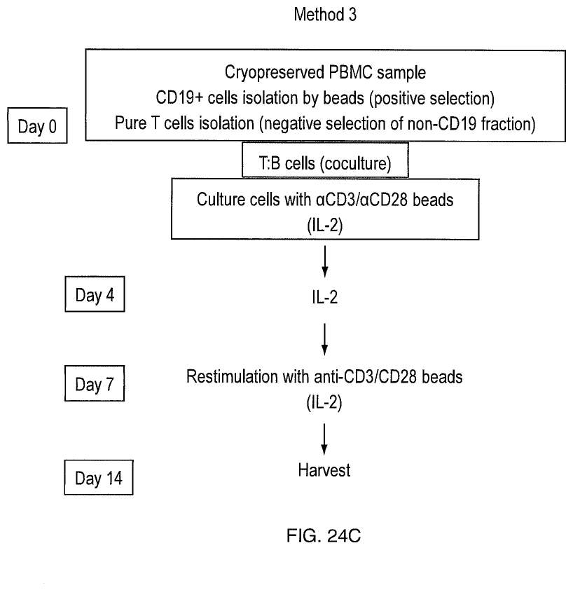

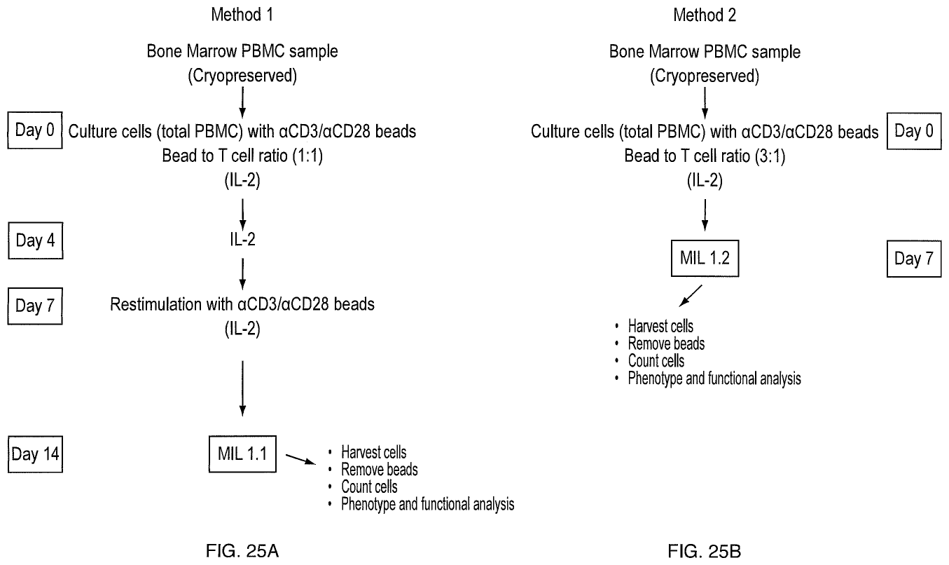

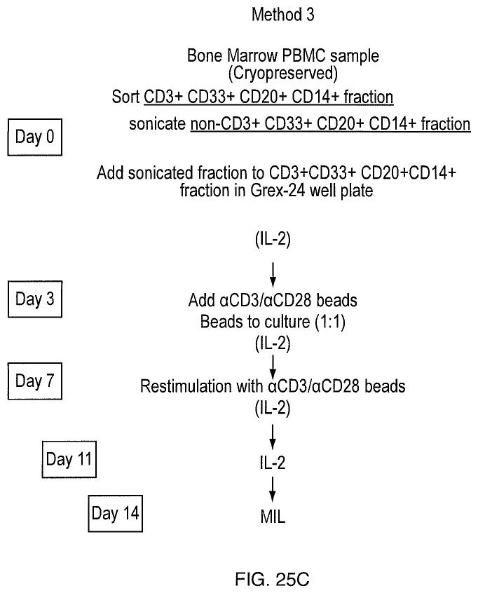

FIG. 19 shows that NHL TIL and melanoma TIL have similar cytotoxic functionality against both allogeneic and autologous tumors at 4 hours (FIG. 19A) and 24 hours (FIG. 19B).

Gene expression analysis was also performed on the TILs using the nCounter GX Human Immunology V2 panel (NanoString, Seattle). 100 ng total RNA was assayed per the manufacturer's instructions. Data were normalized by scaling with geometric mean of the built-in control gene probes for each sample. The data was mapped against and compared to melanoma gene expression.

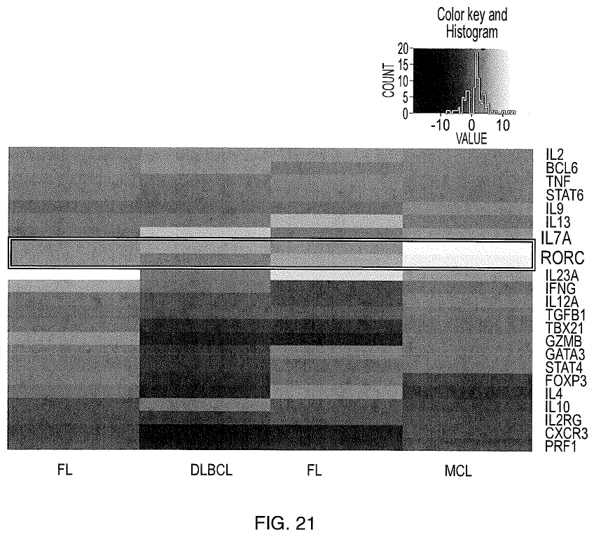

FIG. 21 demonstrates the results of the gene expression analysis. The heat map shows fold change in gene expression over melanoma TIL. IL17A and RORC expression from lymphoma-derived TIL had higher expression as compared with melanoma-derived TIL.

Overall, the results of this experiment demonstrated that the functional characteristics of lymphoma-derived TIL are similar to melanoma-derived TIL, indicating that use of lymphoma-derived TIL would be successful in treating lymphoma cancers.

### Example 5

### Phenotypic and Functional Characterization of Peripheral Blood Lymphocytes (PBLs) Grown from Peripheral Blood of Patients With Chronic Lymphocytic Leukemia (CLL).

PBMCs were collected from patients with CLL pre- and post-treatment with three rounds of ibrutinib.

T-cells were expanded using three different methods, PBL Method 1, PBL Method 2, and PBL Method 3, as described in FIG. 24 and elsewhere herein. Certain samples were derived from Fresh PBMCs and certain samples were derived from cryopreserved PBMCs. Once the cells were expanded and harvested, they were phenotyped and functionally characterized using the methods described in Example 4, above, and elsewhere herein. The goals of this Example were to determine an optimal expansion process for PBLs and to determine whether PBLs expanded from ibrutinib treated samples are more potent than PBLs expanded from untreated samples.

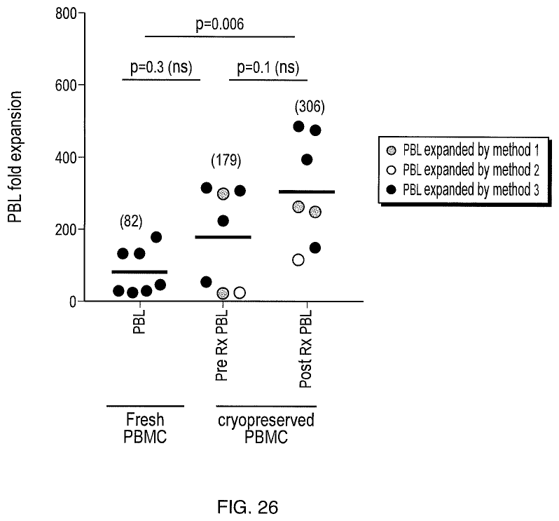

PBL fold expansion is shown in FIG. 26. Results for PBLs expanded using PBL Method 1, PBL Method 2, and PBL Method 3 are shown. Untreated PBLs (PreRx PBL) showed a mean 179-fold expansion and ibrutinib treated PBLs (PostRx PBL) showed a mean 306-fold expansion. PBLs derived from Fresh PBMC (PBL) showed only a mean 82-fold expansion. As between PBLs and PostRx PBLs, p=0.006. As between PBLs and PreRx PBLs, p=0.3, and as between PreRx PBLs and PostRx PBLs, p=0.1. Overall, an increase in the mean fold-expansion is seen for all PostRx PBL groups over all groups in both PBLs and PreRx PBLs.

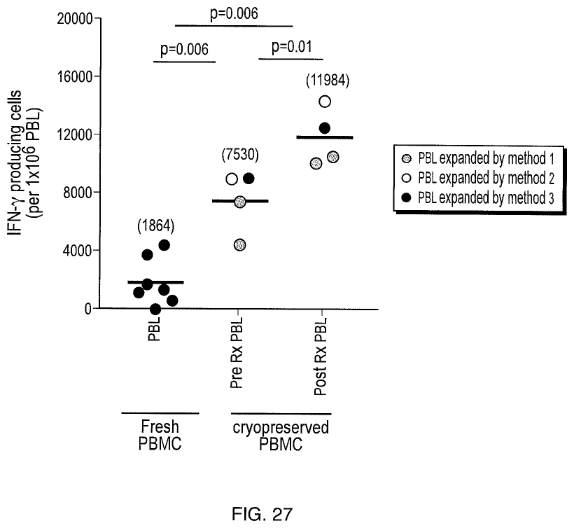

FIG. 27 demonstrates interferon-gamma (IFN-γ) producing cells in PBL, PreRx PBL, and PostRx PBL. For PBL, the mean number of IFN-γ producing cells was about 1864. For PreRx PBL, the mean number of IFN-g producing cells was about 7530, and for PostRx PBL, the mean number of IFN-γ producing cells was about 11984. As between PBLs and PostRx PBLs, p=0.006. As between PBLs and PreRx PBLs, p=0.006, and as between PreRx PBLs and PostRx PBLs, p=0.01. Overall, a significant increase in the mean number of IFN-γ producing cells is seen for all PostRx PBL groups over all groups in both PBLs and PreRx PBLs.

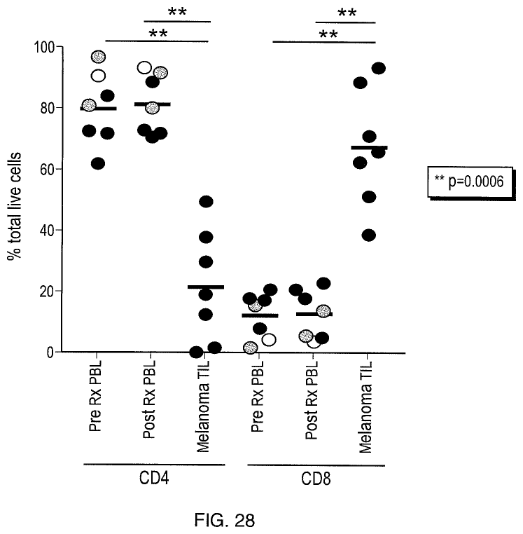

Phenotypic characterization was performed on each of the samples. FIG. 28 represents the proportion of CD4+ and CD8+ T cell subsets in PreRx PBL and PostRx PBL, and uses melanoma TIL as a comparator. Here, the data show that CD4 subsets (shown on the left) were comparable between both PreRx PBL and PostRx PBL, regardless of which method was used to expand the cells. CD4 subsets in PreRx PBLs and PostRx PBLs were shown to be higher than melanoma TIL (p=0.0006 for each). CD8 subsets (shown on the right) were lower in both PreRX PBL and PostRX PBL, regardless of the process used to expand the cells. CD8 subsets in PreRx PBLs and PostRx PBLs were shown to be lower than melanoma TIL (p=0.0006 for each). It is hypothesized that the lower CD8 subsets are merely a derivative of the type of cancer (i.e., in CLL, CD4 subsets are typically expanded).

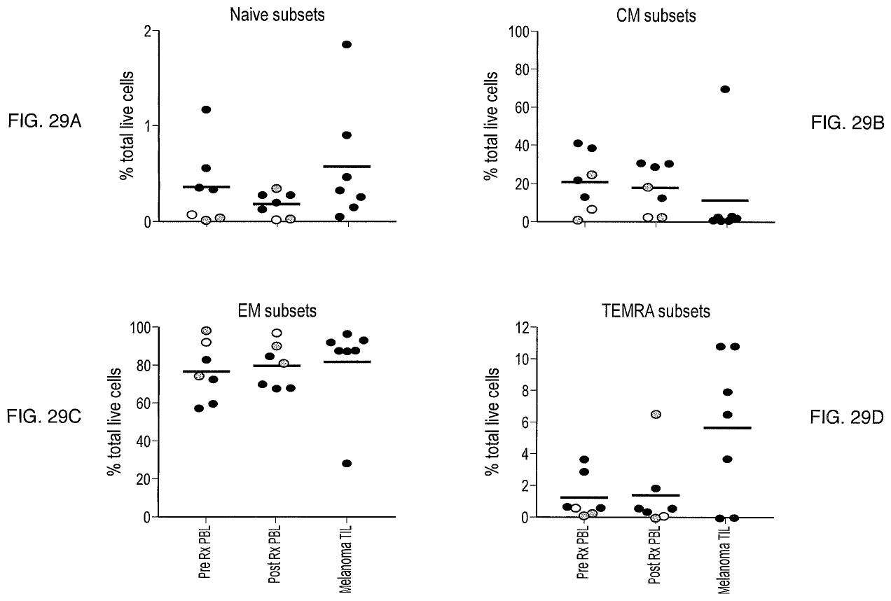

FIGS. 29A-29D represent a comparison between CD4 memory subset of PreRx PBLs and PostRx PBLs, using melanoma TIL as a comparator. FIG. 29A shows data for naïve (CCR7+/CD45RA+); FIG. 29B shows data for central memory t-cells (CM) (CCR7+/CD45RA−); FIG. 29C shows data for effector memory T-cells (EM) (CCR7−/CD45RA−); and FIG. 29D shows data for terminally differentiated effector memory cells (TEMRA) (CCR7−/CD45RA+). FIG. 29 demonstrates that the CD4 memory subsets for PreRx PBLs and PostRx PBLs are comparable to that seen for melanoma TIL.

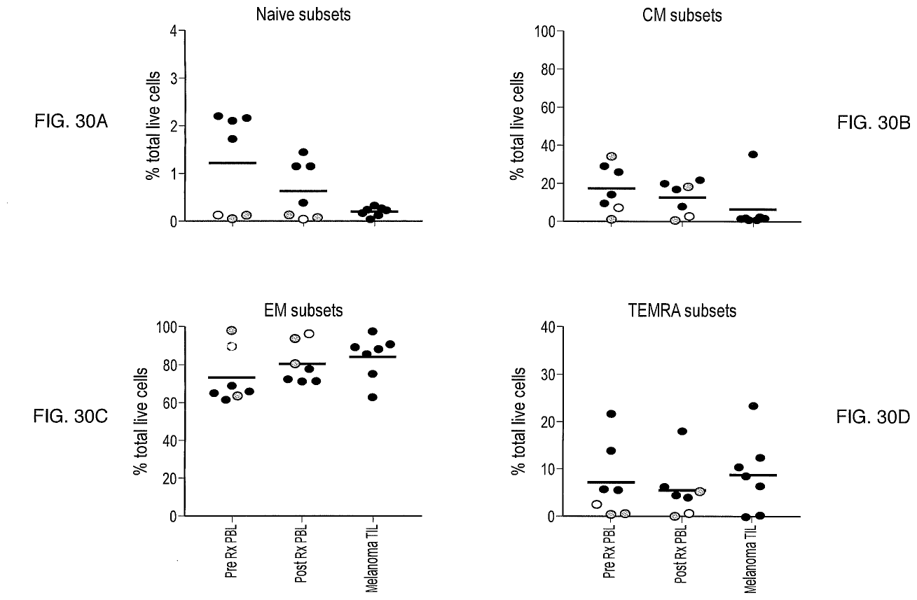

FIGS. 30A-30D represent a comparison between CD8 memory subset of PreRx PBLs and PostRx PBLs, using melanoma TIL as a comparator. FIG. 30A shows data for naïve (CCR7+/CD45RA+); FIG. 30B shows data for central memory t-cells (CM) (CCR7+/CD45RA−); FIG. 30C shows data for effector memory T-cells (EM) (CCR7−/CD45RA−); and FIG. 30D shows data for terminally differentiated effector memory cells (TEMRA) (CCR7−/CD45RA+). FIG. 30 demonstrates that the CD8 memory subsets for PreRx PBLs and PostRx PBLs are comparable to that seen for melanoma TIL.

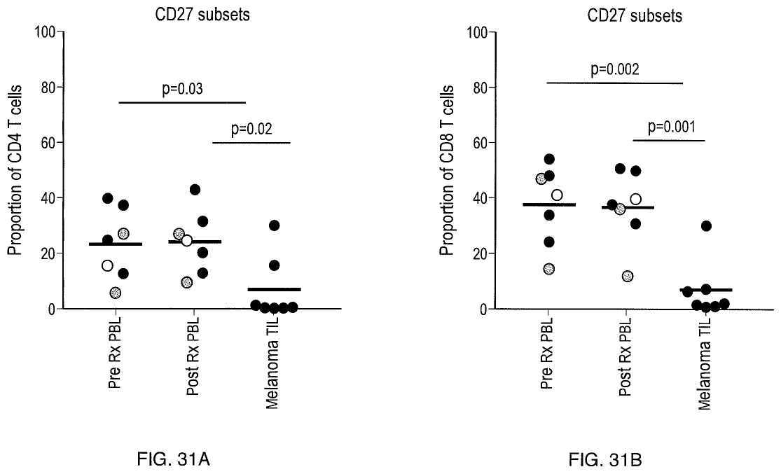

FIGS. 31A and 31B represent a comparison of CD27 subsets of CD4 cells (FIG. 31A) and CD8 cells (FIG. 31B), using melanoma TIL as a comparator. CD4CD27 cell subsets were significantly higher in both the PreRx PBL (p=0.03) and PostRx PBL (p=0.02) as compared to melanoma TIL. CD8CD27 cell subsets were significantly higher in both the PreRx PBL (p=0.002) and PostRx PBL (p=0.001) as compared to melanoma TIL.

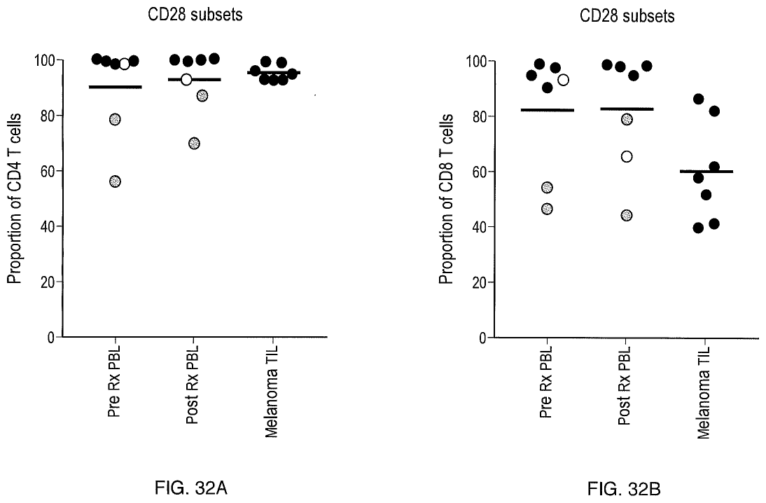

FIGS. 32A and 32B represent a comparison of CD28 subsets of CD4 cells (FIG. 30A) and CD8 cells (FIG. 30B), using melanoma TIL as a comparator. CD4CD28 cell subsets and CD8CD28 cell subsets were shown to be comparable in both the PreRx PBL and PostRx PBL as compared to melanoma TIL.

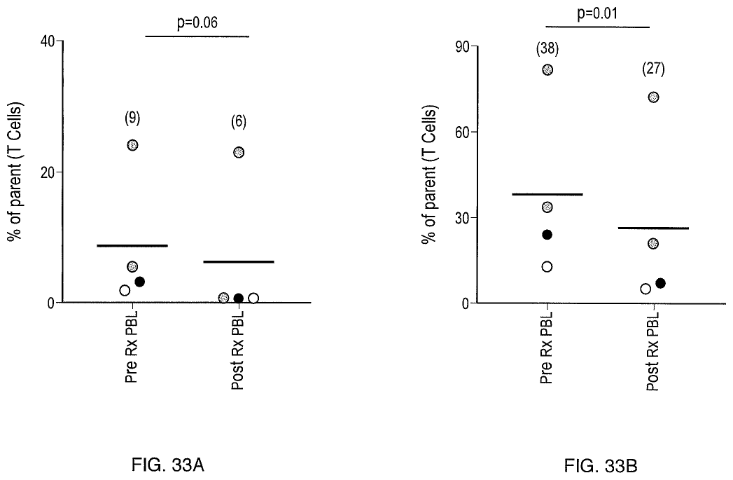

FIGS. 33A and 33B represent a comparison of LAG3+ subsets within the CD4+ (FIG. 33A) and CD8+ (FIG. 33B) populations for PreRx PBLs and PostRx PBLs. The data show a significant mean decrease in LAG3+ subsets in both CD4+ (p=0.06) and CD8+ (p=0.01) populations in the PostRx PBLs.

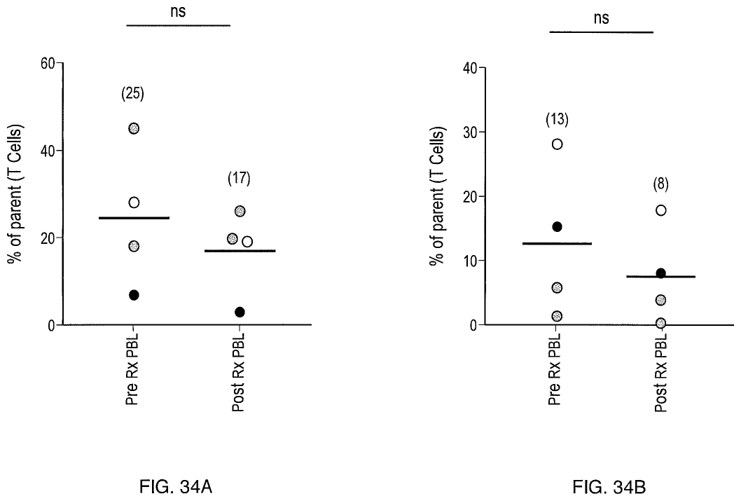

FIGS. 34A and 34B represent a comparison of PD1+ subsets within the CD4+ (FIG. 34A) and CD8+ (FIG. 34B) populations for PreRx PBLs and PostRx PBLs. The data show a mean decrease in PD1+ subsets in both CD4+ and CD8+ populations in the PostRx PBLs, but the decrease was not significant.

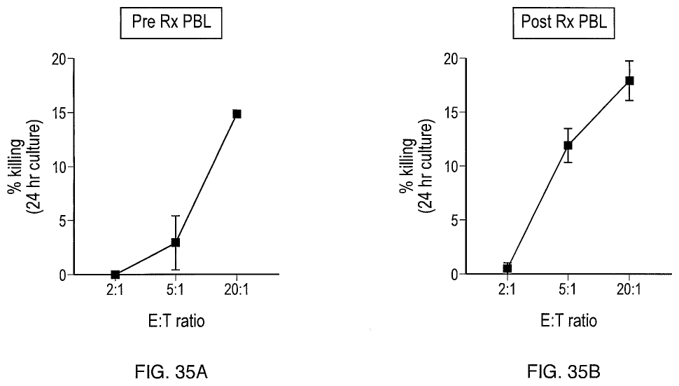

FIGS. 35A and 35B show results of cytolytic activity of PreRx PBLs (FIG. 35A) and PostRx PBLs (FIG. 35B), measured using a Bioluminescent Redirected Lysis Assay (BRLA). The assay was performed using the Cell1Trace™ Violet Cell Proliferation Kit (Invitrogen) as follows: The Effector Cells, which are the PBLs, were labeled with carboxyfluorescein succinimidyl ester (CFSE). The Target Cells (autologous CD19+ tumor cells) were incubated with mitocyin C, and then labeled with CellTrace™ Violet (CTV) in accordance with the CellTrace™ Violet Cell Proliferation Kit instructions. The Effector and Target cells were incubated for 24 hours at ratios of 2:1, 5:1 and 20:1 (E:T cells). The countbright beads were added, the cells were stained with Annexin V-PI, and then analyzed for CTV+/Annexin-V PI+ cells (which provides the number of dead cells). The PostRx PBLs appear to be more potent because less cells are required to kill 50% of the target tumor cells (i.e., the LU50 is lower for PostRx PBLs than for PreRx PBLs).

The experiments performed in this Example demonstrated the following results: PBLs expanded from fresh CLL PBMCs showed lower fold expansion and significantly less IFN-□ production as compared to PBLs expanded from cryopreserved PBMCs (PreRx PBLs and PostRx PBLs); PostRx PBLs showed consistently higher fold expansion and significant increase in IFN-□ production as compared with PreRx PBLs; and both PreRx PBLs and PostRx PBLs showed lytic activity against autologous (CD19+) tumor cells, although PostRx PBLs had a lower LU50 than PreRx PBLs.

### Example 6

### An Exemplary Embodiment of Selecting and Expanding PBLs from PBMCs in CLL Patients

PBMCs are collected from patients (optionally pretreated with an ITK inhibitor such as ibrutinib) and either frozen prior to use, or used fresh. Enough volume of peripheral blood is collected to yield at least about 400,000,000 (400×106) PBMCs for starting material in the method of the present invention. On Day 0 of the method, IL-2 at 6×106 IU/mL is either prepared fresh or thawed, and stored at 4° C. or on ice until ready to use. 200 mL of CM2 medium is prepared by combining 100 mL of CM1 medium (containing GlutaMAX®), then diluting it with 100 mL (1:1) with AIM-V to make CM2. The CM2 is protected from light, and sealed tightly when not in use.

All of the following steps are performed under sterile cell culture conditions. An aliquot of 50 mL of CM2 is warmed in a 50 mL conical tube in a 37° C. water bath for use in thawing and/or washing a frozen PBMC sample. If a frozen PBMC sample is used, the sample is removed from freezer storage and kept on dry ice until ready to thaw. When ready to thaw the PBMC cryovial, 5 mL of CM2 medium is placed in a sterile 50 mL conical tube. The PBMC sample cryovial is placed in a 37° C. water bath until only a few ice crystals remain. Warmed CM2 medium is added, dropwise, to the sample vial in a 1:1 volume ratio of sample:medium (about 1 mL). The entire contents is removed from the cryovial and transferred to the remaining CM2 medium in the 50 mL conical tube. An additional 1-2 mL of CM2 medium is used to rinse the cryovial and the entire contents of the cryovial is removed and transferred to the 50 mL conical tube. The volume in the conical tube is then adjusted with additional CM2 medium to 15 mL, and swirled gently to rinse the cells. The conical tube is then centrifuged at 400 g for 5 minutes at room temperature in order to collect the cell pellet.

The supernatant is removed from the pellet, the conical tube is capped, and then the cell pellet is disrupted by, for example, scraping the tube along a rough surface. About 1 mL of CM2 medium is added to the cell pellet, and the pellet and medium are aspirated up and down 5-10 times with a pipette to break up the cell pellet. An additional 3-5 mL of CM2 medium is added to the tube and mixed via pipette to suspend the cells. At this point, the volume of the cell suspension is recorded. Remove 100 mL of the cell suspension from the tube for cell counting with an automatic cell counter, for example, a Nexcelom Cellometer K2. The number of live cells in the sample is determined and record.

Reserve a minimum of 5×106 cells for phenotyping and other characterization experiments. Spin the reserved cells at 400 g for 5 minutes at room temperature to collect the cell pellet. Resuspend the cell pellet in freezing medium (sterile, heat-inactivated FBS containing 20% DMSO). Freeze one or two aliquots of the reserved cells in freezing medium, each aliquot consisting of 2-5×106 cells in 1 mL of freezing medium in a cryovial, and slow-freeze the aliquots in a cell freezer (Mr. Frosty) in a −80° C. freezer. Transfer to liquid nitrogen storage after a minimum of 24 hours at −80° C.

For the following steps, use pre-cooled solutions, work quickly, and keep the cells cold. The next step is to purify the T-cell fraction of the PBMC sample. This is completed using a Pan T-cell Isolation Kit (Miltenyi, catalog #130-096-535). Prepare the cells for purification by washing the cells with a sterile-filtered wash buffer containing PBS, 0.5% BSA, and 2 mM EDTA at pH 7.2. The PBMC sample is centrifuged at 400 g for 5 minutes to collect the cell pellet. The supernatant is aspirated off and the cell pellet is resuspended in 40 uL of wash buffer for every 107 cells. Add 10 uL of Pan T Cell Biotin-Antibody Cocktail for every 107 cells. Mix well and incubate for 5 minutes in refrigerator or on ice. Add 30 uL of wash buffer for every 107 cells. Add 20 uL of Pan T-cell MicroBead Cocktail for every 107 cells. Mix well and incubate for 10 minutes in refrigerator or on ice. Prepare an LS column and magnetically separate cells from the microbeads. The LS column is placed in the QuadroMACS magnetic field. The LS column is washed with 3 mL of cold wash buffer, and the wash is collected and discarded. The cell suspension is applied to the column and the flow-through (unlabeled cells) is collected. This flow-through is the enriched T-cell fraction (PBLs). Wash the column with 3 mL of wash buffer and collect the flow-through in the same tube as the initial flow-through. Cap the tube and place on ice. This is the T-cell fraction, or PBLs. Remove the LS column from the magnetic field, wash the column with 5 mL of wash buffer, and collect the non-T-cell fraction (magnetically labeled cells) into another tube. Centrifuge both fractions at 400 g for 5 minutes to collect the cell pellets. Supernatants are aspirated from both samples, disrupt the pellet, and resuspend the cells in 1 mL of CM2 medium supplemented with 3000 IU/mL IL-2 to each pellet, and pipette up and down 5-10 times to break up the pellets. Add 1-2 mL of CM2 to each sample, and mix each sample well, and store in tissue culture incubator for next steps. Remove about a 50 uL aliquot from each sample, count cells, and record count and viability.

The T-cells (PBLs) are then cultured with Dunabeads™ Human T-Expander CD3/CD28. A stock vial of Dynabeads is vortexed for 30 seconds at medium spead. A required aliquot of beads is removed from the stock vial into a sterile 1.5 mL microtube. The beads are washed with bead wash solution by adding 1 mL of bead wash to the 1.5 mL microtube containing the beads. Mix gently. Place the tube onto the DynaMag™-2 magnet and let sit for 30 minutes while beads draw toward the magnet. Aspirate the wash solution off the beads and remove tube from the magnet. 1mL of CM2 medium supplemented with 3000 IU/mL IL-2 is added to the beads. The entire contents of the microtube is transferred to a 15 or 50 mL conical tube. Bring the beads to a final concentration of about 500,000/mL using CM2 medium with IL-2.

The T-cells (PBLs) and beads are cultured together as follows. On day 0: In a G-Rex 24 well plate, in a total of 7 mL per well, add 500,000 T-cells, 500,000 CD3/CD28 Dynabeads, and CM2 supplemented with IL-2. The G-Rex plate is placed into a humidified 37° C., 5% CO2 incubator until the next step in the process (on Day 4). Remaining cells are frozen in CS10 cryopreservation medium using a Mr. Frosty cell freezer. The non-T-cell fraction of cells are frozen in CS10 cryopreservation medium using a Mr. Frosty cell freezer. On day 4, medium is exchanged. Half of the medium (about 3.5 mL) is removed from each well of the G-rex plate. A sufficient volume (about 3.5 mL) of CM4 medium supplemented with 3000 IU/mL IL-2 warmed to 37° C. is added to replace the medium removed from each sample well. The G-rex plate is returned to the incubator.

On day 7, cells are prepared for expansion by REP. The G-rex plate is removed from the incubator and half of medium is removed from each well and discarded. The cells are resuspended in the remaining medium and transferred to a 15 mL conical tube. The wells are washed with 1 mL each of CM4 supplemented with 3000 IU/mL IL-2 warmed to 37° C. and the wash medium is transferred to the same 15 mL tube with the cells. A representative sample of cells is removed and counted using an automated cell counter. If there are less than 1×106 live cells, the Dynabead expansion process at Day 0 is repeated. The remainder of the cells are frozen for back-up expansion or for phenotyping and other characterization studies. If there are 1×106 live cells or more, the REP expansion is set up in replicate according to the protocol from Day 0. Alternatively, with enough cells, the expansion may be set up in a G-rex 10M culture flask using 10-15×106 PBLs per flask and a 1:1 ratio of Dynabeads:PBLs in a final volume of 100 mL/well of CM4 medium supplemented with 3000 IU/mL IL-2. The plate and/or flask is returned to the incubator. Excess PBLs may be aliquotted and slow-frozen in a Mr. Frosty cell freezer in a −80° C. freezer, and the transferred to liquid nitrogen storage after a minimum of 24 hours at −80° C. These PBLs may be used as back-up samples for expansion or for phenotyping or other characterization studies.

On Day 11, the medium is exchanged. Half of the medium is removed from either each well of the G-rex plate or the flask and replaced with the same amount of fresh CM4 medium supplemented with 3000 IU/mL IL-2 at 37° C.

On Day 14, the PBLs are harvested. If the G-rex plate is used, about half of the medium is removed from each well of the plate and discarded. The PBLs and beads are suspended in the remaining medium and transferred to a sterile 15 mL conical tube (Tube 1). The wells are washed with 1-2 mL of fresh AIM-V medium warmed to 37° C., and the wash is transferred to Tube 1. Tube 1 is capped and placed in the DynaMag™-15 Magnet for 1 minute to allow the beads to be drawn to the magnet. The cell suspension is transferred into a new 15 mL tube (Tube 2), and the beads are washed with 2 mL of fresh AIM-V at 37° C. Tube 1 is placed back in the magnet for an additional 1 minute, and the wash medium is then transferred to Tube 2. The wells may be combined if desired, after the final washing step. Remove a representative sample of cells and count, record count and viability. Tubes may be placed in the incubator while counting. Additional AIM-V medium may be added to the Tube 2 if cells appear very dense. If a flask is used, the volume in the flask should be reduced to about 10 mL. The contents of the flask is mixed and transferred to a 15 mL conical tube (Tube A). The flask is washed with 2 mL of the AIM-V medium as described above and the wash medium is also transferred to Tube A. Tube A is capped and placed in the DynaMag™-15 Magnet for 1 minute to allow the beads to be drawn to the magnet. The cell suspension is transferred into a new 15 mL tube (Tube B), and the beads are washed with 2 mL of fresh AIM-V at 37° C. Tube A is placed back in the magnet for an additional 1 minute, and the wash medium is then transferred to Tube B. The wells may be combined if desired, after the final washing step. Remove a representative sample of cells and count, record count and viability. Tubes may be placed in the incubator while counting. Additional AIM-V medium may be added to the Tube B if cells appear very dense. Cells may be used fresh or frozen in CS10 preservation medium at desired concentrations.

### Example 7

### An Exemplary Embodiment of Selecting and Expanding PBLs from PBMCs

For the expansion of PBLs from PBMCs obtained from CLL patients or patients with other diseases described herein, including CLL patients having previously received ibrutinb or an ITK inhibitor or with ibrutinib-relapsed or refractory CLL, the following procedure may be used. All steps require the use of sterile technique in a biological safety cabinet (BSC) or similar enclosure.

On day 0, prepare 6×106 IU/mL IL-2. If aliquots are available, thaw a fresh aliquot and leave it at 4° C. in refrigerator or on ice until ready to use. Prepare small volume (e.g. 200 mL) of CM2 media. First prepare 100 mL of CM1 media, substituting GlutaMAX for glutamine in the procedure, then dilute it 1:1 with AIM V to make CM2. While performing this experiment, keep the CM2 warm in a 37° C. water bath, protected from light, with cap closed tightly. When it is being used in the hood, do not leave the cap off or loose. In a 37° C. water bath, warm an aliquot of CM2 (without IL-2) in a 50 mL conical tube to use for thawing and washing CLL sample. Remove the cryovial containing the CLL PBMC sample from LN2 freezer storage, keeping sample on dry ice until ready to thaw, or use fresh CLL PBMCs. Just prior to beginning thaw, place warmed media aliquot into BSC. Add 5 mL of media into a fresh, sterile, labeled 50 mL conical tube. Thaw sample by placing the cryovial in 37° C. water bath until only a few ice crystals remain in the cryovial. Transfer cryovial containing thawed samples to the BSC. Using a sterile transfer pipet, add an equal volume of warmed media dropwise to CLL sample cryovial (˜1 mL). Using the same transfer pipet, remove the sample from the cryovial and add it dropwise to the prepared 50 mL conical tube. Rinse tube with an additional 1-2 mL of CM2 and transfer that to the 50 mL conical tube. Bring the volume to 15 mL, swirl sample gently to rinse cells well, then spin sample in high speed centrifuge to collect cell pellet, at 400× g for 5 min at room temperature. Return sample to BSC and aspirate off supernatant from pellet, being careful not to disturb cell pellet. Cap tube and scrape it along a rough surface (such as a tube rack) to help break up cell pellet. Using a 1 mL pipettor and tip, add 1 mL of fresh CM2 to the cell pellet and gently aspirate the cells up-and-down 5-10 times to break up cell pellet. Add an additional 3-5 mL of CM2 to cell suspension; pipet up-and-down several times to mix sample well. Record volume of cell suspension. Remove a representative volume of cell suspension from the tube for counting (e.g., 100 μL). Using an automatic cell counter, such as Nexcelom Cellometer K2, count cells using appropriate procedure. Determine the total number of live cells in the sample. Reserve a minimum of 5×106 cells for phenotyping and other experiments. Spin reserved sample at 400× g for 5 min at room temperature to collect pellet. Freeze one or two aliquots of the reserved sample in freezing medium (sterile, heat-inactivated fetal bovine serum containing 20% DMSO). Slow-freeze cell sample in a Mr. Frosty cell freezer placed in a −80° C. freezer. Transfer to LN2 storage after a minimum of 24 hours at −80° C.

For the proceeding separation steps, work quickly, keeping the cells cold; use pre-cooled solutions. Purify the T-cell fraction of the CLL sample using Pan T-cell Isolation Kit (Miltenyi: Catalogue #130-096-535). Prepare wash buffer prior to beginning procedure. Wash buffer: phosphate-buffered saline, pH 7.2, containing 0.5% bovine serum albumin and 2 mM EDTA; read pH and adjust if necessary; sterile filter; store at 4° C. Spin sample at 400× g for 5 min to collect cell pellet. Aspirate off media supernatant and resuspend the cell pellet in 40 of wash buffer for every 107 total cells. Add 10 μL of Pan T Cell Biotin-Antibody Cocktail for every 107 total cells. Mix sample well and incubate in the refrigerator or on ice for 5 min. Add 30 μL of wash buffer to sample for every 107 total cells. Add 20 μL of Pan T Cell MicroBead Cocktail for every 107 total cells. Mix well and incubate for 10 min in the refrigerator or on ice. Proceed to magnetic cell separation. Use LS column and QuadroMACS magnet for this procedure. Each LS column has a maximum capacity of 2×109 total cells. Prepare LS column for use; always wait until column reservoir is empty before proceeding to the next step. Place LS column in magnetic field of QuadroMACS magnet. Rinse LS column with 3 mL of prepared, cold wash buffer. Collect wash into a 15 mL conical tube. Discard wash. Place fresh tube labeled “T cell fraction” under LS column. Apply cell suspension onto the column. Collect flow-through containing the unlabeled cells—this is the enriched T-cell fraction. Wash column with 3 mL of wash buffer. Collect the unlabeled cells that wash through the column into the same 15 mL conical “T cell fraction” tube. Cap tube and place on ice. Remove LS column from QuadroMACS magnet and place it onto a fresh 15-ml conical tube labeled “non-T cell fraction.” Pipet 5 mL of wash buffer onto the column and immediately flush out the magnetically labeled non-T cells by firmly pushing plunger (provided with the LS column) into the column. Place both “T-cell fraction” and “Non-T cell fraction” tubes into centrifuge and spin at 400× g for 5 min to collect cell pellet. Aspirate off supernatant from both samples, cap tubes, and resuspend each pellet by scraping tube against a rough surface. Using a 1 mL pipettor and tips, add 1 mL of CM2 medium supplemented with 3000 IU/ml IL-2 to each pellet. Resuspend each pellet by gently pipetting up-and-down 5-10 times to break pellets up further. Add 1-2 mL of fresh medium to each sample, mixing each sample well. Remove a small representative aliquot from each sample (e.g., 50 μL). Place cell samples into tissue culture incubator, loosening cap. Count cells; record counts and viability. Prepare a small amount of Dynabeads™ Human T-Expander CD3/CD28 for use. Vortex stock vial of CD3/CD28 Dynabeads for 30 sec at medium speed on a vortex mixer. Remove required aliquot of beads from stock vial to a sterile 1.5 mL microtube Wash beads with bead wash solution by adding 1 mL of wash to the 1.5 mL microtube containing the beads. Tap tube to mix sample. Place tube containing beads onto DynaMag™-2 magnet and let tube sit for 30 sec while beads are drawn to magnet. Aspirate off wash solution from the side of the microtube opposite the DynaMag magnet. Remove microtube from magnet and place in a tube rack. Using a 1 mL pipettor and tip, add 1 ml of CM2 supplemented with IL-2 to the beads. Transfer the bead solution to a fresh 15 mL (or 50 mL) conical tube labeled “beads, 500,000/mL.” Bring beads to a final volume that will give a concentration of 500,000 beads/ml (e.g., 10×106 beads brought to a final volume of 20 mL). Set up cell culture as follows, a minimum of 1 well per sample. More wells can be set up if there are enough cells. In a G-Rex 24-well plate, in a total of 7 mL per well, add 500,000 T cells, 500,000 CD3/CD28 Dynabeads (1 mL of 500,000 beads/mL suspension), and CM2 supplemented with 3000 IU/ml IL-2. Place G-Rex 24-well plate into humidified 37° C., 5% CO2 incubator. If there are enough cells, retain a small portion of the T-cell fraction for repeat of REP, or for other experiments, freezing the sample in CS10 cryopreservation medium using a Mr. Frosty cell freezer. Count the non-T cell fraction of cells and freeze them in CS10 cryopreservation medium using a Mr. Frosty cell freezer.

On day 4, media exchange is performed as follows. Prepare a sufficient volume of CM4 (supplemented with 3000 IU/mL IL-2) to replace half the media from the sample wells and warm it to 37° C. in water bath. Remove G-Rex 24-well plate from incubator to BSC. Remove half the volume of media from each well (3.5 mL). Add equivalent volume (3.5 mL) of fresh media to each well. Return G-Rex 24-well plate to humidified, 37° C., 5% CO2 incubator.

On day 7, expansion using REP is performed as follows. Prepare a small volume of CM4 (supplemented with 3000 IU/mL IL-2) to perform washes of culture wells. Keep warm in 37° C. water bath. Remove G-Rex 24-well plate from the incubator to BSC. Remove half the volume of media from each well and discard. Resuspend remaining cells and transfer to a labeled, sterile 15 mL conical tube. Wash well with 1 mL of prepared CM4 and transfer wash solution to the same 15 mL conical tube. Retain the G-Rex 24-well plate in the tissue culture hood—unused wells of the same plate can be used for the expansion of the PBL sample. Remove a representative volume of cells and count using automated cell counter. Determine number of wells or culture vessels required to expand PBL. If ≤1×106 total live cells: Set up expansion in one well of a G-Rex 24-well plate using 500,000 PBL and a 1:1 ratio of Dynabeads™ Human T-Expander CD3/CD28 prepared as in Step 9.16 in 7 mL CM4 (supplemented with 3000 IU/mL IL-2). Freeze remainder of cells for back-up expansion or for phenotyping and other subsidiary procedures. If 1×106 or more total live cells: set up expansion in replicate wells in a G-Rex 24-well plate using 500,000 PBL per well and a 1:1 ratio of Dynabeads™ Human T-Expander CD3/CD28 prepared as in Step 9.16 in final volume of 7 ml/well CM4 (supplemented with 3000 IU/mL IL-2). Alternately, with excess sample, set up expansion in a G-Rex10M culture flask using 10-15×106 PBL per flask and a 1:1 ratio of Dynabeads™ Human T-Expander CD3/CD28 prepared as in Step 9.16 in a final volume of 100 ml/well CM4 (supplemented with 3000 IU/mL IL-2). Slow-freeze excess Day 7 PBL samples in labeled cryovials placed in a Mr. Frosty cell freezer at −80° C. Transfer to LN2 storage after a minimum 24 hours at −80° C. These samples can be used as back-up samples for expansion or for phenotyping and other subsidiary procedures. (Recommended minimum number of cells to retain on Day 7: 2×106 to 5×106.) Place culture plates or flasks into humidified, 37° C., 5% CO2 incubator.

On day 11, media exchange is performed as follows. Prepare a sufficient volume of CM4 (supplemented with 3000 IU/mL IL-2) to replace half of the volume in each culture well or vessel and keep it warm in a 37° C. water bath. Remove the culture vessels from the incubator to the BSC. Remove half the media from each well or flask and discard. Add equivalent volume to each culture well or flask. Return culture vessels to humidified, 37° C., 5% CO2 incubator.

On day 14, REP harvest is performed as follows. Warm a small volume of AIM V media in a 37° C. water bath to use for washes in the following steps. Transfer to BSC when ready to harvest samples. Remove the culture vessels from the incubator to the BSC. If culture is in G-Rex 24-well plate, remove about half of the volume from each well and discard. For larger cultures, proceed to the “REP is complete” step in the next paragraph. Mix sample with serological pipet and transfer cells to labeled, sterile 15 mL conical tube. Wash well with 1-2 mL of fresh, warmed media. Cap 15 mL conical tube and place in DynaMag™-15 Magnet. Allow sample to sit for 1 min in magnet to allow magnetic beads to be drawn to magnet. Using a 5 mL serological pipet, remove the cell suspension to a fresh, labeled 15 mL conical tube. Remove first 15-ml tube from magnet and wash the beads with a minimum of 2 ml of fresh AIM V. Place tube back on magnet and allow it to sit for 1 min. Using a 5 mL serological pipet, remove the wash media to the second labeled 15 mL conical tube. If more than one well per sample was prepared, all wells of the same condition can be combined after washing the beads. If culture is in G-Rex10M flask, reduce volume by aspiration to about 10 mL total. Mix sample using 10 mL serological pipet and transfer cells to labeled, sterile 15 mL conical tube. Wash flask with 2 mL of fresh, warmed media. Cap 15 mL conical tube and place in DynaMag™-15 Magnet. Allow sample to sit for 1 min in magnet to allow magnetic beads to be drawn to magnet. Using a 5 mL serological pipet, remove the cell suspension to a fresh, labeled 15 mL conical tube. Remove first 15 mL tube from magnet and wash the beads with a minimum of 2 mL of fresh AIM V. Place tube back on magnet and allow it to sit for 1 min. Using a 5 mL serological pipet, remove the wash media to the second labeled 15 mL conical tube. If more than one flask per sample was prepared, all can be combined after washing the beads. If cells appear to be extremely dense, extra pre-warmed AIM V media can be added to the culture. Remove a representative volume of cells and count using automated cell counter. Record cell number and viability. Place tubes containing cells into humidified, 37° C., 5% CO2 incubator, with cap loosened, while counting cells.

At this point, the REP is complete. Post-REP testing of PBL can be done on fresh or frozen samples. Freeze PBL samples in CS10 cryopreservation medium, or prepare as needed in alternative formulations for delivery to a patient. Lower concentrations of cells (e.g., 5×106 cells/vial) can be used for phenotyping by flow cytometry and co-culture assays, so it is recommended to reserve 6-10 vials at low concentration, with the remainder at a higher concentration (30-50×106 cells/vial). The foregoing procedure may be scaled, adjusted, or optimized, and adapted as needed for regulatory compliance (including to satisfy good manufacturing practices and International Conference on Harmonization guidance, as adapted by the U.S. Food and Drug Administration and other regulatory authorities), as will be apparent to the skilled artisan.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

We claim:

1. A method for expanding peripheral blood lymphocytes (PBLs) from peripheral blood comprising:
a. Obtaining a sample of PBMCs from peripheral blood of a patient, wherein said sample is optionally cryopreserved and the patient is optionally pretreated with an ITK inhibitor;
b. Isolating PBLs from said sample by selecting and removing CD19+ B cells and optionally pretreating said PBLs with an ITK inhibitor at a concentration of between 0.1 nM and 200 nM;
c. Co-culturing said PBLs with said CD19+ B cells for a period of between 2 to 5 days;
d. Adding from about 2.5×10⁵ to about 5×10⁵ cells to a gas-permeable container in a first cell culture medium and stimulating said PBLs with 3000 IU/ml IL-2 and anti-CD3/anti-CD28 antibodies immobilized on beads, and and optionally an ITK inhibitor at a concentration of between 0.1 nM and 200 nM, for a period of between 3 to 6 days;
e. Exchanging the first cell culture medium with a second cell culture medium and additional IL-2 at a concentration of about 3000 IU/ml and optionally an ITK inhibitor at a concentration of between 0.1 nM and 200 nM;
f. Culturing the PBLs from step (e) for an additional period of between 3 to 6 days with IL-2 and anti-CD3/anti-CD28 antibodies immobilized on beads and optionally an ITK inhibitor at a concentration of between 0.1 nM and 200 nM;
g. Exchanging the second cell culture medium with a third cell culture medium and adding additional IL-2 at a concentration of 3000 IU/mL, and optionally an ITK inhibitor at a concentration of between 0.1 nM and 200 nM, and culturing the cells for an additional period of between 2 to 5 days;
h. Isolating the antibody-bound PBLs from the culture in step (g);
i. Removing the antibodies from the PBLs isolated in step (h); and
j. Harvesting the PBLs,
wherein the ITK inhibitor is optionally an ITK inhibitor that covalently binds to ITK, and further wherein the PBLs may be optionally transduced with an expression vector comprising a nucleic acid encoding a chimeric antigen receptor (CAR) comprising a single chain variable fragment antibody fused with at least one endodomain of a T-cell signaling molecule.

2. A method for treating a hematological malignancy, the method comprising:
a. Obtaining a sample of PBMCs from peripheral blood of a patient suffering from a hematological malignancy;
b. Isolating PBLs from said sample by selecting and removing CD19+ B cells;
c. Optionally co-culturing said PBLs with said CD19+ B cells;
d. Stimulating said PBLs in a first cell culture medium with IL-2 and anti-CD3/anti-CD28 antibodies for a period of from about 2 to about 6 days in a gas permeable container;
e. Culturing the PBLs from step (d) for a period of from about 2 to about 6 days with IL-2 and anti-CD3/anti-CD28 antibodies;
f. Isolating the antibody-bound PBLs from the culture in step (e);
g. Removing the antibodies from the PBLs isolated in step (f); and
h. Harvesting the PBLs;
i. Optionally transducing the PBLs with an expression vector comprising a nucleic acid encoding a chimeric antigen receptor (CAR) comprising a single chain variable fragment antibody fused with at least one endodomain of a T-cell signaling molecule or optionally transducing the PBLs with an expression vector comprising a genetically-engineered T-cell receptor; and
j. Administering the PBLs to the patient in a therapeutically effective amount to treat said hematological malignancy.

3. The method of claim 2, wherein the patient is pre-treated with an ITK inhibitor prior to obtaining a PBMC sample.

4. The method of claim 3, wherein the ITK inhibitor is selected from the group consisting of aminothiazole-based ITK inhibitors, benzimidazole-based ITK inhibitors, aminopyrimidine-based ITK inhibitors, 3-aminopyride-2-ones-based ITK inhibitors, indolylndazole-based ITK inhibitors, pyrazolyl-indole-based inhibitors, thienopyrazole inhibitors, and ITK inhibitors targeting cysteine-442 in the ATP pocket.

5. The method of claim3, wherein the ITK inhibitor is selected from the group consisting of ibrutinib, dasatinib, bosutinib, nilotinib, erlotinib BMS509744, CTA056, GSK2250665A, PF06465469 ((R)-3-(1-(1-acryloylpiperidin-3 -yl)-4-amino-1H-pyrazolo[3,4-d]pyrimidin-3-yl)-N-(3-methyl-4-(1-methylethyl))benzamide), and combinations thereof.

6. The method of claim 5, wherein the ITK inhibitor is ibrutinib.

7. The method of claim 2, wherein the patient is pre-treated with at least one round of an ibrutinib regimen.

8. The method of claim 2, wherein the hematological malignancy is selected from the group consisting of acute myeloid leukemia (AML), mantle cell lymphoma (MCL), follicular lymphoma (FL), diffuse large B cell lymphoma (DLBCL), activated B cell (ABC) DLBCL, germinal center B cell (GCB) DLBCL, chronic lymphocytic leukemia (CLL), CLL with Richter's transformation (or Richter's syndrome), small lymphocytic leukemia (SLL), non-Hodgkin's lymphoma (NHL), Hodgkin's lymphoma, relapsed and/or refractory Hodgkin's lymphoma, B cell acute lymphoblastic leukemia (B-ALL), mature B-ALL, Burkitt's lymphoma, Waldenström's macroglobulinemia (WM), multiple myeloma, myelodysplatic syndromes, myelofibrosis, chronic myelocytic leukemia, follicle center lymphoma, indolent NHL, human immunodeficiency virus (HIV) associated B cell lymphoma, and Epstein-Barr virus (EBV) associated B cell lymphoma.

9. The method of claim 8, wherein the hematological malignancy is chronic lymphocytic leukemia (CLL).

10. The method of claim 2, wherein the first cell culture medium is selected from the group consisting of CM-2, CM-4, and AIM-V.

11. The method of claim 2, wherein after step (d), additional IL-2 is added and the cell culture medium is exchanged with a second cell culture medium.

12. The method of claim 11 wherein after step (e), additional IL-2 is added and the second cell culture medium is exchanged with a third cell culture medium.

13. The method of claim 11, wherein the second cell culture medium is selected from the group consisting of CM-2, CM-4, and AIM-V.

14. The method of claim 11, wherein the third cell culture medium is selected from the group consisting of CM-2, CM-4, and AIM-V.

15. The method of claim 11, wherein the first cell culture medium and the second cell culture medium are different.

16. The method of claim 11, wherein the first cell culture medium and the second cell culture medium are the same.

17. The method of claim 2, wherein the ratio of B-cells to PBLs in step (c) is from about 0.1:1 to about 10:1 (B-cells:PBLs).

18. The method of claim 2, wherein the ratio of B-cells to PBLs in step (c) is selected from the group consisting of 0.1:1, 1:1, and 10:1 (B-cells:PBLs).

19. The method of claim 2, wherein there are at least from about 1×10⁵ to about 10×10⁵ PBLs in the gas permeable container at the beginning of step (d).

20. The method of claim 2, wherein there are at least from about 2.5×10⁵ to 10×10⁵ PBLs in the gas permeable container at the beginning of step (d).

21. The method of claim 2, wherein there are at least 5×10⁵ PBLs in the gas permeable container at the beginning of step (d).

22. The method of claim 2, wherein the IL-2 is present in a concentration of between 1000 IU/ml and 6000 IL/ml in steps (d) and (e).

23. The method of claim 22, wherein the IL-2 is present in a concentration of about 3000 IU/ml.

24. The method of claim 2, wherein the anti-CD3/anti-CD28 antibodies are coated onto beads and the PBLs:bead ratio is about 1:1 in each of steps (d) and (e).

25. The method of claim 2, wherein the PBLs are administered in an amount of from about 0.1×10⁹ to about 15×10⁹ PBLs.

26. The method of claim 8, wherein the hematological malignancy is CLL with Richter's transformation (or Richter's syndrome).
